SEQUENCE LISTING (398 sequences)

SEQ ID NO: 1 - protein, 113 aa, Ginglymostoma cirratum
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVN

SEQ ID NO: 2 - protein, 110 aa, Ginglymostoma cirratum
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVN

SEQ ID NO: 3 - protein, 118 aa, Ginglymostoma cirratum
ARVDQTPRSVTKETGESLTINCVLRSASYALGSTFWYRKKSGSTNEESISKGGRYVETVNSGSKSFSLRINDLTVEDGGTYRCGVIRDELVWGIAGYNNRPCAPPYAACGDGTAVTVN

SEQ ID NO: 4 - protein, 108 aa, Ginglymostoma cirratum
ARVDQTPQTITKETGESLTINCGLRDSNCALSSTYWYRKKSGSTNEESISKGGRYVETVNSGPLTFSLRINDLTVEDSGTYRCKVSCLAIPIGPHYDVYGGGTVVTVN

SEQ ID NO: 5 - protein, 111 aa, Ginglymostoma cirratum
ARVDQTPQTITKETGESLTINCVVRDSNCALGSTYWYRKKSGSTNEESITKGGRSVETVNSGSKSFSLRINDLTVEDSGTYRCKVLSIGWHDPMGLRGCDVYGGGTVVTVN

SEQ ID NO: 6 - protein, 114 aa, Ginglymostoma cirratum
ARVDQTPRSVTKETGESLTINCVLRDASYALGSTCWYRKKSGSTNEESISKGGRYVETVNSGSKSFSLRINDLTVEDGGTYRCGVGRGLGVGCDYPLCSYSYAACGDGTAVTVN

SEQ ID NO: 7 - protein, 114 aa, Ginglymostoma cirratum
ARVDQTPQTITKETGESLTINCVLRDASYALGSTCWYRKKSGSTNWESIGKGGRYVETVNSGSKSFSLRINDLTVEDGGTYRCGVGRGAGVGCDYPLCSYSYAACGGGTVVTVN

SEQ ID NO: 8 - protein, 108 aa, Ginglymostoma cirratum
ARVDQTPKTITKETGESLTINCVLRDSNCAASSTYWYRKKSGSNNEESISKGGRYVETVNSGSKSFSLRINDLTVEDSGTYRCNVEGYSCGLYSVEDYYGGGTVVTVN

SEQ ID NO: 9 - protein, 113 aa, Ginglymostoma cirratum
ARVDQTPQTITKETGESLTIDCVLRDSNCALSSTHWYRKKSGSTNEERILQGRRYVETVNSGSKSFSLRINDLRVEDSGTYRCKVYWGNSWQDKFCPGLGSYEYGGGTVVTVN

SEQ ID NO: 10 - protein, 109 aa, Ginglymostoma cirratum
ARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVN

SEQ ID NO: 11 - protein, 109 aa, Ginglymostoma cirratum
ARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVN

SEQ ID NO: 12 - protein, 107 aa, Ginglymostoma cirratum
ARVDQTPRSVTKETGESLTINCVLRDTNCPLYAAYWYRKSPGSTNEETIWATGRYVQTVNSGLKTFSLRINDLTSEDSGTYRCNGQTWCPATKNNDISGGGTVVTVN

SEQ ID NO: 13 - protein, 113 aa, Ginglymostoma cirratum
ARVDQTPRSVTKETGESLTINCVLRDASYALGSTCWYRKKSGSTNEERISKGGRYVETVNSGSKSFSLRINDLTVEDGGTYRCGVWPGYNWCGSCDYRGDYNFCGDGTAVTVN

SEQ ID NO: 14 - protein, 119 aa, Ginglymostoma cirratum
ARVDQTPQTITKETGESLTINCVLRDSNCALSRTYWYRKKSGSTDEESISKGGRYVETVNSGSKSFSLRINDLTVEDSGTYRCNVYSWYGGDHNSWDWRSDPAMVCGDVYGGGTVVTVN

SEQ ID NO: 15 - protein, 114 aa, Ginglymostoma cirratum
ARVDQTPRSVTKETGESLTINCVLRDANYALGSTCWYRKKSGSTNGESISKGGRYVETVNSGSKSFSLRINDLTVEDGGTYRCGVGVVFGRCDFCALERKNYAVCGGGTVVTVN

SEQ ID NO: 16 - protein, 115 aa, Ginglymostoma cirratum
ARVDQTPRSVTKETGESLTINCVLRDATYALGNTCWYRKKSGSSTEESISKGGRYVETVNSGSKSFSLRINDLTVEDGGTYRCGVWPGYNWCGSCDYVHDALHVFCGGGTVVTVN

SEQ ID NO: 17 - protein, 113 aa, Ginglymostoma cirratum
ARVDQTPRSVTKETGESLTINCVLRDASYTLDNTCWYRKKSGSTKEEEISKGGRYVETVNSRSKSFSLRINDLIVEDGGTYRCGVGYFCGHSLTQESCGLYTTCGGGTVVTVN

SEQ ID NO: 18 - protein, 118 aa, Ginglymostoma cirratum
ARVDQTPRSVTKETGESLTINCVLREARYELGKTCWYRKRAGSTNEESISAGGRYVETVISGSKSFSLRINDLTVEDGGTYRCGVPPAGWCSKVSCDHVGAGVLEPAACGGGTVVTVN

SEQ ID NO: 19 - protein, 110 aa, Ginglymostoma cirratum
ARVDQTPQTITKATGESLTINCVLRDASYALGRTCWYRKKSGSTNEESISLGGRYVETVNSGSKSFSLRINDLTVEDGGTYRCGAHRWWTVCGMPAPYAESGGGTVVTVN

SEQ ID NO: 20 - protein, 116 aa, Ginglymostoma cirratum
ARVDQTPQTITKETGESLTINCVLRDSNCVLESTYWLRKKSGSTNEESISKGGRYVETVYSGSKSFSLRINDLTVEDSGTYRCKGLVWPYIDGRYRDDCVLGGSAVYGGGTVVTVN

SEQ ID NO: 21 - protein, 113 aa, Ginglymostoma cirratum
ARVDQTPRSVTKETGESLTINCVLREARYELGRTCWYRKKSGSRNEESISEGGRYVETVNSGSKSFSLRINDLTVEDGGTYRCGVPAAGWCPERCAPDPPYAVCGGGTVVTVN

SEQ ID NO: 22 - protein, 110 aa, Ginglymostoma cirratum
ARVDQTPQTITKETGESLTINCVLRDNNCALSSTYWYREKSGSTNLESISLGGRYVETVNSGSKSFSLRINDLTVEDSGTYRCKVWGNEGSYDCGLESAVYGGGTVVTVN

SEQ ID NO: 23 - protein, 113 aa, Ginglymostoma cirratum
ARVDQTPQTITKETGESLTINCVLRDSNCALGSTYWYRKKSGSTNEENISKGGRYVETVRSGSKSLSLRINDLTVEDSGTYRCKVSRLSWYPESRWNCDNYDVYGDGTAVTVN

SEQ ID NO: 24 - protein, 112 aa, Ginglymostoma cirratum
ARVDQTPQTITKETGESLTINCVLRDSNCPLSSTYWYRKKSGSTNEESIPKAGRYVETVNHGSKSFSLRINDLTVEDSGTYRCKTASNVCRSGGRTSYHYDVYGDGTAVTVN

SEQ ID NO: 25 - protein, 109 aa, Ginglymostoma cirratum
ARVDQTPRSVTKETGESLTINCVLRDSNCALPSTYWYRKKSGSTNEESISKGGRYVETVNSGSKSFSLRINDLTVEDSGTYRCKVESLVFGDCPWTGRVYGDGTAVTVN

SEQ ID NO: 26 - protein, 117 aa, Ginglymostoma cirratum
ARVDQTPRSVTKETGESLTINCVLRDASNVMGSTCWYRKKSGSTNEESISLGGRYVETVNSGSKSLFSLEEINDLTVEDGGTYRCGVSPLVMFGGCDYEICALDYAGCGDGTAVTVN

SEQ ID NO: 27 - protein, 112 aa, Ginglymostoma cirratum
ARVDQTPQTITKETGESLTINCVLRDSNCALSSTYWYRKKSGSSNDESISKGGRYVETVNSGSKSFSLRINDLTVEDSGTYRCKVRIIGIGHDCPWMGWDDVYGDGTAVTVN

SEQ ID NO: 28 - protein, 116 aa, Ginglymostoma cirratum
ARVDQTPQTITKETGESLTINCVLRDSNCALSSTYWYRKKSGSANEESISKGGRYVETVNSGSKSFSLRINDLTVEDSGTYRCNLYPGAPPSRTPMRSYDCSPQLVFGGGTVVTVN

SEQ ID NO: 29 - protein, 119 aa, Ginglymostoma cirratum; identical to SEQ ID NO: 14

SEQ ID NO: 30 - protein, 117 aa, Ginglymostoma cirratum; identical to SEQ ID NO: 26

SEQ ID NO: 31 - DNA, 339 bp, Ginglymostoma cirratum
gccagggtggaccaaacaccgagatcagtaacaaaggagacgggcgaatcactgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaat

SEQ ID NO: 32 - DNA, 330 bp, Ginglymostoma cirratum
gctcgcgtcgaccagactccacagacaatcactaaagaaacaggggaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggagggggaaccgtcgtcacagtcaac

SEQ ID NO: 33 - DNA, 354 bp, Ginglymostoma cirratum
gctcgagtggaccaaacaccgagatcagtaacaaaggagacgggcgaatcactgaccatcaactgtgtcctacgctctgcgagctatgcattgggcagcacgttttggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcatccgtgatgagctggtgtggggtatagcaggttacaataaccggccctgtgctcccccctatgctgcatgcggagatggcactgccgtgactgtgaat

SEQ ID NO: 34 - DNA, 324 bp, Ginglymostoma cirratum
gctcgagtggaccaaacaccgcaaacaataacaaaggagacgggcgaatcactgaccatcaactgtggactacgagatagtaactgtgcattgtccagcacgtactggtatcgcaaaaaatcgggctcaacaaacgaggagagcatatcgaaaggtggacgatatgttgaaacagttaacagcggaccactgaccttttctttgagaattaatgatctaacagttgaagacagtggcacgtatcgatgcaaggtatcttgtctggcgatcccgatagggccgcattatgatgtatacggaggtggcactgtcgtgactgtgaat

SEQ ID NO: 35 - DNA, 333 bp, Ginglymostoma cirratum
gctcgagtggaccaaacaccgcaaacaataacaaaggagacgggcgaatcactgaccatcaactgtgtagtacgagatagtaactgtgcattgggcagcacgtactggtatcgcaaaaaatcgggctcaacaaacgaggagagcataacgaaaggtggacgatccgttgaaacagttaacagcggatcaaagtccttttctttgagaattaatgatctaacagttgaagacagtggcacgtatcgatgcaaggtactctcgattggctggcatgaccctatggggctacgggggtgtgatgtatacggaggtggcactgtcgtgactgtgaat

SEQ ID NO: 36 - DNA, 342 bp, Ginglymostoma cirratum
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcgggcgcggacttggtgtgggctgtgactatcccctgtgttcttactcgtatgctgcatgcggagatggcactgccgtgactgtgaat

SEQ ID NO: 37 - DNA, 342 bp, Ginglymostoma cirratum
gctcgagtggaccaaacaccgcaaacaataacaaaggagacgggcgaatcactgaccatcaactgtgtcctacgagatgcgagctatgcattgggcagcacgtgctggtatcgaaaaaaatcgggctcaacaaactgggagagcatagggaaaggtggacgatatgttgaaacagttaacagcggatcaaagtccttttctttgagaattaatgatctaacagttgaagacggtggcacgtatcgttgcggtgtcgggcgcggagctggtgtgggctgtgactatcccctgtgctcttactcgtatgctgcatgcggaggtggcactgtcgtgactgtgaat

SEQ ID NO: 38 - DNA, 324 bp, Ginglymostoma cirratum
gctcgagtggaccaaacaccgaaaacaataacaaaggagacgggcgaatcactgaccatcaactgtgtcctacgagatagtaactgtgccgcgtccagcacgtactggtatcgcaaaaaatcgggctcaaataacgaggagagcatatcgaaaggtggacgatatgttgaaacagttaacagcggatcaaagtccttttctttgagaattaatgatctaacagttgaagacagtggcacgtatcgatgcaacgtagaaggatatagctgtggactatatagcgtagaagattattacggaggtggcactgtcgtgactgtgaat

SEQ ID NO: 39 - DNA, 339 bp, Ginglymostoma cirratum
gctcgagtggaccaaacaccgcaaacaataacaaaggagacgggcgaatcactgaccatcgactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggaggtggcactgtcgtgactgtgaat

SEQ ID NO: 40 - DNA, 327 bp, Ginglymostoma cirratum
gctcgagtggaccaaacaccgcaaacaataacaaaggagacgggcgaatcactgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaat

SEQ ID NO: 41 - DNA, 327 bp, Ginglymostoma cirratum
gctcgagtggaccaaacaccgcaaacaataacaaaggagacgggcgaatcactgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaat

SEQ ID NO: 42 - DNA, 321 bp, Ginglymostoma cirratum
gctcgagtggaccaaacaccgagatcagtaacaaaggagacgggcgaatcactgaccatcaactgtgtcctacgagatacgaactgcccattgtatgcggcctactggtatcgcaaatcaccgggatcaacaaacgaggagaccatatgggcaactggacgatatgttcaaacagttaacagcggacttaagaccttttctttgagaattaatgatctaacttctgaagacagcggcacgtatcgatgcaacggccagacctggtgtcccgccactaagaacaatgatatttcaggaggtggcactgtcgtgactgtgaat

SEQ ID NO: 43 - DNA, 339 bp, Ginglymostoma cirratum
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagaggatatcgaaaggtggacgatatgttgaaacagttaacagcggatcaaagtccttttctttgagaattaatgatctaacagttgaagacggtggcacgtatcgttgcggtgtctggcccggctataactggtgtgggagttgtgattaccgtggcgactataatttttgcggagatggcactgccgtgactgtgaat

SEQ ID NO: 44 - DNA, 351 bp, Ginglymostoma cirratum
gctcgagtggaccaaacaccgagatcagtaacaaaggagacgggcgaatcactgaccatcaactgtgtcctacgagatgcgagttatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtctaccccggatatagttggtgtgcggccagctgtgactctgtgcccgcggaagtctatgctacattttgcggaggtggcactgtcgtgactgtgaat

SEQ ID NO: 45 - DNA, 342 bp, Ginglymostoma cirratum
gctcgagtggaccaaacaccgagatcagtaacaaaggagacgggcgaatcactgaccatcaactgtgtcctacgagatgcgaactatgcattgggcagcacgtgctggtatcgaaaaaaatcgggctcaacaaacggcgagagcatatcgaaaggtggacgatatgttgaaacagttaacagcggatcaaagtccttttctttgagaattaatgatctaacagttgaagacggtggcacgtatcgttgcggtgtcggggtggtgtttgggcgctgtgacttctgtgctcttgagagaaaaaactatgctgtatgcggaggtggcactgtcgtgactgtgaat

SEQ ID NO: 46 - DNA, 345 bp, Ginglymostoma cirratum
gctcgagtggaccaaacaccgagatcagtaacaaaggagacgggcgaatcactgaccatcaactgtgtcctacgagatgcgacctatgcattgggcaacacgtgctggtatcgaaaaaaatcgggctcatcaactgaggagagcatatcgaaaggtggacgatatgttgaaacagttaacagcggatcaaagtccttttctttgagaattaatgatctaacagttgaagacggtggcacgtatcgttgcggtgtctggcccggatataactggtgtggatcctgtgactacgttcatgatgctctacatgtattctgcggaggtggcactgtcgtgactgtgaat

SEQ ID NO: 47 - DNA, 339 bp, Ginglymostoma cirratum
gctcgagtggaccaaacaccgagatcagtaacaaaggagacgggcgaatcactgaccatcaactgtgtcctacgagatgcgagctatacattggacaacacgtgctggtatcgaaaaaaatcgggctcaacaaaggaggaggaaatatcgaaaggtggacgatatgttgaaacagttaacagccggtcaaagtccttttctttgagaattaatgatctaatagttgaagacggtggcacgtatcgttgcggtgtcggatacttctgtggacactctctgacccaagagtcttgcgggctctatactacatgcggaggtggcactgtcgtgactgtgaat

SEQ ID NO: 48 - DNA, 354 bp, Ginglymostoma cirratum
gctcgagtggaccaaacaccgagatcagtaacaaaggagacgggcgaatcactgaccatcaactgtgtcctacgtgaagcgaggtatgaattgggcaagacgtgctggtatcgaaaaagagcgggctcaacaaacgaggagagcatatccgcaggtggacgatatgttgaaacagttatcagcggatcaaagtccttttctttgagaattaatgatctaacagttgaagacggtggcacgtatcgttgtggtgtcccccctgccggctggtgttccaaggttagctgtgaccacgtcggcgcaggtgtgcttgaaccagctgcatgcggaggtggcactgtcgtgactgtgaat

SEQ ID NO: 49 - DNA, 330 bp, Ginglymostoma cirratum
gctcgagtggaccaaacaccgcaaacaataacaaaggccacgggcgaatcactgaccatcaactgtgtcctacgagatgcgagttatgcattgggcagaacgtgctggtatcgaaaaaaatcgggctcaacaaacgaggagagcatatcgctaggtggacgatatgttgaaacagttaacagcggatcaaagtccttttctttgagaattaatgatctaacagttgaagacggtggcacgtatcgttgcggtgcccaccgctggtggacggtgtgcgggatgccggccccctatgctgagagcggaggtggcactgtcgtgactgtgaat

SEQ ID NO: 50 - DNA, 348 bp, Ginglymostoma cirratum
gctcgagtggaccaaacaccgcaaacaataacaaaggagacgggcgaatcactgaccatcaactgtgttctccgagatagtaattgtgtattggagagcacgtactggcttcgcaaaaaatcgggctcaacaaacgaggagagtatatcgaaaggtggacgatatgttgaaacagtttacagcggatcaaagtccttttctttgagaattaatgatctaacagttgaagacagtggcacgtatcgatgcaagggattggtatggccatatatagatggccggtatagggatgactgcgtgctgggcggatccgcagtatacggaggtggcactgtcgtgactgtgaat

SEQ ID NO: 51 - DNA, 339 bp, Ginglymostoma cirratum
gctcgagtggaccaaacaccgagatcagtaacaaaggagacgggcgaatcattgaccatcaactgtgtcctacgagaggcgagatatgagttgggcagaacgtgttggtatcgaaaaaaatcgggctcaagaaacgaggagagcatatcggaaggtggacgatatgttgaaacagttaacagcggatcaaagtccttttctttgagaattaatgatctaacagttgaagacggtggcacgtatcgttgcggtgtccccgccgccggctggtgccccgagcggtgcgctcctgatcccccgtatgctgtatgcggaggtggcactgtcgtgactgtgaat

SEQ ID NO: 52 - DNA, 330 bp, Ginglymostoma cirratum
gctcgagtggaccaaacaccgcaaacaataacaaaggagacgggcgaatcactgaccatcaactgtgtcctacgagataataactgtgcattgtccagcacgtactggtatcgcgaaaaatcgggctcaacaaacttggagagcatatcgctaggtgggagatatgttgaaacagttaacagcggatcaaagtccttttctttgagaattaatgatctaacagttgaagacagtggcacgtatcgatgcaaggtatggggcaacgaggggagctatgactgcggactggagagtgctgtatacggaggtggcactgtcgtgactgtgaat

SEQ ID NO: 53 - DNA, 339 bp, Ginglymostoma cirratum
gctcgagtggaccaaacaccgcaaacaataacaaaggagacgggcgaatcactgaccatcaactgtgtcctacgagatagtaactgtgcattgggcagcacgtactggtatcgcaaaaaatcgggctcaacaaacgaggagaacatatcgaaaggtggacgatatgttgaaacagttaggagcggatcaaagtccttgtctttgagaattaatgatctaacagttgaagactctggcacgtatcgatgcaaggtgtcccgactaagctggtatccagagagtcgctggaactgcgacaactatgatgtatacggagatggcactgccgtgactgtgaat

SEQ ID NO: 54 - DNA, 336 bp, Ginglymostoma cirratum
gctcgagtggaccaaacaccgcaaacaataacaaaggagacgggcgaatcactgaccatcaactgtgtcctacgagatagtaactgtccattgtccagcacgtactggtatcgcaaaaaatcgggctcaacaaacgaggagagcataccgaaagcgggacgatatgttgaaacagttaaccacggatcaaagtccttttctttgagaattaatgatctaacagttgaagacagtggcacgtatcgatgcaagacggcctcgaacgtatgtcgatccggtggccgaacatcttaccactatgatgtatacggagatggcactgccgtgactgtgaat

SEQ ID NO: 55 - DNA, 327 bp, Ginglymostoma cirratum
gctcgagtggaccaaacaccgagatcagtaacaaaggagacgggcgaatcactgaccatcaactgtgtcctacgagatagtaactgtgcattgcccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaaggtagagtccctggtattcggtgactgtccttggactggtagagtatacggagatggcactgccgtgactgtgaat

SEQ ID NO: 56 - DNA, 345 bp, Ginglymostoma cirratum
gctcgagtggaccaaacaccgagatcagtaacaaaggagacgggcgaatcactgaccatcaactgtgtcctacgagatgcgagcaatgtaatgggcagcacgtgctggtatcgaaaaaaatcgggctcaacaaacgaggagagcatatctttaggtggacgatatgttgaaacagttaacagcggatcaaagtccttttctttgagaattaatgatctaacagttgaagacggtggcacgtatcgttgcggtgtctcacccctggtgatgttcgggggctgtgactacgaaatctgtgctcttgactatgctggctgcggagatggcactgccgtgactgtgaat

SEQ ID NO: 57 - DNA, 336 bp, Ginglymostoma cirratum
gctcgagtggaccaaacaccgcaaacaataacaaaggagacgggcgaatcactgaccatcaactgtgtcctacgagatagtaactgtgcattgtccagcacgtactggtatcgcaaaaaatcgggctcatcaaacgatgagagcatatcgaaaggtggacgatatgttgaaacagttaacagcggatcaaagtcctttagtttgagaattaatgatctaacagttgaagacagtggcacgtatcgatgcaaggtacgaataattggtatcggtcatgactgcccatggatgggttgggatgatgtatacggagatggcactgccgtgactgtgaat

SEQ ID NO: 58 - DNA, 348 bp, Ginglymostoma cirratum
gctcgagtggaccaaacaccgcaaacaataacaaaggagacgggcgaatcactgaccatcaactgtgtcctacgagatagtaactgtgcattgtccagcacgtactggtatcgcaaaaaatcgggctcagcaaacgaggagagcatatcgaaaggtggacgatatgttgaaacagttaacagcggatcaaagtccttttctttgagaattaatgatctaacagttgaagacagtggcacatatcgatgcaacttatacccaggagcccccccgtcgcggactcctatgcgatcatatgactgcagtccccaactggtcttcggaggtggcactgtcgtgactgtgaat

SEQ ID NO: 59 - DNA, 357 bp, Ginglymostoma cirratum
gctcgagtggaccaaacaccgcaaacaataacaaaggagacgggcgaatcactgaccatcaactgtgtcctacgagatagtaactgtgcattgtccagaacgtactggtatcgcaaaaaatcgggctcaacagacgaggagagcatatcgaaaggtggacgatatgttgaaacagttaacagcggatcaaagtccttttctttgagaattaatgatctaacagttgaagacagtggcacgtatcgatgcaacgtatatagctggtatggcggggatcacaactcatgggactggagatcggatccagcgatggtgtgtggggatgtatacggaggtggcactgtcgtgactgtgaat

SEQ ID NO: 60 - DNA, 1083 bp, Ginglymostoma cirratum
gctcgagtggaccaaacaccgagatcagtaacaaaggagacgggcgaatcactgaccatcaactgtgtcctacgagatgcgagcaatgtaatgggcagcacgtgctggtatcgaaaaaaatcgggctcaacaaacgaggagagcatatctttaggtggacgatatgttgaaacagttaacagcggatcaaagtcccttttttctttggaggaaattaatgatctaacagttgaagacggtggcacgtatcgttgcggtgtctcacccctggtgatgttcgggggctgtgactacgaaatctgtgctcttgactatgctggctgcggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 61 - protein, 347 aa, Artificial Sequence
  Description of Artificial Sequence Synthetic sequence
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 356 aa, Artificial Sequence
  Description of Artificial Sequence Synthetic sequence
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 131 aa, Artificial Sequence
  Description of Artificial Sequence Synthetic sequence
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGPLEVLFQGPHHHHHHHH

SEQ ID NO: 64 - protein, 344 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 353 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 128 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGPLEVLFQGPHHHHHHHH

SEQ ID NO: 67 - protein, 309 aa, Artificial Sequence
  Description of Artificial Sequence Synthetic sequence
HHHHHHHHGPLEVLFQGPARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 68 - protein, 288 aa, Artificial Sequence
  Description of Artificial Sequence Synthetic sequence
HHHHHHHHGPLEVLFQGPARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGSGGGGMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 69 - protein, 297 aa, Artificial Sequence
  Description of Artificial Sequence Synthetic sequence
MQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLRGGSGGSGGSGGSGGGARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGPLEVLFQGPHHHHHHHH

SEQ ID NO: 70 - protein, 352 aa, Artificial Sequence
  Description of Artificial Sequence Synthetic sequence
ARVDQTPRSVTKETGESLTINCVLRSASYALGSTFWYRKKSGSTNEESISKGGRYVETVNSGSKSFSLRINDLTVEDGGTYRCGVIRDELVWGIAGYNNRPCAPPYAACGDGTAVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 361 aa, Artificial Sequence
  Description of Artificial Sequence Synthetic sequence
ARVDQTPRSVTKETGESLTINCVLRSASYALGSTFWYRKKSGSTNEESISKGGRYVETVNSGSKSFSLRINDLTVEDGGTYRCGVIRDELVWGIAGYNNRPCAPPYAACGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 342 aa, Artificial Sequence
  Description of Artificial Sequence Synthetic sequence
ARVDQTPQTITKETGESLTINCGLRDSNCALSSTYWYRKKSGSTNEESISKGGRYVETVNSGPLTFSLRINDLTVEDSGTYRCKVSCLAIPIGPHYDVYGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 351 aa, Artificial Sequence
  Description of Artificial Sequence Synthetic sequence
ARVDQTPQTITKETGESLTINCGLRDSNCALSSTYWYRKKSGSTNEESISKGGRYVETVNSGPLTFSLRINDLTVEDSGTYRCKVSCLAIPIGPHYDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 345 aa, Artificial Sequence
  Description of Artificial Sequence Synthetic sequence
ARVDQTPQTITKETGESLTINCVVRDSNCALGSTYWYRKKSGSTNEESITKGGRSVETVNSGSKSFSLRINDLTVEDSGTYRCKVLSIGWHDPMGLRGCDVYGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 354 aa, Artificial Sequence
  Description of Artificial Sequence Synthetic sequence
ARVDQTPQTITKETGESLTINCVVRDSNCALGSTYWYRKKSGSTNEESITKGGRSVETVNSGSKSFSLRINDLTVEDSGTYRCKVLSIGWHDPMGLRGCDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 348 aa, Artificial Sequence
  Description of Artificial Sequence Synthetic sequence
ARVDQTPRSVTKETGESLTINCVLRDASYALGSTCWYRKKSGSTNEESISKGGRYVETVNSGSKSFSLRINDLTVEDGGTYRCGVGRGLGVGCDYPLCSYSYAACGDGTAVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 357 aa, Artificial Sequence
  Description of Artificial Sequence Synthetic sequence
ARVDQTPRSVTKETGESLTINCVLRDASYALGSTCWYRKKSGSTNEESISKGGRYVETVNSGSKSFSLRINDLTVEDGGTYRCGVGRGLGVGCDYPLCSYSYAACGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 131 aa, Artificial Sequence
  Description of Artificial Sequence Synthetic sequence
ARVDQTPRSVTKETGESLTINCVLRDASYALGSTCWYRKKSGSTNEESISKGGRYVETVNSGSKSFSLRINDLTVEDGGTYRCGVGRGLGVGCDYPLCSYSYAACGDGTAVTVNGSLEVLFQGPGHHHHHH

SEQ ID NO: 79 - protein, 348 aa, Artificial Sequence
  Description of Artificial Sequence Synthetic sequence
ARVDQTPQTITKETGESLTINCVLRDASYALGSTCWYRKKSGSTNWESIGKGGRYVETVNSGSKSFSLRINDLTVEDGGTYRCGVGRGAGVGCDYPLCSYSYAACGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 357 aa, Artificial Sequence
  Description of Artificial Sequence Synthetic sequence
ARVDQTPQTITKETGESLTINCVLRDASYALGSTCWYRKKSGSTNWESIGKGGRYVETVNSGSKSFSLRINDLTVEDGGTYRCGVGRGAGVGCDYPLCSYSYAACGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 131 aa, Artificial Sequence
  Description of Artificial Sequence Synthetic sequence
ARVDQTPQTITKETGESLTINCVLRDASYALGSTCWYRKKSGSTNWESIGKGGRYVETVNSGSKSFSLRINDLTVEDGGTYRCGVGRGAGVGCDYPLCSYSYAACGGGTVVTVNGSLEVLFQGPGHHHHHH

SEQ ID NO: 82 - protein, 342 aa, Artificial Sequence
  Description of Artificial Sequence Synthetic sequence
ARVDQTPKTITKETGESLTINCVLRDSNCAASSTYWYRKKSGSNNEESISKGGRYVETVNSGSKSFSLRINDLTVEDSGTYRCNVEGYSCGLYSVEDYYGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 351 aa, Artificial Sequence
  Description of Artificial Sequence Synthetic sequence
ARVDQTPKTITKETGESLTINCVLRDSNCAASSTYWYRKKSGSNNEESISKGGRYVETVNSGSKSFSLRINDLTVEDSGTYRCNVEGYSCGLYSVEDYYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 347 aa, Artificial Sequence
  Description of Artificial Sequence Synthetic sequence
ARVDQTPQTITKETGESLTIDCVLRDSNCALSSTHWYRKKSGSTNEERILQGRRYVETVNSGSKSFSLRINDLRVEDSGTYRCKVYWGNSWQDKFCPGLGSYEYGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 356 aa, Artificial Sequence
  Description of Artificial Sequence Synthetic sequence
ARVDQTPQTITKETGESLTIDCVLRDSNCALSSTHWYRKKSGSTNEERILQGRRYVETVNSGSKSFSLRINDLRVEDSGTYRCKVYWGNSWQDKFCPGLGSYEYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 343 aa, Artificial Sequence
  Description of Artificial Sequence Synthetic sequence
ARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 352 aa, Artificial Sequence
  Description of Artificial Sequence Synthetic sequence
ARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 126 aa, Artificial Sequence
  Description of Artificial Sequence Synthetic sequence
ARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVNGSLEVLFQGPGHHHHHH

SEQ ID NO: 89 - protein, 343 aa, Artificial Sequence
  Description of Artificial Sequence Synthetic sequence
ARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNSREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 352 aa, Artificial Sequence
  Description of Artificial Sequence Synthetic sequence
ARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 126 aa, Artificial Sequence
  Description of Artificial Sequence Synthetic sequence
ARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNGSLEVLFQGPGHHHHHH

SEQ ID NO: 92 - protein, 350 aa, Artificial Sequence
  Description of Artificial Sequence Synthetic sequence
ARVDQTPRSVTKETGESLTINCVLRDTNCPLYAAYWYRKSPGSTNEETIWATGRYVQTVNSGLKTFSLRINDLTSEDSGTYRCNGQTWCPATKNNDISGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 356 aa, Artificial Sequence
  Description of Artificial Sequence Synthetic sequence
ARVDQTPRSVTKETGESLTINCVLRDASYALGSTCWYRKKSGSTNEERISKGGRYVETVNSGSKSFSLRINDLTVEDGGTYRCGVWPGYNWCGSCDYRGDYNFCGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 362 aa, Artificial Sequence
  Description of Artificial Sequence Synthetic sequence
ARVDQTPQTITKETGESLTINCVLRDSNCALSRTYWYRKKSGSTDEESISKGGRYVETVNSGSKSFSLRINDLTVEDSGTYRCNVYSWYGGDHNSWDWRSDPAMVCGDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 357 aa, Artificial Sequence
  Description of Artificial Sequence Synthetic sequence
ARVDQTPRSVTKETGESLTINCVLRDANYALGSTCWYRKKSGSTNGESISKGGRYVETVNSGSKSFSLRINDLTVEDGGTYRCGVGVVFGRCDFCALERKNYAVCGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 358 aa, Artificial Sequence
  Description of Artificial Sequence Synthetic sequence
ARVDQTPRSVTKETGESLTINCVLRDATYALGNTCWYRKKSGSSTEESISKGGRYVETVNSGSKSFSLRINDLTVEDGGTYRCGVWPGYNWCGSCDYVHDALHVFCGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 356 aa, Artificial Sequence
  Description of Artificial Sequence Synthetic sequence
ARVDQTPRSVTKETGESLTINCVLRDASYTLDNTCWYRKKSGSTKEEEISKGGRYVETVNSRSKSFSLRINDLIVEDGGTYRCGVGYFCGHSLTQESCGLYTTCGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 130 aa, Artificial Sequence
  Description of Artificial Sequence Synthetic sequence
ARVDQTPRSVTKETGESLTINCVLRDASYTLDNTCWYRKKSGSTKEEEISKGGRYVETVNSRSKSFSLRINDLIVEDGGTYRCGVGYFCGHSLTQESCGLYTTCGGGTVVTVNGSLEVLFQGPGHHHHHH

SEQ ID NO: 99 - protein, 361 aa, Artificial Sequence
  Description of Artificial Sequence Synthetic sequence
ARVDQTPRSVTKETGESLTINCVLREARYELGKTCWYRKRAGSTNEESISAGGRYVETVISGSKSFSLRINDLTVEDGGTYRCGVPPAGWCSKVSCDHVGAGVLEPAACGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 353 aa, Artificial Sequence
  Description of Artificial Sequence Synthetic sequence
ARVDQTPQTITKATGESLTINCVLRDASYALGRTCWYRKKSGSTNEESISLGGRYVETVNSGSKSFSLRINDLTVEDGGTYRCGAHRWWTVCGMPAPYAESGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 127 aa, Artificial Sequence
  Description of Artificial Sequence Synthetic sequence
ARVDQTPQTITKATGESLTINCVLRDASYALGRTCWYRKKSGSTNEESISLGGRYVETVNSGSKSFSLRINDLTVEDGGTYRCGAHRWWTVCGMPAPYAESGGGTVVTVNGSLEVLFQGPGHHHHHH

SEQ ID NO: 102 - protein, 359 aa, Artificial Sequence
  Description of Artificial Sequence Synthetic sequence
ARVDQTPQTITKETGESLTINCVLRDSNCVLESTYWLRKKSGSTNEESISKGGRYVETVYSGSKSFSLRINDLTVEDSGTYRCKGLVWPYIDGRYRDDCVLGGSAVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 133 aa, Artificial Sequence
  Description of Artificial Sequence Synthetic sequence
ARVDQTPQTITKETGESLTINCVLRDSNCVLESTYWLRKKSGSTNEESISKGGRYVETVYSGSKSFSLRINDLTVEDSGTYRCKGLVWPYIDGRYRDDCVLGGSAVYGGGTVVTVNGSLEVLFQGPGHHHHHH

SEQ ID NO: 104 - protein, 356 aa, Artificial Sequence
  Description of Artificial Sequence Synthetic sequence
ARVDQTPRSVTKETGESLTINCVLREARYELGRTCWYRKKSGSRNEESISEGGRYVETVNSGSKSFSLRINDLTVEDGGTYRCGVPAAGWCPERCAPDPPYAVCGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 353 aa, Artificial Sequence
  Description of Artificial Sequence Synthetic sequence
ARVDQTPQTITKETGESLTINCVLRDNNCALSSTYWYREKSGSTNLESISLGGRYVETVNSGSKSFSLRINDLTVEDSGTYRCKVWGNEGSYDCGLESAVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 356 aa, Artificial Sequence
  Description of Artificial Sequence Synthetic sequence
ARVDQTPQTITKETGESLTINCVLRDSNCALGSTYWYRKKSGSTNEENISKGGRYVETVRSGSKSLSLRINDLTVEDSGTYRCKVSRLSWYPESRWNCDNYDVYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 130 aa, Artificial Sequence
  Description of Artificial Sequence Synthetic sequence
ARVDQTPQTITKETGESLTINCVLRDSNCALGSTYWYRKKSGSTNEENISKGGRYVETVRSGSKSLSLRINDLTVEDSGTYRCKVSRLSWYPESRWNCDNYDVYGDGTAVTVNGSLEVLFQGPGHHHHHH

SEQ ID NO: 108 - protein, 355 aa, Artificial Sequence
  Description of Artificial Sequence Synthetic sequence
ARVDQTPQTITKETGESLTINCVLRDSNCPLSSTYWYRKKSGSTNEESIPKAGRYVETVNHGSKSFSLRINDLTVEDSGTYRCKTASNVCRSGGRTSYHYDVYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 129 aa, Artificial Sequence
  Description of Artificial Sequence Synthetic sequence
ARVDQTPQTITKETGESLTINCVLRDSNCPLSSTYWYRKKSGSTNEESIPKAGRYVETVNHGSKSFSLRINDLTVEDSGTYRCKTASNVCRSGGRTSYHYDVYGDGTAVTVNGSLEVLFQGPGHHHHHH

SEQ ID NO: 110 - protein, 352 aa, Artificial Sequence
  Description of Artificial Sequence Synthetic sequence
ARVDQTPRSVTKETGESLTINCVLRDSNCALPSTYWYRKKSGSTNEESISKGGRYVETVNSGSKSFSLRINDLTVEDSGTYRCKVESLVFGDCPWTGRVYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 360 aa, Artificial Sequence
  Description of Artificial Sequence Synthetic sequence
ARVDQTPRSVTKETGESLTINCVLRDASNVMGSTCWYRKKSGSTNEESISLGGRYVETVNSGSKSLFSLEEINDLTVEDGGTYRCGVSPLVMFGGCDYEICALDYAGCGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 355 aa, Artificial Sequence
  Description of Artificial Sequence Synthetic sequence
ARVDQTPQTITKETGESLTINCVLRDSNCALSSTYWYRKKSGSSNDESISKGGRYVETVNSGSKSFSLRINDLTVEDSGTYRCKVRIIGIGHDCPWMGWDDVYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 359 aa, Artificial Sequence
  Description of Artificial Sequence Synthetic sequence
ARVDQTPQTITKETGESLTINCVLRDSNCALSSTYWYRKKSGSANEESISKGGRYVETVNSGSKSFSLRINDLTVEDSGTYRCNLYPGAPPSRTPMRSYDCSPQLVFGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 362 aa, Artificial Sequence; identical to SEQ ID NO: 94
  Description of Artificial Sequence Synthetic sequence

SEQ ID NO: 115 - protein, 360 aa, Artificial Sequence; identical to SEQ ID NO: 111
  Description of Artificial Sequence Synthetic sequence

SEQ ID NO: 116 - protein, 491 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 562 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSNITEENVQNMNNAGDKWSAFLKEQSTLAQMYPLQEIQNGGGSSALEEQYKTFLDFFMIQLEDLLYQLALGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 571 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGSALEEQYKTFLDFFMIQLEDLLYQLALASWNYNTNITEENVQNMNNAGDKWSAFLKEQSTLAQMYPLQEIQNGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 526 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGSALEEQYKTFLDFFMIQLEDLLYQLALGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 323 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNKESGSVSSEQLAQFRSGDGYIPEAPRDGQAYVRKDGEWVLLSTFLGRSLEVLFQGPGHHHHHHHHSAWSHPQFEK

SEQ ID NO: 121 - protein, 487 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 487 aa, Artificial Sequence
  Description of Artificial Sequence Synthetic sequence
ARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVNGGGGSGGGGSGGGGSGGGGSGGGGSARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 487 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 487 aa, Artificial Sequence
  Description of Artificial Sequence Synthetic sequence
ARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNGGGGSGGGGSGGGGSGGGGSGGGGSARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 188 aa, Artificial Sequence
  Description of Artificial Sequence Synthetic sequence
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNKESGSVSSEQLAQFRSGDGYIPEAPRDGQAYVRKDGEWVLLSTFLGRSLEVLFQGPGHHHHHHHHSAWSHPQFEK

SEQ ID NO: 126 - protein, 185 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNKESGSVSSEQLAQFRSGDGYIPEAPRDGQAYVRKDGEWVLLSTFLGRSLEVLFQGPGHHHHHHHHSAWSHPQFEK

SEQ ID NO: 127 - protein, 629 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGGGGSGGGGSGGGGSGGGGSGGGGARVDQTPRSVTKETGESLTINCVLRDASYALGSTCWYRKKSGSTNEESISKGGRYVETVNSGSKSFSLRINDLTVEDGGTYRCGVGRGLGVGCDYPLCSYSYAACGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 629 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGGGGSGGGGSGGGGSGGGGSGGGGARVDQTPQTITKETGESLTINCVLRDASYALGSTCWYRKKSGSTNWESIGKGGRYVETVNSGSKSFSLRINDLTVEDGGTYRCGVGRGAGVGCDYPLCSYSYAACGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 624 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGGGGSGGGGSGGGGSGGGGSGGGGARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 624 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGGGGSGGGGSGGGGSGGGGSGGGGARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 494 aa, Artificial Sequence
  Description of Artificial Sequence Synthetic sequence
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 488 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 302 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNKESGSVSSEQLAQFRSGDGSLELIKLRIMEIEARIAKIEKDRAIKESGSVSSEQLAQFRSGDARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGSLEVLFQGPGHHHHHH

SEQ ID NO: 134 - DNA, 1044 bp, Artificial Sequence
  Description of Artificial Sequence Synthetic sequence
gccagggtggaccaaacaccgagatcagtaacaaaggagacgggcgaatcactgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 135 - DNA, 1071 bp, Artificial Sequence
  Description of Artificial Sequence Synthetic sequence
gccagggtggaccaaacaccgagatcagtaacaaaggagacgggcgaatcactgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 136 - DNA, 396 bp, Artificial Sequence
  Description of Artificial Sequence Synthetic sequence
gccagggtggaccaaacaccgagatcagtaacaaaggagacgggcgaatcactgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaatggacctctggaagtgctgtttcagggaccacaccaccaccaccaccaccaccattga

SEQ ID NO: 137 - DNA, 1035 bp, Artificial Sequence
  Description of Artificial Sequence Synthetic sequence
gctcgcgtcgaccagactccacagacaatcactaaagaaacaggggaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggagggggaaccgtcgtcacagtcaactctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 138 - DNA, 1062 bp, Artificial Sequence
  Description of Artificial Sequence Synthetic sequence
gctcgcgtcgaccagactccacagacaatcactaaagaaacaggggaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggagggggaaccgtcgtcacagtcaactctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 139 - DNA, 387 bp, Artificial Sequence
  Description of Artificial Sequence Synthetic sequence
gcaagagtggaccagacccctcagactattaccaaagaaaccggagagagcctgaccatcaactgcgtgctgagggattcaaactgtgccctggcctccaccgactggtaccggaagaagagcggctccacaaacgaggagtctatcagcaagggcggcaggtatgtggagacagtgaactccggctctaagagcttctccctgcgcatcaatgacctgaccgtggaggattctggcacataccggtgcaacgcctgggatagctgggagacaagacagctgaagtgtgactacgacgtgtacggaggaggaaccgtggtgacagtgaatggacctctggaagtgctgtttcagggaccacaccaccaccaccaccaccaccattga

SEQ ID NO: 140 - DNA, 930 bp, Artificial Sequence
  Description of Artificial Sequence Synthetic sequence
caccatcatcaccaccaccaccacggacccctggaggtgctgttccagggaccagctcgcgtcgaccagactccacagacaatcactaaagaaacaggggaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggagggggaaccgtcgtcacagtcaacggctctggaggaggaggagagagccaggtgcgccagcagttcagcaaggatatcgagaagctgctgaacgagcaagtgaataaggagatgcagtctagcaacctgtatatgtccatgtcctcttggtgttacacccactctctggacggagcaggactgttcctgtttgatcacgccgccgaggagtacgagcacgccaagaagctgatcatctttctgaatgagaacaatgtgcctgtgcagctgaccagcatctccgccccagagcacaagttcgagggcctgacacagatctttcagaaggcctatgagcacgagcagcacatctctgagagcatcaacaatatcgtggaccacgccatcaagtccaaggatcacgccacattcaactttctgcagtggtacgtggccgagcagcacgaggaggaggtgctgttcaaggacatcctggataagatcgagctgatcggcaacgagaatcacggcctgtatctggccgatcagtacgtgaagggcatcgccaagagccggaagtccggctcttga

SEQ ID NO: 141 - DNA, 867 bp, Artificial Sequence
  Description of Artificial Sequence Synthetic sequence
caccatcatcaccaccaccaccacggacccctggaggtgctgttccagggaccagctcgcgtcgaccagactccacagacaatcactaaagaaacaggggaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggagggggaaccgtcgtcacagtcaacggctctggaggaggaggaatgcagatctacgaaggcaagctgaccgccgaggggctgagattcggaattgtggcaagcaggtttaaccacgcactggtggaccgactggtcgaaggagctatcgatgcaattgtgcggcatggcgggagagaggaagacatcacactggtgagagtcccaggcagctgggagattccagtggcagctggagaactggcacggaaagaggacatcgatgccgtgatcgctattggcgtcctgattagaggggctactccccacttcgactatatcgcatcagaagtgagcaagggcctggccgatctgtctctggagctgcgaaaacctatcacttttggggtcattactgccgataccctggaacaggcaatcgagcgcgccggcaccaagcatggaaacaaaggctgggaagcagccctgtccgctattgagatggcaaatctgttcaagtctctgaggtga

SEQ ID NO: 142 - DNA, 894 bp, Artificial Sequence
  Description of Artificial Sequence Synthetic sequence
atgcagatctacgaaggcaagctgaccgccgaggggctgagattcggaattgtggcaagcaggtttaaccacgcactggtggaccgactggtcgaaggagctatcgatgcaattgtgcggcatggcgggagagaggaagacatcacactggtgagagtcccaggcagctgggagattccagtggcagctggagaactggcacggaaagaggacatcgatgccgtgatcgctattggcgtcctgattagaggggctactccccacttcgactatatcgcatcagaagtgagcaagggcctggccgatctgtctctggagctgcgaaaacctatcacttttggggtcattactgccgataccctggaacaggcaatcgagcgcgccggcaccaagcatggaaacaaaggctgggaagcagccctgtccgctattgagatggcaaatctgttcaagtctctgaggggaggctccggaggatctggagggagtggaggctcaggaggaggcgcaagagtggaccagaccccccagactattaccaaagaaaccggagagagcctgaccatcaactgcgtgctgagggattcaaactgtgccctggcctccaccgactggtaccggaagaagagcggctccacaaacgaggagtctatcagcaagggcggcaggtatgtggagacagtgaactccggctctaagagcttctccctgcgcatcaatgacctgaccgtggaggattctggcacataccggtgcaacgcctgggatagctgggagacaagacagctgaagtgtgactacgacgtgtacggaggaggaaccgtggtgacagtgaatggacctctggaagtgctgtttcagggaccacaccaccaccaccaccaccaccattga

SEQ ID NO: 143 - DNA, 1059 bp, Artificial Sequence
  Description of Artificial Sequence Synthetic sequence
gctcgagtggaccaaacaccgagatcagtaacaaaggagacgggcgaatcactgaccatcaactgtgtcctacgctctgcgagctatgcattgggcagcacgttttggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcatccgtgatgagctggtgtggggtatagcaggttacaataaccggccctgtgctcccccctatgctgcatgcggagatggcactgccgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 144 - DNA, 1086 bp, Artificial Sequence
  Description of Artificial Sequence Synthetic sequence
gctcgagtggaccaaacaccgagatcagtaacaaaggagacgggcgaatcactgaccatcaactgtgtcctacgctctgcgagctatgcattgggcagcacgttttggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcatccgtgatgagctggtgtggggtatagcaggttacaataaccggccctgtgctcccccctatgctgcatgcggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 145 - DNA, 1029 bp, Artificial Sequence
  Description of Artificial Sequence Synthetic sequence
gctcgagtggaccaaacaccgcaaacaataacaaaggagacgggcgaatcactgaccatcaactgtggactacgagatagtaactgtgcattgtccagcacgtactggtatcgcaaaaaatcgggctcaacaaacgaggagagcatatcgaaaggtggacgatatgttgaaacagttaacagcggaccactgaccttttctttgagaattaatgatctaacagttgaagacagtggcacgtatcgatgcaaggtatcttgtctggcgatcccgatagggccgcattatgatgtatacggaggtggcactgtcgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 146 - DNA, 1056 bp, Artificial Sequence
  Description of Artificial Sequence Synthetic sequence
gctcgagtggaccaaacaccgcaaacaataacaaaggagacgggcgaatcactgaccatcaactgtggactacgagatagtaactgtgcattgtccagcacgtactggtatcgcaaaaaatcgggctcaacaaacgaggagagcatatcgaaaggtggacgatatgttgaaacagttaacagcggaccactgaccttttctttgagaattaatgatctaacagttgaagacagtggcacgtatcgatgcaaggtatcttgtctggcgatcccgatagggccgcattatgatgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 147 - DNA, 1038 bp, Artificial Sequence
  Description of Artificial Sequence Synthetic sequence
gctcgagtggaccaaacaccgcaaacaataacaaaggagacgggcgaatcactgaccatcaactgtgtagtacgagatagtaactgtgcattgggcagcacgtactggtatcgcaaaaaatcgggctcaacaaacgaggagagcataacgaaaggtggacgatccgttgaaacagttaacagcggatcaaagtccttttctttgagaattaatgatctaacagttgaagacagtggcacgtatcgatgcaaggtactctcgattggctggcatgaccctatggggctacgggggtgtgatgtatacggaggtggcactgtcgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 148 - DNA, 1065 bp, Artificial Sequence
  Description of Artificial Sequence Synthetic sequence
gctcgagtggaccaaacaccgcaaacaataacaaaggagacgggcgaatcactgaccatcaactgtgtagtacgagatagtaactgtgcattgggcagcacgtactggtatcgcaaaaaatcgggctcaacaaacgaggagagcataacgaaaggtggacgatccgttgaaacagttaacagcggatcaaagtccttttctttgagaattaatgatctaacagttgaagacagtggcacgtatcgatgcaaggtactctcgattggctggcatgaccctatggggctacgggggtgtgatgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 149 - DNA, 1047 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcgggcgcggacttggtgtgggctgtgactatcccctgtgttcttactcgtatgctgcatgcggagatggcactgccgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 150 - DNA, 1074 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcgggcgcggacttggtgtgggctgtgactatcccctgtgttcttactcgtatgctgcatgcggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 151 - DNA, 396 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcgggcgcggacttggtgtgggctgtgactatcccctgtgttcttactcgtatgctgcatgcggagatggcactgccgtgactgtgaatggatccctggaggtgctgttccagggcccaggccaccaccaccaccaccactga

SEQ ID NO: 152 - DNA, 1047 bp, Artificial Sequence
  Description of Artificial Sequence Synthetic sequence
gctcgagtggaccaaacaccgcaaacaataacaaaggagacgggcgaatcactgaccatcaactgtgtcctacgagatgcgagctatgcattgggcagcacgtgctggtatcgaaaaaaatcgggctcaacaaactgggagagcatagggaaaggtggacgatatgttgaaacagttaacagcggatcaaagtccttttctttgagaattaatgatctaacagttgaagacggtggcacgtatcgttgcggtgtcgggcgcggagctggtgtgggctgtgactatcccctgtgctcttactcgtatgctgcatgcggaggtggcactgtcgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 153 - DNA, 1074 bp, Artificial Sequence
  Description of Artificial Sequence Synthetic sequence
gctcgagtggaccaaacaccgcaaacaataacaaaggagacgggcgaatcactgaccatcaactgtgtcctacgagatgcgagctatgcattgggcagcacgtgctggtatcgaaaaaaatcgggctcaacaaactgggagagcatagggaaaggtggacgatatgttgaaacagttaacagcggatcaaagtccttttctttgagaattaatgatctaacagttgaagacggtggcacgtatcgttgcggtgtcgggcgcggagctggtgtgggctgtgactatcccctgtgctcttactcgtatgctgcatgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 154 - DNA, 396 bp, Artificial Sequence
  Description of Artificial Sequence Synthetic sequence
gctcgagtggaccaaacaccgcaaacaataacaaaggagacgggcgaatcactgaccatcaactgtgtcctacgagatgcgagctatgcattgggcagcacgtgctggtatcgaaaaaaatcgggctcaacaaactgggagagcatagggaaaggtggacgatatgttgaaacagttaacagcggatcaaagtccttttctttgagaattaatgatctaacagttgaagacggtggcacgtatcgttgcggtgtcgggcgcggagctggtgtgggctgtgactatcccctgtgctcttactcgtatgctgcatgcggaggtggcactgtcgtgactgtgaatggatccctggaggtgctgttccagggcccaggccaccaccaccaccaccactga

SEQ ID NO: 155 - DNA, 1029 bp, Artificial Sequence
  Description of Artificial Sequence Synthetic sequence
gctcgagtggaccaaacaccgaaaacaataacaaaggagacgggcgaatcactgaccatcaactgtgtcctacgagatagtaactgtgccgcgtccagcacgtactggtatcgcaaaaaatcgggctcaaataacgaggagagcatatcgaaaggtggacgatatgttgaaacagttaacagcggatcaaagtccttttctttgagaattaatgatctaacagttgaagacagtggcacgtatcgatgcaacgtagaaggatatagctgtggactatatagcgtagaagattattacggaggtggcactgtcgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 156 - DNA, 1056 bp, Artificial Sequence
  Description of Artificial Sequence Synthetic sequence
gctcgagtggaccaaacaccgaaaacaataacaaaggagacgggcgaatcactgaccatcaactgtgtcctacgagatagtaactgtgccgcgtccagcacgtactggtatcgcaaaaaatcgggctcaaataacgaggagagcatatcgaaaggtggacgatatgttgaaacagttaacagcggatcaaagtccttttctttgagaattaatgatctaacagttgaagacagtggcacgtatcgatgcaacgtagaaggatatagctgtggactatatagcgtagaagattattacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 157 - DNA, 1044 bp, Artificial Sequence
  Description of Artificial Sequence Synthetic sequence
gctcgagtggaccaaacaccgcaaacaataacaaaggagacgggcgaatcactgaccatcgactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggaggtggcactgtcgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 158 - DNA, 1071 bp, Artificial Sequence
  Description of Artificial Sequence Synthetic sequence
gctcgagtggaccaaacaccgcaaacaataacaaaggagacgggcgaatcactgaccatcgactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 159 - DNA, 1032 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 160 - DNA, 1059 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 161 - DNA, 381 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaatggatccctggaggtgctgttccagggcccaggccaccaccaccaccaccactga

SEQ ID NO: 162 - DNA, 1032 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaattctagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 163 - DNA, 1059 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 164 - DNA, 381 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaatggatccctggaggtgctgttccagggcccaggccaccaccaccaccaccactga

SEQ ID NO: 165 - DNA, 1053 bp, Artificial Sequence
  Description of Artificial Sequence Synthetic sequence
gctcgagtggaccaaacaccgagatcagtaacaaaggagacgggcgaatcactgaccatcaactgtgtcctacgagatacgaactgcccattgtatgcggcctactggtatcgcaaatcaccgggatcaacaaacgaggagaccatatgggcaactggacgatatgttcaaacagttaacagcggacttaagaccttttctttgagaattaatgatctaacttctgaagacagcggcacgtatcgatgcaacggccagacctggtgtcccgccactaagaacaatgatatttcaggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 166 - DNA, 1071 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagaggatatcgaaaggtggacgatatgttgaaacagttaacagcggatcaaagtccttttctttgagaattaatgatctaacagttgaagacggtggcacgtatcgttgcggtgtctggcccggctataactggtgtgggagttgtgattaccgtggcgactataatttttgcggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 167 - DNA, 1083 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagttatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtctaccccggatatagttggtgtgcggccagctgtgactctgtgcccgcggaagtctatgctacattttgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 168 - DNA, 1074 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgaactatgcattgggcagcacgtgctggtatcgaaaaaaatcgggctcaacaaacggcgagagcatatcgaaaggtggacgatatgttgaaacagttaacagcggatcaaagtccttttctttgagaattaatgatctaacagttgaagacggtggcacgtatcgttgcggtgtcggggtggtgtttgggcgctgtgacttctgtgctcttgagagaaaaaactatgctgtatgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 169 - DNA, 1077 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgacctatgcattgggcaacacgtgctggtatcgaaaaaaatcgggctcatcaactgaggagagcatatcgaaaggtggacgatatgttgaaacagttaacagcggatcaaagtccttttctttgagaattaatgatctaacagttgaagacggtggcacgtatcgttgcggtgtctggcccggatataactggtgtggatcctgtgactacgttcatgatgctctacatgtattctgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 170 - DNA, 1071 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatacattggacaacacgtgctggtatcgaaaaaaatcgggctcaacaaaggaggaggaaatatcgaaaggtggacgatatgttgaaacagttaacagccggtcaaagtccttttctttgagaattaatgatctaatagttgaagacggtggcacgtatcgttgcggtgtcggatacttctgtggacactctctgacccaagagtcttgcgggctctatactacatgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 171 - DNA, 393 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatacattggacaacacgtgctggtatcgaaaaaaatcgggctcaacaaaggaggaggaaatatcgaaaggtggacgatatgttgaaacagttaacagccggtcaaagtccttttctttgagaattaatgatctaatagttgaagacggtggcacgtatcgttgcggtgtcggatacttctgtggacactctctgacccaagagtcttgcgggctctatactacatgcggaggtggcactgtcgtgactgtgaatggatccctggaggtgctgttccagggcccaggccaccaccaccaccaccactga

SEQ ID NO: 172 - DNA, 1086 bp, Artificial Sequence
  Description of Artificial Sequence Synthetic sequence
gctcgagtggaccaaacaccgagatcagtaacaaaggagacgggcgaatcactgaccatcaactgtgtcctacgtgaagcgaggtatgaattgggcaagacgtgctggtatcgaaaaagagcgggctcaacaaacgaggagagcatatccgcaggtggacgatatgttgaaacagttatcagcggatcaaagtccttttctttgagaattaatgatctaacagttgaagacggtggcacgtatcgttgtggtgtcccccctgccggctggtgttccaaggttagctgtgaccacgtcggcgcaggtgtgcttgaaccagctgcatgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 173 - DNA, 1062 bp, Artificial Sequence
  Description of Artificial Sequence Synthetic sequence
gctcgagtggaccaaacaccgcaaacaataacaaaggccacgggcgaatcactgaccatcaactgtgtcctacgagatgcgagttatgcattgggcagaacgtgctggtatcgaaaaaaatcgggctcaacaaacgaggagagcatatcgctaggtggacgatatgttgaaacagttaacagcggatcaaagtccttttctttgagaattaatgatctaacagttgaagacggtggcacgtatcgttgcggtgcccaccgctggtggacggtgtgcgggatgccggccccctatgctgagagcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 174 - DNA, 384 bp, Artificial Sequence
  Description of Artificial Sequence Synthetic sequence
gctcgagtggaccaaacaccgcaaacaataacaaaggccacgggcgaatcactgaccatcaactgtgtcctacgagatgcgagttatgcattgggcagaacgtgctggtatcgaaaaaaatcgggctcaacaaacgaggagagcatatcgctaggtggacgatatgttgaaacagttaacagcggatcaaagtccttttctttgagaattaatgatctaacagttgaagacggtggcacgtatcgttgcggtgcccaccgctggtggacggtgtgcgggatgccggccccctatgctgagagcggaggtggcactgtcgtgactgtgaatggatccctggaggtgctgttccagggcccaggccaccaccaccaccaccactga

SEQ ID NO: 175 - DNA, 1080 bp, Artificial Sequence
  Description of Artificial Sequence Synthetic sequence
gctcgagtggaccaaacaccgcaaacaataacaaaggagacgggcgaatcactgaccatcaactgtgttctccgagatagtaattgtgtattggagagcacgtactggcttcgcaaaaaatcgggctcaacaaacgaggagagtatatcgaaaggtggacgatatgttgaaacagtttacagcggatcaaagtccttttctttgagaattaatgatctaacagttgaagacagtggcacgtatcgatgcaagggattggtatggccatatatagatggccggtatagggatgactgcgtgctgggcggatccgcagtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 176 - DNA, 402 bp, Artificial Sequence
  Description of Artificial Sequence Synthetic sequence
gctcgagtggaccaaacaccgcaaacaataacaaaggagacgggcgaatcactgaccatcaactgtgttctccgagatagtaattgtgtattggagagcacgtactggcttcgcaaaaaatcgggctcaacaaacgaggagagtatatcgaaaggtggacgatatgttgaaacagtttacagcggatcaaagtccttttctttgagaattaatgatctaacagttgaagacagtggcacgtatcgatgcaagggattggtatggccatatatagatggccggtatagggatgactgcgtgctgggcggaagcgcagtatacggaggtggcactgtcgtgactgtgaatggatccctggaggtgctgttccagggcccaggccaccaccaccaccaccactga

SEQ ID NO: 177 - DNA, 1071 bp, Artificial Sequence
  Description of Artificial Sequence Synthetic sequence
gctcgagtggaccaaacaccgagatcagtaacaaaggagacgggcgaatcattgaccatcaactgtgtcctacgagaggcgagatatgagttgggcagaacgtgttggtatcgaaaaaaatcgggctcaagaaacgaggagagcatatcggaaggtggacgatatgttgaaacagttaacagcggatcaaagtccttttctttgagaattaatgatctaacagttgaagacggtggcacgtatcgttgcggtgtccccgccgccggctggtgccccgagcggtgcgctcctgatcccccgtatgctgtatgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 178 - DNA, 1062 bp, Artificial Sequence
  Description of Artificial Sequence Synthetic sequence
gctcgagtggaccaaacaccgcaaacaataacaaaggagacgggcgaatcactgaccatcaactgtgtcctacgagataataactgtgcattgtccagcacgtactggtatcgcgaaaaatcgggctcaacaaacttggagagcatatcgctaggtgggagatatgttgaaacagttaacagcggatcaaagtccttttctttgagaattaatgatctaacagttgaagacagtggcacgtatcgatgcaaggtatggggcaacgaggggagctatgactgcggactggagagtgctgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 179 - DNA, 1071 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgggcagcacgtactggtatcgcaaaaaatcgggctcaacaaacgaggagaacatatcgaaaggtggacgatatgttgaaacagttaggagcggatcaaagtccttgtctttgagaattaatgatctaacagttgaagactctggcacgtatcgatgcaaggtgtcccgactaagctggtatccagagagtcgctggaactgcgacaactatgatgtatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 180 - DNA, 393 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgggcagcacgtactggtatcgcaaaaaatcgggctcaacaaacgaggagaacatatcgaaaggtggacgatatgttgaaacagttaggagcggatcaaagtccttgtctttgagaattaatgatctaacagttgaagactctggcacgtatcgatgcaaggtgtcccgactaagctggtatccagagagtcgctggaactgcgacaactatgatgtatacggagatggcactgccgtgactgtgaatggatccctggaggtgctgttccagggcccaggccaccaccaccaccaccactga

SEQ ID NO: 181 - DNA, 1068 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtccattgtccagcacgtactggtatcgcaaaaaatcgggctcaacaaacgaggagagcataccgaaagcgggacgatatgttgaaacagttaaccacggatcaaagtccttttctttgagaattaatgatctaacagttgaagacagtggcacgtatcgatgcaagacggcctcgaacgtatgtcgatccggtggccgaacatcttaccactatgatgtatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 182 - DNA, 390 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtccattgtccagcacgtactggtatcgcaaaaaatcgggctcaacaaacgaggagagcataccgaaagcgggacgatatgttgaaacagttaaccacggatcaaagtccttttctttgagaattaatgatctaacagttgaagacagtggcacgtatcgatgcaagacggcctcgaacgtatgtcgatccggtggccgaacatcttaccactatgatgtatacggagatggcactgccgtgactgtgaatggatccctggaggtgctgttccagggcccaggccaccaccaccaccaccactga

SEQ ID NO: 183 - DNA, 1059 bp, Artificial Sequence
  Description of Artificial Sequence Synthetic sequence
gctcgagtggaccaaacaccgagatcagtaacaaaggagacgggcgaatcactgaccatcaactgtgtcctacgagatagtaactgtgcattgcccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaaggtagagtccctggtattcggtgactgtccttggactggtagagtatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 184 - DNA, 1077 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagcaatgtaatgggcagcacgtgctggtatcgaaaaaaatcgggctcaacaaacgaggagagcatatctttaggtggacgatatgttgaaacagttaacagcggatcaaagtccttttctttgagaattaatgatctaacagttgaagacggtggcacgtatcgttgcggtgtctcacccctggtgatgttcgggggctgtgactacgaaatctgtgctcttgactatgctggctgcggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 185 - DNA, 1068 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgtccagcacgtactggtatcgcaaaaaatcgggctcatcaaacgatgagagcatatcgaaaggtggacgatatgttgaaacagttaacagcggatcaaagtcctttagtttgagaattaatgatctaacagttgaagacagtggcacgtatcgatgcaaggtacgaataattggtatcggtcatgactgcccatggatgggttgggatgatgtatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 186 - DNA, 1080 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgtccagcacgtactggtatcgcaaaaaatcgggctcagcaaacgaggagagcatatcgaaaggtggacgatatgttgaaacagttaacagcggatcaaagtccttttctttgagaattaatgatctaacagttgaagacagtggcacatatcgatgcaacttatacccaggagcccccccgtcgcggactcctatgcgatcatatgactgcagtccccaactggtcttcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 187 - DNA, 1089 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgtccagaacgtactggtatcgcaaaaaatcgggctcaacagacgaggagagcatatcgaaaggtggacgatatgttgaaacagttaacagcggatcaaagtccttttctttgagaattaatgatctaacagttgaagacagtggcacgtatcgatgcaacgtatatagctggtatggcggggatcacaactcatgggactggagatcggatccagcgatggtgtgtggggatgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 188 - DNA, 1083 bp, Artificial Sequence; identical to SEQ ID NO: 60
  Description of Artificial Sequence Synthetic sequence

SEQ ID NO: 189 - DNA, 1476 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtcgtgacagtgaatggtggtggaggatctggcggtggaggaagtggtggcggaggtagtggtggaggcggatctggtggcggaggttccgctagggtggatcagacaccgagatcagtaacaaaggagacgggcgaatctctgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 190 - DNA, 1689 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtcgtgacagtgaatggtggtggaggatctggcggtggtggaagtggtggaggaggtagtaacatcaccgaagaaaatgtgcagaacatgaataatgcgggtgacaagtggagcgcctttttgaaagaacagtcaactttggctcaaatgtacccacttcaggaaattcagaacggaggcggatcttctgctttggaggaacaatacaagacattcctcgacttttttatgattcaactggaagatttgctctaccagttggctctgggtagcggaggcggaggttctggtggcggaggttccgctagggtggatcagacaccgagatcagtaacaaaggagacgggcgaatctctgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 191 - DNA, 1716 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtcgtgacagtgaatggtggtggaggatctggcggtggtggaagtggtggaggaggtagtggcggaggcggatctggttctgctttggaggaacaatacaagacattcctcgacttttttatgattcaactggaagatttgctctaccagttggctctggcatcttggaactataacacaaacatcaccgaagaaaatgtgcagaacatgaataatgcgggtgacaagtggagcgcctttttgaaagaacagtcaactttggctcaaatgtacccacttcaggaaattcagaacggtagcggaggcggaggttctggtggcggaggttccgctagggtggatcagacaccgagatcagtaacaaaggagacgggcgaatctctgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 192 - DNA, 1581 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtcgtgacagtgaatggtggtggaggatctggcggtggtggaagtggtggaggaggtagtggcggaggcggatctggttctgctttggaggaacaatacaagacattcctcgacttttttatgattcaactggaagatttgctctaccagttggctctgggtagcggaggcggaggttctggtggcggaggttccgctagggtggatcagacaccgagatcagtaacaaaggagacgggcgaatctctgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 193 - DNA, 972 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtcgtgacagtgaatggtggtggaggatctggcggtggtggaagtggtggaggaggtagtggcggaggcggatctggtggcggaggttccgctagggtggatcagacaccgagatcagtaacaaaggagacgggcgaatctctgaccatcaactgtgtgctgagggacagcaactgcgccctgtctagcacccactggtacagaaagaaaagcggctctaccaacgaggaaagaatcctgcaaggccgcagatacgtcgagacagtgaattctggctcaaagtccttcagcctgagaattaacgacctgcgggtggaagatagcggaacataccggtgcaaggtgtactggggcaacagctggcaggacaagttctgtcctggactgggcagctacgagtacggcgacggcacagccgtgacagtgaacaaggaaagcggcagcgtgtccagcgagcagctggcccagtttcggagcggcgatggatatatccccgaggcccctagagatggccaggcctacgtgcggaaagacggagagtgggtcctgctgagcaccttcctcggcagaagcctggaagtgctgtttcagggccccggccaccaccaccaccaccaccaccattctgcttggagccacccacagttcgagaagtga

SEQ ID NO: 194 - DNA, 1464 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtggtcacagtgaatggtggtggaggatctggcggtggtggaagtggtggaggaggtagtggcggaggcggatctggtggcggaggttccgctcgggtggatcagacacctcaaacaataacaaaggagacgggcgagtccctgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 195 - DNA, 1464 bp, Artificial Sequence
  Description of Artificial Sequence Synthetic sequence
gctcgagtggaccaaacaccgcaaacaataacaaaggagacgggcgagtccctgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaatggtggtggaggatctggcggtggtggaagtggtggaggaggtagtggcggaggcggatctggtggcggaggttccgctcgggtggatcagacacctcagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtggtcacagtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 196 - DNA, 1464 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtggtcacagtgaatggtggtggaggatctggcggtggtggaagtggtggaggaggtagtggcggaggcggatctggtggcggaggttccgctcgggtggatcagacacctcaaacaataacaaaggagacgggcgagtccctgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 197 - DNA, 1464 bp, Artificial Sequence
  Description of Artificial Sequence Synthetic sequence
gctcgagtggaccaaacaccgcaaacaataacaaaggagacgggcgagtccctgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaatggtggtggaggatctggcggtggtggaagtggtggaggaggtagtggcggaggcggatctggtggcggaggttccgctcgggtggatcagacacctcagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtggtcacagtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 198 - DNA, 567 bp, Artificial Sequence
  Description of Artificial Sequence Synthetic sequence
gctagagtggaccagacccctagatctgtgaccaaggaaaccggcgagagcctgaccatcaattgcgtgctgagggacagcaactgcgccctgtctagcacccactggtacagaaagaaaagcggctctaccaacgaggaaagaatcctgcaaggccgcagatacgtcgagacagtgaattctggctcaaagtccttcagcctgagaattaacgacctgcgggtggaagatagcggaacataccggtgcaaggtgtactggggcaacagctggcaggacaagttctgtcctggactgggcagctacgagtacggcgacggcacagccgtgacagtgaacaaggaaagcggcagcgtgtccagcgagcagctggcccagtttcggagcggcgatggatatatccccgaggcccctagagatggccaggcctacgtgcggaaagacggagagtgggtcctgctgagcaccttcctcggcagaagcctggaagtgctgtttcagggccccggccaccaccaccaccaccaccaccattctgcttggagccacccacagttcgagaagtga

SEQ ID NO: 199 - DNA, 558 bp, Artificial Sequence
  Description of Artificial Sequence Synthetic sequence
gccagagtggaccagacccctcagacaattaccaaggagaccggcgagtccctgaccatcaactgcgtgctgcgggacagcaattgcgccctggccagcacagattggtatagaaagaaatctggatctaccaatgaggaatccatcagcaaaggcggaagatacgtggaaaccgtcaacagcggctctaagagcttcagcctgaggatcaacgacctgaccgtggaagattctggaacataccggtgcaacgcctgggacagctgggagacaagacagctgaagtgcgactacgacgtgtacggcggcggcaccgtggttacagtgaacaaggaaagcggcagcgtgtccagcgagcagctcgctcagttccgcagcggagatggctacatccccgaggcccctagagatggccaggcctacgtgcggaaggacggcgagtgggtcctgctgagcaccttcctgggcagaagcctggaagtgctgttccagggcccaggccaccatcaccaccaccaccaccacagcgcctggagccaccctcaatttgagaagtga

SEQ ID NO: 200 - DNA, 1890 bp, Artificial Sequence
  Description of Artificial Sequence Synthetic sequence
gcccgggtggaccagacccctcagaccatcaccaaggagaccggcgagagcctgacaatcaattgcgtgctgcgggacagcaactgcgctctggcctccactgattggtaccggaagaagagcggcagcacaaacgaggaatccatctctaagggcggcagatacgtggagaccgtgaacagcggcagcaagagctttagcctgagaatcaacgacctgaccgtcgaggactctggcacttatcggtgcaacgcctgggacagctgggaaaccagacagctgaagtgcgactacgacgtgtacggcggcggaaccgtggtgaccgtcaatggcggcggaggaagcggcggcggcggctctggcgggggaggaagcggcggcggcggctctggcggcggcggctccgccagagtggaccaaacacctagaagcgtgaccaaggaaaccggagaatcactgaccatcaactgcgtgctgagagactccaactgtgctctgtctagcacacactggtacaggaagaaaagcggcagcacaaacgaggaaagaatcctgcagggaagacggtacgtggagacagtgaacagcggcagcaagagcttcagcctgcggattaatgacctgcgcgtggaagatagcggcacatatcggtgtaaagtgtactggggcaatagctggcaagataaattctgcccaggcctgggctcttacgagtacggcgatggcacagccgtgaccgtgaacggcggaggcggcagtggaggaggcggttccggcggcggcggcagcggaggcggcggaagcgggggcggcggagctagagtggaccagaccccacgcagcgtgacaaaggaaacaggcgagagcctgaccatcaactgcgtgctgagagacgccagctacgccctgggctccacatgttggtatagaaagaaatctggctccaccaatgaggaaagcatcagcaagggtggaagatacgtggaaaccgtgaacagcggctctaagagtttcagccttagaatcaacgacctgacggtggaggacggcggcacatacagatgcggcgttggaagaggcctgggcgtgggatgtgattaccctctgtgcagctacagctacgccgcttgcggcgacggcaccgccgttacagtcaactctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 201 - DNA, 1890 bp, Artificial Sequence
  Description of Artificial Sequence Synthetic sequence
gctagagtggaccagacccctcagaccattacaaaagagaccggagaatctctgacaatcaactgcgtgctgagagacagcaactgcgccctggcctccaccgactggtaccggaagaaaagtggcagcaccaacgaggaaagcatcagcaaaggcggaagatacgtcgaaaccgtgaacagcggcagtaagagcttcagcctgagaatcaatgacctgactgtcgaagatagcgggacctacagatgcaacgcctgggacagctgggagacaagacagctgaagtgcgactacgacgtgtacggcggaggcacggtcgtgaccgtgaacggcggcggaggctccggcggcggtgggagcggcggcggcggcagcggcggcgggggcagtggcggcggaggctccgcccgggtggatcagacacctcggagcgtgaccaaggaaacaggagagagcctgaccatcaattgcgtgctgagagattccaattgcgccctgagcagcacccactggtacagaaagaagagcggcagcaccaatgaggaaagaatcctgcagggcaggcggtacgtggaaacagtgaacagcggcagcaaaagctttagcctgcggatcaacgacctgcgggtggaagattctggaacttaccggtgcaaggtgtactggggcaacagctggcaggacaagttctgccccggcctgggcagctacgagtacggcgacggtaccgccgtgaccgtgaatggcggcggcggcagcggaggaggaggctcaggcggcggaggaagcggaggcggcggctccggcggaggcggcgctagagtggaccagaccccacaaaccatcaccaaggagaccggcgaatctctgaccatcaactgcgtgctgagagacgccagctacgccctgggctctacatgctggtaccgcaagaagagcggcagcaccaactgggagtctatcggcaaaggcggcagatacgtggagaccgtgaacagcggctccaagtctttttctctgcggatcaacgacctgaccgtggaagatggcgggacgtacagatgtggcgttggacgtggagctggcgtaggttgtgactaccctctgtgcagctacagctatgccgcctgcggcggcggcaccgtggtgacagtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 202 - DNA, 1875 bp, Artificial Sequence
  Description of Artificial Sequence Synthetic sequence
gcccgggtggaccagacccctcagaccatcaccaaggagaccggcgagagcctgacaatcaattgcgtgctgcgggacagcaactgcgctctggcctccactgattggtaccggaagaagagcggcagcacaaacgaggaatccatctctaagggcggcagatacgtggagaccgtgaacagcggcagcaagagctttagcctgagaatcaacgacctgaccgtcgaggactctggcacttatcggtgcaacgcctgggacagctgggaaaccagacagctgaagtgcgactacgacgtgtacggcggcggaaccgtggtgaccgtcaatggcggcggaggaagcggcggcggcggctctggcgggggaggaagcggcggcggcggctctggcggcggcggctccgccagagtggaccaaacacctagaagcgtgaccaaggaaaccggagaatcactgaccatcaactgcgtgctgagagactccaactgtgctctgtctagcacacactggtacaggaagaaaagcggcagcacaaacgaggaaagaatcctgcagggaagacggtacgtggagacagtgaacagcggcagcaagagcttcagcctgcggattaatgacctgcgcgtggaagatagcggcacatatcggtgtaaagtgtactggggcaatagctggcaagataaattctgcccaggcctgggctcttacgagtacggcgatggcacagccgtgaccgtgaacggcggaggcggcagtggaggaggcggttccggcggcggcggcagcggaggcggcggatccggaggcggcggagctcgagtggaccaaacaccgcaaacaataacaaaggagacgggcgaatcactgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 203 - DNA, 1875 bp, Artificial Sequence
  Description of Artificial Sequence Synthetic sequence
gcccgggtggaccagacccctcagaccatcaccaaggagaccggcgagagcctgacaatcaattgcgtgctgcgggacagcaactgcgctctggcctccactgattggtaccggaagaagagcggcagcacaaacgaggaatccatctctaagggcggcagatacgtggagaccgtgaacagcggcagcaagagctttagcctgagaatcaacgacctgaccgtcgaggactctggcacttatcggtgcaacgcctgggacagctgggaaaccagacagctgaagtgcgactacgacgtgtacggcggcggaaccgtggtgaccgtcaatggcggcggaggaagcggcggcggcggctctggcgggggaggaagcggcggcggcggctctggcggcggcggctccgccagagtggaccaaacacctagaagcgtgaccaaggaaaccggagaatcactgaccatcaactgcgtgctgagagactccaactgtgctctgtctagcacacactggtacaggaagaaaagcggcagcacaaacgaggaaagaatcctgcagggaagacggtacgtggagacagtgaacagcggcagcaagagcttcagcctgcggattaatgacctgcgcgtggaagatagcggcacatatcggtgtaaagtgtactggggcaatagctggcaagataaattctgcccaggcctgggctcttacgagtacggcgatggcacagccgtgaccgtgaacggcggaggcggcagtggaggaggcggttccggcggcggcggcagcggaggcggcggatccggaggcggcggagctcgagtggaccaaacaccgcaaacaataacaaaggagacgggcgaatcactgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 204 - DNA, 1485 bp, Artificial Sequence
  Description of Artificial Sequence Synthetic sequence
gctcgagtggaccaaacaccgaggtctgtcaccaaggagaccggcgagtccctcaccattaattgcgtcctgcgagattctaattgtgctttgtctagcactcactggtaccgcaagaaatccggctctacaaatgaagagaggatactgcaaggacgacggtatgtagaaactgttaatagcgggtcaaaatccttctctctgagaatcaatgacctacgagttgaggacagcggcacatatcggtgcaaagtatactggggaaatagttggcaagataaattctgtcctggtttgggctcctatgagtacggagacggcacagccgttacagtgaatggtggtggaggatctggcggtggaggaagtggtggcggaggtagtggtggaggcggatctggtggcggaggttccgcaagagtcgatcaaactcccagatcagtaacaaaggagacgggcgaatctctgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 205 - DNA, 1467 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtggtacagttgtaaccgtcaatggtggtggaggatctggcggtggaggaagtggtggcggaggtagtggtggaggcggatctggtggcggaggttccgcacgcgtcgatcagactccacaaacaattactaaggaaactggcgagtcactcacaattaactgtgtgctcagagattccaattgcgctctggcttccacagattggtataggaaaaagagtggctctacaaatgaggaatctattagcaaaggcggacggtacgtggaaacagtcaacagcggctccaagagtttctctctgcgcatcaacgacctcaccgtcgaagattccggcacctacaggtgcaacgcctgggacagctgggaaacaagacagctcaagtgtgattacgatgtgtatggcggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 206 - DNA, 909 bp, Artificial Sequence
  Description of Artificial Sequence Synthetic sequence
gctcgagtggaccaaacaccacagacaatcactaaagaaacaggcgaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggaggtgggactgtcgtgacagtgaataaggaaagcggcagcgtgtccagcgagcagctggcccagtttcggagcggcgatggcagcctggagctgatcaagctgcggatcatggaaatcgaagccaggatcgctaagattgagaaagaccgggctattaaggagagcggaagcgtctccagcgaacagctcgctcagttccggtccggcgatgctagggtggatcagacaccgagatcagtaacaaaggagacgggcgaatctctgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaatggatccctggaggtgctgttccagggcccaggccaccaccaccaccaccactga

SEQ ID NO: 207 - protein, 24 aa, Artificial Sequence
  Description of Artificial Sequence Synthetic sequence
GGGGSGGGGSGGGGSGGGGSGGGG

SEQ ID NO: 208 - protein, 25 aa, Artificial Sequence
  Description of Artificial Sequence Synthetic sequence
GGGGSGGGGSGGGGSGGGGSGGGGS

SEQ ID NO: 209 - protein, 19 aa, Artificial Sequence
  Description of Artificial Sequence Synthetic sequence
KESGSVSSEQLAQFRSGDG

SEQ ID NO: 210 - protein, 96 aa, Artificial Sequence
  Description of Artificial Sequence Synthetic sequence
GGGGSGGGGSGGGGSNITEENVQNMNNAGDKWSAFLKEQSTLAQMYPLQEIQNGGGSSALEEQYKTFLDFFMIQLEDLLYQLALGSGGGGSGGGGS

SEQ ID NO: 211 - protein, 105 aa, Artificial Sequence
  Description of Artificial Sequence Synthetic sequence
GGGGSGGGGSGGGGSGGGGSGSALEEQYKTFLDFFMIQLEDLLYQLALASWNYNTNITEENVQNMNNAGDKWSAFLKEQSTLAQMYPLQEIQNGSGGGGSGGGGS

SEQ ID NO: 212 - protein, 60 aa, Artificial Sequence
  Description of Artificial Sequence Synthetic sequence
GGGGSGGGGSGGGGSGGGGSGSALEEQYKTFLDFFMIQLEDLLYQLALGSGGGGSGGGGS

SEQ ID NO: 213 - protein, 26 aa, Artificial Sequence
  Description of Artificial Sequence Synthetic sequence
GSLELIKLRIMEIEARIAKIEKDRAI

SEQ ID NO: 214 - protein, 30 aa, Artificial Sequence
  Description of Artificial Sequence Synthetic sequence
MDSKGSSQKGSRLLLLLVVSNLLLPQGVVG

SEQ ID NO: 215 - protein, 19 aa, Artificial Sequence
  Description of Artificial Sequence Synthetic sequence
MGWSCIILFLVATATGVHS

SEQ ID NO: 216 - DNA, 90 bp, Artificial Sequence
  Description of Artificial Sequence Synthetic sequence
atggacagcaagggcagctcccagaagggctcccggctgctgctgctgctggtggtgtccaacctgctgctgcctcagggcgtggtgggc

SEQ ID NO: 217 - DNA, 57 bp, Artificial Sequence
  Description of Artificial Sequence Synthetic sequence
atgggatggtcatgtatcatcctttttctagtagcaactgcaaccggtgtacattcc

SEQ ID NO: 218 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSHHHHHH

SEQ ID NO: 219 - protein, 1318 aa, Artificial Sequence
  Description of Artificial Sequence Synthetic sequence
SSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKNGSGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 220 - protein, 396 aa, Artificial Sequence
  Description of Artificial Sequence Synthetic sequence
HHHHHHHHGPLEVLFQGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLRRLFRKSNLKPFERDISTEIYQAGSTPCNGVEGFNCYFPLQSYGFQPTNGVGYQPYRVVVLSFENKSAPATVCGPGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 221 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPGSHHHHHH

SEQ ID NO: 222 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGSHHHHHH

SEQ ID NO: 223 - protein, 205 aa, Artificial Sequence
  Description of Artificial Sequence Synthetic sequence
NITNLCPFGEVFNATRFASVYAWNRERISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSHHHHHH

SEQ ID NO: 224 - protein, 205 aa, Artificial Sequence
  Description of Artificial Sequence Synthetic sequence
NITNLCPFGEVFNATRFASVYAWNRKRISNCVADYSVLYNSASFSTSECYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSHHHHHH

SEQ ID NO: 225 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDKFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSHHHHHH

SEQ ID NO: 226 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGSHHHHHH

SEQ ID NO: 227 - protein, 513 aa, Artificial Sequence
  Description of Artificial Sequence Synthetic sequence
HHHHHHHHGPLEVLFQGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGSGGSGSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTL

SEQ ID NO: 228 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPGSHHHHHH

SEQ ID NO: 229 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYSPLQSYGFQPTNGVGYQPYRVVVLSFELLHAPATVCGPGSHHHHHH

SEQ ID NO: 230 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYFPLQSYGFQPTNGVGYQPYRVVVLSFELLHAPATVCGPGSHHHHHH

SEQ ID NO: 231 - protein, 205 aa, Artificial Sequence
  Description of Artificial Sequence Synthetic sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGLNCYFPLQSYGFQPTNGVGYQPYRVVVLSFELLHAPATVCGPGSHHHHHH

SEQ ID NO: 232 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatctgtacaggctgttccgcaagtccaacctgaagccatttgagagggacatctctaccgagatctatcaggcaggcagcacaccatgcaacggagtggagggcttcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 233 - DNA, 3957 bp, Artificial Sequence
  Description of Artificial Sequence Synthetic sequence
agcagccagtgcgtgaacctgaccacccgaacccagctgccccctgcctacaccaatagctttacaaggggcgtgtactatcccgataaggtgttccgctctagcgtgctgcactccacacaggacctgtttctgcctttcttttctaatgtgacctggttccacgccatccacgtgagcggcaccaacggcacaaagaggtttgataatccagtgctgccctttaacgacggcgtgtatttcgccagcaccgagaagtccaacatcatccgcggctggatcttcggcaccacactggattctaagacacagagcctgctgatcgtgaacaatgccaccaacgtggtcatcaaggtgtgcgagttccagttttgtaacgacccctttctgggcgtgtactatcacaagaacaataagtcctggatggagtctgagttccgggtgtactcctctgccaacaattgcacatttgagtatgtgagccagcctttcctgatggatctggagggcaagcagggcaacttcaagaacctgcgggagttcgtgtttaagaatatcgacggctacttcaagatctattctaagcacacccctatcaacctggtgagagatctgccacagggctttagcgccctggagcctctggtggacctgccaatcggcatcaacatcaccaggttccagacactgctggccctgcaccgctcctacctgacaccaggcgatagctcctctggatggaccgcaggagcagcagcctactatgtgggctacctgcagccccggacctttctgctgaagtataacgagaatggcaccatcacagacgccgtggattgcgccctggaccctctgtctgagaccaagtgtacactgaagagcttcaccgtggagaagggcatctaccagacaagcaattttcgggtgcagccaaccgagtccatcgtgagattcccaaatatcacaaacctgtgcccctttggcgaggtgttcaacgccaccagattcgccagcgtgtatgcctggaatcggaagagaatctccaactgcgtggccgattactctgtgctgtataactccgcctctttcagcacctttaagtgctacggcgtgtccccaacaaagctgaatgacctgtgctttaccaacgtgtatgccgattctttcgtgatcaggggcgacgaagtgagacagatcgcaccaggacagaccggcaagatcgcagactacaactataagctgcctgacgacttcaccggctgcgtgatcgcctggaatagcaacaatctggattccaaagtgggcggcaactacaattatctgtacaggctgttccgcaagtccaacctgaagccatttgagagggacatctccacagagatctaccaggccggctctaccccctgcaatggcgtggagggctttaactgttacttccccctgcagtcctatggctttcagcctacaaatggcgtgggctaccagccatatcgcgtggtggtgctgagcttcgagctgctgcacgcaccagcaacagtgtgcggacctaagaagtccaccaatctggtgaagaacaagtgcgtgaacttcaacttcaacggactgaccggaacaggcgtgctgaccgagtccaacaagaagttcctgccttttcagcagttcggccgggacatcgccgataccacagacgccgtgagagatccacagaccctggagatcctggacatcacaccctgcagcttcggcggcgtgtccgtgatcacaccaggcaccaatacatctaaccaggtggccgtgctgtaccaggatgtgaattgtaccgaggtgcctgtggcaatccacgcagaccagctgaccccaacatggagggtgtattccaccggctctaacgtgtttcagacaagggcaggatgcctgatcggagcagagcacgtgaacaatagctacgagtgcgatatccccatcggcgccggcatctgtgcctcctatcagacccagacaaacagccctgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggtacccactggttcgtgacacaaagaaacttctacgaaccccagattatcaccaccgacaacaccttcgtgagtggcaattgtgacgtggtcatcggaatcgtgaacaacacagtgtacgaccctctgcaatctgagctggactctattaaggaagagctggacaagatccataaaaacggctctggaggttctggagatatcatcaagctgctgaacgagcaagtgaataaggagatgcagtctagcaacctgtatatgtccatgtcctcttggtgttacacccactctctggacggagcaggactgttcctgtttgatcacgccgccgaggagtacgagcacgccaagaagctgatcatctttctgaatgagaacaatgtgcctgtgcagctgaccagcatctccgccccagagcacaagttcgagggcctgacacagatctttcagaaggcctatgagcacgagcagcacatctctgagagcatcaacaatatcgtggaccacgccatcaagtccaaggatcacgccacattcaactttctgcagtggtacgtggccgagcagcacgaggaggaggtgctgttcaaggacatcctggataagatcgagctgatcggcaacgagaatcacggcctgtatctggccgatcagtacgtgaagggcatcgccaagagcaggaagtccggctcttga

SEQ ID NO: 234 - DNA, 1191 bp, Artificial Sequence
  Description of Artificial Sequence Synthetic sequence
caccatcatcaccaccaccaccacggacccctggaggtgctgttccagggaccaaacatcaccaatctgtgccccttcggcgaggtgtttaacgccacaagatttgccagcgtgtacgcctggaaccggaagagaatctccaattgcgtggccgactattctgtgctgtacaatagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgatttcacaggctgcgtgatcgcctggaatagcaacaatctggattccaaagtgggcggcaactacaattatctgcgcaggctgttccgcaagtccaacctgaagccttttgagagggacatctctaccgagatctatcaggccggcagcacaccatgcaacggcgtggagggcttcaattgttattttcctctgcagtcttacggcttccagccaaccaatggcgtgggctatcagccctacagggtggtggtgctgagctttgagaacaagtccgcccccgccacagtgtgcggacctggctctggaggaggaggagagagccaggtgcgccagcagttcagcaaggatatcgagaagctgctgaacgagcaagtgaataaggagatgcagtctagcaacctgtatatgtccatgtcctcttggtgttacacccactctctggacggagcaggactgttcctgtttgatcacgccgccgaggagtacgagcacgccaagaagctgatcatctttctgaatgagaacaatgtgcctgtgcagctgaccagcatctccgccccagagcacaagttcgagggcctgacacagatctttcagaaggcctatgagcacgagcagcacatctctgagagcatcaacaatatcgtggaccacgccatcaagtccaaggatcacgccacattcaactttctgcagtggtacgtggccgagcagcacgaggaggaggtgctgttcaaggacatcctggataagatcgagctgatcggcaacgagaatcacggcctgtatctggccgatcagtacgtgaagggcatcgccaagagccggaagtccggctcttga

SEQ ID NO: 235 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatcggtacaggctgttccgcaagtccaacctgaagccatttgagagggacatctctaccgagatctatcaggcaggcagcacaccatgcaacggagtgcagggcttcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 236 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaccatcgcagactacaactataagctgcctgacgacttcaccggctgcgtgatcgcctggaatagcaacaatctggattccaaagtgggcggcaactacaattatctgtacaggctgttccgcaagtccaacctgaagccatttgagagggacatctctaccgagatctatcaggcaggcagcacaccatgcaacggagtgaagggcttcaattgttattttccactgcagtcttacggcttccagcccacctacggcgtgggctatcagccttacagggtggtggtgctgagctttgagctgctgcacgcccccgccacagtgtgcggacctggcagccatcatcaccaccaccactga

SEQ ID NO: 237 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccgggagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatctgtacaggctgttccgcaagtccaacctgaagccatttgagagggacatctctaccgagatctatcaggcaggcagcacaccatgcaacggagtggagggcttcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 238 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctccgagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatctgtacaggctgttccgcaagtccaacctgaagccatttgagagggacatctctaccgagatctatcaggcaggcagcacaccatgcaacggagtggagggcttcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 239 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacaagttcaccggctgcgtgatcgcctggaatagcaacaatctggattccaaagtgggcggcaactacaattatctgtacaggctgttccgcaagtccaacctgaagccatttgagagggacatctctaccgagatctatcaggcaggcagcacaccatgcaacggagtggagggcttcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 240 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatcggtacaggctgttccgcaagtccaacctgaagccatttgagagggacatctctaccgagatctatcaggcaggcagcaagccatgcaacggagtggagggcttcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 241 - DNA, 1542 bp, Artificial Sequence
  Description of Artificial Sequence Synthetic sequence
caccatcatcaccaccaccaccacggacccctggaggtgctgttccagggaccaaacatcaccaatctgtgccccttcggcgaggtgtttaacgccacaagatttgccagcgtgtacgcctggaaccggaagagaatctccaattgcgtggccgactattctgtgctgtacaatagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgatttcacaggctgcgtgatcgcctggaatagcaacaatctggattccaaagtgggcggcaactacaattatctgtacaggctgttccgcaagtccaacctgaagccttttgagagggacatctctaccgagatctatcaggccggcagcacaccatgcaacggcgtggagggcttcaattgttattttcctctgcagtcttacggcttccagccaaccaatggcgtgggctatcagccctacagggtggtggtgctgagctttgagctgctgcacgcccccgccacagtgtgcggacctggctctggaggaagcggaagcagccagtgcgtgaatctgaccacaagaacccagctgccccctgcctacaccaatagctttacaaggggcgtgtactatcccgataaggtgttccgctctagcgtgctgcactccacacaggacctgtttctgcctttcttttctaatgtgacctggttccacgccatccacgtgagcggcaccaacggcacaaagaggtttgataatccagtgctgccctttaacgacggcgtgtatttcgccagcaccgagaagtccaacatcatccgcggctggatcttcggcaccacactggattctaagacacagagcctgctgatcgtgaacaatgccaccaacgtggtcatcaaggtgtgcgagttccagttttgtaacgacccctttctgggcgtgtactatcacaagaacaataagtcctggatggagtctgagttccgggtgtactcctctgccaacaattgcacatttgagtatgtgagccagcctttcctgatggatctggagggcaagcagggcaacttcaagaacctgcgggagttcgtgtttaagaatatcgacggctacttcaagatctattctaagcacacccctatcaacctggtgagagatctgccacagggctttagcgccctggagcctctggtggacctgccaatcggcatcaacatcaccaggttccagacactgctggccctgcaccgctcctacctgacaccaggcgatagctcctctggatggaccgcaggagcagcagcctactatgtgggctacctgcagccccggacctttctgctgaagtataacgagaatggcaccatcacagacgccgtggattgcgccctggaccctctgtctgagaccaagtgtacactgtga

SEQ ID NO: 242 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatcagtacaggctgttccgcaagtccaacctgaagccatttgagagggacatctctaccgagatctatcaggcaggcagcacaccatgcaacggagtggagggcttcaattgttattccccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 243 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatctgtacaggctgttccgcaagtccaacctgaagccatttgagagggacatctctaccgagatctatcaggcaggcagcacaccatgcaacggagtggagggcttcaattgttattccccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 244 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatcagtacaggctgttccgcaagtccaacctgaagccatttgagagggacatctctaccgagatctatcaggcaggcagcacaccatgcaacggagtggagggcttcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 245 - DNA, 618 bp, Artificial Sequence
  Description of Artificial Sequence Synthetic sequence
aacatcaccaatctgtgcccattcggcgaggtgtttaatgccacaagattcgccagcgtgtacgcctggaaccggaagagaatctccaattgcgtggccgactattctgtgctgtacaacagcgcctccttctctacctttaagtgctatggcgtgagccctaccaagctgaacgacctgtgcttcacaaacgtgtacgccgactcctttgtgatccggggcgatgaggtgagacagatcgcaccaggacagaccggcaagatcgcagactacaactataagctgcctgacgacttcaccggctgcgtgatcgcctggaatagcaacaatctggattccaaagtgggcggcaactacaattatctgtacaggctgttccgcaagtccaacctgaagccatttgagagggacatctctaccgagatctatcaggcaggcagcacaccatgcaacggagtggagggcctcaattgttattttccactgcagtcttacggcttccagcccaccaatggcgtgggctatcagccttacagggtggtggtgctgagctttgagctgctgcacgcccccgccacagtgtgcggacctggcagccatcatcaccaccaccactga

SEQ ID NO: 246 - protein, 8 aa, Ginglymostoma cirratum
DSNCALSS

SEQ ID NO: 247 - protein, 8 aa, Ginglymostoma cirratum
DSNCALAS

SEQ ID NO: 248 - protein, 8 aa, Ginglymostoma cirratum
SASYALGS

SEQ ID NO: 249 - protein, 8 aa, Ginglymostoma cirratum
DSNCALSS

SEQ ID NO: 250 - protein, 8 aa, Ginglymostoma cirratum
DSNCALGS

SEQ ID NO: 251 - protein, 8 aa, Ginglymostoma cirratum
DASYALGS

SEQ ID NO: 252 - protein, 8 aa, Ginglymostoma cirratum
DASYALGS

SEQ ID NO: 253 - protein, 8 aa, Ginglymostoma cirratum
DSNCAASS

SEQ ID NO: 254 - protein, 8 aa, Ginglymostoma cirratum
DSNCALSS

SEQ ID NO: 255 - protein, 8 aa, Ginglymostoma cirratum
DSNCALSS

SEQ ID NO: 256 - protein, 8 aa, Ginglymostoma cirratum
DSNCTLSR

SEQ ID NO: 257 - protein, 8 aa, Ginglymostoma cirratum
DTNCPLYA

SEQ ID NO: 258 - protein, 8 aa, Ginglymostoma cirratum
DASYALGS

SEQ ID NO: 259 - protein, 8 aa, Ginglymostoma cirratum
DSNCALSR

SEQ ID NO: 260 - protein, 8 aa, Ginglymostoma cirratum
DANYALGS

SEQ ID NO: 261 - protein, 8 aa, Ginglymostoma cirratum
DATYALGN

SEQ ID NO: 262 - protein, 8 aa, Ginglymostoma cirratum
DASYTLDN

SEQ ID NO: 263 - protein, 8 aa, Ginglymostoma cirratum
EARYELGK

SEQ ID NO: 264 - protein, 8 aa, Ginglymostoma cirratum
DASYALGR

SEQ ID NO: 265 - protein, 8 aa, Ginglymostoma cirratum
DSNCVLES

SEQ ID NO: 266 - protein, 8 aa, Ginglymostoma cirratum
EARYELGR

SEQ ID NO: 267 - protein, 8 aa, Ginglymostoma cirratum
DNNCALSS

SEQ ID NO: 268 - protein, 8 aa, Ginglymostoma cirratum
DSNCALGS

SEQ ID NO: 269 - protein, 8 aa, Ginglymostoma cirratum
DSNCPLSS

SEQ ID NO: 270 - protein, 8 aa, Ginglymostoma cirratum
DSNCALPS

SEQ ID NO: 271 - protein, 8 aa, Ginglymostoma cirratum
DASNVMGS

SEQ ID NO: 272 - protein, 8 aa, Ginglymostoma cirratum
DSNCALSS

SEQ ID NO: 273 - protein, 8 aa, Ginglymostoma cirratum
DSNCALSS

SEQ ID NO: 274 - protein, 8 aa, Ginglymostoma cirratum
DSNCALSR

SEQ ID NO: 275 - protein, 8 aa, Ginglymostoma cirratum
DASNVMGS

SEQ ID NO: 276 - protein, 20 aa, Ginglymostoma cirratum
KVYWGNSWQDKFCPGLGSYE

SEQ ID NO: 277 - protein, 17 aa, Ginglymostoma cirratum
NAWDSWETRQLKCDYDV

SEQ ID NO: 278 - protein, 25 aa, Ginglymostoma cirratum
GVIRDELVWGIAGYNNRPCAPPYAA

SEQ ID NO: 279 - protein, 15 aa, Ginglymostoma cirratum
KVSCLAIPIGPHYDV

SEQ ID NO: 280 - protein, 18 aa, Ginglymostoma cirratum
KVLSIGWHDPMGLRGCDV

SEQ ID NO: 281 - protein, 21 aa, Ginglymostoma cirratum
GVGRGLGVGCDYPLCSYSYAA

SEQ ID NO: 282 - protein, 21 aa, Ginglymostoma cirratum
GVGRGAGVGCDYPLCSYSYAA

SEQ ID NO: 283 - protein, 15 aa, Ginglymostoma cirratum
NVEGYSCGLYSVEDY

SEQ ID NO: 284 - protein, 20 aa, Ginglymostoma cirratum
KVYWGNSWQDKFCPGLGSYE

SEQ ID NO: 285 - protein, 16 aa, Ginglymostoma cirratum
HVRLRYDCESVIDFRV

SEQ ID NO: 286 - protein, 16 aa, Ginglymostoma cirratum
ASKIRYDCQGIIAYDI

SEQ ID NO: 287 - protein, 14 aa, Ginglymostoma cirratum
NGQTWCPATKNNDI

SEQ ID NO: 288 - protein, 21 aa, Ginglymostoma cirratum
GVWPGYNWCGSCDYRGDYNFC

SEQ ID NO: 289 - protein, 26 aa, Ginglymostoma cirratum
NVYSWYGGDHNSWDWRSDPAMVCGDV

SEQ ID NO: 290 - protein, 21 aa, Ginglymostoma cirratum
GVGVVFGRCDFCALERKNYAV

SEQ ID NO: 291 - protein, 22 aa, Ginglymostoma cirratum
GVWPGYNWCGSCDYVHDALHVF

SEQ ID NO: 292 - protein, 21 aa, Ginglymostoma cirratum
GVGYFCGHSLTQESCGLYTTC

SEQ ID NO: 293 - protein, 25 aa, Ginglymostoma cirratum
GVPPAGWCSKVSCDHVGAGVLEPAA

SEQ ID NO: 294 - protein, 18 aa, Ginglymostoma cirratum
GAHRWWTVCGMPAPYAES

SEQ ID NO: 295 - protein, 23 aa, Ginglymostoma cirratum
KGLVWPYIDGRYRDDCVLGGSAV

SEQ ID NO: 296 - protein, 20 aa, Ginglymostoma cirratum
GVPAAGWCPERCAPDPPYAV

SEQ ID NO: 297 - protein, 17 aa, Ginglymostoma cirratum
KVWGNEGSYDCGLESAV

SEQ ID NO: 298 - protein, 20 aa, Ginglymostoma cirratum
KVSRLSWYPESRWNCDNYDV

SEQ ID NO: 299 - protein, 19 aa, Ginglymostoma cirratum
KTASNVCRSGGRTSYHYDV

SEQ ID NO: 300 - protein, 16 aa, Ginglymostoma cirratum
KVESLVFGDCPWTGRV

SEQ ID NO: 301 - protein, 22 aa, Ginglymostoma cirratum
GVSPLVMFGGCDYEICALDYAG

SEQ ID NO: 302 - protein, 19 aa, Ginglymostoma cirratum
KVRIIGIGHDCPWMGWDDV

SEQ ID NO: 303 - protein, 23 aa, Ginglymostoma cirratum
NLYPGAPPSRTPMRSYDCSPQLV

SEQ ID NO: 304 - protein, 26 aa, Ginglymostoma cirratum
NVYSWYGGDHNSWDWRSDPAMVCGDV

SEQ ID NO: 305 - protein, 22 aa, Ginglymostoma cirratum
GVSPLVMFGGCDYEICALDYAG

SEQ ID NO: 306 - protein, 9 aa, Ginglymostoma cirratum
TNEERILQG

SEQ ID NO: 307 - protein, 9 aa, Ginglymostoma cirratum
TNEESISKG

SEQ ID NO: 308 - protein, 9 aa, Ginglymostoma cirratum
TNEESISKG

SEQ ID NO: 309 - protein, 9 aa, Ginglymostoma cirratum
TNEESISKG

SEQ ID NO: 310 - protein, 9 aa, Ginglymostoma cirratum
TNEESITKG

SEQ ID NO: 311 - protein, 9 aa, Ginglymostoma cirratum
TNEESISKG

SEQ ID NO: 312 - protein, 9 aa, Ginglymostoma cirratum
TNWESIGKG

SEQ ID NO: 313 - protein, 9 aa, Ginglymostoma cirratum
NNEESISKG

SEQ ID NO: 314 - protein, 9 aa, Ginglymostoma cirratum
TNEERILQG

SEQ ID NO: 315 - protein, 9 aa, Ginglymostoma cirratum
TNEESISKG

SEQ ID NO: 316 - protein, 9 aa, Ginglymostoma cirratum
TNLESISKG

SEQ ID NO: 317 - protein, 9 aa, Ginglymostoma cirratum
TNEETIWAT

SEQ ID NO: 318 - protein, 9 aa, Ginglymostoma cirratum
TNEERISKG

SEQ ID NO: 319 - protein, 9 aa, Ginglymostoma cirratum
TDEESISKG

SEQ ID NO: 320 - protein, 9 aa, Ginglymostoma cirratum
TNGESISKG

SEQ ID NO: 321 - protein, 9 aa, Ginglymostoma cirratum
STEESISKG

SEQ ID NO: 322 - protein, 9 aa, Ginglymostoma cirratum
TKEEEISKG

SEQ ID NO: 323 - protein, 9 aa, Ginglymostoma cirratum
TNEESISAG

SEQ ID NO: 324 - protein, 9 aa, Ginglymostoma cirratum
TNEESISLG

SEQ ID NO: 325 - protein, 9 aa, Ginglymostoma cirratum
TNEESISKG

SEQ ID NO: 326 - protein, 9 aa, Ginglymostoma cirratum
RNEESISEG

SEQ ID NO: 327 - protein, 9 aa, Ginglymostoma cirratum
TNLESISLG

SEQ ID NO: 328 - protein, 9 aa, Ginglymostoma cirratum
TNEENISKG

SEQ ID NO: 329 - protein, 9 aa, Ginglymostoma cirratum
TNEESIPKA

SEQ ID NO: 330 - protein, 9 aa, Ginglymostoma cirratum
TNEESISKG

SEQ ID NO: 331 - protein, 9 aa, Ginglymostoma cirratum
TNEESISLG

SEQ ID NO: 332 - protein, 9 aa, Ginglymostoma cirratum
SNDESISKG

SEQ ID NO: 333 - protein, 9 aa, Ginglymostoma cirratum
ANEESISKG

SEQ ID NO: 334 - protein, 9 aa, Ginglymostoma cirratum
TDEESISKG

SEQ ID NO: 335 - protein, 9 aa, Ginglymostoma cirratum
TNEESISLG

SEQ ID NO: 336 - protein, 4 aa, Ginglymostoma cirratum
SGSK

SEQ ID NO: 337 - protein, 4 aa, Ginglymostoma cirratum
SGSK

SEQ ID NO: 338 - protein, 4 aa, Ginglymostoma cirratum
SGSK

SEQ ID NO: 339 - protein, 4 aa, Ginglymostoma cirratum
SGPL

SEQ ID NO: 340 - protein, 4 aa, Ginglymostoma cirratum
SGSK

SEQ ID NO: 341 - protein, 4 aa, Ginglymostoma cirratum
SGSK

SEQ ID NO: 342 - protein, 4 aa, Ginglymostoma cirratum
SGSK

SEQ ID NO: 343 - protein, 4 aa, Ginglymostoma cirratum
SGSK

SEQ ID NO: 344 - protein, 4 aa, Ginglymostoma cirratum
SGSK

SEQ ID NO: 345 - protein, 4 aa, Ginglymostoma cirratum
SGSK

SEQ ID NO: 346 - protein, 4 aa, Ginglymostoma cirratum
SGSK

SEQ ID NO: 347 - protein, 4 aa, Ginglymostoma cirratum
SGLK

SEQ ID NO: 348 - protein, 4 aa, Ginglymostoma cirratum
SGSK

SEQ ID NO: 349 - protein, 4 aa, Ginglymostoma cirratum
SGSK

SEQ ID NO: 350 - protein, 4 aa, Ginglymostoma cirratum
SGSK

SEQ ID NO: 351 - protein, 4 aa, Ginglymostoma cirratum
SGSK

SEQ ID NO: 352 - protein, 4 aa, Ginglymostoma cirratum
SRSK

SEQ ID NO: 353 - protein, 4 aa, Ginglymostoma cirratum
SGSK

SEQ ID NO: 354 - protein, 4 aa, Ginglymostoma cirratum
SGSK

SEQ ID NO: 355 - protein, 4 aa, Ginglymostoma cirratum
SGSK

SEQ ID NO: 356 - protein, 4 aa, Ginglymostoma cirratum
SGSK

SEQ ID NO: 357 - protein, 4 aa, Ginglymostoma cirratum
SGSK

SEQ ID NO: 358 - protein, 4 aa, Ginglymostoma cirratum
SGSK

SEQ ID NO: 359 - protein, 4 aa, Ginglymostoma cirratum
HGSK

SEQ ID NO: 360 - protein, 4 aa, Ginglymostoma cirratum
SGSK

SEQ ID NO: 361 - protein, 4 aa, Ginglymostoma cirratum
SGSK

SEQ ID NO: 362 - protein, 4 aa, Ginglymostoma cirratum
SGSK

SEQ ID NO: 363 - protein, 4 aa, Ginglymostoma cirratum
SGSK

SEQ ID NO: 364 - protein, 4 aa, Ginglymostoma cirratum
SGSK

SEQ ID NO: 365 - protein, 4 aa, Ginglymostoma cirratum
SGSK

SEQ ID NO: 366 - protein, 356 aa, Artificial Sequence
  Description of Artificial Sequence Synthetic sequence
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 367 - protein, 353 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 357 aa, Artificial Sequence
  Description of Artificial Sequence Synthetic sequence
ARVDQTPRSVTKETGESLTINCVLRDASYALGSTCWYRKKSGSTNEESISKGGRYVETVNSGSKSFSLRINDLTVEDGGTYRCGVGRGLGVGCDYPLCSYSYAACGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 369 - protein, 357 aa, Artificial Sequence
  Description of Artificial Sequence Synthetic sequence
ARVDQTPRSVTKETGESLTINCVLRDASYALGSTCWYRKKSGSTNEESISKGGRYVETVNSGSKSFSLRINDLTVEDGGTYRCGVGRGLGVGCDYPLCSYSYAACGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 352 aa, Artificial Sequence
  Description of Artificial Sequence Synthetic sequence
ARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 371 - protein, 352 aa, Artificial Sequence
  Description of Artificial Sequence Synthetic sequence
ARVDQTPQTITKETGESLTINCVLRDSNCTLSRTYWYRKKSGSTNLESISKGGRYVETVNSGSKSFSLRINDLTVEDSGTYRCASKIRYDCQGIIAYDIYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 357 aa, Artificial Sequence
  Description of Artificial Sequence Synthetic sequence
ARVDQTPQTITKETGESLTINCVLRDASYALGSTCWYRKKSGSTNWESIGKGGRYVETVNSGSKSFSLRINDLTVEDGGTYRCGVGRGAGVGCDYPLCSYSYAACGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 351 aa, Artificial Sequence
  Description of Artificial Sequence Synthetic sequence
ARVDQTPKTITKETGESLTINCVLRDSNCAASSTYWYRKKSGSNNEESISKGGRYVETVNSGSKSFSLRINDLTVEDSGTYRCNVEGYSCGLYSVEDYYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 374 - protein, 356 aa, Artificial Sequence
  Description of Artificial Sequence Synthetic sequence
ARVDQTPQTITKETGESLTIDCVLRDSNCALSSTHWYRKKSGSTNEERILQGRRYVETVNSGSKSFSLRINDLRVEDSGTYRCKVYWGNSWQDKFCPGLGSYEYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 375 - protein, 352 aa, Artificial Sequence
  Description of Artificial Sequence Synthetic sequence
ARVDQTPQTITKETGESLTINCVLRDSNCALSSTYWYRKKSGSTNEESISKGGRYVETVNSGSKSFSLRINDLTVEDSGTYRCHVRLRYDCESVIDFRVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 353 aa, Artificial Sequence
  Description of Artificial Sequence Synthetic sequence
ARVDQTPQTITKATGESLTINCVLRDASYALGRTCWYRKKSGSTNEESISLGGRYVETVNSGSKSFSLRINDLTVEDGGTYRCGAHRWWTVCGMPAPYAESGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 356 aa, Artificial Sequence
  Description of Artificial Sequence Synthetic sequence
ARVDQTPQTITKETGESLTINCVLRDSNCALGSTYWYRKKSGSTNEENISKGGRYVETVRSGSKSLSLRINDLTVEDSGTYRCKVSRLSWYPESRWNCDNYDVYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 378 - DNA, 1071 bp, Artificial Sequence
  Description of Artificial Sequence Synthetic sequence
gccagggtggaccaaacaccgagatcagtaacaaaggagacgggcgaatcactgaccatcaactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 379 - DNA, 1062 bp, Artificial Sequence
  Description of Artificial Sequence Synthetic sequence
gctcgcgtcgaccagactccacagacaatcactaaagaaacaggggaatcactgacaatcaactgcgtgctgagagactccaactgcgccctggcctccaccgactggtacaggaagaagagcggctccacaaacgaggagtctatcagcaagggcggccggtatgtggagacagtgaactccggctctaagagcttctccctgcggatcaatgacctgaccgtggaggattctggcacatacagatgcaatgcctgggatagctgggagacaaggcagctgaagtgcgattatgatgtctatggagggggaaccgtcgtcacagtcaactctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 380 - DNA, 1074 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcgggcgcggacttggtgtgggctgtgactatcccctgtgttcttactcgtatgctgcatgcggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 381 - DNA, 1074 bp, Artificial Sequence
  Description of Artificial Sequence Synthetic sequence
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtcgggcgcggacttggtgtgggctgtgactatcccctgtgttcttactcgtatgctgcatgcggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 382 - DNA, 1059 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 383 - DNA, 1059 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtacattgtccaggacgtactggtatcgcaaaaaatcgggctcaacaaacctggagagcatatcgaaaggtggacgatatgttgaaacagttaacagcggatcaaagtccttttctttgagaattaatgatctaacagttgaggacagtggcacgtatcgatgcgcctcaaagatacgttatgactgccaggggattatcgcctatgatatatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 384 - DNA, 1074 bp, Artificial Sequence
  Description of Artificial Sequence Synthetic sequence
gctcgagtggaccaaacaccgcaaacaataacaaaggagacgggcgaatcactgaccatcaactgtgtcctacgagatgcgagctatgcattgggcagcacgtgctggtatcgaaaaaaatcgggctcaacaaactgggagagcatagggaaaggtggacgatatgttgaaacagttaacagcggatcaaagtccttttctttgagaattaatgatctaacagttgaagacggtggcacgtatcgttgcggtgtcgggcgcggagctggtgtgggctgtgactatcccctgtgctcttactcgtatgctgcatgcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 385 - DNA, 1056 bp, Artificial Sequence
  Description of Artificial Sequence Synthetic sequence
gctcgagtggaccaaacaccgaaaacaataacaaaggagacgggcgaatcactgaccatcaactgtgtcctacgagatagtaactgtgccgcgtccagcacgtactggtatcgcaaaaaatcgggctcaaataacgaggagagcatatcgaaaggtggacgatatgttgaaacagttaacagcggatcaaagtccttttctttgagaattaatgatctaacagttgaagacagtggcacgtatcgatgcaacgtagaaggatatagctgtggactatatagcgtagaagattattacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 386 - DNA, 1071 bp, Artificial Sequence
  Description of Artificial Sequence Synthetic sequence
gctcgagtggaccaaacaccgcaaacaataacaaaggagacgggcgaatcactgaccatcgactgtgtcctacgagatagtaactgtgcattgtccagcacgcactggtatcgcaaaaaatcgggctcaacaaacgaggagagaatattgcaaggtcgacgatatgttgaaacagttaacagcggatcaaagtccttttctttgagaattaatgatctaagagttgaagacagtggcacgtatcgatgcaaggtatattggggcaatagctggcaggataagttctgccctggattgggcagctatgaatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 387 - DNA, 1059 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgccacgtccgcttgcggtatgactgcgagagcgtgatagattttcgtgtatacggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 388 - DNA, 1062 bp, Artificial Sequence
  Description of Artificial Sequence Synthetic sequence
gctcgagtggaccaaacaccgcaaacaataacaaaggccacgggcgaatcactgaccatcaactgtgtcctacgagatgcgagttatgcattgggcagaacgtgctggtatcgaaaaaaatcgggctcaacaaacgaggagagcatatcgctaggtggacgatatgttgaaacagttaacagcggatcaaagtccttttctttgagaattaatgatctaacagttgaagacggtggcacgtatcgttgcggtgcccaccgctggtggacggtgtgcgggatgccggccccctatgctgagagcggaggtggcactgtcgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 389 - DNA, 1071 bp, Artificial Sequence
  Description of Artificial Sequence Synthetic sequence
gctcgagtggaccaaacaccgcaaacaataacaaaggagacgggcgaatcactgaccatcaactgtgtcctacgagatagtaactgtgcattgggcagcacgtactggtatcgcaaaaaatcgggctcaacaaacgaggagaacatatcgaaaggtggacgatatgttgaaacagttaggagcggatcaaagtccttgtctttgagaattaatgatctaacagttgaagactctggcacgtatcgatgcaaggtgtcccgactaagctggtatccagagagtcgctggaactgcgacaactatgatgtatacggagatggcactgccgtgactgtgaattctagagagcccaaatcttgtgacaaaggtctcgaagtgctattccaaggtcctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 390 - protein, 631 aa, Artificial Sequence
  Description of Artificial Sequence Synthetic sequence
ARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGGGGSGGGGSGGGGSGGGGSGGGGSARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNGGGGSGGGGSGGGGSGGGGSGGGGARVDQTPRSVTKETGESLTINCVLRDSNCALSSTHWYRKKSGSTNEERILQGRRYVETVNSGSKSFSLRINDLRVEDSGTYRCKVYWGNSWQDKFCPGLGSYEYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 391 - protein, 622 aa, Artificial Sequence
  Description of Artificial Sequence Synthetic sequence
ARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGSARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNGGGGSGGGGSGGGGSGGGGSGGGGARVDQTPQTITKETGESLTINCVLRDSNCALASTDWYRKKSGSTNEESISKGGRYVETVNSGSKSFSLRINDLTVEDSGTYRCNAWDSWETRQLKCDYDVYGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 392 - protein, 622 aa, Artificial Sequence
  Description of Artificial Sequence Synthetic sequence
ARVDQTPQTITKATGESLTINCVLRDASYALGRTCWYRKKSGSTNEESISLGGRYVETVNSGSKSFSLRINDLTVEDGGTYRCGAHRWWTVCGMPAPYAESGGGTVVTVNGGGGSGGGGSGGGGSGGGGSGGGGSARVDQTPQTITKATGESLTINCVLRDASYALGRTCWYRKKSGSTNEESISLGGRYVETVNSGSKSFSLRINDLTVEDGGTYRCGAHRWWTVCGMPAPYAESGGGTVVTVNGGGGSGGGGSGGGGSGGGGSGGGGARVDQTPQTITKATGESLTINCVLRDASYALGRTCWYRKKSGSTNEESISLGGRYVETVNSGSKSFSLRINDLTVEDGGTYRCGAHRWWTVCGMPAPYAESGGGTVVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 393 - protein, 631 aa, Artificial Sequence
  Description of Artificial Sequence Synthetic sequence
ARVDQTPQTITKETGESLTINCVLRDSNCALGSTYWYRKKSGSTNEENISKGGRYVETVRSGSKSLSLRINDLTVEDSGTYRCKVSRLSWYPESRWNCDNYDVYGDGTAVTVNGGGGSGGGGSGGGGSGGGGSGGGGSARVDQTPQTITKETGESLTINCVLRDSNCALGSTYWYRKKSGSTNEENISKGGRYVETVRSGSKSLSLRINDLTVEDSGTYRCKVSRLSWYPESRWNCDNYDVYGDGTAVTVNGGGGSGGGGSGGGGSGGGGSGGGGARVDQTPQTITKETGESLTINCVLRDSNCALGSTYWYRKKSGSTNEENISKGGRYVETVRSGSKSLSLRINDLTVEDSGTYRCKVSRLSWYPESRWNCDNYDVYGDGTAVTVNSREPKSCDKGLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 394 - protein, 110 aa, Ginglymostoma cirratum
ARVDQTPQTITKETGESLTINCVLRDSNCGVASTDWYRKKSGSTNEESISKGGRYVETVNSRSKSFSLRINDLTVEDSGTYRCNAWDRWETRQLNCDYDVYGGGTVVTVN

SEQ ID NO: 395 - protein, 8 aa, Ginglymostoma cirratum
DSNCGVAS

SEQ ID NO: 396 - protein, 17 aa, Ginglymostoma cirratum
NAWDRWETRQLNCDYDV

SEQ ID NO: 397 - protein, 9 aa, Ginglymostoma cirratum
TNEESISKG

SEQ ID NO: 398 - protein, 4 aa, Ginglymostoma cirratum
SRSK

(19) United States
(12) Patent Application Publication
(10) Pub. No.: US 2023/0331823 A1
(43) Pub. Date: Oct. 19, 2023

(54) SHARK-DERIVED BINDING MOLECULES FOR SARS-COV-2 CORONAVIRUS AND USES THEREOF

(71) Applicants: THE HENRY M. JACKSON FOUNDATION FOR THE ADVANCEMENT OF MILITARY MEDICINE, INC., Bethesda, MD (US); THE GOVERNMENT OF THE UNITED STATES, AS REPRESENTED BY THE SECRETARY OF THE ARMY, Fort Detrick, MD (US); UNIVERSITY OF MARYLAND, BALTIMORE, Baltimore, MD (US)

(72) Inventors: Michael Gordon JOYCE, Silver Spring, MD (US); Kayvon MODJARRAD, Silver Spring, MD (US); Helen DOOLEY, Baltimore, MD (US); Agnes HAJDUCZKI, Silver Spring, MD (US); Wei-Hung CHEN, Silver Spring, MD (US)

(21) Appl. No.: 18/026,199

(22) PCT Filed: Sep. 15, 2021
(86) PCT No.: PCT/US2021/050545

§ 371 Date: Mar. 14, 2023

Related U.S. Application Data
(60) Provisional application No. 63/078,677, filed on Sep. 15, 2020.

(51) Int. Cl. C07K 16/10; A61P 31/14; G01N 33/569

(52) U.S. Cl. CPC C07K 16/10; A61P 31/14; G01N 33/56983; C07K 2317/565; C07K 2317/569; C07K 2317/24; C07K 2317/92

(57) ABSTRACT

Disclosed herein are shark-derived antibodies and binding molecules with high binding affinity for SARS-CoV-2 coronavirus and the use thereof. Some of the shark-derived antibodies and binding molecules also demonstrate cross-reactivity with and neutralization of other related sarbecoviruses. The complementarity determining region (CDR) sequences and binding characteristics for these antibodies and binding molecules are provided. Also disclosed are the use of these antibodies and binding molecules to detect SARS-CoV-2 coronavirus or other related sarbecoviruses or to prevent or treat SARS-CoV-2 coronavirus or other related sarbecovirus infections.

### CROSS-REFERENCE TO RELATED APPLICATIONS

This application claims priority to U.S. Provisional Application No. 63/078,677 filed 15 Sep. 2020, the entire contents of which are hereby incorporated by reference in their entirety into the present application.

### STATEMENT REGARDING FEDERALLY SPONSORED RESEARCH

This invention was made with government support under W81XWH-18-2-0040 awarded by the United States Army Medical Research and Materiel Command. The government has certain rights in the invention.

### SUBMISSION OF SEQUENCE LISTING

The Sequence Listing associated with this application is filed in electronic format as a text file and hereby incorporated by reference into the specification in its entirety. The name of the text file containing the Sequence Listing is HMJ_173_PCT_SL and the size of the text file is 544 KB.

### FIELD

This application discloses shark-derived antibodies, antigen-binding fragments thereof, and other binding molecules derived therefrom that specifically bind to proteins of SARS-CoV-2 coronavirus and other coronaviruses. It also discloses the use of these antibodies, antigen-binding fragments thereof, and binding molecules derived therefrom.

### BACKGROUND

The emergence of SARS-CoV-2 marks the seventh coronavirus to be isolated from humans, and the third to cause a severe disease-named COVID-19-after severe acute respiratory syndrome (SARS) and Middle East respiratory syndrome (MERS). The rapid spread of SARS-CoV-2, and the grave risk it poses to global health, prompted the World Health Organization to declare the COVID-19 outbreak to be a public health emergency of international concern on 30 Jan. 2020 and a pandemic on 11 Mar. 2020. The rapidly evolving epidemiology of the pandemic and absence of licensed prophylactics or therapeutics for the disease have accelerated the need to elucidate the molecular biology of this novel coronavirus and translate the information gained into the rapid development of vaccines and treatments.

The surface Spike (S) glycoprotein of SARS-CoV-2 binds host receptor angiotensin-converting enzyme 2 (ACE-2) to mediate cell entry. Coronavirus S glycoproteins contain three segments: a large ectodomain, a single-pass transmembrane anchor and a short intracellular tail. The ectodomain consists of a receptor-binding subunit, S1, which contains two sub-domains: one at the N-terminus and the other at the C-terminus. The latter comprises the receptor-binding domain (RBD), which serves the vital function of attaching the virus to the host receptor and triggering a conformational change in the protein that results in fusion with the host cell membrane through the S2 subunit.

There is an urgent need for antibodies and binding molecules that bind with high affinity to SARS-CoV-2 S glycoproteins. These antibodies and binding molecules are important for accurate diagnosis and effective treatment of sarbecovirus associated diseases, such as SARS-CoV-2 associated diseases, including COVID-19.

### SUMMARY

Disclosed herein are single variable domain antibodies (nanobodies or VNARs) derived from sharks and antigen-binding fragments thereof. These nanobodies and antigen-binding fragments thereof specifically bind to SARS-CoV-2 Spike glycoprotein as well as proteins of other coronaviruses. These specific nanobodies and antigen-binding fragments thereof can be used for diagnosing SARS-CoV-2 infection and associated diseases, including COVID-19, as well as for preventing and/or treating SARS-CoV-2 coronavirus infection. These nanobodies and antigen-binding fragments thereof can also be utilized for immunogen testing and validation, potential viral inhibitors, and other applications related to SARS-CoV-2. These nanobodies and antigen-binding fragments thereof may be used for the development of antibody therapeutics and/or use in vaccine immunogen development and product release. Sequences encoding the antigen specific antigen-binding domain are derived from a variable region of the immunoglobulin isotype IgNAR found in cartilaginous fishes. The nanobodies and antigen-binding fragments thereof can be linked together and/or conjugated to other proteins to form derivative binding molecules, including binding molecules with multi-specific reactivity against different epitopes of the SARS-CoV-2 Spike glycoprotein, the Spike glycoprotein from different SARS-CoV-2 variants, including, for example, the B.1.1.7 variant (aka, alpha variant), B.1.351 variant (aka, beta variant), and B.1.617.2 variant (aka, delta variant), and other coronavirus epitopes. For example, the nanobodies and antigen-binding fragments thereof can be conjugated to an Fe domain, such as a human Fc domain (e.g., IgG1 Fc domain or IgM Fc domain), or a protein with self-assembling multimerization properties (e.g., ferritin or lumazine synthase).

This application describes the isolation and characterization of multiple shark-derived single variable domain antibodies and antigen-binding fragments thereof that specifically target multiple epitopes in the receptor binding domain (RBD) or N-terminus domain (NTD) of the SARS-CoV-2 Spike glycoprotein and binding molecules derived therefrom. In certain embodiments, the shark-derived single variable domain antibodies and antigen-binding fragments thereof bind the SARS-CoV-2 Spike glycoprotein (e.g., RBD or NTD) with high affinity in the nM range, including, for example 1-100 nM or even less. In certain embodiments, the shark-derived single variable domain antibodies and antigen-binding fragments thereof bind the SARS-CoV-2 Spike glycoprotein from the W-1 strain as well as variants thereof (e.g., one or more of the B.1.1.7 variant (aka, alpha variant), B.1.351 variant (aka, beta variant), and B.1.617.2 variant (aka, delta variant)) with high affinity. In addition to high binding affinity, shark-derived single variable domain antibodies disclosed herein have improved properties over conventional antibodies having both heavy and light chains, including increased production levels, reduced size (much smaller than typical antibodies), high stability and solubility, and the ability to easily link nanobodies together to create multi-specific or repetitive arrays to increase binding capacity.

### BRIEF DESCRIPTION OF THE DRAWINGS

FIG. 1 depicts a typical shark immunization schedule used for the SARS-CoV-2 immunogens.

FIG. 2 depicts the shark immunization plasma ELISA binding against SARS-CoV-2 RBD. Sharks were immunized with a SARS-CoV-2 RBD immunogen (left), RBD-Ferritin immunogen (pCoV131) (middle) or a Spike trimer-Ferritin immunogen (pCoV-1B-06-PL) (right).

FIG. 3A-3C depict binding kinetics of shark nanobodies MoB3-3D8 (FIG. 3A) & MoB3-3D8_Fc (FIG. 3B) and AliB5-2D8 (FIG. 3C) to the SARS-CoV-2 RBD measured by biolayer interferometry. Kinetic constants were calculated using a minimum of four dilutions of the RBD (concentration shown on the right of the figures) and fitted using a 1:1 Langmuir binding model.

FIG. 4A-4D depict shark nanobody AliB5-2D8 competition studies using a set of SARS-CoV-2 antibodies. SARS-CoV-2 antibody is loaded onto a probe, followed by binding to the SARS-CoV-2 RBD. This Antibody-RBD complex is then incubated with a shark nanobody to assess binding. A control nanobody (5A7) and PBS-only is used to indicate no binding. Measurements were carried out using Biolayer Interferometry. Both CR3022 (FIG. 4A) and 240CD (FIG. 4B) antibodies show competition against AliB5-2D8, while CV1 (FIG. 4C) and CVH1 (FIG. 4D) antibodies do not compete with AliB5-2D8.

FIG. 5A-5C depict shark nanobody MoB3-3D8 competition studies using a set of SARS-CoV-2 antibodies. SARS-CoV-2 antibody is loaded onto a probe, followed by binding to the SARS-CoV-2 RBD. This Antibody-RBD complex is then incubated with a shark nanobody to assess binding. A control nanobody (5A7) and PBS-only is used to indicate no binding. Measurements were carried out using Biolayer Interferometry. Both CR3022 (FIG. 5A) and CVH1 (FIG. 5B) antibodies show no competition against MoB3-3D8, while CV1 (FIG. 5C) antibody competes with MoB3-3D8.

FIG. 6A-6C depict oB3-3D8_Fc fusion antibody binding studies carried out using Biolayer Interferometry. FIG. 6A: The MoB3-3D8_Fc fusion antibody binds to a Spike Ferritin nanoparticle (SpFN) vaccine candidate at five concentrations ranging from 30 μg/ml to 2 μg/ml. FIG. 6B: The MoB3-3D8_Fc fusion antibody binds to the SARS-CoV-2 RBD at 30 μg/ml. FIG. 6C: The MoB3-3D8_Fc fusion antibody binds to the SARS-CoV-2 stabilized Spike trimer (S-2P) at concentrations ranging from 30 μg/ml to 10 μg/ml. AHC denotes the anti-human capture probe used in the Biolayer Interferometry binding assays.

FIG. 7 depicts MoB3-3D8 nanobody binding studies carried out using Biolayer Interferometry. The MoB3-3D8 nanobody was assessed for binding to three Coronavirus stabilized spike trimers (S-2P) at 30 μg/ml. Clear binding is seen to the SARS-CoV-2 stabilized trimer, with minimal binding seen to the HKU9 stabilized trimer. No binding was observed to the SARS-CoV stabilized trimer molecule.

FIG. 8A-8B depict the results of ACE-2 blocking assay. Shark nanobody MoB3-3D8_Fc chimera antibody was assessed for ACE-2 blocking alongside a set of SARS-CoV-2 antibodies. SARS-CoV-2 antibodies were loaded onto an AHC probe, followed by binding to the SARS-CoV-2 RBD. This Antibody-RBD complex is then incubated with human ACE-2 receptor to assess ACE-2 blocking. Measurements were carried out using Biolayer Interferometry. FIG. 8A: CV1 shows no competition against ACE-2 receptor, while CR3022 shows partial blocking and CVH1 and MoB3-3D8_Fc show blocking of the RBD-ACE-2 receptor interaction. FIG. 8B: ACE-2 blocking is plotted as a graph.

FIG. 9A-9F depict design of shark antibodies in multiple presentation modalities. FIG. 9A: Knob-in-hole design allows two different shark nanobodies to be merged into a single antibody. FIG. 9B: Beads on a string format, with multiple shark antibodies merged onto a single molecule. FIG. 9C: Beads on a string with a linked Fc domain. FIG. 9D: Addition of a shark nanobody onto a human antibody. FIG. 9E: Ferritin-shark nanobody with a single nanobody. FIG. 9F: Ferritin-shark nanobody with multiple nanobodies.

FIG. 10A-10B depict the structure (FIG. 10A) and sequence (FIG. 10B) of SARS-CoV-2 RBD immunogen.

FIG. 11A-11E depict the structure-based development of the SpFN_pCoV1B-06-PL immunogen. FIG. 11A: Sequence of the SpFN_pCoV1B-06-PL vaccine candidate. The sequence of the SARS-CoV-2 Spike glycoprotein ectodomain is shown in black, with the altered furin cleavage site (GSAS) highlighted in bold and underlined. The two stabilizing proline mutations (PP) are also highlighted in bold and underlined. The six amino acid linker sequence (GSGGSG) connecting the S glycoprotein ectodomain to the Helicobacter pylori ferritin sequence is highlighted in bold. FIG. 11B: A schematic of the SpFN_pCoV1B-06-PL vaccine candidate is shown with numbering based on the SARS-CoV-2 Spike glycoprotein. The number of residues, and percentage content of the SARS-CoV-2 residues in the SpFN_pCoV1B-06-PL vaccine candidate is shown. FIG. 11C: Model of the SpFN_pCoV1B-06-PL vaccine candidate is shown in ribbon format in two views, (left) view down the four-fold axis, (right) view down the three-fold axis. FIG. 11D: Negative-stain electron microscope image of the SpFN_pCoV1B-06-PL vaccine candidate. FIG. 11E: Three-dimensional negative stain map of the SpFN_pCoV1B-06-PL vaccine candidate determined using about 10,000 particles.

FIG. 12A-12E depict structure-based development of the SpFN_pCoV131 (His8-3c-RBD-Ferritin-Y453R-518LLH to NKS) immunogen as a vaccine candidate. FIG. 12A: Sequence of the SpFN_pCoV131 vaccine candidate. The sequence of the SARS-CoV-2 RBD is in bold. The linker connecting the RBD to the ferritin sequence of Helicobacter pylori ferritin sequence comprises the amino acid sequence GSGGGGESQVRQQFSK. The mutations are shown in bold and underlined. FIG. 12B: A schematic of the SpFN_pCoV131 vaccine candidate is shown with numbering based on the SARS-CoV-2 Spike glycoprotein. The number of residues, and percentage content of the SARS-CoV-2 residues in the SpFN_pCoV131 vaccine candidate is shown. FIG. 12C: Model of the SpFN_pCoV131 vaccine candidate is shown in ribbon format shaded as in FIG. 12B. FIG. 12D: Negative-stain electron microscope image of the SpFN_pCoV131 vaccine candidate. FIG. 12E: Two-dimensional class averages of the SpFN_pCoV131 vaccine candidate.

FIG. 13A-13M depict identification and characterization of cross-reactive and sarbecovirus neutralizing shark IgNAR antibodies. FIG. 13A: Immunization schedule of two nurse sharks (“Pink” and “Red”) for antibody generation. SARS-CoV-2 protein was used to immunize sharks, followed by antibody panning and identification of the variable domain of shark IgNAR: ShAb01 and ShAb02 VNARs. FIG. 13B: ELISA of shark IgNAR responses against SARS-CoV-2 RBD at three timepoints following immunizations. FIG. 13C: ELISA of ShAb01a and ShAb02a against SARS-CoV-2 S-2P trimer (left) and RBD (right). FIG. 13D: Biolayer interferometry (BLI) measurement of ShAb01 and ShAb02 affinity to SARS-CoV-2 RBD. FIG. 13E: BLI measurement of ShAb01 and ShAb02 ability to block binding of human ACE2 receptor to SARS-CoV-2 S-2P trimer (left) and RBD (right). FIG. 13F: Neutralization of SARS-CoV-2 variants and SARS-CoV-1 pseudoviruses by ShAb01 and ShAB02. Data are representative of two independent experiments. FIG. 13G-I: Epitope binning of RBD-directed ShAb molecules via a BLI-based competition assay. Values represent the % residual binding of the indicated second antibody (ShAb01 or ShAb02) after saturation of the antigen (RBD molecule) with the indicated first antibody (left column). Shading from dark to light indicates competition strength ranging from strong competition (0-33%) to minimal competition (>75%). Competition groups are indicated by black boxes. Human ACE2-Fc and CR3022 were used as controls. ACE2 binding is completely blocked by ShAb01a, ShAb09a, and ShAb23a. FIG. 13J: K18-hACE2 mice SARS-CoV-2 challenge study (n=13/group, 7 females and 6 males) received isotype control IgG (black), or ShAb01 (triangle) or ShAb02 (circle), one day prior to challenge with 1.25×10⁴ PFU of SARS-CoV-2 (WA-1/2020). A subset of mice was sacrificed 2 days post-challenge for BAL viral load measurements. The remaining mice (n=8/group) were assessed daily for weight and clinical symptoms. Percentage of initial weight is plotted. FIG. 13K: Survival of K18-hACE2 mice for the 3 study groups. Legend is shown in panel. Survival curves were compared individually to the isotype control using a Mantel-Cox log-rank test. FIG. 13L: Clinical score of the K18-hACE2 study groups. FIG. 13M: SARS-CoV-2 viral loads in BAL were measured 2 days post-challenge in a subset of animals (n=5/group) by plaque assay. Asterisks indicate significance compared to the antibody isotype control group by one-way ANOVA with Dunnett's multiple comparisons test, ****P<0.0001; **P<0.01.

FIG. 14A-14H depict structure-based design of dual-specificity ShAb-based immunotherapeutics. FIG. 14A: Crystal structure of ShAb01 and ShAb02 VNARs in complex with SARS-CoV-2 RBD. The important residues of ShAb01 are indicated and highlighted in two zoom-in panels, with extensive contacts made by the CDR 3 loop. FIG. 14B: The important residues of ShAb02 are indicated and highlighted in two zoom-in panels, with extensive contacts made by the VNAR CDR loops 1-3. FIG. 14C: Epitope mapping by alanine mutagenesis scanning analysis identified SARS-CoV-2 RBD residue Tyr369 as an important contact residue for ShAb01 binding. FIG. 14D: Epitope mapping by alanine mutagenesis scanning analysis identified SARS-CoV-2 RBD residues Arg346, Asn354, and Lys356 as important contact residues for ShAb02 binding. FIG. 14E: SARS-CoV-2 RBD (gray) is shown in surface representation, with ShAb01 and ShAb02 and ten other VNARs shown in smooth surface representation. FIG. 14F: SARS-CoV-2 RBD is shown in surface representation with ShAb01 and ShAb02 VNARs in ribbon representation. The distance from ShAb02 VNAR C-terminus to either the N- or C-terminus of ShAb01 VNAR is shown. FIG. 14G: Design schematics of bispecific molecules BiShAb21a and ShAb1H2K “knob-in-hole” constructs, and the trivalent ShAb-foldon constructs. FIG. 14H: Pseudovirus neutralization of SARS-CoV-2 WA-1 and Alpha and Beta variants, and SARS-CoV-1 by ShAb1H2K, BiShAb21a, ShAb01Folden and Shab02Folden. IC50 values are shown in ng mL⁻¹.

FIG. 15A-15D depict immunization of nurse sharks with SARS-CoV-2 immunogens. FIG. 15A: Nurse sharks “Purple” and “Blue” were immunized with 100 μg soluble SARS-CoV-2 Spike-ferritin nanoparticle (pCoV-1B-06-PL) as shown in the immunization schedule, followed by panning and identification of ShAb03-16 and ShAb22-29 VNAR molecules. FIG. 15B: Nurse sharks “Green” and “Yellow” were immunized with 100 μg soluble SARS-CoV-2 RBD-ferritin nanoparticle (pCoV131) as shown in the immunization schedule, followed by panning and identification of ShAb17-21 VNAR molecules. FIG. 15C: ELISA analysis of SARS-CoV-2 Spike IgNAR responses for “Purple” (left) and “Blue” (right) at week 0 (PB), 2 (B1), 6 (B2), and 10 (B5) at different plasma dilutions, with SARS-CoV-2 RBD as the antigen. FIG. 15D: ELISA analysis of SARS-CoV-2 Spike IgNAR responses for “Green” (left) and “Yellow” (right) at week 0 (PB), 6 (B4), 10 (B5), and 36 (B6) at different plasma dilutions, with SARS-CoV-2 RBD as the antigen.

FIG. 16A-16B depict crystal structure of ShAb01 and ShAb02 VNAR domains in complex with SARS-CoV-2 RBD. FIG. 16A: Top view of RBD-ShAb01-ShAb02 structure complex. Two notable contact areas of ShAb02 VNAR with SARS-CoV-2 RBD. SARS CoV-2 RBD is shown in surface presentation and ShAb02 and ShAb01 VNARs are shown in ribbon presentation. Contact residues are indicated in the two zoom-in panels. FIG. 16B: Epitope footprint of hACE2, ShAb01 VNAR, and ShAb02 VNAR with different views. Overlay of footprints are shown as enclosed dashed line (right column). SARS-CoV-2 RBD is shown in surface presentation and hACE2, ShAb01, and ShAb02 footprints on the RBD are indicated. ShAb02 VNAR epitope overlaps marginally with the hACE2 epitope explaining the observed blocking.

FIG. 17 depicts the structural and sequence analysis of the ShAb01 and ShAb02 epitopes. Analysis of the ShAb01 and ShAb02 binding footprint across betacoronaviruses. The ShAb01 (top left) and ShAb02 (middle left) epitopes on SARS-CoV-2 (China.Wuhan.30Dec19.402132) RBD is compared across betacoronaviruses. The epitope is numbered according to the Wuhan reference sequence. The strength of the interaction between the ShAbs and the spike protein is indicated by the height of the histogram bars above the sequence alignment (top center). Sequences are ordered according to their phylogenetic relationships based on a maximum likelihood phylogenetic tree derived from amino acid RBD sequences. The RBD structure is shown in surface representation and depicts mutations between SARS-CoV-1 and SARS-CoV-2 in dark shading. The ShAb01 and ShAb02 epitopes are outlined.

FIG. 18A-18H depict schematic representations of VNAR-conjugate immunotherapeutic molecules. Immunotherapeutic designs are shown in two representations: 2D cartoon format, and linear diagram format. FIG. 18A-18E: Immunotherapeutics based on VNAR-dimerization using a human Fc domain, including “knob-in-hole” designs (FIG. 18A: Single VNAR domain; FIG. 18B: Single VNAR domain, bispecific and incorporating “knob-in-hole”; FIG. 18C: Multi VNAR domains, bispecific; FIG. 18D: Multi domain, tri-specific; FIG. 18E: Multi domain, quad-specific and incorporating “knob-in-hole”). FIG. 18F-18H: Immunotherapeutics shown in panels utilize a trimerization domain (T4 fibritin (foldon)) to increase avidity. Single or multiple VNARs can be linked in series to further increase immunotherapeutic affinity and avidity binding effects and enables targeting of multiple epitopes (FIG. 18F: Single VNAR domain; FIG. 18G: Multi VNAR domains, bispecific; FIG. 18H: Multi VNAR domains, tri-specific).

FIG. 19A-19C depict design of linker length for VNAR-conjugate immunotherapeutic molecules. FIG. 19A: A model of a bispecific-VNAR molecule is shown with ShAb VNARs linked in series (e.g., BiShAb21a). The SARS-CoV-2 RBD molecule is shown in surface representation with ShAb02 and ShAb01 VNARs shown in ribbon representation. A model of the flexible Gly-Ser linker and distance between ShAb02 C-terminus and ShAb01 N-terminus is indicated with cartoon representation of the human Fe domain and the second ShAb02-ShAb01 “‘arm” of the immunotherapeutic. FIG. 19B: A model of a bispecific-VNAR domain is shown utilizing two chains with Fc domain “knob-in-hole” to allow ShAb01 and ShAb02 VNARs to be incorporated into a single immunotherapeutic. FIG. 19C: The SARS-CoV-2 S-2P molecule is shown in an “open” conformation with the RBD domains in an “up” conformation. The ShAb02 VNAR molecule is shown in ribbon representation and measurements between the location of three ShAb02 VNAR molecules bound to the S-2P trimer are indicated by dotted lines. The 24 amino acid linkers between the foldon domain and each VNAR equates to about 84 Å in distance, ensuring sufficient distance to allow binding of all three RBDs.

FIG. 20A-20D depict biolayer interferometry affinity measurements of four examples of ShAb-based immunotherapeutics binding to SARS-CoV-2 RBD variant molecules and SARS-CoV-1 RBD (FIG. 20A: ShAb01-Fc fusion molecule (ShAb01a); FIG. 20B: ShAb02-Fc fusion molecule (ShAb02a); FIG. 20C: ShAb01-ShAb02 knob-in-hole Fc fusion molecule (ShAb01H02K); FIG. 20D: two VNARs in series and linked to a human Fc domain (BiShAb21a)). ShAb-based immunotherapeutics were loaded onto a AHC probe and incubated with four concentrations of each RBD molecule. Affinities were determined by applying a 1:1 Langmuir fitting equation and global fitting.

FIG. 21A-21D depict biolayer interferometry (BLI) affinity measurements of ShAb-based immunotherapeutics binding to SARS-CoV-2 RBD and SARS-CoV-1 RBD. FIG. 21A: BLI affinity measurement of ShAb04a, ShAb06a, ShAb7a, ShAb08a, ShAb10a, and ShAb1 1a to SARS-CoV-2 NTD. FIG. 21B: BLI affinity measurement of ShAb01H06K (“1H6K”) and ShAb06H02K (“6H2K”) to SARS-CoV-2 NTD. FIG. 21C: BLI affinity measurement of ShAb01Folden (“ShAb01F”) and ShAb02Folden (“ShAb02F”) binding to SARS-CoV-2 S-2P. FIG. 21D: BLI assessment of binding of RBD-targeting ShAbs to SARS-CoV-1 RBD. VNARs that are part of the ShAb01a epitope competition group all show binding to SARS-CoV-1 RBD (left panel). Most VNARs that fall into the ShAb02a epitope competition group do not show binding to SARS-CoV-1 RBD with the exception of ShAb19a and ShAb20a, which showed strong binding to SARS-CoV-1 RBD.

FIG. 22A-22C depict ACE2 blocking activity of RBD-specific ShAbs (FIG. 22A-22B) and NTD-specific ShAbs (FIG. 22C). ShAbs were assessed for their ability to block hACE2 binding to SARS-CoV-2 RBD (FIG. 22B) or SARS-CoV-2 S-2P (FIG. 22A and FIG. 22C) in a BLI-based assay. The half maximal effective concentration (EC50) in μg ml⁻¹ is indicated in parentheses.

FIG. 23A-23D depict the characterization of ShAb-Fc fusion molecules. FIG. 23A: ELISA assessing ShAbs binding to SARS-CoV-2 proteins (RBD, NTD, and S-2P) coated on the plate. The control antibody is CR3022 for the RBD and S-2P binding experiments, and WRAIR-2039 for the NTD binding experiment. Background binding against the blocking agent BSA has been subtracted from all data points at the corresponding antibody dilutions. FIG. 23B: Biolayer interferometry (Octet) determining binding of SARS-CoV-2 proteins to immobilized ShAbs. FIG. 23C: Pseudovirus neutralization assay using SARS-CoV-1 and SARS-CoV-2 variants pseudoviruses assessing the inhibition of viral cell entry by RBD-targeting ShAbs. FIG. 23D: Pseudovirus neutralization assay using NTD-targeting ShAbs. For the ELISA and neutralization assays, error bars indicate standard deviation for duplicate wells.

FIG. 24A-24E depict the characterization of bispecific ShAb immunotherapeutics. FIG. 24A: ELISA assessing combinations of ShAb01 and ShAb02 binding to SARS-CoV-2 proteins (RBD and S-2P) coated on the plate. The original ShAb01 and ShAb02 are included for reference. FIG. 24B: Pseudovirus neutralization assay using SARS-CoV-1 and SARS-CoV-2 variant pseudoviruses assessing the inhibition of viral cell entry by various bispecific ShAb01-ShAb02 combinations. Neutralizing antibody P2B-2F6 is included for reference. FIG. 24C: ELISA using ShAb01-ShAb06 and ShAb02-ShAB06 knob-in-hole combinations binding to SARS-CoV-2 S-2P. Data uses BSA coated plates as a negative control. FIG. 24D: Pseudovirus neutralization assay using knob-in-hole ShAb combinations. FIG. 24E: Pseudovirus neutralization assay using Tri-specific ShAb molecules: TriShAb216a and TriShAb217a. For the ELISA and neutralization assays, error bars indicate standard deviation for duplicate wells.

FIG. 25A-25D depict shark nanobodies protective immunity in K18-ACE2 transgenic mice. NTD-targeting antibody ShAb06a alone or in combination with ShAb02a was given to K18-hACE2 mice one day prior to challenge with 1.25×10⁴ PFU of SARS-CoV-2 (WA-1/2020). In addition, one group of mice were given a combination of ShAb02a and ShAb06a one day after infection, as a therapeutic intervention. Mice received isotype control IgG (black), ShAb06a (gray triangle), ShAb02a+ShAB06a (circle), or therapeutic ShAb02a+ShAb06a (square). FIG. 25A: Survival curves of K18-ACE2 mice (n=8/group, 4 females and 4 males). Groups were compared to each other using a Gehan-Breslow-Wilcoxon test (extra weight is given to early timepoints). Statistical comparisons to the isotype control group are indicated on the graph, ** P<0.01; *** P<0.001. All three study groups were significantly different than the isotype control group. There is no significant statistical difference between the three ShAb study groups. FIG. 25B: SARS-CoV-2 viral loads in BAL were measured 2 days post-challenge in a subset of animals (n=5/group) by plaque assay. Asterisks indicate significance compared to the antibody isotype control group assessed by one-way ANOVA with Dunnett's multiple comparisons test, ***P<0.001. FIG. 25C: Body weight changes were measured daily for each study mice. Percentage of initial weight is plotted. FIG. 25D: Clinical score of the K18-hACE2 study groups.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of +20%, +10%, +5%, +1%, ±0.9%, ±0.8%, ±0.7%, ±0.6%, +0.5%, ±0.4%, ±0.3%, +0.2% or +0.1% from the specified value as such variations are appropriate to perform the disclosed methods and/or to make and use the disclosed devices. When “about” is present before a series of numbers or a range, it is understood that “about” can modify each of the numbers in the series or ranges.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “antibody” or “antibodies” as used in this disclosure refers to an immunoglobulin or an antigen-binding fragment thereof. As will be understood by those in the art, the immunological binding reagents encompassed by the term “antibody” or “antibodies” extend to all antibodies from all species, and antigen binding fragments thereof and include, unless otherwise specified, polyclonal, monoclonal, monospecific, bispecific, trispecific, quad-specific, polyspecific, shark, humanized, human, camelised, mouse, non-human primates, single domain, single chain, chimeric, synthetic, recombinant, hybrid, mutated, CDR-grafted, and in vitro generated antibodies. In certain embodiments, the antibody is shark new antigen receptor immunoglobulins (also called IgNAR or NAR) having a single variable domain. The antibody can include a constant region, or a portion thereof, such as the kappa, lambda, alpha, gamma, delta, epsilon, and mu constant region genes. For example, heavy chain constant regions of the various isotypes can be used, including: IgG1, IgG2, IgG3, IgG4, IgM, IgA1, IgA2, IgD, and IgE. By way of example, the light chain constant region can be kappa or lambda.

The term “antigen” refers to any substance that is capable of generating an immune response (e.g., the production of antibodies).

The terms “antigen-binding domain” and “antigen-binding fragment” refer to a part of an antibody molecule that comprises amino acids responsible for the specific binding between antibody and antigen. For certain antigens, the antigen-binding domain or antigen-binding fragment of an antibody molecule may only bind to a part of the antigen. The part of the antigen that is specifically recognized and bound by the antibody is referred to as the “epitope” or “antigenic determinant.” Antigen-binding domains and antigen-binding fragments include Fab (Fragment antigen-binding); a F(ab′)₂ fragment, a bivalent fragment having two Fab fragments linked by a disulfide bridge at the hinge region; Fv fragment; a single chain Fv fragment (scFv) see e.g., Bird et al. (1988) Science 242:423-426; and Huston et al. (1988) Proc. Natl. Acad. Sci. USA 85:5879-5883); a Fd fragment having the two V_{H} and C_{H}1 domains; dAb (Ward et al., (1989) Nature 341:544-546), and other antibody fragments that retain antigen-binding function. The Fab fragment has V_{H}-C_{H} and V_{L}-C_{L} domains covalently linked by a disulfide bond between the constant regions. The Fv fragment is smaller and has V_{H} and V_{L} domains non-covalently linked. To overcome the tendency of non-covalently linked domains to dissociate, a scFv can be constructed. The scFv contains a flexible polypeptide that links (1) the C-terminus of V_{H} to the N-terminus of V_{L}, or (2) the C-terminus of V_{L} to the N-terminus of V_{H}. These antibody fragments are obtained using conventional techniques known to those with skill in the art, and the fragments are evaluated for function in the same manner as are intact antibodies.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

As used herein, the terms “binds” or “binding” refer to the interaction between a binding agent, such as an antibody, or an antigen-binding fragment thereof, and an antigen, or an antigenic fragment.

The term “COVID-19” refers to coronavirus disease 2019, the disease caused by SARS-CoV-2 coronavirus.

The term “diagnosing” or “diagnosis” as used herein refers to the use of information (e.g., antibody binding or data from tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

The term “Fc domain” or “Fc region” or the like refers to the portion of an immunoglobulin, e.g., an IgG molecule that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor. In some embodiments, the Fc domain is a human Fc domain, such as a human IgG Fe domain, including, for example, a human IgG1 Fe domain, or a human IgM Fc domain.

The term “effective amount” refers to a dosage or amount that is sufficient for treating and/or preventing an indicated disease or condition, such as SARS-CoV-2 coronavirus infection.

The term “identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988).

Typical methods to determine identity are designed to give the largest match between the sequences tested. Methods to determine identity and similarity are codified in publicly available computer programs. Typical computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity. IgBlast may also be used to determine germline V, D and J gene matches to a query sequence, which is available on the world wide web at ncbi.nlm.nih.gov/igblast/.

The term “in need thereof” means that the subject has been identified or suspected as having a need for the particular method or treatment. In some embodiments, the identification can be by any means of diagnosis or observation. In any of the methods described herein, the subject can be in need thereof. In some embodiments, the subject in need thereof is a human diagnosed with a SARS-CoV-2 infection. In some embodiments, the subject in need thereof is a human suspected of having a SARS-CoV-2 infection.

As used herein, the term “in some embodiments” refers to embodiments of all aspects of the disclosure, unless the context clearly indicates otherwise.

The term “isolated antibody,” refers to an antibody that is substantially free of its natural environment, including other antibodies having different antigenic specificities (e.g., an isolated antibody that specifically binds SARS-CoV-2 is substantially free of antibodies that specifically bind other epitopes or other antigens than SARS-CoV-2, unless the isolated antibody is combined with one or more isolated antibodies of interest, such as an antibody that specifically binds a second coronavirus).

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset, such as exposure to SARS-CoV-2 coronavirus.

The term “protein” refers to a polymer of amino acids, peptide nucleic acids (PNAs) or mimetics, of no specific length and to all fragments, isoforms, variants, derivatives and modifications (glycosylation, phosphorylation, post-translational modifications, etc.) thereof.

The term “recombinant antibody” refers to an antibody produced or expressed using a recombinant expression vector, where the expression vector comprises a nucleic acid encoding the recombinant antibody, such that introduction of the expression vector into an appropriate host cell or transgenic animal results in the production or expression of the recombinant antibody.

As is known in the art, recombinant antibodies, are not merely proteins isolated from a human donor, but are proteins that are produced in a host cell or transgenic animal. Appropriate host cells and suitable transgenic animals for production of the antibodies of the disclosure are described in Gene Expression Systems, Academic Press, eds. Fernandez et al., 1999. Suitable production hosts include yeast, mammalian, bacterial or insect cells or transgenic animals such as transgenic Drosophila or mice. The recombinant antibodies of the disclosure are glycosylated. The amount of glycosylation by weight for the IgG, IgM, IgA, IgD and IgE is typically about 3% a 12%, 10%, 13% and 12%, respectively. The glycosylation pattern of a recombinant human protein varies from the glycosylation pattern of its natural human protein counterpart since glycosylation is dependent upon the type of host cell or organism used to express the recombinant protein.

As is also known in the art, the glycosylation patterns of recombinant antibodies are not the same as those of any existing natural counterparts, even when the antibodies are expressed in human cells. See Nallet et al., New Biotechnology, 2012, 29: 471-476 who report that IgG expressed in a human embryonic kidney cell line results in similar, but not identical, glycosylation patterns in comparison to those expressed in humans. Further, Luac et al., Biochimica et Biophysica Acta, 2015, 1860: 1574-1582 reports that variation in glycosylation patterns for IgG differ between and within humans. Accordingly, the recombinant monoclonal antibodies of the instant disclosure are structurally distinguishable from antibodies obtained from human donors.

The term “sample” is used herein in the broadest sense and can be obtained from any source in the body. A sample can encompass fluids, solids and/or tissues. In some embodiments, a sample can include one or more of the following fluids: aural fluid, nasal fluid, or ear drainage. A sample can also include other fluids, such as serous fluid, urine, saliva, tears, blood, plasma, and serum.

The term “single variable domain,” “nanobody,” “VNAR,” or “VNAR domain” are used herein interchangeably and refers to the single variable domain of an IgNAR antibody.

As used herein, the term “therapeutic” means an agent utilized to treat, combat, ameliorate, prevent or improve an unwanted condition or disease of a patient, such as SARS-CoV-2 infection.

The terms “treatment” or “treating” and the like refer to any treatment of any disease or condition in an animal, such as a bird or mammal, e.g. particularly a human or a mouse, and includes inhibiting a disease, condition, or symptom of a disease or condition, e.g., arresting its development and/or delaying its onset or manifestation in the patient or relieving a disease, condition, or symptom of a disease or condition, e.g., causing regression of the condition or disease and/or its symptoms.

The terms “subject,” “host,” “patient,” and “individual” are used interchangeably herein to refer to any subject for whom diagnosis or therapy is desired, particularly mammals, such as humans.

The term “pharmaceutically acceptable excipient” means solvents, diluents, dispersion media, coatings, antibacterial agents and antifungal agents, isotonic agents, solid and liquid fillers, and absorption delaying agents, and the like, that are suitable for administration into a human. The use of such media and agents for pharmaceutically active substances is well known in the art.

Single Variable Domain of IgNAR (VNAR)

There are three main isotypes of immunoglobulins or antibodies found in cartilaginous fishes. Two of the isotypes contain two standard heavy and light chains and are called IgM and IgW (also called IgX or IgNARC). The third isotype is called IgNAR and contains a homodimer of heavy chains that are not associated with light chains. Each chain of the secretory form consists of one variable domain followed by five constant domains, the last four being homologous to IgW constant domains. The shark new antigen receptor immunoglobulins (also called IgNAR or NAR) have a single variable domain (called VNARs or nanobodies). Unlike mammalian variable domains, the IgNAR variable domain contains only two—not three-Complementarity Determining Regions (CDRs), namely CDR1 and CDR3. High rates of somatic mutation after antigen contact are observed in CDR1, at the CDR2 truncation site, where the remaining loop forms a belt-like structure around the middle of the molecule, and in a loop which corresponds to HV4 in T-cell receptors. Accordingly, these mutation-prone regions have been named HV2 and HV4, respectively (see e.g., Dooley et al., PNAS (USA) 2006; 103:1846-51, which is hereby incorporated by reference in its entirety). A more detailed discussion of VNARs and their structure can be found in U.S. Patent Publication No. 2018/0171020, which is hereby incorporated by reference in its entirety.

Disclosed herein are nanobodies (or VNARs) isolated from sharks that were immunized with the Spike glycoprotein of SARS-CoV-2 coronavirus, particularly the receptor binding domain (RBD) and N-terminus domain (NTD) of the SARS-CoV-2 Spike glycoprotein. The nanobodies (or VNARs) disclosed herein bind to the RBD and NTD of the SARS-CoV-2 Spike glycoprotein and other related sarbecoviruses, such as SARS-CoV-1, with high affinity and are useful in various applications, including diagnostics and therapeutics.

In one aspect, the VNAR is an AliB5-2D8 nanobody (renamed ShAb01). In certain embodiments, the ShAb01 nanobody comprises a single variable domain comprising the amino acid sequence of SEQ ID NO: 1. In certain embodiments, the ShAb01 nanobody comprises a single variable domain having a CDR1 and a CDR3, wherein the CDR1 comprises the amino acid sequence of SEQ ID NO: 246 and the CDR3 comprises the amino acid sequence of SEQ ID NO: 276. In certain embodiments, the ShAb01 nanobody comprises a single variable domain having a CDR1, a CDR3, an HV2, and an HV4, wherein the CDR1 comprises the amino acid sequence of SEQ ID NO: 246, the CDR3 comprises the amino acid sequence of SEQ ID NO: 276, the HV2 comprises the amino acid sequence of SEQ ID NO: 306, and the HV4 comprises the amino acid sequence of SEQ ID NO: 336.

In another aspect, the VNAR is a MoB3-3D8 nanobody (renamed ShAb02). In certain embodiments, the ShAb02 nanobody comprises a single variable domain comprising the amino acid sequence of SEQ ID NO: 2. In certain embodiments, the ShAb02 nanobody comprises a single variable domain having a CDR1 and a CDR3, wherein the CDR1 comprises the amino acid sequence of SEQ ID NO: 247 and the CDR3 comprises the amino acid sequence of SEQ ID NO: 277. In certain embodiments, the ShAb02 nanobody comprises a single variable domain having a CDR1, a CDR3, an HV2, and an HV4, wherein the CDR1 comprises the amino acid sequence of SEQ ID NO: 247, the CDR3 comprises the amino acid sequence of SEQ ID NO: 277, the HV2 comprises the amino acid sequence of SEQ ID NO: 307, and the HV4 comprises the amino acid sequence of SEQ ID NO: 337.

In another aspect, the VNAR is a ShAb03 nanobody. In certain embodiments, the ShAb03 nanobody comprises a single variable domain comprising the amino acid sequence of SEQ ID NO: 3. In certain embodiments, the ShAb03 nanobody comprises a single variable domain having a CDR1 and a CDR3, wherein the CDR1 comprises the amino acid sequence of SEQ ID NO: 248 and the CDR3 comprises the amino acid sequence of SEQ ID NO: 278. In certain embodiments, the ShAb03 nanobody comprises a single variable domain having a CDR1, a CDR3, an HV2, and an HV4, wherein the CDR1 comprises the amino acid sequence of SEQ ID NO: 248, the CDR3 comprises the amino acid sequence of SEQ ID NO: 278, the HV2 comprises the amino acid sequence of SEQ ID NO: 308, and the HV4 comprises the amino acid sequence of SEQ ID NO: 338.

In another aspect, the VNAR is a ShAb04 nanobody. In certain embodiments, the ShAb04 nanobody comprises a single variable domain comprising the amino acid sequence of SEQ ID NO: 4. In certain embodiments, the ShAb04 nanobody comprises a single variable domain having a CDR1 and a CDR3, wherein the CDR1 comprises the amino acid sequence of SEQ ID NO: 249 and the CDR3 comprises the amino acid sequence of SEQ ID NO: 279. In certain embodiments, the ShAb04 nanobody comprises a single variable domain having a CDR1, a CDR3, an HV2, and an HV4, wherein the CDR1 comprises the amino acid sequence of SEQ ID NO: 249, the CDR3 comprises the amino acid sequence of SEQ ID NO: 279, the HV2 comprises the amino acid sequence of SEQ ID NO: 309, and the HV4 comprises the amino acid sequence of SEQ ID NO: 339.

In another aspect, the VNAR is a ShAb05 nanobody. In certain embodiments, the ShAb05 nanobody comprises a single variable domain comprising the amino acid sequence of SEQ ID NO: 5. In certain embodiments, the ShAb05 nanobody comprises a single variable domain having a CDR1 and a CDR3, wherein the CDR1 comprises the amino acid sequence of SEQ ID NO: 250 and the CDR3 comprises the amino acid sequence of SEQ ID NO: 280. In certain embodiments, the ShAb05 nanobody comprises a single variable domain having a CDR1, a CDR3, an HV2, and an HV4, wherein the CDR1 comprises the amino acid sequence of SEQ ID NO: 250, the CDR3 comprises the amino acid sequence of SEQ ID NO: 280, the HV2 comprises the amino acid sequence of SEQ ID NO: 310, and the HV4 comprises the amino acid sequence of SEQ ID NO: 340.

In another aspect, the VNAR is a ShAb06 nanobody. In certain embodiments, the ShAb06 nanobody comprises a single variable domain comprising the amino acid sequence of SEQ ID NO: 6. In certain embodiments, the ShAb06 nanobody comprises a single variable domain having a CDR1 and a CDR3, wherein the CDR1 comprises the amino acid sequence of SEQ ID NO: 251 and the CDR3 comprises the amino acid sequence of SEQ ID NO: 281. In certain embodiments, the ShAb06 nanobody comprises a single variable domain having a CDR1, a CDR3, an HV2, and an HV4, wherein the CDR1 comprises the amino acid sequence of SEQ ID NO: 251, the CDR3 comprises the amino acid sequence of SEQ ID NO: 281, the HV2 comprises the amino acid sequence of SEQ ID NO: 311, and the HV4 comprises the amino acid sequence of SEQ ID NO: 341.

In another aspect, the VNAR is a ShAb07 nanobody. In certain embodiments, the ShAb07 nanobody comprises a single variable domain comprising the amino acid sequence of SEQ ID NO: 7. In certain embodiments, the ShAb07 nanobody comprises a single variable domain having a CDR1 and a CDR3, wherein the CDR1 comprises the amino acid sequence of SEQ ID NO: 252 and the CDR3 comprises the amino acid sequence of SEQ ID NO: 282. In certain embodiments, the ShAb07 nanobody comprises a single variable domain having a CDR1, a CDR3, an HV2, and an HV4, wherein the CDR1 comprises the amino acid sequence of SEQ ID NO: 252, the CDR3 comprises the amino acid sequence of SEQ ID NO: 282, the HV2 comprises the amino acid sequence of SEQ ID NO: 312, and the HV4 comprises the amino acid sequence of SEQ ID NO: 342.

In another aspect, the VNAR is a ShAb08 nanobody. In certain embodiments, the ShAb08 nanobody comprises a single variable domain comprising the amino acid sequence of SEQ ID NO: 8. In certain embodiments, the ShAb08 nanobody comprises a single variable domain having a CDR1 and a CDR3, wherein the CDR1 comprises the amino acid sequence of SEQ ID NO: 253 and the CDR3 comprises the amino acid sequence of SEQ ID NO: 283. In certain embodiments, the ShAb08 nanobody comprises a single variable domain having a CDR1, a CDR3, an HV2, and an HV4, wherein the CDR1 comprises the amino acid sequence of SEQ ID NO: 253, the CDR3 comprises the amino acid sequence of SEQ ID NO: 283, the HV2 comprises the amino acid sequence of SEQ ID NO: 313, and the HV4 comprises the amino acid sequence of SEQ ID NO: 343.

In another aspect, the VNAR is a ShAb09 nanobody. In certain embodiments, the ShAb09 nanobody comprises a single variable domain comprising the amino acid sequence of SEQ ID NO: 9. In certain embodiments, the ShAb09 nanobody comprises a single variable domain having a CDR1 and a CDR3, wherein the CDR1 comprises the amino acid sequence of SEQ ID NO: 254 and the CDR3 comprises the amino acid sequence of SEQ ID NO: 284. In certain embodiments, the ShAb09 nanobody comprises a single variable domain having a CDR1, a CDR3, an HV2, and an HV4, wherein the CDR1 comprises the amino acid sequence of SEQ ID NO: 254, the CDR3 comprises the amino acid sequence of SEQ ID NO: 284, the HV2 comprises the amino acid sequence of SEQ ID NO: 314, and the HV4 comprises the amino acid sequence of SEQ ID NO: 344.

In another aspect, the VNAR is a ShAb10 nanobody. In certain embodiments, the ShAb10 nanobody comprises a single variable domain comprising the amino acid sequence of SEQ ID NO: 10. In certain embodiments, the ShAb10 nanobody comprises a single variable domain having a CDR1 and a CDR3, wherein the CDR1 comprises the amino acid sequence of SEQ ID NO: 255 and the CDR3 comprises the amino acid sequence of SEQ ID NO: 285. In certain embodiments, the ShAb10 nanobody comprises a single variable domain having a CDR1, a CDR3, an HV2, and an HV4, wherein the CDR1 comprises the amino acid sequence of SEQ ID NO: 255, the CDR3 comprises the amino acid sequence of SEQ ID NO: 285, the HV2 comprises the amino acid sequence of SEQ ID NO: 315, and the HV4 comprises the amino acid sequence of SEQ ID NO: 345.

In another aspect, the VNAR is a ShAb11 nanobody. In certain embodiments, the ShAb11 nanobody comprises a single variable domain comprising the amino acid sequence of SEQ ID NO: 11. In certain embodiments, the ShAb11 nanobody comprises a single variable domain having a CDR1 and a CDR3, wherein the CDR1 comprises the amino acid sequence of SEQ ID NO: 256 and the CDR3 comprises the amino acid sequence of SEQ ID NO: 286. In certain embodiments, the ShAb11 nanobody comprises a single variable domain having a CDR1, a CDR3, an HV2, and an HV4, wherein the CDR1 comprises the amino acid sequence of SEQ ID NO: 256, the CDR3 comprises the amino acid sequence of SEQ ID NO: 286, the HV2 comprises the amino acid sequence of SEQ ID NO: 316, and the HV4 comprises the amino acid sequence of SEQ ID NO: 346.

In another aspect, the VNAR is a ShAb12 nanobody. In certain embodiments, the ShAb12 nanobody comprises a single variable domain comprising the amino acid sequence of SEQ ID NO: 12. In certain embodiments, the ShAb12 nanobody comprises a single variable domain having a CDR1 and a CDR3, wherein the CDR1 comprises the amino acid sequence of SEQ ID NO: 257 and the CDR3 comprises the amino acid sequence of SEQ ID NO: 287. In certain embodiments, the ShAb12 nanobody comprises a single variable domain having a CDR1, a CDR3, an HV2, and an HV4, wherein the CDR1 comprises the amino acid sequence of SEQ ID NO: 257, the CDR3 comprises the amino acid sequence of SEQ ID NO: 287, the HV2 comprises the amino acid sequence of SEQ ID NO: 317, and the HV4 comprises the amino acid sequence of SEQ ID NO: 347.

In another aspect, the VNAR is a ShAb13 nanobody. In certain embodiments, the ShAb13 nanobody comprises a single variable domain comprising the amino acid sequence of SEQ ID NO: 13. In certain embodiments, the ShAb13 nanobody comprises a single variable domain having a CDR1 and a CDR3, wherein the CDR1 comprises the amino acid sequence of SEQ ID NO: 258 and the CDR3 comprises the amino acid sequence of SEQ ID NO: 288. In certain embodiments, the ShAb13 nanobody comprises a single variable domain having a CDR1, a CDR3, an HV2, and an HV4, wherein the CDR1 comprises the amino acid sequence of SEQ ID NO: 258, the CDR3 comprises the amino acid sequence of SEQ ID NO: 288, the HV2 comprises the amino acid sequence of SEQ ID NO: 318, and the HV4 comprises the amino acid sequence of SEQ ID NO: 348.

In another aspect, the VNAR is a ShAb14 nanobody. In certain embodiments, the ShAb14 nanobody comprises a single variable domain comprising the amino acid sequence of SEQ ID NO: 14. In certain embodiments, the ShAb14 nanobody comprises a single variable domain having a CDR1 and a CDR3, wherein the CDR1 comprises the amino acid sequence of SEQ ID NO: 259 and the CDR3 comprises the amino acid sequence of SEQ ID NO: 289. In certain embodiments, the ShAb14 nanobody comprises a single variable domain having a CDR1, a CDR3, an HV2, and an HV4, wherein the CDR1 comprises the amino acid sequence of SEQ ID NO: 259, the CDR3 comprises the amino acid sequence of SEQ ID NO: 289, the HV2 comprises the amino acid sequence of SEQ ID NO: 319, and the HV4 comprises the amino acid sequence of SEQ ID NO: 349.

In another aspect, the VNAR is a ShAb15 nanobody. In certain embodiments, the ShAb15 nanobody comprises a single variable domain comprising the amino acid sequence of SEQ ID NO: 15. In certain embodiments, the ShAb15 nanobody comprises a single variable domain having a CDR1 and a CDR3, wherein the CDR1 comprises the amino acid sequence of SEQ ID NO: 260 and the CDR3 comprises the amino acid sequence of SEQ ID NO: 290. In certain embodiments, the ShAb15 nanobody comprises a single variable domain having a CDR1, a CDR3, an HV2, and an HV4, wherein the CDR1 comprises the amino acid sequence of SEQ ID NO: 260, the CDR3 comprises the amino acid sequence of SEQ ID NO: 290, the HV2 comprises the amino acid sequence of SEQ ID NO: 320, and the HV4 comprises the amino acid sequence of SEQ ID NO: 350.

In another aspect, the VNAR is a ShAb16 nanobody. In certain embodiments, the ShAb16 nanobody comprises a single variable domain comprising the amino acid sequence of SEQ ID NO: 16. In certain embodiments, the ShAb16 nanobody comprises a single variable domain having a CDR1 and a CDR3, wherein the CDR1 comprises the amino acid sequence of SEQ ID NO: 261 and the CDR3 comprises the amino acid sequence of SEQ ID NO: 291. In certain embodiments, the ShAb16 nanobody comprises a single variable domain having a CDR1, a CDR3, an HV2, and an HV4, wherein the CDR1 comprises the amino acid sequence of SEQ ID NO: 261, the CDR3 comprises the amino acid sequence of SEQ ID NO: 291, the HV2 comprises the amino acid sequence of SEQ ID NO: 321, and the HV4 comprises the amino acid sequence of SEQ ID NO: 351.

In another aspect, the VNAR is a ShAb17 nanobody. In certain embodiments, the ShAb17 nanobody comprises a single variable domain comprising the amino acid sequence of SEQ ID NO: 17. In certain embodiments, the ShAb17 nanobody comprises a single variable domain having a CDR1 and a CDR3, wherein the CDR1 comprises the amino acid sequence of SEQ ID NO: 262 and the CDR3 comprises the amino acid sequence of SEQ ID NO: 292. In certain embodiments, the ShAb17 nanobody comprises a single variable domain having a CDR1, a CDR3, an HV2, and an HV4, wherein the CDR1 comprises the amino acid sequence of SEQ ID NO: 262, the CDR3 comprises the amino acid sequence of SEQ ID NO: 292, the HV2 comprises the amino acid sequence of SEQ ID NO: 322, and the HV4 comprises the amino acid sequence of SEQ ID NO: 352.

In another aspect, the VNAR is a ShAb18 nanobody. In certain embodiments, the ShAb18 nanobody comprises a single variable domain comprising the amino acid sequence of SEQ ID NO: 18. In certain embodiments, the ShAb18 nanobody comprises a single variable domain having a CDR1 and a CDR3, wherein the CDR1 comprises the amino acid sequence of SEQ ID NO: 263 and the CDR3 comprises the amino acid sequence of SEQ ID NO: 293. In certain embodiments, the ShAb18 nanobody comprises a single variable domain having a CDR1, a CDR3, an HV2, and an HV4, wherein the CDR1 comprises the amino acid sequence of SEQ ID NO: 263, the CDR3 comprises the amino acid sequence of SEQ ID NO: 293, the HV2 comprises the amino acid sequence of SEQ ID NO: 323, and the HV4 comprises the amino acid sequence of SEQ ID NO: 353.

In another aspect, the VNAR is a ShAb19 nanobody. In certain embodiments, the ShAb19 nanobody comprises a single variable domain comprising the amino acid sequence of SEQ ID NO: 19. In certain embodiments, the ShAb19 nanobody comprises a single variable domain having a CDR1 and a CDR3, wherein the CDR1 comprises the amino acid sequence of SEQ ID NO: 264 and the CDR3 comprises the amino acid sequence of SEQ ID NO: 294. In certain embodiments, the ShAb19 nanobody comprises a single variable domain having a CDR1, a CDR3, an HV2, and an HV4, wherein the CDR1 comprises the amino acid sequence of SEQ ID NO: 264, the CDR3 comprises the amino acid sequence of SEQ ID NO: 294, the HV2 comprises the amino acid sequence of SEQ ID NO: 324, and the HV4 comprises the amino acid sequence of SEQ ID NO: 354.

In another aspect, the VNAR is a ShAb20 nanobody. In certain embodiments, the ShAb20 nanobody comprises a single variable domain comprising the amino acid sequence of SEQ ID NO: 20. In certain embodiments, the ShAb20 nanobody comprises a single variable domain having a CDR1 and a CDR3, wherein the CDR1 comprises the amino acid sequence of SEQ ID NO: 265 and the CDR3 comprises the amino acid sequence of SEQ ID NO: 295. In certain embodiments, the ShAb20 nanobody comprises a single variable domain having a CDR1, a CDR3, an HV2, and an HV4, wherein the CDR1 comprises the amino acid sequence of SEQ ID NO: 265, the CDR3 comprises the amino acid sequence of SEQ ID NO: 295, the HV2 comprises the amino acid sequence of SEQ ID NO: 325, and the HV4 comprises the amino acid sequence of SEQ ID NO: 355.

In another aspect, the VNAR is a ShAb21 nanobody. In certain embodiments, the ShAb21 nanobody comprises a single variable domain comprising the amino acid sequence of SEQ ID NO: 21. In certain embodiments, the ShAb21 nanobody comprises a single variable domain having a CDR1 and a CDR3, wherein the CDR1 comprises the amino acid sequence of SEQ ID NO: 266 and the CDR3 comprises the amino acid sequence of SEQ ID NO: 296. In certain embodiments, the ShAb21 nanobody comprises a single variable domain having a CDR1, a CDR3, an HV2, and an HV4, wherein the CDR1 comprises the amino acid sequence of SEQ ID NO: 266, the CDR3 comprises the amino acid sequence of SEQ ID NO: 296, the HV2 comprises the amino acid sequence of SEQ ID NO: 326, and the HV4 comprises the amino acid sequence of SEQ ID NO: 356.

In another aspect, the VNAR is a ShAb22 nanobody. In certain embodiments, the ShAb22 nanobody comprises a single variable domain comprising the amino acid sequence of SEQ ID NO: 22. In certain embodiments, the ShAb22 nanobody comprises a single variable domain having a CDR1 and a CDR3, wherein the CDR1 comprises the amino acid sequence of SEQ ID NO: 267 and the CDR3 comprises the amino acid sequence of SEQ ID NO: 297. In certain embodiments, the ShAb22 nanobody comprises a single variable domain having a CDR1, a CDR3, an HV2, and an HV4, wherein the CDR1 comprises the amino acid sequence of SEQ ID NO: 267, the CDR3 comprises the amino acid sequence of SEQ ID NO: 297, the HV2 comprises the amino acid sequence of SEQ ID NO: 327, and the HV4 comprises the amino acid sequence of SEQ ID NO: 357.

In another aspect, the VNAR is a ShAb23 nanobody. In certain embodiments, the ShAb23 nanobody comprises a single variable domain comprising the amino acid sequence of SEQ ID NO: 23. In certain embodiments, the ShAb23 nanobody comprises a single variable domain having a CDR1 and a CDR3, wherein the CDR1 comprises the amino acid sequence of SEQ ID NO: 268 and the CDR3 comprises the amino acid sequence of SEQ ID NO: 298. In certain embodiments, the ShAb23 nanobody comprises a single variable domain having a CDR1, a CDR3, an HV2, and an HV4, wherein the CDR1 comprises the amino acid sequence of SEQ ID NO: 268, the CDR3 comprises the amino acid sequence of SEQ ID NO: 298, the HV2 comprises the amino acid sequence of SEQ ID NO: 328, and the HV4 comprises the amino acid sequence of SEQ ID NO: 358.

In another aspect, the VNAR is a ShAb24 nanobody. In certain embodiments, the ShAb24 nanobody comprises a single variable domain comprising the amino acid sequence of SEQ ID NO: 24. In certain embodiments, the ShAb24 nanobody comprises a single variable domain having a CDR1 and a CDR3, wherein the CDR1 comprises the amino acid sequence of SEQ ID NO: 269 and the CDR3 comprises the amino acid sequence of SEQ ID NO: 299. In certain embodiments, the ShAb24 nanobody comprises a single variable domain having a CDR1, a CDR3, an HV2, and an HV4, wherein the CDR1 comprises the amino acid sequence of SEQ ID NO: 269, the CDR3 comprises the amino acid sequence of SEQ ID NO: 299, the HV2 comprises the amino acid sequence of SEQ ID NO: 329, and the HV4 comprises the amino acid sequence of SEQ ID NO: 359.

In another aspect, the VNAR is a ShAb25 nanobody. In certain embodiments, the ShAb25 nanobody comprises a single variable domain comprising the amino acid sequence of SEQ ID NO: 25. In certain embodiments, the ShAb25 nanobody comprises a single variable domain having a CDR1 and a CDR3, wherein the CDR1 comprises the amino acid sequence of SEQ ID NO: 270 and the CDR3 comprises the amino acid sequence of SEQ ID NO: 300. In certain embodiments, the ShAb25 nanobody comprises a single variable domain having a CDR1, a CDR3, an HV2, and an HV4, wherein the CDR1 comprises the amino acid sequence of SEQ ID NO: 270, the CDR3 comprises the amino acid sequence of SEQ ID NO: 300, the HV2 comprises the amino acid sequence of SEQ ID NO: 330, and the HV4 comprises the amino acid sequence of SEQ ID NO: 360.

In another aspect, the VNAR is a ShAb26 nanobody. In certain embodiments, the ShAb26 nanobody comprises a single variable domain comprising the amino acid sequence of SEQ ID NO: 26. In certain embodiments, the ShAb26 nanobody comprises a single variable domain having a CDR1 and a CDR3, wherein the CDR1 comprises the amino acid sequence of SEQ ID NO: 271 and the CDR3 comprises the amino acid sequence of SEQ ID NO: 301. In certain embodiments, the ShAb26 nanobody comprises a single variable domain having a CDR1, a CDR3, an HV2, and an HV4, wherein the CDR1 comprises the amino acid sequence of SEQ ID NO: 271, the CDR3 comprises the amino acid sequence of SEQ ID NO: 301, the HV2 comprises the amino acid sequence of SEQ ID NO: 331, and the HV4 comprises the amino acid sequence of SEQ ID NO: 361.

In another aspect, the VNAR is a ShAb27 nanobody. In certain embodiments, the ShAb27 nanobody comprises a single variable domain comprising the amino acid sequence of SEQ ID NO: 27. In certain embodiments, the ShAb27 nanobody comprises a single variable domain having a CDR1 and a CDR3, wherein the CDR1 comprises the amino acid sequence of SEQ ID NO: 272 and the CDR3 comprises the amino acid sequence of SEQ ID NO: 302. In certain embodiments, the ShAb27 nanobody comprises a single variable domain having a CDR1, a CDR3, an HV2, and an HV4, wherein the CDR1 comprises the amino acid sequence of SEQ ID NO: 272, the CDR3 comprises the amino acid sequence of SEQ ID NO: 302, the HV2 comprises the amino acid sequence of SEQ ID NO: 332, and the HV4 comprises the amino acid sequence of SEQ ID NO: 362.

In another aspect, the VNAR is a ShAb28 nanobody. In certain embodiments, the ShAb28 nanobody comprises a single variable domain comprising the amino acid sequence of SEQ ID NO: 28. In certain embodiments, the ShAb28 nanobody comprises a single variable domain having a CDR1 and a CDR3, wherein the CDR1 comprises the amino acid sequence of SEQ ID NO: 273 and the CDR3 comprises the amino acid sequence of SEQ ID NO: 303. In certain embodiments, the ShAb28 nanobody comprises a single variable domain having a CDR1, a CDR3, an HV2, and an HV4, wherein the CDR1 comprises the amino acid sequence of SEQ ID NO: 273, the CDR3 comprises the amino acid sequence of SEQ ID NO: 303, the HV2 comprises the amino acid sequence of SEQ ID NO: 333, and the HV4 comprises the amino acid sequence of SEQ ID NO: 363.

In another aspect, the VNAR is a ShAb29 nanobody. In certain embodiments, the ShAb29 nanobody comprises a single variable domain comprising the amino acid sequence of SEQ ID NO: 29. In certain embodiments, the ShAb29 nanobody comprises a single variable domain having a CDR1 and a CDR3, wherein the CDR1 comprises the amino acid sequence of SEQ ID NO: 274 and the CDR3 comprises the amino acid sequence of SEQ ID NO: 304. In certain embodiments, the ShAb29 nanobody comprises a single variable domain having a CDR1, a CDR3, an HV2, and an HV4, wherein the CDR1 comprises the amino acid sequence of SEQ ID NO: 274, the CDR3 comprises the amino acid sequence of SEQ ID NO: 304, the HV2 comprises the amino acid sequence of SEQ ID NO: 334, and the HV4 comprises the amino acid sequence of SEQ ID NO: 364.

In another aspect, the VNAR is a ShAb30 nanobody. In certain embodiments, the ShAb30 nanobody comprises a single variable domain comprising the amino acid sequence of SEQ ID NO: 30. In certain embodiments, the ShAb30 nanobody comprises a single variable domain having a CDR1 and a CDR3, wherein the CDR1 comprises the amino acid sequence of SEQ ID NO: 275 and the CDR3 comprises the amino acid sequence of SEQ ID NO: 305. In certain embodiments, the ShAb30 nanobody comprises a single variable domain having a CDR1, a CDR3, an HV2, and an HV4, wherein the CDR1 comprises the amino acid sequence of SEQ ID NO: 275, the CDR3 comprises the amino acid sequence of SEQ ID NO: 305, the HV2 comprises the amino acid sequence of SEQ ID NO: 335, and the HV4 comprises the amino acid sequence of SEQ ID NO: 365.

In another aspect, the VNAR is a MoB5-1D4 nanobody (renamed ShAb31). In certain embodiments, the ShAb31 nanobody comprises a single variable domain comprising the amino acid sequence of SEQ ID NO: 394. In certain embodiments, the ShAb31 nanobody comprises a single variable domain having a CDR1 and a CDR3, wherein the CDR1 comprises the amino acid sequence of SEQ ID NO: 395 and the CDR3 comprises the amino acid sequence of SEQ ID NO: 396. In certain embodiments, the ShAb31 nanobody comprises a single variable domain having a CDR1, a CDR3, an HV2, and an HV4, wherein the CDR1 comprises the amino acid sequence of SEQ ID NO: 395, the CDR3 comprises the amino acid sequence of SEQ ID NO: 396, the HV2 comprises the amino acid sequence of SEQ ID NO: 397, and the HV4 comprises the amino acid sequence of SEQ ID NO: 398.

Modified versions of the single variable domains of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies are also provided. Typically, modifications to an antibody can be introduced through the nucleic acids that encode the variable domain of the antibody. These modifications can include deletions, insertions, point mutations, truncations, and amino acid substitutions and addition of amino acids or non-amino acid moieties. For example, random mutagenesis of the disclosed variable domain sequences can be used to generate variant variable domains still capable of binding the SARS-CoV-2 RBD. A technique using error-prone PCR is described by Gram et al. (Proc. Nat. Acad. Sci. U.S.A. (1992) 89: 3576 3580). Another method uses direct mutagenesis of the disclosed variable domain sequences. Modifications can also be made directly to the amino acid sequence, such as by cleavage, addition of a linker molecule or addition of a detectable moiety, such as biotin, addition of a fatty acid, and the like.

### SARS-CoV-2 Coronavirus Binding Agents

This disclosure provides SARS-CoV-2 coronavirus binding agents that bind to a Spike glycoprotein of SARS-CoV-2 coronavirus. The SARS-CoV-2 coronavirus binding agents disclosed herein have been shown to bind to the Spike glycoprotein of SARS-CoV-2 coronavirus with nM level or pM level affinity as measured using BioLayer Interferometry. Some of the SARS-CoV-2 coronavirus binding agents have been shown to possess broad cross-reactivity against various SARS-CoV-2 variants, including variants B.1.1.7 (Alpha), B.1.351 (Beta), and/or B.1.617.2 variant (Delta), as well as other related sarbecoviruses, such as SARS-CoV-1. The SARS-CoV-2 coronavirus binding agents of the disclosure may be used to neutralize SARS-CoV-2 coronavirus, the SARS-CoV-1 coronavirus, and/or other related sarbecoviruses, including other pandemic sarbecovirus strains. The SARS-CoV-2 coronavirus binding agents of the disclosure may also be used therapeutically for prevention or treatment of a disease, such as COVID-19, that is caused by SARS-CoV-2 infection or other coronavirus infection. In some embodiments, the SARS-CoV-2 coronavirus binding agents of the disclosure bind to the Spike (S) glycoprotein of SARS-CoV-2 coronavirus or other coronaviruses, thereby inhibiting viral entry into host cells. In other embodiments, the SARS-CoV-2 coronavirus binding agents of the disclosure have capability of being used commercially in antigen-capture SARS-CoV-2 diagnostic assays.

In some embodiments, the SARS-CoV-2 coronavirus binding agent of the disclosure is one or more of the nanobodies disclosed herein, including but not limited to, ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies.

In some embodiments, the SARS-CoV-2 coronavirus binding agent of the disclosure binds to SARS-CoV-2 spike glycoprotein and comprises at least one targeting moiety that is at least about 80%, 85%, 90%, 95%, 96%, 97%, 98%, or 99% identical to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 394. In some embodiments, the SARS-CoV-2 coronavirus binding agent of the disclosure binds to SARS-CoV-2 spike glycoprotein and comprises at least one targeting moiety comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 394.

In some embodiments, the SARS-CoV-2 coronavirus binding agent of the disclosure binds to SARS-CoV-2 spike glycoprotein and comprises at least one targeting moiety that is identical to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 394, except for 1, up to 2, up to 3, up to 4, up to 5, up to 6, up to 7, and in certain cases, up to about 10 amino acid modifications (e.g., substitutions). In certain embodiments, the modified amino acids are located within at least one of the CDR regions. In certain embodiments, the modifications are located within at least one of the HV regions. In certain embodiments, the one or more modifications are located within at least one of the CDR regions and at least one of the HV regions. For example, SEQ ID NO: 13 is about 90% identical to SEQ ID NO: 16 (identity between 102 out of 113 amino acid residues), with 6 of the amino acid differences occurring in the CDR regions and 3 of the amino acid differences occurring in the HV region. Thus, in some embodiments, the one or more modifications are located within CDR1 and CDR3. In some embodiments, the one or more modifications are located within CDR1 and HV2. In some embodiments, the one or more modifications are located within CDR1 and HV4. In some embodiments, the one or more modifications are located within CDR3 and HV2. In some embodiments, the one or more modifications are located within CDR3 and HV4. In some embodiments, the one or more modifications are located within CDR1, CDR3, and HV2. In some embodiments, the one or more modifications are located within CDR1, CDR3, and HV4. In some embodiments, the one or more modifications are located within CDR3, HV2, and HV4. In some embodiments, the one or more modifications are located within CDR1, CDR3, HV2, and HV4. In certain embodiments, the one or more modifications are located outside the CDR and HV regions.

Typically, modification of the amino acid sequence involves substitution of an amino acid with an amino acid having similar charge, hydrophobic, or stereochemical characteristics. More drastic substitutions in regions outside of the CDRs or HVs may also be made as long as they do not adversely affect (e.g., reduce affinity by more than 50% as compared to unsubstituted antibody) the binding properties of the antibody.

Modified versions of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies can also be screened to identify which mutation provides a modified VNAR that retains a desired property, such as high affinity binding of the parent nanobody for the SARS-CoV-2 spike glycoprotein and/or SARS-CoV-2 neutralization. Such modified versions of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies can also be used as SARS-CoV-2 coronavirus binding agents according to the disclosure.

It is also possible to generate SARS-CoV-2 coronavirus binding agents of the disclosure using antigen-binding fragments of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies, such as those described above (e.g., a Fv, a scFv, a Fab, a Fab′, a F(ab′)₂), using techniques known in the art. Alternatively, using recombinant techniques, the CDRs of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies can be inserted into other binding or targeting moiety scaffolds to generate a SARS-CoV-2 coronavirus binding agent derived from the shark antibody, including, for example, a shark antibody fused to an Fc domain, a full-length antibody comprising heavy and light chains, a recombinant heavy-chain-only antibody (V_{HH}), a Camelid heavy-chain-only antibody, a microprotein, a darpin, an anticalin, an adnectin, an aptamer, a peptide mimetic molecule, a natural ligand for a receptor, or a synthetic molecule.

Accordingly, in some embodiments, the SARS-CoV-2 coronavirus binding agent of the disclosure comprises at least one targeting moiety comprising a CDR1 and a CDR3, wherein the CDR1 comprises an amino acid sequence selected from any one of SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, and SEQ ID NO: 395, and wherein the CDR3 comprises an amino acid sequence selected from any one of SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, and SEQ ID NO: 396.

In some embodiments, the at least one targeting moiety in the SARS-CoV-2 coronavirus binding agent of the disclosure comprises a CDR1 comprising the amino acid sequence of SEQ ID NO: 246 and a CDR3 comprising the amino acid sequence of SEQ ID NO: 276. In some embodiments, the at least one targeting moiety comprises a CDR1 comprising the amino acid sequence of SEQ ID NO: 247 and a CDR3 comprising the amino acid sequence of SEQ ID NO: 277. In some embodiments, the at least one targeting moiety comprises a CDR1 comprising the amino acid sequence of SEQ ID NO: 248 and a CDR3 comprising the amino acid sequence of SEQ ID NO:278. In some embodiments, the at least one targeting moiety comprises a CDR1 comprising the amino acid sequence of SEQ ID NO: 249 and a CDR3 comprising the amino acid sequence of SEQ ID NO: 279. In some embodiments, the at least one targeting moiety comprises a CDR1 comprising the amino acid sequence of SEQ ID NO: 250 and a CDR3 comprising the amino acid sequence of SEQ ID NO: 280. In some embodiments, the at least one targeting moiety comprises a CDR1 comprising the amino acid sequence of SEQ ID NO: 251 and a CDR3 comprising the amino acid sequence of SEQ ID NO: 281. In some embodiments, the at least one targeting moiety comprises a CDR1 comprising the amino acid sequence of SEQ ID NO: 252 and a CDR3 comprising the amino acid sequence of SEQ ID NO: 282. In some embodiments, the at least one targeting moiety comprises a CDR1 comprising the amino acid sequence of SEQ ID NO: 253 and a CDR3 comprising the amino acid sequence of SEQ ID NO: 283. In some embodiments, the at least one targeting moiety comprises a CDR1 comprising the amino acid sequence of SEQ ID NO: 254 and a CDR3 comprising the amino acid sequence of SEQ ID NO: 284. In some embodiments, the at least one targeting moiety comprises a CDR1 comprising the amino acid sequence of SEQ ID NO: 255 and a CDR3 comprising the amino acid sequence of SEQ ID NO: 285. In some embodiments, the at least one targeting moiety comprises a CDR1 comprising the amino acid sequence of SEQ ID NO: 256 and a CDR3 comprising the amino acid sequence of SEQ ID NO: 286. In some embodiments, the at least one targeting moiety comprises a CDR1 comprising the amino acid sequence of SEQ ID NO: 257 and a CDR3 comprising the amino acid sequence of SEQ ID NO: 287. In some embodiments, the at least one targeting moiety comprises a CDR1 comprising the amino acid sequence of SEQ ID NO: 258 and a CDR3 comprising the amino acid sequence of SEQ ID NO: 288. In some embodiments, the at least one targeting moiety comprises a CDR1 comprising the amino acid sequence of SEQ ID NO: 259 and a CDR3 comprising the amino acid sequence of SEQ ID NO: 289. In some embodiments, the at least one targeting moiety comprises a CDR1 comprising the amino acid sequence of SEQ ID NO: 260 and a CDR3 comprising the amino acid sequence of SEQ ID NO: 290. In some embodiments, the at least one targeting moiety comprises a CDR1 comprising the amino acid sequence of SEQ ID NO: 261 and a CDR3 comprising the amino acid sequence of SEQ ID NO: 291. In some embodiments, the at least one targeting moiety comprises a CDR1 comprising the amino acid sequence of SEQ ID NO: 262 and a CDR3 comprising the amino acid sequence of SEQ ID NO: 292. In some embodiments, the at least one targeting moiety comprises a CDR1 comprising the amino acid sequence of SEQ ID NO: 263 and a CDR3 comprising the amino acid sequence of SEQ ID NO: 293. In some embodiments, the at least one targeting moiety comprises a CDR1 comprising the amino acid sequence of SEQ ID NO: 264 and a CDR3 comprising the amino acid sequence of SEQ ID NO: 294. In some embodiments, the at least one targeting moiety comprises a CDR1 comprising the amino acid sequence of SEQ ID NO: 265 and a CDR3 comprising the amino acid sequence of SEQ ID NO: 295. In some embodiments, the at least one targeting moiety comprises a CDR1 comprising the amino acid sequence of SEQ ID NO: 266 and a CDR3 comprising the amino acid sequence of SEQ ID NO: 296. In some embodiments, the at least one targeting moiety comprises a CDR1 comprising the amino acid sequence of SEQ ID NO: 267 and a CDR3 comprising the amino acid sequence of SEQ ID NO: 297. In some embodiments, the at least one targeting moiety comprises a CDR1 comprising the amino acid sequence of SEQ ID NO: 268 and a CDR3 comprising the amino acid sequence of SEQ ID NO: 298. In some embodiments, the at least one targeting moiety comprises a CDR1 comprising the amino acid sequence of SEQ ID NO: 269 and a CDR3 comprising the amino acid sequence of SEQ ID NO: 299. In some embodiments, the at least one targeting moiety comprises a CDR1 comprising the amino acid sequence of SEQ ID NO: 270 and a CDR3 comprising the amino acid sequence of SEQ ID NO: 300. In some embodiments, the at least one targeting moiety comprises a CDR1 comprising the amino acid sequence of SEQ ID NO: 271 and a CDR3 comprising the amino acid sequence of SEQ ID NO: 301. In some embodiments, the at least one targeting moiety comprises a CDR1 comprising the amino acid sequence of SEQ ID NO: 272 and a CDR3 comprising the amino acid sequence of SEQ ID NO: 302. In some embodiments, the at least one targeting moiety comprises a CDR1 comprising the amino acid sequence of SEQ ID NO: 273 and a CDR3 comprising the amino acid sequence of SEQ ID NO: 303. In some embodiments, the at least one targeting moiety comprises a CDR1 comprising the amino acid sequence of SEQ ID NO: 274 and a CDR3 comprising the amino acid sequence of SEQ ID NO: 304. In some embodiments, the at least one targeting moiety comprises a CDR1 comprising the amino acid sequence of SEQ ID NO: 275 and a CDR3 comprising the amino acid sequence of SEQ ID NO: 305. In some embodiments, the at least one targeting moiety comprises a CDR1 comprising the amino acid sequence of SEQ ID NO: 395 and a CDR3 comprising the amino acid sequence of SEQ ID NO: 396.

In some embodiments, the at least one targeting moiety in the SARS-CoV-2 coronavirus binding agent of the disclosure may further comprise an HV2 and/or an HV4, wherein the HV2 comprises an amino acid sequence selected from any one of SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO:318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO:333, SEQ ID NO: 334, SEQ ID NO: 335, and SEQ ID NO: 397, and wherein the HV4 comprises an amino acid sequence selected from any one of SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, and SEQ ID NO: 398.

In some embodiments, the at least one targeting moiety having the CDR1 of SEQ ID NO: 246 and the CDR3 of SEQ ID NO: 276 further comprises a HV2 comprising the amino acid sequence of SEQ ID NO: 306 and an HV4 comprising the amino acid sequence of SEQ ID NO: 336. In some embodiments, the at least one targeting moiety having the CDR1 of SEQ ID NO: 247 and the CDR3 of SEQ ID NO: 277 further comprises a HV2 comprising the amino acid sequence of SEQ ID NO: 307 and an HV4 comprising the amino acid sequence of SEQ ID NO: 337. In some embodiments, the at least one targeting moiety having the CDR1 of SEQ ID NO: 248 and the CDR3 of SEQ ID NO: 278 further comprises a HV2 comprising the amino acid sequence of SEQ ID NO: 308 and an HV4 comprising the amino acid sequence of SEQ ID NO: 338. In some embodiments, the at least one targeting moiety having the CDR1 of SEQ ID NO: 249 and the CDR3 of SEQ ID NO: 279 further comprises a HV2 comprising the amino acid sequence of SEQ ID NO: 309 and an HV4 comprising the amino acid sequence of SEQ ID NO: 339. In some embodiments, the at least one targeting moiety having the CDR1 of SEQ ID NO: 250 and the CDR3 of SEQ ID NO: 280 further comprises a HV2 comprising the amino acid sequence of SEQ ID NO: 310 and an HV4 comprising the amino acid sequence of SEQ ID NO: 340. In some embodiments, the at least one targeting moiety having the CDR1 of SEQ ID NO: 251 and the CDR3 of SEQ ID NO: 281 further comprises a HV2 comprising the amino acid sequence of SEQ ID NO: 311 and an HV4 comprising the amino acid sequence of SEQ ID NO: 341. In some embodiments, the at least one targeting moiety having the CDR1 of SEQ ID NO: 252 and the CDR3 of SEQ ID NO: 282 further comprises a HV2 comprising the amino acid sequence of SEQ ID NO: 312 and an HV4 comprising the amino acid sequence of SEQ ID NO: 342. In some embodiments, the at least one targeting moiety having the CDR1 of SEQ ID NO: 253 and the CDR3 of SEQ ID NO: 283 further comprises a HV2 comprising the amino acid sequence of SEQ ID NO: 313 and an HV4 comprising the amino acid sequence of SEQ ID NO: 343. In some embodiments, the at least one targeting moiety having the CDR1 of SEQ ID NO: 254 and the CDR3 of SEQ ID NO: 284 further comprises a HV2 comprising the amino acid sequence of SEQ ID NO: 314 and an HV4 comprising the amino acid sequence of SEQ ID NO: 344. In some embodiments, the at least one targeting moiety having the CDR1 of SEQ ID NO: 255 and the CDR3 of SEQ ID NO: 285 further comprises a HV2 comprising the amino acid sequence of SEQ ID NO: 315 and an HV4 comprising the amino acid sequence of SEQ ID NO: 345. In some embodiments, the at least one targeting moiety having the CDR1 of SEQ ID NO: 256 and the CDR3 of SEQ ID NO: 286 further comprises a HV2 comprising the amino acid sequence of SEQ ID NO: 316 and an HV4 comprising the amino acid sequence of SEQ ID NO: 346. In some embodiments, the at least one targeting moiety having the CDR1 of SEQ ID NO: 257 and the CDR3 of SEQ ID NO: 287 further comprises a HV2 comprising the amino acid sequence of SEQ ID NO: 317 and an HV4 comprising the amino acid sequence of SEQ ID NO: 347. In some embodiments, the at least one targeting moiety having the CDR1 of SEQ ID NO: 258 and the CDR3 of SEQ ID NO: 288 further comprises a HV2 comprising the amino acid sequence of SEQ ID NO: 318 and an HV4 comprising the amino acid sequence of SEQ ID NO: 348. In some embodiments, the at least one targeting moiety having the CDR1 of SEQ ID NO: 259 and the CDR3 of SEQ ID NO: 289 further comprises a HV2 comprising the amino acid sequence of SEQ ID NO: 319 and an HV4 comprising the amino acid sequence of SEQ ID NO: 349. In some embodiments, the at least one targeting moiety having the CDR1 of SEQ ID NO: 260 and the CDR3 of SEQ ID NO: 290 further comprises a HV2 comprising the amino acid sequence of SEQ ID NO: 320 and an HV4 comprising the amino acid sequence of SEQ ID NO: 350. In some embodiments, the at least one targeting moiety having the CDR1 of SEQ ID NO: 261 and the CDR3 of SEQ ID NO: 291 further comprises a HV2 comprising the amino acid sequence of SEQ ID NO: 321 and an HV4 comprising the amino acid sequence of SEQ ID NO: 351. In some embodiments, the at least one targeting moiety having the CDR1 of SEQ ID NO: 262 and the CDR3 of SEQ ID NO: 292 further comprises a HV2 comprising the amino acid sequence of SEQ ID NO: 322 and an HV4 comprising the amino acid sequence of SEQ ID NO: 352. In some embodiments, the at least one targeting moiety having the CDR1 of SEQ ID NO: 263 and the CDR3 of SEQ ID NO: 293 further comprises a HV2 comprising the amino acid sequence of SEQ ID NO: 323 and an HV4 comprising the amino acid sequence of SEQ ID NO: 353. In some embodiments, the at least one targeting moiety having the CDR1 of SEQ ID NO: 264 and the CDR3 of SEQ ID NO: 294 further comprises a HV2 comprising the amino acid sequence of SEQ ID NO: 324 and an HV4 comprising the amino acid sequence of SEQ ID NO: 354. In some embodiments, the at least one targeting moiety having the CDR1 of SEQ ID NO: 265 and the CDR3 of SEQ ID NO: 295 further comprises a HV2 comprising the amino acid sequence of SEQ ID NO: 325 and an HV4 comprising the amino acid sequence of SEQ ID NO: 355. In some embodiments, the at least one targeting moiety having the CDR1 of SEQ ID NO: 266 and the CDR3 of SEQ ID NO: 296 further comprises a HV2 comprising the amino acid sequence of SEQ ID NO: 326 and an HV4 comprising the amino acid sequence of SEQ ID NO: 356. In some embodiments, the at least one targeting moiety having the CDR1 of SEQ ID NO: 267 and the CDR3 of SEQ ID NO: 297 further comprises a HV2 comprising the amino acid sequence of SEQ ID NO: 327 and an HV4 comprising the amino acid sequence of SEQ ID NO: 357. In some embodiments, the at least one targeting moiety having the CDR1 of SEQ ID NO: 268 and the CDR3 of SEQ ID NO: 298 further comprises a HV2 comprising the amino acid sequence of SEQ ID NO: 328 and an HV4 comprising the amino acid sequence of SEQ ID NO: 358. In some embodiments, the at least one targeting moiety having the CDR1 of SEQ ID NO: 269 and the CDR3 of SEQ ID NO: 299 further comprises a HV2 comprising the amino acid sequence of SEQ ID NO: 329 and an HV4 comprising the amino acid sequence of SEQ ID NO: 359. In some embodiments, the at least one targeting moiety having the CDR1 of SEQ ID NO: 270 and the CDR3 of SEQ ID NO: 300 further comprises a HV2 comprising the amino acid sequence of SEQ ID NO: 330 and an HV4 comprising the amino acid sequence of SEQ ID NO: 360. In some embodiments, the at least one targeting moiety having the CDR1 of SEQ ID NO: 271 and the CDR3 of SEQ ID NO: 301 further comprises a HV2 comprising the amino acid sequence of SEQ ID NO: 331 and an HV4 comprising the amino acid sequence of SEQ ID NO: 361. In some embodiments, the at least one targeting moiety having the CDR1 of SEQ ID NO: 272 and the CDR3 of SEQ ID NO: 302 further comprises a HV2 comprising the amino acid sequence of SEQ ID NO: 332 and an HV4 comprising the amino acid sequence of SEQ ID NO: 362. In some embodiments, the at least one targeting moiety having the CDR1 of SEQ ID NO: 273 and the CDR3 of SEQ ID NO: 303 further comprises a HV2 comprising the amino acid sequence of SEQ ID NO: 333 and an HV4 comprising the amino acid sequence of SEQ ID NO: 363. In some embodiments, the at least one targeting moiety having the CDR1 of SEQ ID NO: 274 and the CDR3 of SEQ ID NO: 304 further comprises a HV2 comprising the amino acid sequence of SEQ ID NO: 334 and an HV4 comprising the amino acid sequence of SEQ ID NO: 364. In some embodiments, the at least one targeting moiety having the CDR1 of SEQ ID NO: 275 and the CDR3 of SEQ ID NO: 305 further comprises a HV2 comprising the amino acid sequence of SEQ ID NO: 335 and an HV4 comprising the amino acid sequence of SEQ ID NO: 365. In some embodiments, the at least one targeting moiety having the CDR1 of SEQ ID NO: 395 and the CDR3 of SEQ ID NO: 396 further comprises a HV2 comprising the amino acid sequence of SEQ ID NO: 397 and an HV4 comprising the amino acid sequence of SEQ ID NO: 398.

In some embodiments, the SARS-CoV-2 coronavirus binding agent of the disclosure comprises at least one targeting moiety that is a full-length antibody, a single-domain antibody, a recombinant heavy-chain-only antibody (Vm), a single-chain antibody (scFv), a shark heavy-chain-only antibody (VNAR), a microprotein, a darpin, an anticalin, an adnectin, an aptamer, a Fv, a Fab, a Fab′, a F(ab′)₂, a peptide mimetic molecule, a natural ligand for a receptor, or a synthetic molecule. In some embodiments, the at least one targeting moiety is a single-domain antibody. In some embodiments, the at least one targeting moiety comprises a V_{HH}. In some embodiments, the at least one targeting moiety comprises a humanized V_{HH}. In some embodiments, the at least one targeting moiety comprises a shark V_{HH}. In some embodiments, the at least one targeting moiety comprises a camelid V_{HH}.

As disclosed above, the SARS-CoV-2 coronavirus binding agents of the disclosure have been shown to bind to the Spike glycoprotein of SARS-CoV-2 coronavirus with high affinity as measured using BioLayer Interferometry. In some embodiments, the SARS-CoV-2 coronavirus binding agents of the disclosure binds to the Spike glycoprotein of SARS-CoV-2 coronavirus (WA-1 strain) with a dissociation constant (K_{D}) of about 500 nM or less, about 400 nM or less, about 300 nM or less, about 200 nM or less, about 150 nM or less, about 100 nM or less, about 50 nM or less, about 10 nM or less, about 1 nM or less, or about 500 pM or less as measured using BioLayer Interferometry, as described herein.

In some embodiments, the SARS-CoV-2 coronavirus binding agents of the disclosure binds to the Spike glycoprotein of SARS-CoV-2 coronavirus (B.1.1.7 variant) with a dissociation constant (K_{D}) of about 500 nM or less, about 400 nM or less, about 300 nM or less, about 200 nM or less, about 150 nM or less, about 100 nM or less, about 50 nM or less, about 10 nM or less, about 1 nM or less, or about 500 pM or less as measured using BioLayer Interferometry, as described herein.

In some embodiments, the SARS-CoV-2 coronavirus binding agents of the disclosure binds to the Spike glycoprotein of SARS-CoV-2 coronavirus (B.1.351 variant) with a dissociation constant (K_{D}) of about 500 nM or less, about 400 nM or less, about 300 nM or less, about 200 nM or less, about 150 nM or less, about 100 nM or less, about 50 nM or less, about 10 nM or less, or about 1 nM or less, or about 500 pM or less as measured using BioLayer Interferometry, as described herein.

In some embodiments, the SARS-CoV-2 coronavirus binding agents of the disclosure binds to the Spike glycoprotein of SARS-CoV-2 coronavirus (B.1.617.2 variant) with a dissociation constant (K_{D}) of about 500 nM or less, about 400 nM or less, about 300 nM or less, about 200 nM or less, about 150 nM or less, about 100 nM or less, about 50 nM or less, about 10 nM or less, or about 1 nM or less, or about 500 pM or less as measured using BioLayer Interferometry, as described herein.

In some embodiments, the SARS-CoV-2 coronavirus binding agents of the disclosure binds to the Spike glycoprotein of SARS-CoV-2 coronavirus (WA-1 strain) and variants thereof, including one or more of the B.1.1.7 variant, the B.1.351 variant, and/or the B.1.617.2 variant, with a dissociation constant (K_{D}) of about 500 nM or less, about 400 nM or less, about 300 nM or less, about 200 nM or less, about 150 nM or less, about 100 nM or less, about 50 nM or less, about 10 nM or less, about 1 nM or less, or about 500 pM or less as measured using BioLayer Interferometry, as described herein.

In some embodiments, the SARS-CoV-2 coronavirus binding agents of the disclosure bind to SARS-CoV-2 coronavirus and the related sarbecovirus SARS-CoV-1 with high affinity. In some embodiments, the SARS-CoV-2 coronavirus binding agents of the disclosure binds to the Spike glycoprotein of both the SARS-CoV-2 coronavirus and the SARS-CoV-1 coronavirus with a dissociation constant (K_{D}) of about 500 nM or less, about 400 nM or less, about 300 nM or less, about 200 nM or less, about 150 nM or less, about 100 nM or less, about 50 nM or less, about 10 nM or less, or about 1 nM or less, or about 500 pM or less as measured using BioLayer Interferometry, as described herein. In some embodiments, the SARS-CoV-2 coronavirus binding agents of the disclosure binds to the Spike glycoprotein of both the SARS-CoV-2 coronavirus and the SARS-CoV-1 coronavirus with a dissociation constant (K_{D}) of about 50-400 nM, about 50-200 nM, or about 100-200 nM or less, as measured using BioLayer Interferometry, as described herein.

In some embodiments, the SARS-CoV-2 coronavirus binding agents of the disclosure binds to the Spike glycoprotein of SARS-CoV-2 coronavirus (WA-1 strain) with a rate of association (kₒₙ-rate) of between about 10¹ M⁻¹s⁻¹ to about 10⁷ M⁻¹s⁻¹, about 10² M⁻¹s⁻¹ to about 10⁷ M⁻¹s⁻¹, about 10³ M⁻¹s⁻¹ to about 10⁷ M⁻¹s⁻¹, about 10⁴ M⁻¹s⁻¹ to about 10⁷ M⁻¹s⁻¹, or about 10⁴ M⁻¹s⁻¹ to about 10⁶ M⁻¹s⁻¹ as measured using BioLayer Interferometry, as described herein. In some embodiments, the SARS-CoV-2 coronavirus binding agents of the disclosure binds to the Spike glycoprotein of SARS-CoV-2 coronavirus (B.1.1.7 variant) with a rate of association (kₒₙ-rate) of between about 10¹ M⁻¹s⁻¹ to about 10⁷M⁻¹s⁻¹, about 10² M⁻¹s⁻¹ to about 10⁷ M⁻¹s⁻¹, about 10³ M⁻¹s⁻¹ to about 10⁷ M⁻¹s⁻¹, about 10⁴ M⁻¹s⁻¹ to about 10⁷ M⁻¹s⁻¹, or about 10⁴ M⁻¹s⁻¹ to about 10⁶ M⁻¹s⁻¹ as measured using BioLayer Interferometry, as described herein. In some embodiments, the SARS-CoV-2 coronavirus binding agents of the disclosure binds to the Spike glycoprotein of SARS-CoV-2 coronavirus (B.1.351 variant) with a rate of association (kₒₙ-rate) of between about 10¹ M⁻¹s⁻¹ to about 10⁷M⁻¹s⁻¹, about 10² M⁻¹s⁻¹ to about 10⁷ M⁻¹s⁻¹, about 10³ M⁻¹s⁻¹ to about 10⁷ M⁻¹s⁻¹, about 10⁴ M⁻¹s⁻¹ to about 10⁷ M⁻¹s⁻¹, or about 10⁴ M⁻¹s⁻¹ to about 10⁶ M⁻¹s⁻¹ as measured using BioLayer Interferometry, as described herein. In some embodiments, the SARS-CoV-2 coronavirus binding agents of the disclosure binds to the Spike glycoprotein of SARS-CoV-2 coronavirus (B.1.617.2 variant) with a rate of association (kₒₙ-rate) of between about 10¹ M⁻¹s⁻¹ to about 10⁷ M⁻¹s⁻¹, about 10² M⁻¹s⁻¹ to about 10⁷ M⁻¹s⁻¹, about 10³ M⁻¹s⁻¹ to about 10⁷ M⁻¹s⁻¹, about 10⁴ M⁻¹s⁻¹ to about 10⁷ M⁻¹s⁻¹, or about 10⁴ M⁻¹s⁻¹ to about 10⁶ M⁻¹s⁻¹ as measured using BioLayer Interferometry, as described herein. In some embodiments, the SARS-CoV-2 coronavirus binding agents of the disclosure binds to the Spike glycoprotein of SARS-CoV-2 coronavirus (WA-1 strain) and variants thereof, including one or more of the B.1.1.7 variant, the B.1.351 variant, and/or the B.1.617.2 variant, with a rate of association (kₒₙ-rate) of between about 10¹ M⁻¹s⁻¹ to about 10⁷ M⁻¹s⁻¹, about 10² M⁻¹s⁻¹ to about 10⁷ M⁻¹s⁻¹, about 10³ M⁻¹s⁻¹ to about 10⁷ M⁻¹s⁻¹, about 10⁴ M⁻¹s⁻¹ to about 10⁷ M⁻¹s⁻¹, or about 10⁴ M⁻¹s⁻¹ to about 10⁶ M⁻¹s⁻¹ as measured using BioLayer Interferometry, as described herein.

In some embodiments, the SARS-CoV-2 coronavirus binding agents of the disclosure binds to the Spike glycoprotein of SARS-CoV-2 coronavirus (WA-1 strain) with a rate of dissociation (k_{off} rate) between about 10⁻¹ s⁻¹ to about 10⁻⁶ s⁻¹, about 10⁻² s⁻¹ to about 10⁻⁶ s⁻¹, about 10⁻³ s⁻¹ to about 10⁻⁶ s⁻¹, about 10-4 s⁻¹ to about 10⁻⁶ s⁻¹, or about 10⁻² s⁻¹ to about 10-5 s⁻¹ as measured using BioLayer Interferometry, as described herein. In some embodiments, the SARS-CoV-2 coronavirus binding agents of the disclosure binds to the Spike glycoprotein of CoV-2 coronavirus (B.1.1.7 variant) with a rate of dissociation (k_{off} rate) between about 10⁻¹ s⁻¹ to about 10⁻⁶ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, or about 10⁻² s⁻¹ to about 10⁻⁵ s⁻¹ as measured using BioLayer Interferometry, as described herein. In some embodiments, the SARS-CoV-2 coronavirus binding agents of the disclosure binds to the Spike glycoprotein of SARS-CoV-2 coronavirus (B.1.351 variant) with a rate of dissociation (k_{off} rate) between about 10⁻¹ s⁻¹ to about 10⁻⁶ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, or about 10⁻² s⁻¹ to about 10-5 s⁻¹ as measured using BioLayer Interferometry, as described herein. In some embodiments, the SARS-CoV-2 coronavirus binding agents of the disclosure binds to the Spike glycoprotein of SARS-CoV-2 coronavirus (B.1.617.2 variant) with a rate of dissociation (k_{off} rate) between about 10⁻¹ s⁻¹ to about 10⁻⁶ s⁻¹, about 10-2 s⁻¹ to about 10-6 s⁻¹, about 10-3 s⁻¹ to about 10⁻⁶ s⁻¹, about 10-4 s⁻¹ to about 10⁻⁶ s⁻¹, or about 10⁻² s⁻¹ to about 10⁻⁵ s⁻¹ as measured using BioLayer Interferometry, as described herein. In some embodiments, the SARS-CoV-2 coronavirus binding agents of the disclosure binds to the Spike glycoprotein of SARS-CoV-2 coronavirus (WA-1 strain) and variants thereof, including one or more of the B.1.1.7 variant, the B.1.351 variant, and/or the B.1.617.2 variant, with a rate of dissociation (k_{off} rate) between about 10⁻¹ s⁻¹ to about 10-6 s⁻¹, about 10⁻² s⁻¹ to about 10-6s⁻¹, about 10-3 s⁻¹ to about 10⁻⁶ s⁻¹, about 10-4 s⁻¹ to about 10⁻⁶ s⁻¹, or about 10⁻² s⁻¹ to about 10-5 s⁻¹ as measured using BioLayer Interferometry, as described herein.

In some embodiments, the SARS-CoV-2 coronavirus binding agents of the disclosure has a pseudovirus neutralization IC50 (μg/mL) value for SARS-CoV-2 coronavirus (WA-1 strain) of about 100 or less, about 50 or less, about 40 or less, about 30 or less, about 20 or less, about 10 or less, about 5 or less, about 3 or less, about 1 or less, about 0.5 or less, about 0.2 or less, about 0.1 or less, about 0.05 or less, or about 0.03 or less. In some embodiments, the SARS-CoV-2 coronavirus binding agents of the disclosure has a pseudovirus neutralization IC50 (μg/mL) value for CoV-2 coronavirus (B.1.1.7 variant) of about 100 or less, about 50 or less, about 40 or less, about 30 or less, about 20 or less, about 10 or less, about 5 or less, about 3 or less, about 1 or less, about 0.5 or less, about 0.2 or less, about 0.1 or less, about 0.05 or less, or about 0.03 or less. In some embodiments, the SARS-CoV-2 coronavirus binding agents of the disclosure has a pseudovirus neutralization IC50 (μg/mL) value for SARS-CoV-2 coronavirus (B.1.351 variant) of about 100 or less, about 50 or less, about 40 or less, about 30 or less, about 20 or less, about 10 or less, about 5 or less, about 3 or less, about 1 or less, about 0.5 or less, about 0.2 or less, about 0.1 or less, about 0.05 or less, or about 0.03 or less. In some embodiments, the SARS-CoV-2 coronavirus binding agents of the disclosure has a pseudovirus neutralization IC50 (μg/mL) value for SARS-CoV-2 coronavirus (B.1.617.2 variant) of about 100 or less, about 50 or less, about 40 or less, about 30 or less, about 20 or less, about 10 or less, about 5 or less, about 3 or less, about 1 or less, about 0.5 or less, about 0.2 or less, about 0.1 or less, about 0.05 or less, or about 0.03 or less. In some embodiments, the SARS-CoV-2 coronavirus binding agents of the disclosure has a pseudovirus neutralization IC50 (μg/mL) value for SARS-CoV-2 coronavirus (WA-1 strain) or variant thereof, including one or more of the B.1.1.7 variant, the B.1.351 variant, and/or the B.1.617.2 variant, of about 100 or less, about 50 or less, about 40 or less, about 30 or less, about 20 or less, about 10 or less, about 5 or less, about 3 or less, about 1 or less, about 0.5 or less, about 0.2 or less, about 0.1 or less, about 0.05 or less, or about 0.03 or less.

In some embodiments, the SARS-CoV-2 coronavirus binding agents of the disclosure neutralizes SARS-CoV-2 coronavirus and the related sarbecovirus SARS-CoV-1. In some embodiments, the SARS-CoV-2 coronavirus binding agents of the disclosure has a pseudovirus neutralization IC50 (μg/mL) value for SARS-CoV-1 coronavirus of about 100 or less, about 50 or less, about 40 or less, about 30 or less, about 20 or less, about 10 or less, about 5 or less, about 3 or less, about 1 or less, about 0.5 or less, about 0.2 or less, about 0.1 or less, or about 0.05 or less.

In some embodiments, the SARS-CoV-2 coronavirus binding agents of the disclosure bind to the receptor binding domain (RBD) of the spike glycoprotein of SARS-CoV-2 coronavirus. In some embodiments, the SARS-CoV-2 coronavirus binding agents of the disclosure bind to the N-terminus domain (NTD) of the spike glycoprotein of SARS-CoV-2 coronavirus. In some embodiments, the SARS-CoV-2 coronavirus binding agents of the disclosure bind to the spike glycoprotein of SARS-CoV-2 coronavirus in a section close to the ACE2 binding site. In some embodiments, the SARS-CoV-2 coronavirus binding agents of the disclosure bind to the spike glycoprotein of SARS-CoV-2 coronavirus in a section comprising amino acid residue Arg346 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residue Asn354 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residue Lys356 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residue Tyr369 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Arg346-Arg357 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Ser375-Ser379 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Phe374-Thr385 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Ala411-Gln414 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Asp427-Phe429 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Asp405-Ala411 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Ser375-Tyr380 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Ile468-Glu417 of the SARS-CoV-2 spike glycoprotein. In some embodiments, the SARS-CoV-2 coronavirus binding agents of the disclosure bind to the spike glycoprotein of SARS-CoV-2 coronavirus in a section comprising amino acid residues Gly447-Asn450 of the SARS-CoV-2 spike glycoprotein.

In some embodiments, the SARS-CoV-2 coronavirus binding agents of the disclosure bind to the receptor binding domain (RBD) of the spike glycoprotein of SARS-CoV-2 coronavirus and the RBD of the related sarbecovirus SARS-CoV-1. In some embodiments, the SARS-CoV-2 coronavirus binding agents of the disclosure bind to the N-terminal domain (NTD) of the spike glycoprotein of SARS-CoV-2 coronavirus and the NTD of the related sarbecovirus SARS-CoV-1.

The SARS-CoV-2 coronavirus binding agents disclosed herein may be used to neutralize SARS-CoV-2 coronavirus. In some embodiments, the SARS-CoV-2 coronavirus binding agent of the disclosure neutralizes SARS-CoV-2 coronavirus in vitro. In some embodiments, the SARS-CoV-2 coronavirus binding agent of the disclosure neutralizes SARS-CoV-2 coronavirus in vivo.

### Chimeric Proteins

The disclosure also provides chimeric proteins that comprise one or a plurality of the SARS-CoV-2 coronavirus binding agents disclosed herein and one or a plurality of heterologous proteins, such as an Fc domain, a ferritin, a lumazine synthase, an antibody, such as an antibody that binds to human serum albumin, or a combination thereof. Because the SARS-CoV-2 coronavirus binding agents of the disclosure bind to the Spike glycoprotein of SARS-CoV-2 coronavirus with high affinity, the chimeric proteins of one or more of the SARS-CoV-2 coronavirus binding agents disclosed herein will also have similar or even higher binding affinity to the Spike glycoprotein of SARS-CoV-2 coronavirus and can be used to neutralize SARS-CoV-2 coronavirus. In some embodiments, therefore, the chimeric proteins of the disclosure neutralize SARS-CoV-2 coronavirus in vitro. In other embodiments, the chimeric proteins of the disclosure neutralize SARS-CoV-2 coronavirus in vivo.

One example of the heterologous proteins useful for preparing the chimeric proteins of the disclosure is ferritin. Ferritin is an iron storage protein found in almost all living organisms. Ferritin has been extensively studied and engineered for a number of potential biochemical/biomedical purposes (Iwahori, K. U.S. Patent 2009/0233377 (2009); Meldrum, F. C. et al. Science 257, 522-523 (1992); Naitou, M. et al. U.S. Patent 2011/0038025 (2011); Yamashita, I. Biochim BiophysActa 1800, 846-857 (2010)), including its use as a vaccine platform for displaying exogenous epitope peptides (Carter, D. C. et al., U.S. Patent 2006/0251679 (2006); Li, C. Q. et al. Industrial Biotechnol 2, 143-147 (2006)). Ferritin protein self-assembles into a globular protein complex comprising multiple individual monomers. The molecular architecture of ferritin, which consists of 24 subunits assembling into an octahedral cage with 432 symmetries, can display multimeric antigens on its surface.

Ferritin genes are found in many species and generally show a conserved highly alpha-helical structure despite sequence variation. As such, any ferritin can be used in the chimeric proteins described herein, including bacterial, insect, and human ferritin, despite its sequence identity to any particularly described ferritin.

In some embodiments, the ferritin is bacterial, insect, fungal, bird, or mammalian. In some embodiments, the ferritin is human ferritin, optionally with one or more mutations. In some embodiments, the ferritin is bacterial ferritin, optionally with one or more mutations. In some embodiments, the ferritin is Helicobacter pylori ferritin, optionally with one or more mutations. In some embodiments, the ferritin is Pyrococcus furiosus ferritin (NCBI seq WP_011011871.1), optionally with one or more mutations. In certain embodiments, a region comprising N-terminal amino acids of the ferritin protein are removed. For example, amino acids 1-4 of the wild-type Helicobacter pylori ferritin protein may be removed. More specific regions are described in Zhang, Y. 2011, Int. J. Mol. Sci., 12, 5406-5421, which is incorporated herein by reference in its entirety. In some embodiments, the ferritin comprises a sequence having greater than about 70%, greater than about 75%, greater than about 80%, greater than about 85%, greater than about 90%, greater than about 95%, greater than about 97%, greater than about 98%, or greater than about 99% identity to a wild-type ferritin, including, but not limited to H. pylori ferritin, P. furiosus ferritin or human ferritin.

In some embodiments, the ferritin comprises one or more mutations. In some embodiments, the one or more mutations comprise changes to the amino acid sequence of a wild-type ferritin and/or an insertion, e.g., at the N- or C-terminus. In some embodiments, one, two, three, four, five, or more different amino acids are mutated in the ferritin as compared to wild-type ferritin (in some embodiments, in addition to any N-terminal insertion). In general, a mutation simply refers to a difference in the sequence (such as a substituted, added, or deleted amino acid residue or residues) relative to the corresponding wild-type ferritin. In some embodiments, the ferritin is a H. pylori ferritin with one or more mutations.

Human-compatible glycosylation can contribute to safety and efficacy in recombinant drug products. Regulatory approval may be contingent on demonstrating appropriate glycosylation as a critical quality attribute (see Zhang et al., Drug Discovery Today 21(5):740-765 (2016)). N-glycans can result from glycosylation of asparagine side chains and can differ in structure between humans and other organisms such as bacteria and yeast. Thus, it may be desirable to reduce or eliminate non-human glycosylation and/or N-glycan formation in ferritin. In some embodiments, controlling glycosylation of ferritin improves the efficacy and/or safety of the composition, especially when used for human vaccination.

In some embodiments, ferritin is mutated to inhibit formation of an N-glycan. In some embodiments, a mutated ferritin has reduced glycosylation as compared to its corresponding wild-type ferritin.

Another example of the heterologous proteins useful for preparing the chimeric proteins of the disclosure is lumazine synthase or a portion thereof. In some embodiments, the SARS-CoV-2 coronavirus binding agent is joined to at least about 50, at least about 100 or least about 150 amino acids from lumazine synthase, wherein the protein construct is capable of forming a nanoparticle. In some embodiments, the SARS-CoV-2 coronavirus binding agent is joined to a protein at least about 85%, at least about 90%, at least about 95%, or at least about 98% identical to lumazine synthase, wherein the protein construct is capable of forming a nanoparticle. In some embodiments, the lumazine synthase or a portion thereof comprised in the chimeric proteins of the disclosure is the lumazine synthase from Aquifex aeolicus.

Another example of the heterologous proteins useful for preparing the chimeric proteins of the disclosure is an antibody that binds to a target of interest, such as a target that would increase the half life or stability of the chimeric protein, particularly in an in vivo setting. In some embodiments, the heterologous protein is an antibody that binds to human serum albumin.

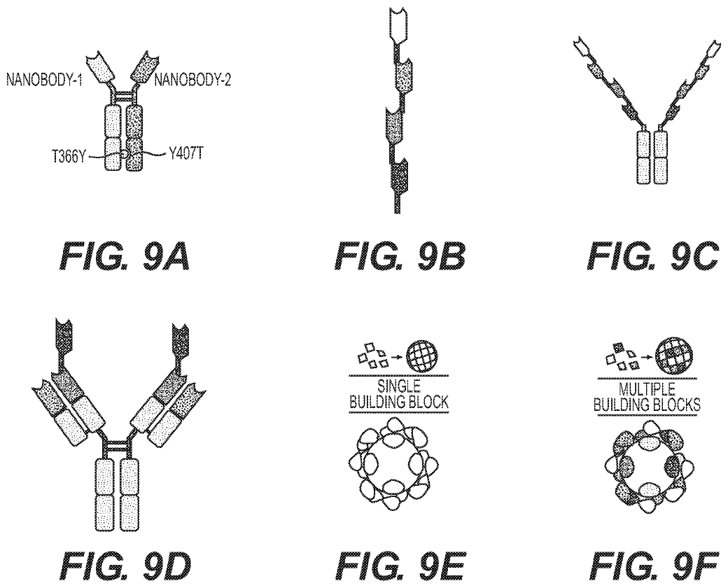

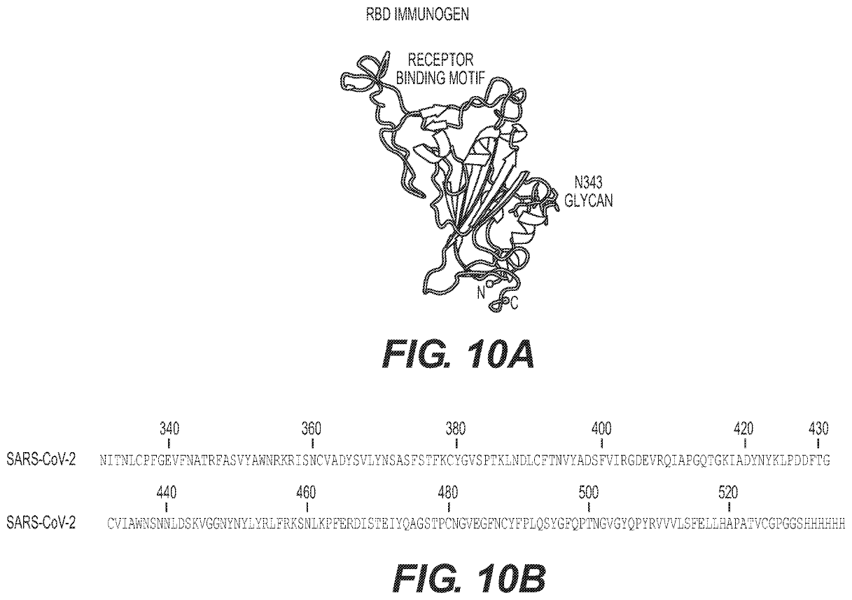

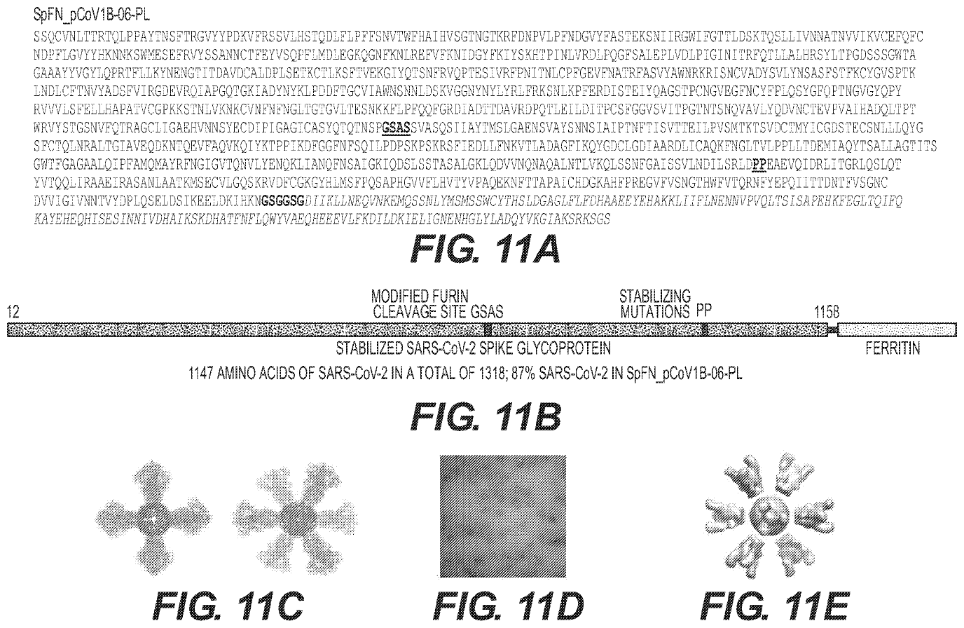

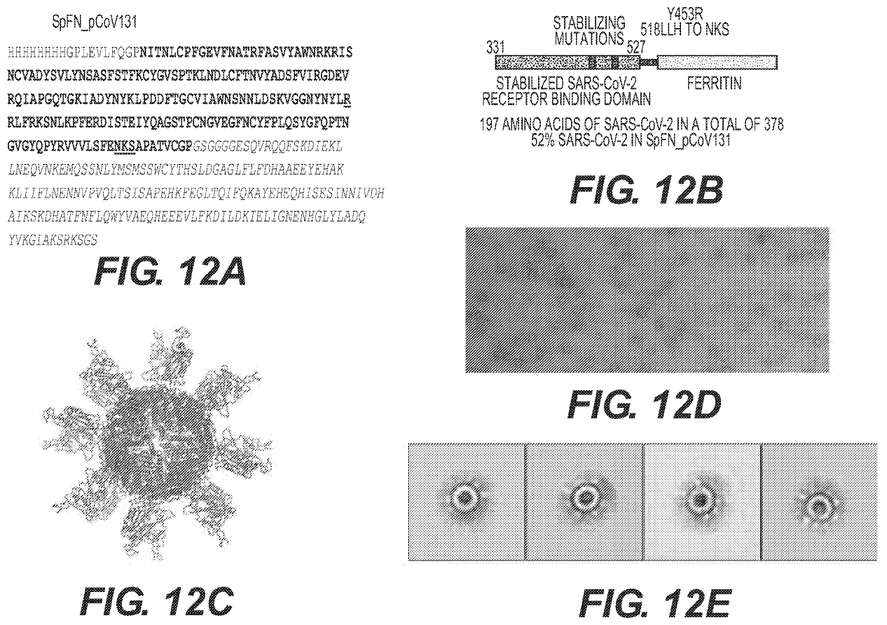

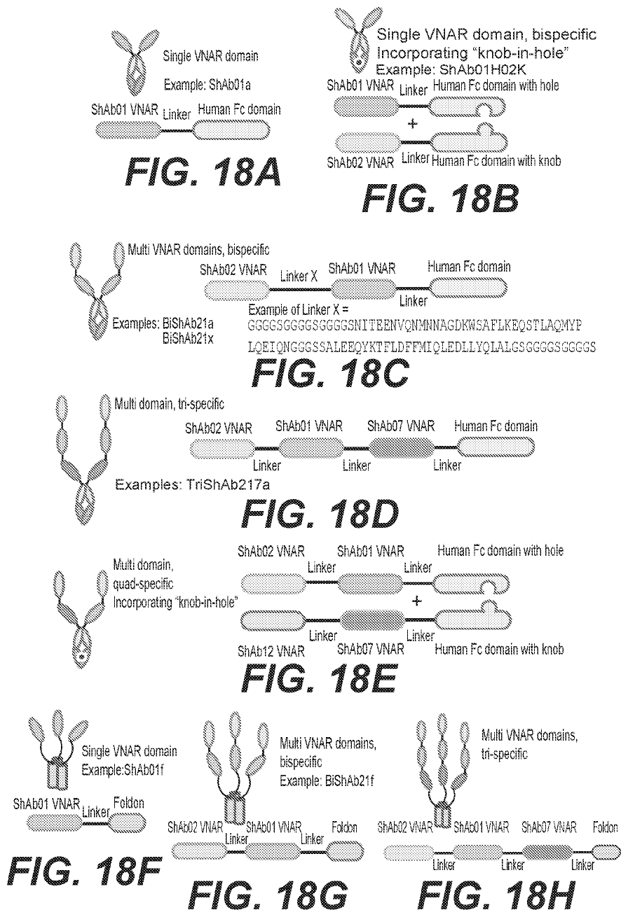

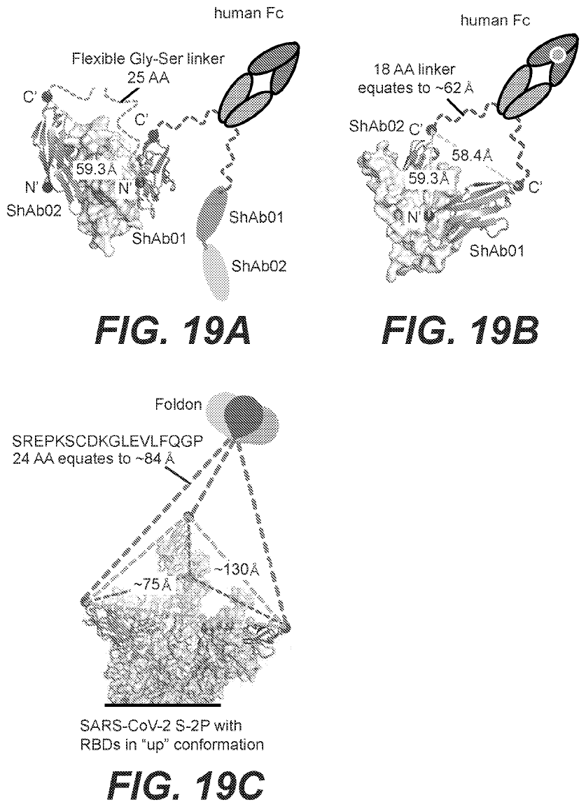

The ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies can be designed in different presentation modalities that are known in the art, including, but not limited to, those shown in FIG. 9A-9F and FIG. 18A-18H, to generate chimeric proteins. For example, a knob-in-hole design allows two different shark nanobodies to be merged into a single antibody (e.g., FIG. 9A). In a “beads on a string” format, different shark nanobodies can be combined into a single molecule (e.g., FIG. 9B). The “beads on a string” format can also be modified to include a linked Fc domain (e.g., FIG. 9C). A shark nanobody can be attached onto a human antibody (e.g., FIG. 9D). Shark nanobodies can also be combined with a protein with self-assembling multimerization properties (e.g., ferritin), using either a single shark nanobody (e.g., FIG. 9E) or multiple shark nanobodies (e.g., FIG. 9F).

In some embodiments, the chimeric protein of the disclosure comprises one single SARS-CoV-2 coronavirus binding agent or one single binding agent that neutralizes SARS-CoV-2 coronavirus in vitro or in vivo, such as any one of the ShAb nanobodies disclosed herein. In some embodiments, the chimeric protein of the disclosure comprises multiple (e.g., 2, 3, 4, 5, or more) SARS-CoV-2 coronavirus binding agents or multiple (e.g., 2, 3, 4, 5, or more) binding agents that neutralizes SARS-CoV-2 coronavirus in vitro or in vivo, such as one or more ShAb nanobodies disclosed herein. In such embodiments, the chimeric protein may comprise more than one heterologous protein, such as an Fc domain, a ferritin, a lumazine synthase, an antibody (e.g., antibody that binds to human serum albumin) or a combination thereof. In some embodiments, the Fc domain is a human Fc domain. In some embodiments, the Fc domain is a human IgG or IgM Fc domain. In some embodiments, the Fc domain is a human IgG1 Fc domain. In some embodiments, the chimeric protein of the disclosure comprises at least two identical Fc domains. In some embodiments, the chimeric protein of the disclosure comprises at least two Fc domains with different structure (e.g., sequence). It is known that cysteine residue(s) may be introduced into the Fc domain, thereby allowing interchain disulfide bond formation in this region. The homodimeric antibody thus generated may have improved internalization capability and/or increased complement-mediated cell killing and antibody-dependent cellular cytotoxicity (ADCC). See, Caron et al., J. Exp Med. 176:1191-95 (1992) and Shopes, B. J. Immunol. 148:2918-22 (1992). Homodimeric antibodies with enhanced anti-viral activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-30 (1989).

In some embodiments, the chimeric protein of the disclosure is a multivalent construct comprising at least two SARS-CoV-2 coronavirus binding agents or at least two binding agents that neutralizes SARS-CoV-2 coronavirus in vitro or in vivo, such as at least two of the ShAb nanobodies disclosed herein. In some embodiments, the chimeric protein of the disclosure is a multivalent construct comprising at least two copies of a single ShAb nanobody as disclosed herein. For example, in some embodiments, the chimeric protein of the disclosure comprises 2 copies of the ShAb01 nanobody (e.g., SEQ ID NO: 131) or 3 copies of the ShAb01 nanobody (e.g., SEQ ID NO: 390). In some embodiments, the chimeric protein of the disclosure comprises 2 copies of ShAb02 nanobody (e.g., SEQ ID NO: 132) or 3 copies of the ShAb02 nanobody (e.g., SEQ ID NO: 391). In some embodiments, the chimeric protein of the disclosure comprises 3 copies of the ShAb19 nanobody (e.g., SEQ ID NO: 392). In some embodiments, the chimeric protein of the disclosure comprises 3 copies of the ShAb23 nanobody (e.g., SEQ ID NO: 393).

In some embodiments, the chimeric protein of the disclosure is a multivalent construct comprising at least two different ShAb nanobodies disclosed herein. For example, in some embodiments, the chimeric protein of the disclosure comprises the ShAb01 nanobody and the ShAb02 nanobody (e.g., SEQ ID NO: 116-120). In some embodiments, the chimeric protein of the disclosure comprises the ShAb02 nanobody and the ShAb10 nanobody (e.g., SEQ ID NO: 121-122). In some embodiments, the chimeric protein of the disclosure comprises the ShAb02 nanobody and the ShAb11 nanobody (e.g., SEQ ID NO: 123-124). In some embodiments, the chimeric protein of the disclosure is a multivalent construct comprising more than two different ShAb nanobodies disclosed herein. For example, in some embodiments, the chimeric protein of the disclosure comprises the ShAb01 nanobody, the ShAb02 nanobody, and the ShAb06 nanobody (e.g., SEQ ID NO: 127). In some embodiments, the chimeric protein of the disclosure comprises the ShAb01 nanobody, the ShAb02 nanobody, and the ShAb07 nanobody (e.g., SEQ ID NO: 128). In some embodiments, the chimeric protein of the disclosure comprises the ShAb01 nanobody, the ShAb02 nanobody, and the ShAb10 nanobody (e.g., SEQ ID NO: 129). In some embodiments, the chimeric protein of the disclosure comprises the ShAb01 nanobody, the ShAb02 nanobody, and the ShAb11 nanobody (e.g., SEQ ID NO: 130). Other ShAb nanobodies disclosed herein can be combined to make a multivalent, chimeric protein.

In some embodiments, the chimeric protein of the disclosure is a multispecific construct that binds to at least two different regions of the spike glycoprotein of SARS-CoV-2 coronavirus. In some embodiments, the chimeric protein of the disclosure is a multispecific construct that binds to both the RBD and NTD of the SARS-CoV-2 spike glycoprotein. For example, in some embodiments, the chimeric protein of the disclosure comprises at least one RBD-binding ShAb nanobody of the disclosure and at least one NTD-binding ShAb nanobody of the disclosure. The RBD-binding ShAb nanobodies of the disclosure include ShAb01, ShAb02, ShAb09, ShAb17, ShAb18, ShAb19, ShAb20, ShAb21, ShAb22, ShAb23, ShAb24, and ShAb29. The NTD-binding ShAb nanobodies of the disclosure include ShAb03, ShAb04, ShAb05, ShAb06, ShAb07, ShAb08, ShAb10, ShAb11, ShAb12, ShAb13, ShAb14, ShAb15, and ShAb16. Examples of such chimeric proteins include, but are not limited to, SEQ ID NO: 116-124 and SEQ ID NO: 127-130 disclosed herein. Because of this multispecificity, the chimeric proteins of the disclosure may have synergistic improvements in antigen-affinity and thus, the ability to neutralize SARS-CoV-2 coronavirus (WA-1 strain and variants thereof, including the B.1.1.7 variant, the B.1.351 variant, and/or the B.1.617.2 variant) in vitro or in vivo. Accordingly, in some embodiments, the chimeric protein of the disclosure has an increased binding affinity to SARS-CoV-2 spike glycoprotein as compared to the binding affinity of the individual ShAb nanobody used in generating the chimeric protein. In some embodiments, the chimeric protein of the disclosure has an increased ability to neutralize SARS-CoV-2 coronavirus in vitro or in vivo as compared to that of the individual ShAb nanobody used in generating the chimeric protein. Examples of such chimeric proteins with improved binding affinity to SARS-CoV-2 spike glycoprotein and/or increased ability to neutralize SARS-CoV-2 coronavirus in vitro or in vivo include, but not limited to, the chimeric proteins of SEQ ID NO: 116-133 and SEQ ID NO: 366-377, or combination thereof.

In some embodiments, the chimeric proteins of the disclosure may further comprise one or a plurality of linkers and/or one or a plurality of leader sequences. The linker sequence can be used to connect the SARS-CoV-2 coronavirus binding agent and the heterologous protein. In embodiments where the chimeric proteins are multivalent constructs, the linker sequence may be used to connect two SARS-CoV-2 coronavirus binding agents. In some embodiments therefore, the chimeric proteins of the disclosure comprise at least one of the ShAb nanobodies disclosed herein and at least one heterologous protein, such as a Fc domain (e.g., a human Fc domain, including but limited to a human IgG Fc domain, a human IgG1 Fc domain or a human IgM Fc domain), a multimerizing protein (e.g., ferritin, lumazine synthase), and antibody (e.g., antibody that binds to human serum albumin) or a combination thereof, wherein the at least one ShAb nanobody is connected to the at least one heterologous protein by at least one linker. In other embodiments, the chimeric proteins of the disclosure comprise at least two ShAb nanobodies disclosed herein and at least one heterologous protein, such as a Fc domain, a multimerizing protein (e.g., ferritin, lumazine synthase), or a combination thereof, wherein the at least 2 nanobodies are connected to each other by at least one linker. In some embodiments, the linker is 15-50, 15-40, 20-40, 15-30, 20-30, 20-25, or 23-25 amino acids in length. In some embodiments, the linker sequence comprises glycine and serine residues. In some embodiments, the linker sequence comprises the amino acid sequence of SEQ ID NO: 207, SEQ ID NO: 208, SEQ ID NO: 209, SEQ ID NO: 210, SEQ ID NO: 211, SEQ ID NO: 212, or SEQ ID NO: 213, or an amino acid sequence having at least about 80%, 85%, 90%, 95%, 96%, 97%, 98%, or 99% sequence identity with one of SEQ ID NO: 207, SEQ ID NO: 208, SEQ ID NO: 209, SEQ ID NO: 210, SEQ ID NO: 211, SEQ ID NO: 212, and SEQ ID NO: 213. In some embodiments, the leader sequence comprises the amino acid sequence of SEQ ID NO: 214 or SEQ ID NO: 215, or an amino acid sequence having at least about 80%, 85%, 90%, 95%, 96%, 97%, 98%, or 99% sequence identity with the amino acid sequence of SEQ ID NO: 214 or SEQ ID NO: 215. Other linker sequences are known and can be used to connect the SARS-CoV-2 coronavirus binding agents and/or the SARS-CoV-2 coronavirus binding agent and the heterologous protein.

In some embodiments, the chimeric proteins of the disclosure comprise an amino acid sequence that is at least about 80%, 85%, 90%, 95%, 96%, 97%, 98%, or 99% identical to the amino acid sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 390, SEQ ID NO: 391, SEQ ID NO: 392, or SEQ ID NO: 393.

In some embodiments, the chimeric proteins of the disclosure comprises the amino acid sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 390, SEQ ID NO: 391, SEQ ID NO: 392, or SEQ ID NO: 393.

### Nucleic Acids, Cloning and Expression Systems

The disclosure further provides isolated nucleic acids encoding the SARS-CoV-2 coronavirus binding agents and the chimeric proteins disclosed herein. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

The nucleic acids provided herein encode at least one CDR, both CDRs (i.e., CDR1 and CDR3), at least one HV, and/or both HVs (i.e. HV2 and HV4) of one of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies.

For example, in some embodiments, the disclosure provides an isolated nucleic acid that encodes the ShAb01 nanobody, wherein the isolated nucleic acid comprises the nucleotide sequence of SEQ ID NO: 31. In some embodiments, the disclosure provides an isolated nucleic acid that encodes the ShAb02 nanobody, wherein the isolated nucleic acid comprises the nucleotide sequence of SEQ ID NO: 32. In some embodiments, the disclosure provides an isolated nucleic acid that encodes the ShAb03 nanobody, wherein the isolated nucleic acid comprises the nucleotide sequence of SEQ ID NO: 33. In some embodiments, the disclosure provides an isolated nucleic acid that encodes the ShAb04 nanobody, wherein the isolated nucleic acid comprises the nucleotide sequence of SEQ ID NO: 34. In some embodiments, the disclosure provides an isolated nucleic acid that encodes the ShAb05 nanobody, wherein the isolated nucleic acid comprises the nucleotide sequence of SEQ ID NO: 35. In some embodiments, the disclosure provides an isolated nucleic acid that encodes the ShAb06 nanobody, wherein the isolated nucleic acid comprises the nucleotide sequence of SEQ ID NO: 36. In some embodiments, the disclosure provides an isolated nucleic acid that encodes the ShAb07 nanobody, wherein the isolated nucleic acid comprises the nucleotide sequence of SEQ ID NO: 37. In some embodiments, the disclosure provides an isolated nucleic acid that encodes the ShAb08 nanobody, wherein the isolated nucleic acid comprises the nucleotide sequence of SEQ ID NO: 38. In some embodiments, the disclosure provides an isolated nucleic acid that encodes the ShAb09 nanobody, wherein the isolated nucleic acid comprises the nucleotide sequence of SEQ ID NO: 39. In some embodiments, the disclosure provides an isolated nucleic acid that encodes the ShAb10 nanobody, wherein the isolated nucleic acid comprises the nucleotide sequence of SEQ ID NO: 40. In some embodiments, the disclosure provides an isolated nucleic acid that encodes the ShAb11 nanobody, wherein the isolated nucleic acid comprises the nucleotide sequence of SEQ ID NO: 41. In some embodiments, the disclosure provides an isolated nucleic acid that encodes the ShAb12 nanobody, wherein the isolated nucleic acid comprises the nucleotide sequence of SEQ ID NO: 42. In some embodiments, the disclosure provides an isolated nucleic acid that encodes the ShAb13 nanobody, wherein the isolated nucleic acid comprises the nucleotide sequence of SEQ ID NO: 43. In some embodiments, the disclosure provides an isolated nucleic acid that encodes the ShAb14 nanobody, wherein the isolated nucleic acid comprises the nucleotide sequence of SEQ ID NO: 44. In some embodiments, the disclosure provides an isolated nucleic acid that encodes the ShAb15 nanobody, wherein the isolated nucleic acid comprises the nucleotide sequence of SEQ ID NO: 45. In some embodiments, the disclosure provides an isolated nucleic acid that encodes the ShAb16 nanobody, wherein the isolated nucleic acid comprises the nucleotide sequence of SEQ ID NO: 46. In some embodiments, the disclosure provides an isolated nucleic acid that encodes the ShAb17 nanobody, wherein the isolated nucleic acid comprises the nucleotide sequence of SEQ ID NO: 47. In some embodiments, the disclosure provides an isolated nucleic acid that encodes the ShAb18 nanobody, wherein the isolated nucleic acid comprises the nucleotide sequence of SEQ ID NO: 48. In some embodiments, the disclosure provides an isolated nucleic acid that encodes the ShAb19 nanobody, wherein the isolated nucleic acid comprises the nucleotide sequence of SEQ ID NO: 49. In some embodiments, the disclosure provides an isolated nucleic acid that encodes the ShAb20 nanobody, wherein the isolated nucleic acid comprises the nucleotide sequence of SEQ ID NO: 50. In some embodiments, the disclosure provides an isolated nucleic acid that encodes the ShAb21 nanobody, wherein the isolated nucleic acid comprises the nucleotide sequence of SEQ ID NO: 51. In some embodiments, the disclosure provides an isolated nucleic acid that encodes the ShAb22 nanobody, wherein the isolated nucleic acid comprises the nucleotide sequence of SEQ ID NO: 52. In some embodiments, the disclosure provides an isolated nucleic acid that encodes the ShAb23 nanobody, wherein the isolated nucleic acid comprises the nucleotide sequence of SEQ ID NO: 53. In some embodiments, the disclosure provides an isolated nucleic acid that encodes the ShAb24 nanobody, wherein the isolated nucleic acid comprises the nucleotide sequence of SEQ ID NO: 54. In some embodiments, the disclosure provides an isolated nucleic acid that encodes the ShAb25 nanobody, wherein the isolated nucleic acid comprises the nucleotide sequence of SEQ ID NO: 55. In some embodiments, the disclosure provides an isolated nucleic acid that encodes the ShAb26 nanobody, wherein the isolated nucleic acid comprises the nucleotide sequence of SEQ ID NO: 56. In some embodiments, the disclosure provides an isolated nucleic acid that encodes the ShAb27 nanobody, wherein the isolated nucleic acid comprises the nucleotide sequence of SEQ ID NO: 57. In some embodiments, the disclosure provides an isolated nucleic acid that encodes the ShAb28 nanobody, wherein the isolated nucleic acid comprises the nucleotide sequence of SEQ ID NO: 58. In some embodiments, the disclosure provides an isolated nucleic acid that encodes the ShAb29 nanobody, wherein the isolated nucleic acid comprises the nucleotide sequence of SEQ ID NO: 59. In some embodiments, the disclosure provides an isolated nucleic acid that encodes the ShAb30 nanobody, wherein the isolated nucleic acid comprises the nucleotide sequence of SEQ ID NO: 60. In some embodiments, the disclosure provides an isolated nucleic acid that encodes the ShAb31 nanobody.

The disclosure also provides expression vectors (or plasmids) comprising at least one nucleic acid encoding at least one CDR, both CDRs (i.e., CDR1 and CDR3), at least one HV, and/or both HVs (i.e. HV2 and HV4) of one of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies, as well as other nucleic acid sequences useful for regulating polypeptide expression. In certain embodiments, the nucleic acid encodes the CDR1, CDR3, HV2, and HV4 of at least one of the ShAb01, ShAb02, ShAb03, ShAb04, ShAb05, ShAb06, ShAb07, ShAb08, ShAb09, ShAb10, ShAb11, ShAb12, ShAb13, ShAb14, ShAb15, ShAb16, ShAb17, ShAb18, ShAb19, ShAb20, ShAb21, ShAb22, ShAb23, ShAb24, ShAb25, ShAb26, ShAb27, ShAb28, ShAb29, ShAb30, and ShAb31 nanobodies. Suitable expression vectors can be chosen or constructed, so that they contain appropriate regulatory sequences, including promoter sequences, terminator sequences, polyadenylation sequences, enhancer sequences, marker genes and other sequences as appropriate.

The expression vectors can be introduced into a host cell to produce the desired SARS-CoV-2 coronavirus binding agent, the desired binding agent that neutralizes SARS-CoV-2 coronavirus in vitro or in vivo, or chimeric proteins thereof. Systems for cloning and expression of a polypeptide in a variety of different host cells are well known in the art. Any protein compatible expression system may be used to produce the disclosed SARS-CoV-2 coronavirus binding agents, the binding agents that neutralizes SARS-CoV-2 coronavirus in vitro or in vivo, or chimeric proteins thereof.

A further aspect of the disclosure provides an isolated host cell comprising a nucleic acid (or expression vector) as disclosed herein. A still further aspect provides a method comprising introducing such nucleic acid (or expression vector) into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction of the nucleic acid into the cells may be followed by causing or allowing expression from the nucleic acid, e.g., by culturing host cells under conditions for expression of the gene. Following production by expression an antibody may be isolated and/or purified using any suitable technique, then used as appropriate.

### Methods of Use

The SARS-CoV-2 coronavirus binding agents or chimeric proteins thereof disclosed herein can be used in a variety of research and medical applications. In some embodiments, the disclosure provides a method of treating or preventing SARS-CoV-2 coronavirus infection, comprising administering to a subject in need thereof an effective amount of one or more of the disclosed SARS-CoV-2 coronavirus binding agents to treat or prevent the SARS-CoV-2 coronavirus infection. In some embodiments, the disclosure provides a method of treating or preventing SARS-CoV-2 coronavirus infection, comprising administering to a subject in need thereof an effective amount of one or more chimeric proteins comprising one or more SARS-CoV-2 coronavirus binding agents disclosed herein to treat or prevent the SARS-CoV-2 coronavirus infection. Subjects that can be treated with the SARS-CoV-2 coronavirus binding agents or chimeric proteins thereof disclosed herein include humans and non-human mammals, including, but not limited to, non-human primates, dogs, cats, horses, cows, sheep, pigs, goats, minks, mice, rats, hamsters, and guinea pigs. In some embodiments, the subject being treated is a human. In some embodiments, the subject being treated is diagnosed with or suspected of having a SARS-CoV-2 infection. In some embodiments, the SARS-CoV-2 coronavirus binding agents or chimeric proteins thereof disclosed herein are administered to the subject in a composition that further comprises a pharmaceutically acceptable excipient as disclosed elsewhere herein. In some embodiments, the composition is formulated for subcutaneous, intravenous, intraarterial, or intramuscular injection as disclosed elsewhere herein. In some embodiments, the composition is formulated for aerosolized administration, including intranasal administration or administration by inhalation as disclosed elsewhere herein.

In some embodiments, one or more of the SARS-CoV-2 coronavirus binding agents or chimeric proteins thereof described herein are used in a method of treating COVID-19. If the disease is COVID-19, the disease can be asymptomatic, mild, moderate, severe, or critical. An asymptomatic form of COVID-19 does not show any symptoms in the subject. A mild form of COVID-19 may show mild form of one or more of: tiredness, fever, cough, breathlessness after moderate exercise, sore throat, muscle ache, headache, and diarrhea. Mild form of COVID-19 may not require management of symptoms. A moderate form of COVID-19 may show moderate form of one or more of: tiredness, fever, cough, breathlessness after slight activity, sore throat, muscle ache, headache, and diarrhea. Moderate form of COVID-19 may require managing the symptoms. A severe form of COVID-19 may show of one or more of: severe tiredness, high fever, cough, breathlessness even at rest, painful breathing, loss of appetite, loss of thirst, sore throat, muscle ache, headache, diarrhea, and confusion. Severe form of COVID-19 would typically require significant intervention for managing symptoms, such as: pneumonia, hypoxemic respiratory failure, acute respiratory distress syndrome (ARDS), sepsis, septic shock, cardiomyopathy, arrhythmia, acute kidney injury, and complications from prolonged hospitalization including secondary bacterial infections, thromboembolism, gastrointestinal bleeding, and critical illness polyneuropathy/myopathy.

In some embodiments, a cocktail of one or more of the SARS-CoV-2 coronavirus binding agents or chimeric proteins thereof described herein are used in a method of treating or preventing SARS-CoV-2 infection or disease, such as COVID-19.

In some embodiments, one or more of the SARS-CoV-2 coronavirus binding agents or chimeric proteins thereof disclosed herein can be administered prophylactically before infection or in order to reduce or prevent transmission, or before any clinical indication of illness, disease or infection. In some embodiments, the one or more SARS-CoV-2 coronavirus binding agents or chimeric proteins thereof disclosed herein can be administered in a time period days before infection or before possible or presumed exposure or risk of exposure as a prophylactic. For example, one or more of the SARS-CoV-2 coronavirus binding agents or chimeric proteins thereof disclosed herein may be administered a day prior or before, 2 days before or prior, 3 days prior or before, 4 days prior or before, 5 days prior or before, 6 days prior or before, 7 days prior or before, a week prior or before, more than 7 days prior or before, more than a week prior or before, up to 9 days prior or before, up to 10 days prior or before expected exposure. The SARS-CoV-2 coronavirus binding agents or chimeric proteins thereof disclosed herein may be used to provide immediate immunity, for example, to avoid an outbreak in a suitable environment, such as a nursing home, military base or hospital or to prevent transmission prior to travel (e.g., entering a plane, train, bus, etc.) or in other instances where social distancing is impractical. In some embodiments, a single administration, e.g., a single injection, may provide immediate immunity that lasts up to about 6 months or longer.

In addition, one or more of the SARS-CoV-2 coronavirus binding agents or chimeric proteins thereof disclosed herein can be used to detect SARS-CoV-2 as described herein in a sample. In some embodiments, the method comprises contacting one or more of the SARS-CoV-2 coronavirus binding agents or chimeric proteins thereof disclosed herein with the sample and analyzing the sample to determine whether the sample contains SARS-CoV-2 coronavirus, wherein binding of the SARS-CoV-2 coronavirus binding agents or chimeric proteins thereof to the spike glycoprotein of SARS-CoV-2 in the sample indicates the presence of SARS-CoV-2 coronavirus in the sample. In some embodiments, the sample is from a subject suspected of having the SARS-CoV-2 coronavirus infection.

In some embodiments, the sample comprises a non-biological sample, such as soil, water, or food products such as meat. In other embodiments, the sample comprises a biological sample, such as blood, serum, mucus (e.g., nasal swab), tissue, cells, urine, or stool. Such methods can be used to detect SARS-CoV-2 coronavirus infection in a patient, wherein binding of the SARS-CoV-2 coronavirus binding agents or chimeric proteins thereof to SARS-CoV-2 coronavirus in a sample from the patient indicates the presence of SARS-CoV-2 coronavirus infection in the patient.

Any appropriate label may be used in the detection methods and compositions described herein. A label is any molecule or composition bound to an agent, or a secondary molecule that is conjugated thereto, and that is detectable by spectroscopic, photochemical, biochemical, immunochemical, electrical, optical or chemical means. Examples of labels, including enzymes, colloidal gold particles, colored latex particles, have been disclosed (U.S. Pat. Nos. 4,275,149; 4,313,734; 4,373,932; and 4,954,452, each incorporated by reference herein). Additional examples of useful labels include, without limitation, haptens (e.g., biotin, digoxigenin (DIG), dintrophenol (DNP), etc.), radioactive isotopes, co-factors, ligands, chemiluminescent or fluorescent agents, protein-adsorbed silver particles, protein-adsorbed iron particles, protein-adsorbed copper particles, protein-adsorbed selenium particles, protein-adsorbed sulphur particles, protein-adsorbed tellurium particles, protein-adsorbed carbon particles, and protein-coupled dye sacs. The attachment of a compound to a label can be through any means, including covalent bonds, adsorption processes, hydrophobic and/or electrostatic bonds, as in chelates and the like, or combinations of these bonds and interactions and/or may involve a linking group.

### Formulations and Administration

The disclosure also provides compositions comprising one or more of the SARS-CoV-2 coronavirus binding agents or chimeric proteins thereof disclosed herein. In some embodiments, the compositions are suitable for pharmaceutical use and administration to patients. In some embodiments, the compositions further comprise a pharmaceutically acceptable excipient.

Pharmaceutically acceptable excipients include, but are not limited to, a carrier or diluent, such as a gum, a starch (e.g. corn starch, pregeletanized starch), a sugar (e.g. lactose, mannitol, sucrose, dextrose), a cellulosic material (e.g. microcrystalline cellulose), an acrylate (e.g. polymethylacrylate), calcium carbonate, magnesium oxide, talc, or mixtures thereof; a binder (e.g. acacia, cornstarch, gelatin, carbomer, ethyl cellulose, guar gum, hydroxypropyl cellulose, hydroxypropyl methyl cellulose, povidone); a disintegrating agent (e.g. cornstarch, potato starch, alginic acid, silicon dioxide, croscarmelose sodium, crospovidone, guar gum, sodium starch glycolate), a buffer (e.g. Tris-HCl, acetate, phosphate) of various pH and ionic strength; and additive such as albumin or gelatin to prevent absorption to surfaces; a detergent (e.g. Tween 20, Tween 80, Pluronic F68, bile acid salts); a protease inhibitor; a surfactant (e.g. sodium lauryl sulfate); a permeation enhancer; a solubilizing agent (e.g. glycerol, polyethylene glycerol); an anti-oxidants (e.g. ascorbic acid, sodium metabisulfite, butylated hydroxyanisole); a stabilizer (e.g. hydroxypropyl cellulose, hydroxypropylmethyl cellulose); a viscosity increasing agent (e.g. carbomer, colloidal silicon dioxide, ethyl cellulose, guar gum); a sweetener (e.g. aspartame, citric acid); a preservative (e.g. Thimerosal, benzyl alcohol, parabens); a lubricant (e.g. stearic acid, magnesium stearate, polyethylene glycol, sodium lauryl sulfate); a flow-aid (e.g. colloidal silicon dioxide), a plasticizer (e.g. diethyl phthalate, triethyl citrate); an emulsifier (e.g. carbomer, hydroxypropyl cellulose, sodium lauryl sulfate); a polymer coating (e.g. poloxamers or poloxamines); a coating and film forming agent (e.g. ethyl cellulose, acrylates, polymethacrylates); an adjuvant; a pharmaceutically acceptable carrier for liquid formulations, such as an aqueous (water, alcoholic/aqueous solution, emulsion or suspension, including saline and buffered media) or non-aqueous (e.g., propylene glycol, polyethylene glycol, and injectable organic esters such as ethyl oleate) solution, suspension, emulsion or oil; and a parenteral vehicle (for subcutaneous, intravenous, intraarterial, or intramuscular injection), including but not limited to, sodium chloride solution, Ringer's dextrose, dextrose and sodium chloride, lactated Ringer's and fixed oils.

Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols, such as propylene glycols or polyethylene glycol, are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

A pharmaceutical composition of the disclosure is formulated to be compatible with its intended route of administration. Methods to accomplish the administration are known to those of ordinary skill in the art. This includes, for example, injections, by parenteral routes such as intravenous, intravascular, intraarterial, subcutaneous, intramuscular, intraperitoneal, intraventricular, intraepidural, or others as well as oral, nasal, ophthalmic, rectal, or topical. Sustained release administration is also specifically contemplated, by such means as depot injections or erodible implants. Localized delivery is particularly contemplated, by such means as delivery via a catheter to one or more arteries, such as the renal artery or a vessel supplying a localized site of interest.

In some embodiments, the compositions of the disclosure may be formulated in nasal sprays or inhalation solutions or suspensions using approaches known and acceptable in the art and in the medical field and clinical practice. The Food and Drug Administration (FDA) provides guideline and guidance with regard to such sprays, solutions and suspensions and spray drug products, including in Guidance for Industry documents available at fda.gov. An exemplary July 2002 Guidance for Industry document entitled Nasal Spray and Inhalation Solution, Suspension and Spray Drug Products—Chemistry, Manufacturing and Controls Documentation includes details regarding formulation components and compositions, specifications therefore, manufacturing, and closed container systems.

Nasal Sprays are drug products that contain active ingredients dissolved or suspended in a formulation, typically aqueous-based, which can contain other excipients and are intended for use by nasal inhalation. Container closure systems for nasal sprays include the container and all components that are responsible for metering, atomization, and delivery of the formulation to the patient. Nasal spray drug products contain therapeutically active ingredients (drug substances) dissolved or suspended in solutions or mixtures of excipients (e.g., preservatives, viscosity modifiers, emulsifiers, buffering agents) in nonpressurized dispensers that deliver a spray containing a metered dose of the active ingredient. The dose can be metered by the spray pump or could have been premetered during manufacture. A nasal spray unit can be designed for unit dosing or can discharge numerous metered sprays of formulation containing the drug substance. Nasal sprays are applied to the nasal cavity for local and/or systemic effects.

In some embodiments, the pharmaceutical compositions of the disclosure are aerosolized administration. A nebulizer is a drug delivery device used to administer medication in the form of aerosol into the respiratory tract. Nebulizers can be used for intranasal and inhalation delivery of therapeutic agents through the mouth and nasal passage and are effective devices for delivery of therapeutic agents to the upper and/or lower respiratory tract. Nebulizers use oxygen, compressed air or ultrasonic power to break up medical solutions and suspensions into small aerosol droplets that can be directly inhaled from the mouthpiece of the device. In some embodiments, a metered-dose inhaler (MDI) device is used to deliver the one or more SARS-CoV-2 coronavirus binding agents or chimeric proteins thereof disclosed herein in a specific amount of medication to the lungs in the form of a short burst of aerosolized medicine that is usually self-administered by the patient via inhalation. Dry powder inhalers, which utilize micronized powder often packaged in single dose quantities in blisters or gel capsules containing the powdered medication, may also be used to deliver the one or more SARS-CoV-2 coronavirus binding agents or chimeric proteins thereof disclosed herein to the lungs. In some embodiments, one or more of the SARS-CoV-2 coronavirus binding agents or chimeric proteins thereof disclosed herein are administered to a patient by intravenous, intramuscular or subcutaneous injection. The SARS-CoV-2 coronavirus binding agents or chimeric proteins thereof disclosed herein may be administered, for example, by bolus injunction or by slow infusion. The dosage may depend on the type and severity of the infection and/or on the characteristics of the individual, such as general health, age, sex, body weight and tolerance to drugs and should be adjusted, as needed, according to individual need and professional judgment. The dosage may also vary depending upon factors, such as route of administration, target site, or other therapies administered. The skilled artisan will be able to determine appropriate doses depending on these and other factors.

Toxicity and therapeutic efficacy of the composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. SARS-CoV-2 coronavirus binding agents or chimeric proteins thereof disclosed herein that exhibit large therapeutic indices may be less toxic and/or more therapeutically effective.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. SARS-CoV-2 Coronavirus Binding Molecules Derived From Shark Nanobodies

This example describes the initial development of SARS-CoV-2 coronavirus binding molecules derived from shark nanobodies. Three shark nanobodies were identified and isolated during this initial development, AliB5-2D8, MoB3-3D8 and MoB5-1D4, also referred to as ShAb01, ShAb02, and ShAb31, respectively. These SARS-CoV-2 coronavirus binding molecules specifically bind to SARS-CoV-2 Spike glycoprotein.

### Generation of an IgNAR Response to SARS-CoV-2 in Sharks

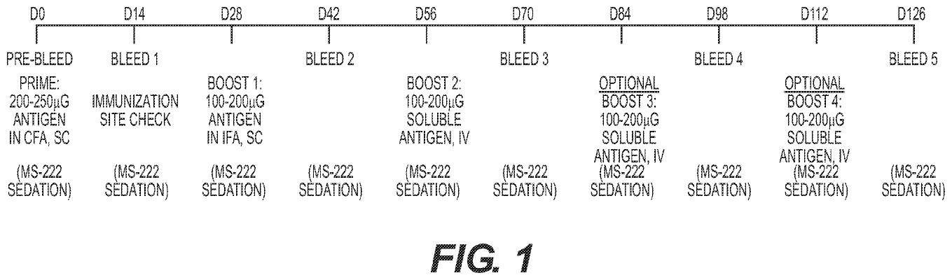

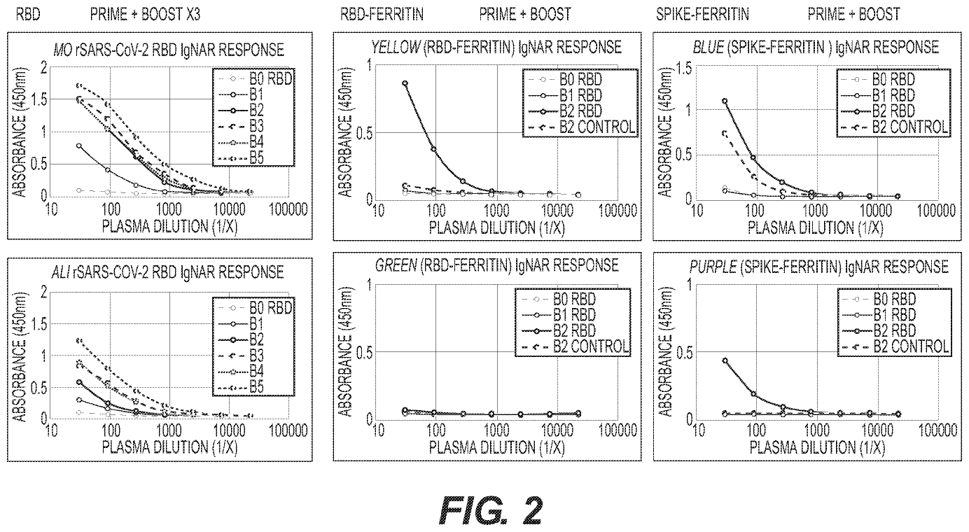

SARS-COV-2 naïve nurse sharks (Ginglymostoma cirratum), each approximately 1-2 meters in length, were procured and housed in the Aquaculture research center at the Institute of Marine & Environmental Technology, Baltimore, USA. Each shark was immunized with immunogens that contain the receptor-binding domain (RBD) of SARS-CoV-2 Spike glycoprotein according to previously published methods (Dooley et al., 2003, Mol. Immunol., 2003, 40(1):25-33; hereby incorporated by reference in its entirety) according to the schedule provided in FIG. 1. Three different immunogens were tested: a SARS-CoV-2 RBD immunogen, a RBD-Ferritin immunogen (pCoV131), and a Spike trimer-Ferritin immunogen (pCoV-1B-06-PL). Test bleed plasma was taken from the tail sinus after each immunization and assayed for SARS-CoV-2 reactive IgNAR antibodies on immobilized SARS-CoV-2 RBD or SARS-CoV-2 Spike protein by binding ELISA with the results shown in FIG. 2.

### Construction of VNAR Phage Display Libraries

RNA was isolated from peripheral blood lymphocytes (PBLs) of immune sharks. Briefly, whole blood was spun at 1000 rpm for 10 minutes to separate the PBLs from other blood constituents, PBLs were moved to a clean tube and lysed using TRIzol® Reagent. Total RNA was extracted from the homogenates via chloroform extraction followed by isopropanol precipitation. To generate VNAR cDNA fragments, 1 μg of RNA was used as template for RT-PCR synthesis then PCR amplification of the VNAR repertoire performed with the primers:

### NARF4For1:

### 5′-ATAATCAAGCTTGCGGCCGCATTCACAGTCACGACAGTGCCACCTC-3′,

### NARF4For2:

### 5′-ATAATCAAGCTTGCGGCCGCATTCACAGTCACGGCAGTGCCATCTC-3′, and

### NARF1Rev:

5′-ATAATAAGGAATTCCATGGCTCGAGTGGACCAAACACCG-3′ as described in Dooley et al., 2003 (Mol. Immunol., 40(1):25-33; hereby incorporated by reference). The RT-PCR product was purified and digested using NcoI and NotI for ligation into similarly digested pHEN2 phage display vector.

Ligated plasmids constituting the pHEN2-VNAR libraries were electroporated into competent TG1 E. coli cells and plated onto 2×YT media containing 1% (w/v) glucose and 100 μg/ml ampicillin. After overnight incubation at 37° C., colonies from each library were then scraped from the plates and stored at −80° C. as glycerol stocks (25% glycerol v/v) for future use.

### Affinity Selection of VNAR Libraries Against SARS-CoV-2 Antigen

Phage carrying VNARs as gene III fusions were generated by infection of bacterial stocks carrying phagemid plasmids (pHEN2) with M13K07 helper phage according to standard protocols (Krebber et al., 1997; Dooley et al., 2003).

Affinity selection was performed on immunotubes coated with SARS-CoV-2 RBD or SARS-CoV-2 Spike protein by addition of the library phage and incubating at room temperature for 2 hours. The contents of the tube were poured away and tubes washed with PBS 0.1% (v/v) Tween 20 or PBS, to remove non-binding phage. With successive selection rounds the concentration of SARS-CoV-2 RBD or SARS-CoV-2 Spike protein was reduced and number of washes increased to increase selection stringency. Bound phage was then eluted using 1 ml 100 mM triethylamine for 10 minutes and neutralized with 1 M Tris-HCl, pH 7.5. The neutralized phage was used to infect E. coli TG1 cells and the number of output phage were titered by serial dilution of a sample of this culture. Remaining cells were plated onto 2×YT media supplemented with 2% glucose and either 100 μg/ml ampicillin and incubated at 37° C. overnight. Bacterial colonies were harvested and a glycerol stock was prepared by resuspending the bacterial pellet in 25% (v/v) glycerol in 2×YT media, to be used in further rounds of selection.

Target-specific clones were identified by monoclonal phage ELISA on immobilized SARS-CoV-2 RBD or SARS-CoV-2 Spike protein according to standard protocols (Dooley et al, 2003, Mol. Immunol., 2003, 40(1):25-33) and positive clones sequenced with the primers LMB3 (5′-CACAGGAAACAGCTATGAC-3′) and pHENseq (5′-CTATGCGGCCCCATTC-3′) (Dooley et al, 2003, Mol. Immunol., 2003, 40(1):25-3).

### Antibody and Nanobody Protein Expression

The sequences of two shark nanobodies identified during this initial development, AliB5-2D8 and MoB3-3D8, as well as their corresponding CDR1, CDR3, HV2 and HV4 sequences are as follows.

```
ShAb01 (AliB5-2D8) single domain VNAR:
(SEQ ID NO: 1)
ARVDQTPRSVTKETGESLTINCVLRDSNCALSSTHWYRKKSGSTNEERIL
                                                        CDR1 HV2
QGRRYVETVNSGSKSFSLRINDLRVEDSGTYRCKVYWGNSWQDKFCPGLG
                      HV4 CDR3
SYEYGDGTAVTVN
(SEQ ID NO: 2)
ShAb02 (MoB3-3D8) single domain VNAR:
ARVDQTPQTITKETGESLTINCVLRDSNCALASTDWYRKKSGSTNEESIS
                                                    CDR1 HV2
KGGRYVETVNSGSKSFSLRINDLTVEDSGTYRCNAWDSWETRQLKCDYDV
                      HV4 CDR3
YGGGTVVTVN
(SEQ ID NO: 394)
ShAb31 (MoB5-1D4) single domain VNAR:
ARVDQTPQTITKETGESLTINCVLRDSNCGVASTDWYRKKSGSTNEESIS
                                                      CDR1 HV2
KGGRYVETVNSRSKSFSLRINDLTVEDSGTYRCNAWDRWETRQLNCDYDV
                      HV4 CDR3
YGGGTVVTVN
```

The sequence of ShAb02 (MoB3-3D8) was synthesized (Genscript) and cloned into a pCMVR expression vector (NIH AIDS reagent program) between a murine Ig leader (GenBank DQ407610) and the constant regions of human IgG1 (GenBank AAA02914). The resulting plasmid was transfected into Expi293F cells (ThermoFisher) according to the manufacturer's instructions. After 5 days, the MoB3-3D8_fc chimera antibody was purified from cleared culture supernatants with Protein A agarose (ThermoFisher) using standard procedures, buffer exchanged into Phosphate-Buffered Saline (PBS) and quantified using calculated E and A280 measurement. Other antibodies used for competition and comparisons in ACE-2 blocking assay were cloned into the pCMVR expression vector, and heavy and light chains were co-transfected into Expi293F cells followed by expression and purification as described above.

Nanobodies were cloned into a pSecTAG or a pCMVR plasmid with a C-terminal His-tag and expressed in mammalian cells as described above. Protein was purified by NiNTA affinity chromatography followed by gel-filtration.

### Biolayer Interferometry

All measurements were monitored on an Octet RED96 instrument (Pall ForteBio) at 30° C. with a shake speed of 1000 rpm. Samples were diluted in kinetics buffer (0.1% [w/v] bovine serum albumin [BSA], 0.02% [v/v] Tween-20 in PBS).

Affinity Measurements of Shark Nanobodies with SARS-CoV-2 Receptor-Binding Domain.

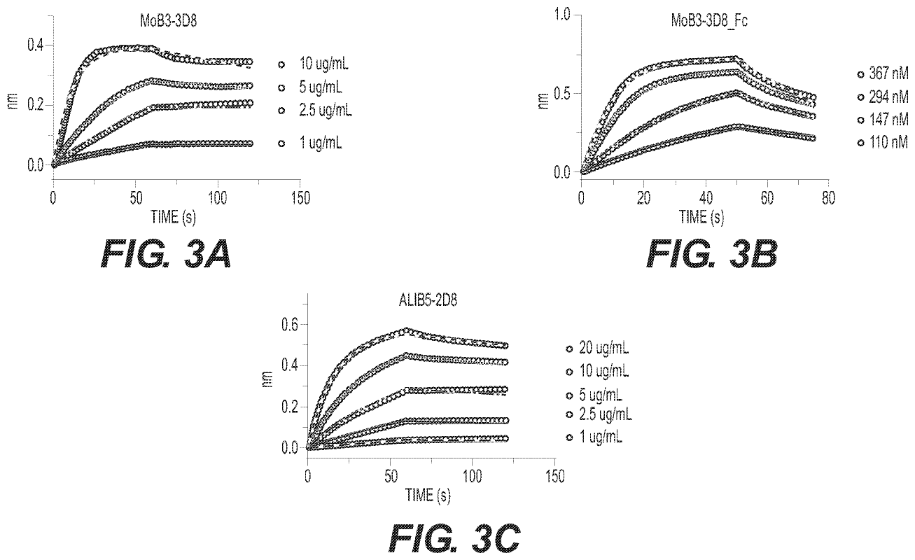

Affinity kinetic constants between SARS-CoV-2 RBD and nanobodies were determined, from at least 4 concentrations of RBD, by fitting the curves to a 1:1 Langmuir binding model using the Data analysis software 9.0 (ForteBio). Nanobodies were loaded at 30 μg/ml onto a His1K probe for 120 seconds followed by baseline incubation for 30 seconds. Binding was allowed to occur for 50-60 seconds followed by a dissociation step for 25-60 seconds. MoB3-3D8_Fc fusion chimera antibody was loaded at 30 μg/ml onto an anti-human Fc capture (AHC) probe for 120 seconds followed by baseline incubation for 30 seconds. Binding to SARS-CoV-2 RBD was allowed to occur for 60 seconds, followed by a dissociation step for 60 seconds. HISIK probe was used to bind to the SARS-COV-2 RBD-His followed by binding to the shark nanobodies. The binding is double referenced width PBS and a control non-binding nanobody 5A7. The binding kinetics of shark nanobodies MoB3-3D8, MoB3-3D8_Fec and AiiB5-2D8 are shown in FIG. 3A, FIG. 3B and FIG. 3C, respectively.

Competition of Shark Nanobodies with SARS-CoV-2 Antibodies

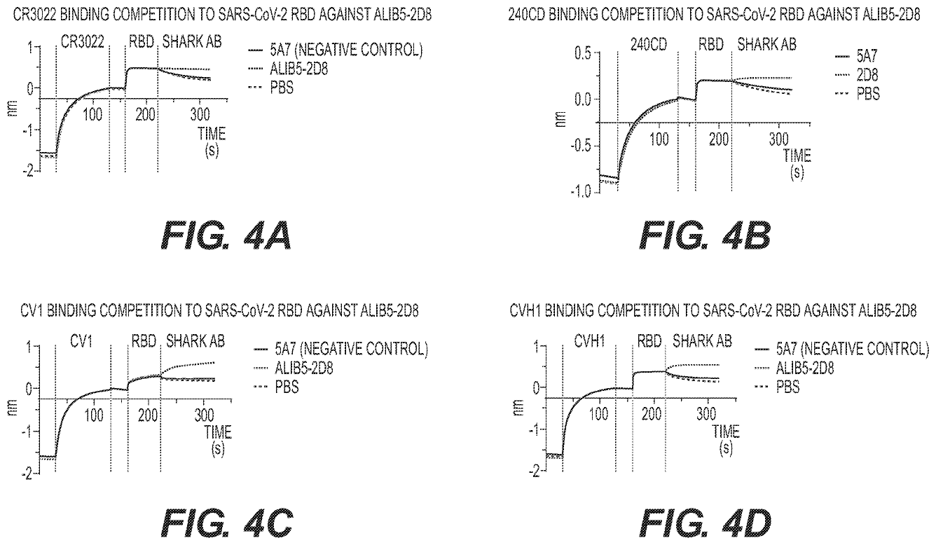

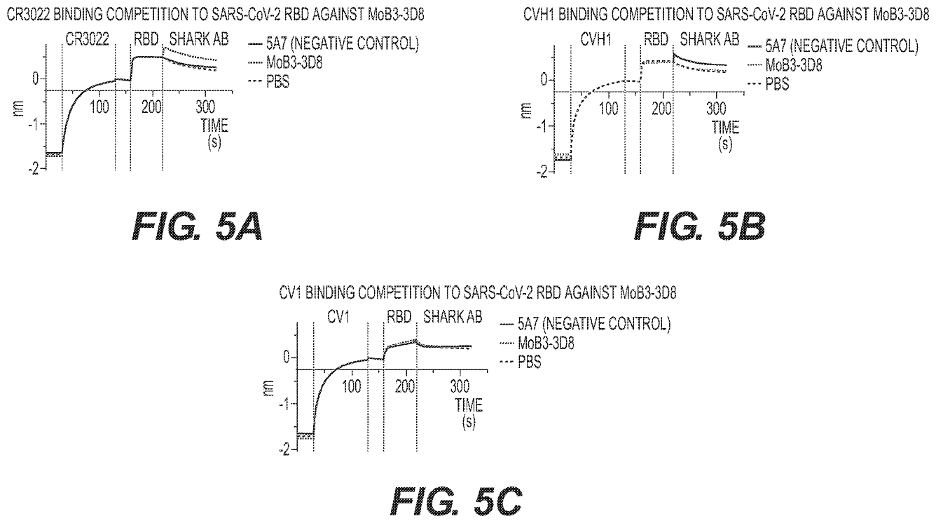

To assess antibody competition, SARS-COV-2 antibodies including CR3022, 240CD, CV1, and CVH1 were incubated with the SARS-CoV-2 RBD prior to assessment of binding to nanobodies. Antibody concentration was 30 μg/ml. Antibodies were loaded onto an AHC probe, followed by a baseline step, followed by binding to SARS-COV-2 RBD, followed by assessment of binding to the shark nanobody. If binding of the nanobody was observed, this indicated no competition, while lack of binding indicated that the initial antibody blocked the shark nanobody, with a likely overlapping epitope. As shown in FIG. 4A-4D, CR3022 (FIG. 4A) and 240CD (FIG. 4B) antibodies show competition against AliB5-2D8, while CV1 (FIG. 4C) and CVH1 (FIG. 4D) antibodies do not compete with AliB5-2D8. For MoB3-3D8, both CR3022 (FIG. 5A) and CVH1 (FIG. 5B) antibodies show no competition against MoB3-3D8, while CV1 (FIG. 5C) antibody competes with MoB3-3D8.

### Binding of Fc-Fusion MoB3-3D8 to SARS-CoV-2 Spike-Ferritin Immunogen (Variant SpFN_pCoV-1B-06-PL) and Other SARS-COV-2 Vaccine Candidates

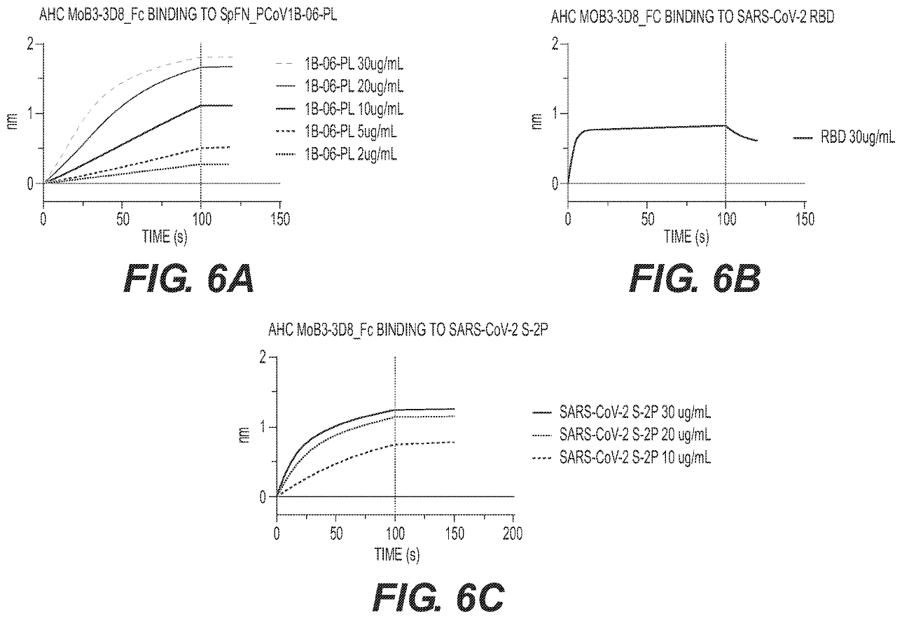

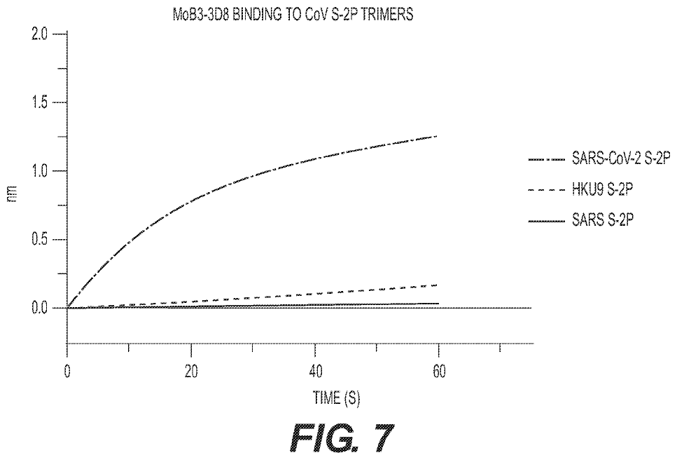

The MoB3-3D8_Fc chimera antibody was used to assess antigenicity of SARS-CoV-2 vaccine candidate immunogens including SpFN_pCoV1B-06-PL, SARS-COV-2 S-2P, and SARS-CoV-2 RBD. The MoB3-3D8_Fc chimera antibody was loaded onto a AHC probe for 120 seconds, followed by a 30 seconds baseline incubation. The loaded probes were then incubated with vaccine candidates to allow binding for 100 seconds, followed by a 25-50 seconds dissociation step. Immunogen candidates ranged from 30 μg/ml to 2 μg/ml. As shown in FIG. 6A-6C, the MoB3-3D8_Fc fusion antibody binds to a Spike Ferritin nanoparticle (SpFN) vaccine candidate at five concentrations ranging from 30 μg/ml to 2 μg/ml (FIG. 6A), to SARS-CoV-2 RBD at 30 μg/ml (FIG. 6B), and to the SARS-CoV-2 stabilized Spike trimer (S-2P) at concentrations ranging from 30 μg/ml to 10 μg/ml (FIG. 6C). MoB3-3D8 were also assessed for binding to other coronavirus stabilized S trimers (S-2P) from SARS-CoV, and HKU9 CoV, but showed little to no binding (FIG. 7).

### ACE-2 Blocking Assay

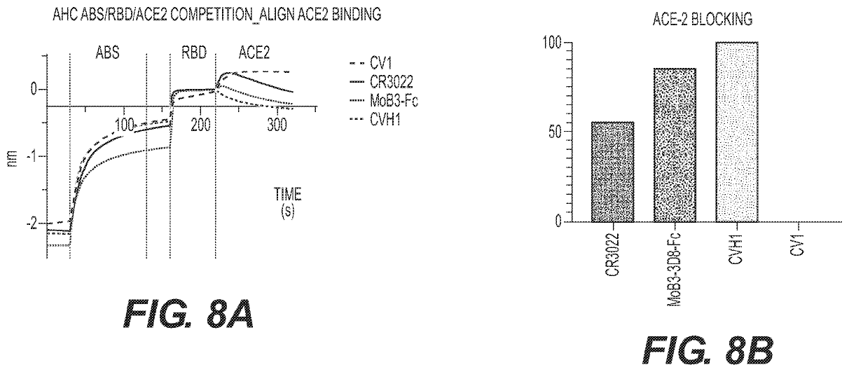

MoB3-3D8_Fc chimera antibody was assessed for ability to block ACE-2 binding to SARS-CoV-2 receptor alongside a set of SARS-CoV-2 antibodies including CR3022, CV1, and CVH1. SARS-CoV-2 antibodies and MoB3-3D8_Fc chimera were loaded onto an AHC probe, followed by binding to the SARS-CoV-2 RBD. This Antibody-RBD complex was then incubated with human ACE-2 receptor to assess ACE-2 blocking. Measurements were carried out using Biolayer Interferometry. As shown in FIG. 8A-8B, CV1 shows no competition against ACE-2 receptor and CR3022 shows partial blocking, while CVH1 and MoB3-3D8_Fc showed blocking of the RBD-ACE-2 receptor interaction.

### Example 2. Isolation and Characterization of Shark-Derived Nanobodies (ShAbs) with Affinity to the SARS-CoV-2 Spike Protein

Reported here is the isolation and characterization of shark-derived nanobodies (ShAb) with nM/pM affinity that target the receptor binding domain (RBD) and N-terminus domain (NTD) of the SARS-CoV-2 spike glycoprotein. These nanobodies were elicited in the sharks by immunization. Certain ShAb molecules potently neutralized SARS-CoV-2 virus, including variants B.1.1.7 (Alpha) and B.1.351 (Beta). A subset of the ShAb molecules also neutralized the related sarbecovirus SARS-CoV-1. Also shown is that multiple ShAb immunotherapeutics provided protection in a K18-ACE2 transgenic mouse model from SARS-CoV-2 challenge, either given as a passive therapy prior to infection, or as a therapeutic given after infection. Competition, mutagenesis, and structural studies define multiple non-overlapping epitopes located on either the RBD or NTD of the SARS-CoV-2 spike glycoprotein. By combining multiple ShAbs into single multivalent immunotherapeutic molecules, synergistic effects including increased ability to target viral variants, affinity, ACE2-blocking and neutralization potency were observed.

1. Materials and Methods

i. Immunogen Design and Production

Following release of the SARS-CoV-2 sequence on 10 Jan. 2020, initial RBD, RBD-Ferritin (RFN) and Spike-Ferritin (SpFN) immunogens were designed. Subsequent iterative immunogen design and optimization utilized atomic models of the SARS-CoV-2 RBD molecule (Joyce et al., 2020), or the SARS-CoV-2 spike trimer structure PDB ID: 6VXX and PDB ID: 3BVE for the Helicobacter pylori Ferritin and PDB ID: 4LQH for the bullfrog linker sequence. PyMOL (The PyMOL Molecular Graphics System; Schrödinger, Inc.) was used to generate the ferritin 24-subunit particle, and a map created in UCSF Chimera (Pettersen et al., 2004) was supplied to “align symmetry” of cisTEM (Grant et al., 2018) to align the ferritin particle to an octahedral symmetry convention. This was supplied to “phenix.map_symmetry” to generate a symmetry file and PDB file, for octahedral (for RBD-fusions) and D4 (for trimer-fusions) symmetry. Spike-domain ferritin nanoparticle fusions were modelled using PyMOL and Coot (Emsley et al., 2010) and expanded using “phenix.apply_ncs” (Liebschner et al., 2019). Visual analysis and figure generation was conducted using ChimeraX and PyMOL.

RBD-Ferritin designs were generated by assessment of the hydrophobic surface of the SARS-CoV-2 RBD surface and determining surface accessible mutations that reduced the hydrophobic surface. Spike-Ferritin designs were created by modeling the coiled-coil region between Spike residues 1140 and 1158 and increasing the coil-coil interaction either by mutagenesis, or by increasing the length of the interaction region.

ii. Shark Immunizations

All research in this study involving animals was conducted in compliance with the Animal Welfare Act, and other federal statutes and regulations relating to animals and experiments involving animals and adhered to the principles stated in the Guide for the Care and Use of Laboratory Animals, NRC Publication, 1996 edition. Sharks were sedated with MS-222 prior to any procedure and all animal procedures were conducted in accordance with University of Maryland, School of Medicine, Institutional Animal Care and Use Committee (IACUC)- and USAMRDC Animal Care and Use Review Office (ACURO)-approved protocols.

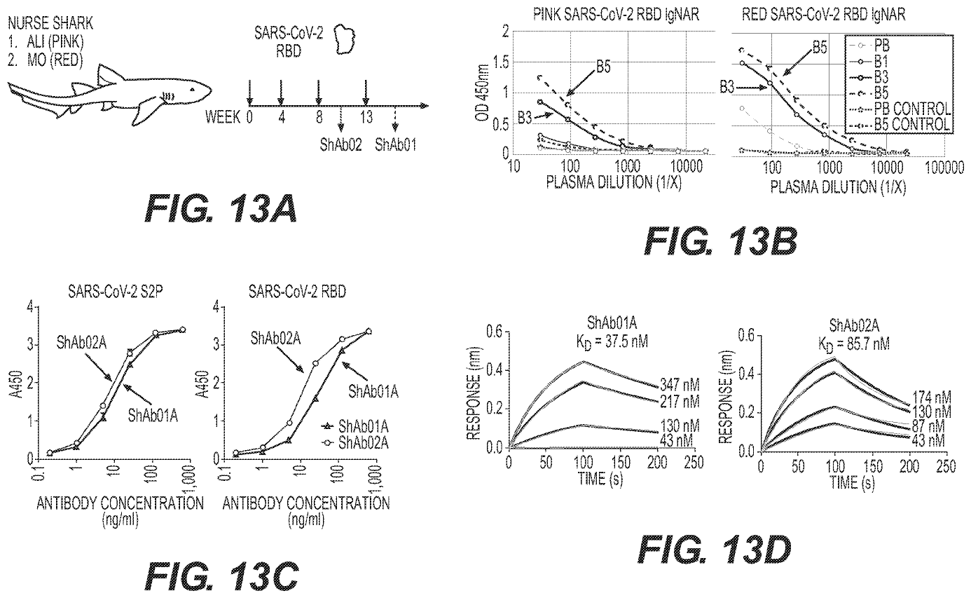

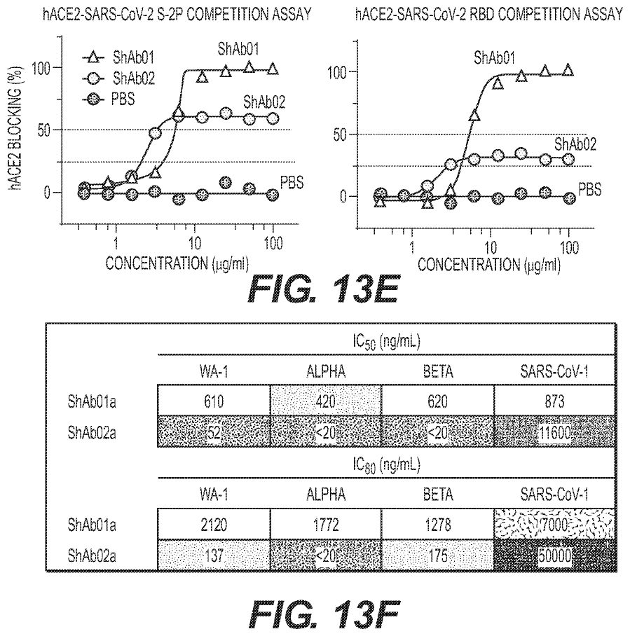

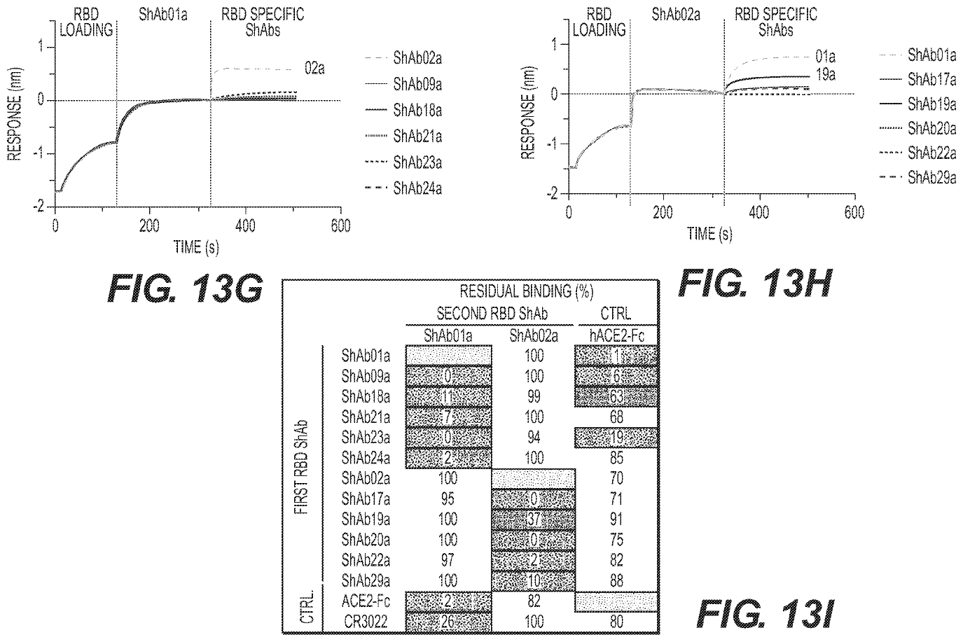

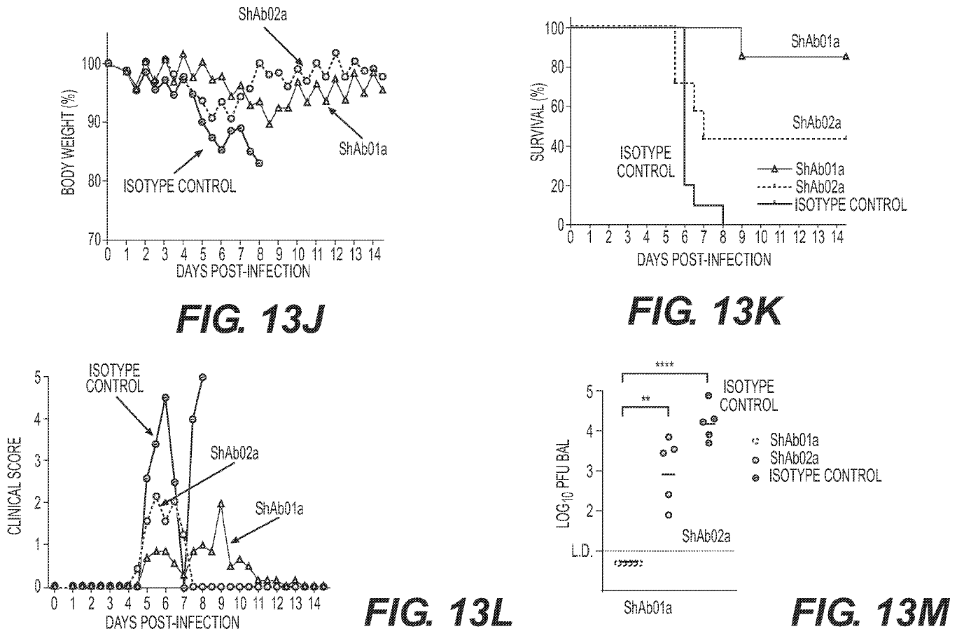

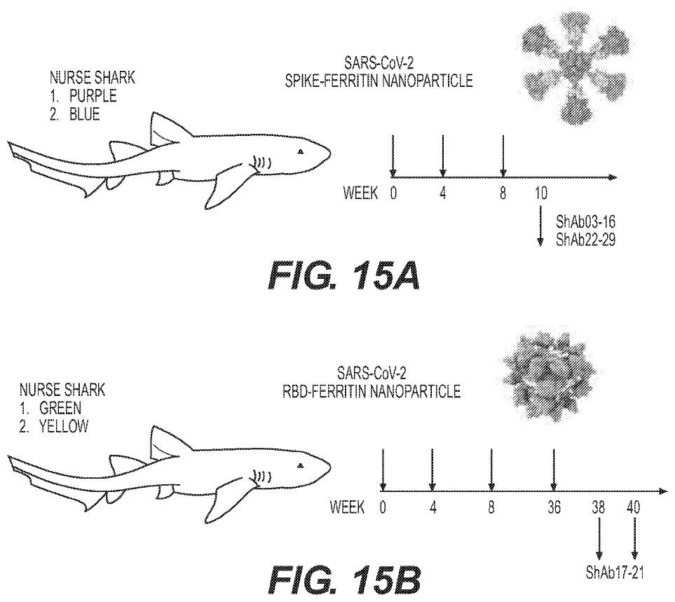

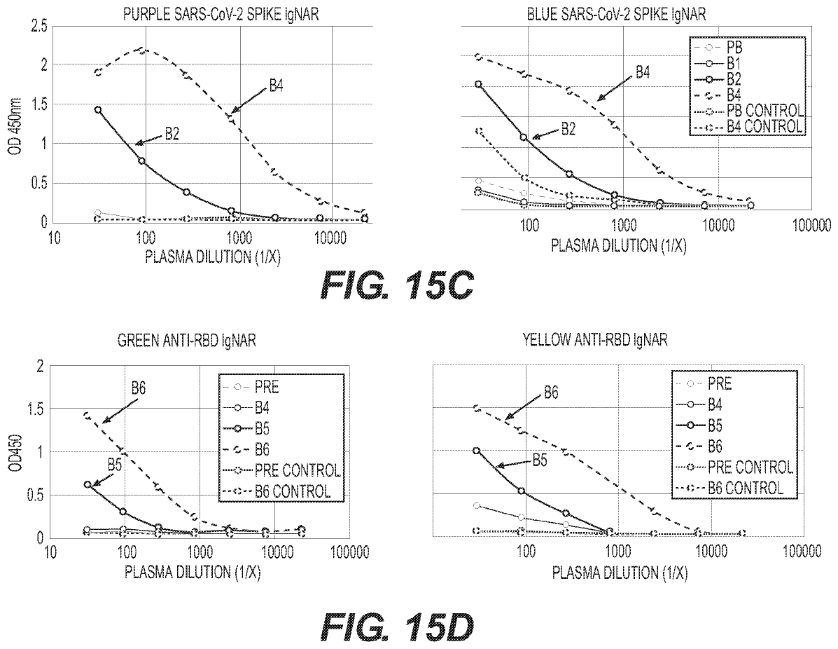

Six juvenile nurse sharks (two males and four females, aged between 2-3 years and weighing between 1.8-3.8 kg) were held in a continuously-recirculating 12,000-L seawater tank maintained at 28° C. in the Aquaculture Research Center at the Institute of Marine & Environmental Technology (IMET), Baltimore, USA. Following a sufficient period of acclimation, animals were primed with 200-250 μg antigen emulsified in complete Freund's adjuvant (CFA) administered subcutaneously into the ventral surface of the lateral fin. Sharks were boosted at 4-week intervals, first with 200-250 μg antigen emulsified in incomplete Freund's adjuvant (IFA) administered subcutaneously into the ventral surface of the lateral fin then intravenously with 100 μg antigen diluted in shark PBS (unadjuvanted) and administered directly into the caudal sinus. Sharks “Pink” and “Red” were immunized four times with SARS-CoV-2 RBD at week 0, 4, 8 and 13 (FIG. 13A); sharks “Green” and “Yellow” were immunized four times with SARS-CoV-2 RBD-Ferritin (RFN) at week 0, 4, 8, and 36 (FIG. 15B); and sharks “Purple” and “Blue” were immunized four times with SARS-CoV-2 Spike-Ferritin (SpFN) at week 0, 4, 8, and 14 (FIG. 15A). Small bleeds were drawn 2 weeks after each immunization, mixed with 1/10 volume of heparin (reconstituted to 1000 U/ml in shark-modified PBS), and centrifuged at 1000 rpm for 10 minutes to separate peripheral blood lymphocytes (PBLs) and blood plasma. Plasma samples were tested for the presence of antigen-specific IgNAR by binding ELISA as previously detailed (Dooley and Flajnik, 2005) but with the following revisions: 96-well Nunc MaxiSorp flat bottom microtiter plates were coated with recombinant SARS-CoV-2 RBD or spike protein diluted in PBS to 1 μg/ml; plates were incubated overnight at 4° C. then blocked with MPBS (PBS containing 5% milk powder) at room temperature for 2 hours; and control wells were blocked without prior antigen coating. The mouse monoclonal antibody GA8 was used to detect IgNAR binding. Once target-specific IgNAR titers had reached sufficient levels a larger blood sample was taken from the animal, and PBLs harvested for RNA isolation.

iii. VNAR Library Construction

VNAR libraries were built for the RBD-immunized animals, Pink and Red, using PBLs harvested at bleed 3 (week 10) then at bleed 5 (week 15); from the RFN-immunized animals, Yellow and Green, using PBLs harvested at bleed 4 (week 10) then at bleeds 5 and 6 (weeks 30 and 40). A single library was built from each SpFN-immunized animal, Blue and Purple, utilizing PBLs harvested at bleed 4 (week 10).

PBLs were lysed in phenol solution and total RNA prepared from each as per standard protocols. Oligo-dT-primed cDNA was prepared and used as the template for PCR amplification of IgNAR variable regions (VNARs) with the handled primers NARFr4-Rev1 (5′-ATAATCAAGCTTGC GGCCGCATTCACAGTCACGACAGTGCCACCTC-3′) and NARFr4-Rev2 (5′-ATAATCAA GCTTGCGGCCGCATTCACAGTCACGGCAGTGCCATCTC-3′) mixed in an equal ratio, and NARFr1-For (5′-ATAATAAGGAATTCCATGGCTCGAGTGGACCAAACACCG-3′). The ˜400 bp PCR products were cleaned, digested overnight with the restriction enzymes NcoI and NotI at sites introduced in the primer handles, cleaned again then cloned into similarly cut, shrimp alkaline phosphatase-treated pHEN2. This phagemid vector has a bacteriophage packaging signal and produces soluble VNAR fused to the phage gene III coat protein thus physically linking the VNAR sequence (genotype) with its antigen binding ability (phenotype). The resultant VNAR libraries were phenol:chloroform cleaned, resuspended in 10 μl DEPC-treated water, and transformed into electrocompetent E. coli TG1 cells (Agilent). Cells were resuspended in 2×TY media and allowed to recover for 1 hour at 37° C., then plated on TYE agar bioassay plates containing 100 μg/ml ampicillin (A100) and 2% glucose (G2). Each of the VNAR libraries produced exceeded 10¹⁰ members in size. Colonies were scraped from the bioassay plates into 2×TY/A100/G2 media containing 30% sterile glycerol and aliquots of the library flash frozen for storage at −80° C.

iv. VNAR Library Selections

Library selections were performed as detailed in Dooley et al., 2003. Briefly, a single aliquot of library stock was added to pre-warmed 2×TY/A100/G2 and grown with shaking at 37° C. to mid-log phase prior to infection with M13K07 helper phage. Cultures were spun and cell pellets resuspended in 2×TY containing 100 μg/ml ampicillin, 50 μg/ml kanamycin, and 0.2% glucose (2×TY/A100/K50/G0.2) then incubated with shaking at 30° C. overnight to permit library expression. Phage were precipitated from the culture supernatant by the addition of 1/3 volume of PEG-NaCl and phage pellets resuspended in PBS and titered ready for use in panning.

Libraries were panned on immunotubes (Maxisorp, Nunc) coated overnight at 4° C. with antigen diluted in PBS to the required concentration, then blocked with 5% MPBS. Selection was conducted by incubating coated immunotubes for 2 hours at room temperature with 1 ml of phage solution in 4 ml of 5% MPBS. Following incubation, unbound phages were discarded, the immunotube washed, then bound phage eluted with 1 ml of 100 mM triethylamine. The phage solution was neutralized by the addition of 0.5 ml of 1M Tris-HCl, pH 7.4. A log phase E. coli TG1 culture was infected with 0.75 ml of eluted phage and grown on TYE/A100/G2 bioassay plates at 30° C. overnight. The resulting colonies were scraped from the plates and grown to log phase in 2×TY/A100/G2 media prior to M13K07 infection. Subsequent rounds of selection and rescue were repeated as above. Enrichment of target-specific clones was evaluated via the binding of polyclonal and monoclonal phage supernatant to ELISA plates coated with antigen at 1 μg/ml and blocked with 5% MPBS. Phage binding was detected with anti-M13 phage coat G8p monoclonal antibody (Invitrogen) followed by anti-mouse HRP antibody (Sigma Aldrich). Plasmid was prepared from individual clones identified as being positive for antigen binding and their VNAR inserts sequenced using the vector-specific primers pHEN Seq (5′-CTATGCGGCCCCATTCA-3′) and LMB3 (5′-CAGGAAACAGCTATGAC-3′).

v. DNA Plasmid Construction and Preparation

SARS-CoV-2 Spike-Ferritin or RBD-Ferritin constructs were derived from the Wuhan-Hu-1 strain genome sequence (GenBank MN9089473), including RBD subunit (residues 331-527) or S ectodomain (residues 12-1158). Constructs were modified to incorporate a N-terminal hexa-histidine tag (His) for purification of the RBD-Ferritin construct.

An S-2P construct was used as a template to generate the Spike-Ferritin nanoparticle. The His-tagged SARS-CoV-2 RBD molecule was generated by amplifying the RBD domain from the RBD-Ferritin plasmid while encoding the 3′ purification tag and subcloned into the CMVR vector. The NTD protein subunit was generated in a similar manner, by amplifying the NTD domain from the Spike-Ferritin construct. For expression of monomeric Spike, RBD, or NTD proteins, the Spike protein domains were cloned into the CMVR expression plasmid using the NotI/BamHI restriction sites. Constructs including the N-terminal region of the Spike protein included the native leader sequence; for constructs not including this region, a prolactin leader (PL) sequence (Boyington et al., 2016) was utilized.

Plasmid DNA generated by subcloning (restriction digest and ligation) were amplified in and isolated from E. coli Top10 cells. The constructs resulting from site-directed mutagenesis were either amplified in and isolated from E. coli Stb13 or Top10 cells. Large-scale DNA isolation was performed using either endo free Maxiprep, Megaprep or Gigaprep kits (Qiagen).

vi. Recombinant Protein Expression

All expression vectors were transiently transfected into Expi293F cells (Thermo Fisher Scientific) using ExpiFectamine 293 transfection reagent (Thermo Fisher Scientific). Cells were grown in polycarbonate baffled shaker flasks at 34° C. or 37° C. and 8% CO₂ at 120 rpm. Cells were harvested 5-6 days post-transfection via centrifugation at 2,862×g for 30 minutes. Culture supernatants were filtered with a 0.22-μm filter and stored at 4° C. prior to purification.

vii. Immunogen and Spike Domain Purification

His-tagged proteins were purified using Ni-NTA affinity chromatography, while untagged proteins were purified with GNA lectin affinity chromatography. Briefly, 25 mL GNA-lectin resin (VectorLabs) was used to purify untagged protein from 1 liter of expression supernatant. GNA resin was equilibrated with 10 column volumes (CV) of phosphate buffered saline (PBS) (pH 7.4) followed by supernatant loading at 4° C. Unbound protein was removed by washing with 20 CV of PBS. Bound protein was eluted with 250 mM methyl-α-D mannopyranoside in PBS buffer (pH 7.4). Histidine-tagged proteins were purified using 1 mL Ni-NTA resin (Thermo Scientific) per 1 liter of expression supernatant. Ni-NTA resin was equilibrated with 5 CV of PBS followed by supernatant loading at room temperature. Unbound protein was removed by washing with 200 CV of PBS, followed by 50 CV 10 mM imidazole in PBS. Purification purity for all the proteins was assessed by SDS-PAGE. RBD proteins were dialyzed (10K molecular weight cutoff) across PBS; immunogens and Spike and NTD proteins were further purified by size-exclusion chromatography using a 16/60 Superdex-200 purification column. Removal of the His-tags for SARS2-CoV-2 S-2P and RBD for use in ELISA were produced using HRV-3C protease. Endotoxin levels for ferritin nanoparticle immunogens were evaluated and 5% v/v glycerol was added prior to filter-sterilization with a 0.22-μm filter, flash-freezing in liquid nitrogen, and storage at −80° C. Ferritin nanoparticle formation was assessed by Dynamic light scattering by determining the hydrodynamic diameter at 25° C. using a Malvern Zetasizer Nano S (Malvern, Worcestershire, UK) equipped with a 633-nm laser.

viii. IgG1 Fc-Fusion Antibody Purification

Fc-fusion antibodies were purified with Protein A affinity chromatography. rProtein A Sepharose™ Fast Flow Affinity Media (GE Healthcare/Cytiva) was used to purify Fc-fusion antibodies from 1 liter of expression supernatant. Protein A resin was equilibrated with 20 CV of PBS followed by supernatant loading at room temperature. Unbound protein was removed by washing with 60 CV of PBS. Bound protein was eluted with IgG Elution Buffer (Thermo Scientific) and neutralized with 0.1 M Tris, pH 8.0. Purification purity was assessed by SDS-PAGE under reducing and nonreducing conditions. For long-term storage, Fc-fusion antibodies were filter-sterilized with a 0.22-μm filter, flash-frozen in liquid nitrogen, and stored at −80° C.

ix. X-Ray Crystallography

Protein Crystallization: All proteins were crystallized by hanging-drop vapor diffusion at 293 K. The SARS-CoV-2 RBD/ShAb01 VNAR/ShAb02 VNAR complex (9.5 mg/ml) were screened for crystallization conditions using an Art Robbins Gryphon crystallization robot, 0.2 μl drops, and a set of 1200 conditions. Crystal drops were observed daily using a Jan Scientific UVEX-PS hotel with automated UV and brightfield drop imaging. Initial crystallization conditions were optimized manually in larger 1 μl drops, and crystals used for data collection grew in the following crystallization conditions: 0.1M HEPES (pH7.4), 24% PEG3350, 7.5% Glycerol.

Diffraction data collection and processing: Single crystals were transferred to mother liquor containing 20-25% glycerol, and cryo-cooled in liquid nitrogen prior to data collection. Diffraction data were collected at Advanced Photon Source (APS), Argonne National Laboratory beamlines. Diffraction data were collected at APS 24-ID-E beamline and measured using a Dectris Eiger 16M PIXEL detector to a final resolution of 2.6 Å. For diffraction data indexing, integration, and scaling were carried out using the HKL2000 suite27.

Structure solution and refinement: Phenix.xtriage was used to analyze all the scaled diffraction data output from HKL2000 and XDS. Primarily, data was analyzed for measurement value significance, completeness, asymmetric unit volume, and possible twinning and/or pseudotranslational pathologies. All crystal structures described in this study were solved by molecular replacement using the program Phaser. Refinement for all structure models was carried out using Phenix refine with positional, global isotropic B-factor refinement and defined TLS groups. Manual model building was performed in Coot. All structure figures were generated using PyMOL (Schrödinger, Inc.).

x. Octet Biolayer Interferometry Binding and ACE2 Inhibition Assays

All biosensors were hydrated in PBS prior to use. All assay steps were performed at 30° C. with agitation set at 1,000 rpm in the Octet RED96 instrument (forteBio). Biosensors were equilibrated in assay buffer (PBS) for 30 seconds before loading of IgG antibodies (30 μg/ml diluted in PBS). ShAb-Fc chimeras were immobilized onto AHC biosensors (forteBio) for 100 seconds, followed by a brief baseline in assay buffer for 15 seconds. Immobilized antibodies were then dipped in various antigens for 100-180 seconds followed by dissociation for 100 seconds.

ACE2 inhibition assays were carried out as follows. SARS-CoV-2 RBD, SARS-CoV-1 RBD, or S-2P (30 μg/ml diluted in PBS) was immobilized on HIS1K biosensors (ForteBio) for 180 seconds followed by baseline equilibration for 30 seconds. Serum was allowed to occur for 180 seconds followed by baseline equilibration (30 seconds). ACE2 protein (30 μg/ml) was then allowed to bind for 120 seconds. Percent inhibition (PI) of RBD binding to ACE2 by serum was determined using the equation: PI=100−((ACE2 binding in the presence of mouse serum)/(mouse serum binding in the absence of competitor mAb)×100).

xi. Enzyme Linked Immunosorbent Assay (ELISA)

96-well “U” Bottom plates were coated with 1 μg/mL of RBD or spike protein (S-2P) antigen in PBS, pH 7.4. Plates were incubated at 4° C. overnight and blocked with blocking buffer (Dulbecco's PBS containing 0.5% milk and 0.1% Tween 20, pH 7.4) at room temperature (RT) for 2 hours. Individual serum samples were serially diluted 2-fold in blocking buffer and added to triplicate wells and the plates were incubated for 1 hour at room temperature. Horseradish peroxidase (HRP)-conjugated sheep anti-mouse IgG, gamma chain specific (The Binding Site) was added and incubated at room temperature for an hour, followed by the addition of 2,2′-Azinobis [3-ethylbenzothiazoline-6-sulfonic acid]-diammonium salt (ABTS) HRP substrate (KPL) for 1 hour at room temperature. The reaction was stopped by the addition of 1% SDS per well and the absorbance (A) was measured at 450 nm using an ELISA reader Spectramax (Molecular Devices, San Jose, CA). Antibody positive (anti-RBD mouse mAb; BEI resources) and negative controls were included on each plate. The results are expressed as end-point titers, defined as the reciprocal dilution that gives an absorbance value that equals twice the background value (wells that did not contain RBD or S-2P protein).

xii. SARS-CoV-2 and SARS-CoV-1 Pseudovirus Neutralization Assay

The S (spike) expression plasmid sequences for SARS-CoV-2 and SARS-CoV-1 were codon optimized and modified to remove an 18-amino acid endoplasmic reticulum retention signal in the cytoplasmic tail in the case of SARS-CoV-2 and a 28-amino acid deletion in the cytoplasmic tail in the case of SARS-CoV. This allowed increased Spike incorporation into pseudovirions (PSV) and thereby enhance infectivity. Virions pseudotyped with the vesicular stomatitis virus (VSV) G protein were used as anon-specific control. SARS-CoV-2 pseudovirions (PSV) were produced by co-transfection of HEK293T/17 cells with a SARS-CoV-2 S plasmid (pcDNA3.4) and an HIV-1 NL4-3 luciferase reporter plasmid. The SARS-CoV-2 S expression plasmid sequence was derived from the Wuhan seafood market pneumonia virus isolate Wuhan-Hu-1, complete genome (GenBank accession MN908947) and the SARS-CoV-1 S expression plasmid was derived from the Urbani S sequence.

Infectivity and neutralization titers were determined using ACE2-expressing HEK293 target cells (Integral Molecular) in a semi-automated assay format using robotic liquid handling (Biomek NXp Beckman Coulter). Test sera were diluted 1:40 in growth medium and serially diluted, then 25 μL/well was added to a white 96-well plate. An equal volume of diluted SARS-CoV-2 PSV was added to each well and plates were incubated for 1 hour at 37° C. Target cells were added to each well (40,000 cells/well) and plates were incubated for an additional 48 hours. RLUs were measured with the EnVision Multimode Plate Reader (Perkin Elmer, Waltham, MA) using the Bright-Glo Luciferase Assay System (Promega Corporation, Madison, WI). Neutralization dose-response curves were fitted by nonlinear regression using the LabKey Server®, and the final titers are reported as the reciprocal of the dilution of serum necessary to achieve 50% neutralization (ID50, 50% inhibitory dilution) and 80% neutralization (ID80, 80% inhibitory dilution). Assay equivalency for SARS-CoV-2 was established by participation in the SARS-CoV-2 Neutralizing Assay Concordance Survey (SNACS) run by the Virology Quality Assurance Program and External Quality Assurance Program Oversite Laboratory (EQAPOL) at the Duke Human Vaccine Institute, sponsored through programs supported by the National Institute of Allergy and Infectious Diseases, Division of AIDS.

xiii. K18-ACE2 Transgenic Mouse Passive Immunization and Challenge

All research in this study involving animals was conducted in compliance with the Animal Welfare Act, and other federal statutes and regulations relating to animals and experiments involving animals and adhered to the principles stated in the Guide for the Care and Use of Laboratory Animals, NRC Publication, 1996 edition. The research protocol was approved by the Institutional Animal Care and Use Committee of the Trudeau Institute. K18-ACE2 transgenic mice were obtained from Jackson Laboratories (Bar Harbor, ME). Mice were housed in the animal facility of the Trudeau Institute and cared for in accordance with local, state, federal, and institutional policies in a National Institutes of Health American Association for Accreditation of Laboratory Animal Care-accredited facility.

For the passive immunization studies, on day −1 (one day before the infection), 200 μg of ShAb Fc chimeras ShAb01a, ShAb02a, ShAb06a, or a combination of ShAb02a and ShAb06a, were injected into the intraperitoneal cavity of five groups of K18-ACE2 mice. On study day 0, all mice were infected with 1.25×10⁴ PFU of SARS-CoV-2 USA-WA1/2020 via intranasal instillation. An additional group of mice received 200 μg total of a combination of ShAb02a and ShAb06a, on day 1 after infection, to assess the ability of the ShAbs to act with therapeutic activity.

All mice were monitored for clinical symptoms and body weight twice daily, every 12 hours, from study day 0 to study day 14. Mice were euthanized if they displayed any signs of pain or distress as indicated by the failure to move after stimulation or presentation of inappetence, or if mice have greater than 25% weight loss compared to their study day 0 body weight. Animals were assigned a clinical score as follows: 0 for normal appearance and movement; 1 for slightly ruffled fur; 2 for slightly ruffled fur and reduced mobility; 3 for slightly ruffled fur, reduced mobility and rapid breathing, 4 for slightly ruffled fur, reduced mobility, rapid breathing and hunched and huddled stance; and 5 for found dead or euthanized due to weight cut-off or being moribund.

xiv. Weighing Epitope Sites Based on Antigen-Antibody Interactions

Epitope sites correspond to antigen sites that are in contact with the antibody in the antigen-antibody complex (i.e., all sites that have non-hydrogen atoms within 4 Å of the antibody). For a given epitope site, the weight, which characterizes the interaction between the epitope site and the antibody (improved based on (Bai et al., 2019)), was defined as:

$$W = \frac{1}{2} ( \frac{{n}_{c}}{〈 {n}_{c} 〉} + \frac{{n}_{nb}}{〈 {n}_{nb} 〉} )$$

in which, n_{c} is the number of contacts with the antibody (i.e., the number of non-hydrogen antibody atoms within 4 Å of the site); n_{nb} is the number of neighboring antibody residues; [symbol] n_{c} [symbol] is the mean number of contacts n_{c} and [symbol] n_{nb} [symbol] is the mean number of neighboring antibody residues n_{nb} across all epitope sites. A weight of 1.0 is attributed to the average interaction across all epitope sites. Neighboring residue pairs were identified by Delaunay tetrahedralization of side-chain centers of residues (C is counted as a side chain atom, pairs further than 8.5 Å were excluded). Quickhull (Barber, 1996) was used for the tetrahedralization and Biopython PDB (Hamelryck and Manderick, 2003) to handle the protein structure.

In the SARS-CoV-2 RBD and SARS-CoV-1 RBD comparison, residues were considered similar for the following residues pairs: RK, RQ, KQ, QE, QN, ED, DN, TS, SA, VI, IL, LM, and FY.

xv. Statistical Analysis

K18-ACE2 mouse survival comparisons were carried out using GraphPad using Gehan-Breslow-Wilcoxon test.

xvi. Epitope Mapping of Antibodies by Alanine Scanning

Epitope mapping was performed essentially as described previously (Davidson et al., 2014) using SARS-CoV-2 (strain Wuhan-Hu-1) Spike protein RBD and NTD shotgun mutagenesis mutation libraries, made using a full-length expression construct for Spike protein. 184 residues of the RBD (between S residues 335 and 526) and 300 residues of the NTD (between residues 2 and 307) were mutated individually to alanine, and alanine residues to serine. Mutations were confirmed by DNA sequencing, and clones arrayed in 384-well plates, one mutant per well. Binding of mAbs to each mutant clone in the alanine scanning library was determined, in duplicate, by high-throughput flow cytometry. Each Spike protein mutant was transfected into HEK-293T cells and allowed to express for 22 hours. Cells were fixed in 4% (v/v) paraformaldehyde (Electron Microscopy Sciences), and permeabilized with 0.1% (w/v) saponin (Sigma-Aldrich) in PBS plus calcium and magnesium (PBS++) before incubation with mAbs diluted in PBS++, 10% normal goat serum (Sigma), and 0.1% saponin. MAb screening concentrations were determined using an independent immunofluorescence titration curve against cells expressing wild-type Spike protein to ensure that signals were within the linear range of detection. Antibodies were detected using 3.75 μg mL⁻¹ of AlexaFluor488-conjugated secondary antibody (Jackson ImmunoResearch Laboratories) in 10% normal goat serum with 0.1% saponin. Cells were washed three times with PBS++/0.1% saponin followed by two washes in PBS and mean cellular fluorescence was detected using a high-throughput Intellicyte iQue flow cytometer (Sartorius). Antibody reactivity against each mutant Spike protein clone was calculated relative to wild-type Spike protein reactivity by subtracting the signal from mock-transfected controls and normalizing to the signal from wild-type Spike-transfected controls. Mutations within clones were identified as important to the mAb epitope if they did not support reactivity of the test MAb, but supported reactivity of other SARS-CoV-2 antibodies. This counter-screen strategy facilitates the exclusion of Spike mutants that are locally misfolded or have an expression defect.

xvii. List of Immunogens and RBD Variants

A list of immunogens and RBD variants used in this study, as well as leader sequences and linkers used for preparation of constructs, is provided below.

| | | Nucleotide | Amino Acid |
|---|---|---|---|
| | | Sequence | Sequence |
| Clone Name | Construct | (SEQ ID NO:) | (SEQ ID NO:) |
| pCoV08 | RBD-His6 | 232 | 218 |
| pCoV1B-06-PL | PrefLead-S2P.1158op1-del-Ferritin | 233 | 219 |
| pCoV131 | His8-3c-RBD-Ferritin-Y453R-518LLH to NKS | 234 | 220 |
| pCoV234 | RBD-His6-L452R-E484Q | 235 | 221 |
| pCoV235 | RBD-His6-E484K-N501Y-K417T | 236 | 222 |
| pCoV236 | RBD-His6-K356E | 237 | 223 |
| pCoV237 | RBD-His6-F377S-K378E | 238 | 224 |
| pCoV238 | RBD-His6-D428K | 239 | 225 |
| pCoV239 | RBD-His6-L452R-T478K | 240 | 226 |
| pCoV240 | His8-3c-RBD-GSGGSG-NTD-SSQC | 241 | 227 |
| pCoV241 | RBD-His6-L452Q-F490S | 242 | 228 |
| pCoV242 | RBD-His6-F490S | 243 | 229 |
| pCoV243 | RBD-His6-L452Q | 244 | 230 |
| pCoV244 | RBD-His6-F486L | 245 | 231 |
| 24 aa linker | | | 207 |
| 25 aa linker | | | 208 |
| KES linker | | | 209 |
| x linker | 15-Helix2(AA53-90)-4-Helix1(AA19-45)-12 | | 210 |
| y linker | 21-Helix1 + 2(AA19-90)-12 | | 211 |
| z linker | 21-Helix1(AA19-45)-12 | | 212 |
| CC linker | | | 213 |
| Leader sequence | | 216 | 214 |
| for Immunogens | | | |
| and RBD variants | | | |
| Leader sequence | | 217 | 215 |
| for ShAbs and | | | |
| related proteins | | | |

xviii. List of Constructs

A list of constructs used in this study is provided below.

| | |
|---|---|
| ID | Description |

Single-domain

| | |
|---|---|
| ShAb01a-ShAb11a | ShAb-3c-Fc |
| ShAb12-16a (NTD) | ShAb-3c-Fc |
| ShAb18a-29a | ShAb-3c-Fc |

Knob-in-hole

| | |
|---|---|
| ShAb01H02K | ShAb01Hole-02Knob |
| ShAb01H06K | ShAb01Hole-06Knob |
| ShAb06H02K | ShAb06Hole-02Knob |
| ShAb01H11K | ShAb01Hole-11Knob |
| ShAb11H02K | ShAb11Hole-02Knob |
| ShAb06H11K | ShAb06Hole-11Knob |
| ShAb_H07K | ShAb07Knob |
| ShAb08H_K | ShAb08Hole |
| ShAb09H_K | ShAb09Hole |
| ShAb_H10K | ShAb10Knob |

Multi-domain: Bi-specific

| | |
|---|---|
| BiShAb21a | ShAb02-25*-ShAb01-3c-Fc |
| BiShAb21x | ShAb02-15*-Helix2(AA53-90)-4-Helix1(AA19-45)- |
| | 12 |
| | ShAb01-3c-Fc |
| BiShAb21y | ShAb02-21*-Helix1 + 2(AA19-90)-12-ShAb01- |
| | 3c-Fc |
| BiShAb21z | ShAb02-21*-Helix1(AA19-45)-12-ShAb01-3c-Fc |
| BiShAb210a | ShAb02-25*-ShAb10-3c-Fc |
| BiShAb102a | ShAb10-25*-ShAb02-3c-Fc |
| BiShAb211a | ShAb02-25*-ShAb11-3c-Fc |
| BiShAb112a | ShAb11-25*-ShAb02-3c-Fc |

Multi-domain: Tri-specific

| | |
|---|---|
| TriShAb216a | ShAb02-25*-ShAb01-24*-ShAb06-3c-Fc |
| TriShAb217a | ShAb02-25*-ShAb01-24*-ShAb07-3c-Fc |

Trimeric

| | |
|---|---|
| ShAb01-Foldon | ShAb01-KESlinker**-Foldon-RS-3c-His8-Strep |
| ShAb02-Foldon | ShAb02-KESlinker**-Foldon-RS-3c-His8-Strep |
| BiShAb21F | ShAb02-25*-ShAb01-Foldon-3c-His8-Strep |

ShAb-3c-His

| | |
|---|---|
| ShAb06b | ShAb06-3c-His6 |
| ShAb07b | ShAb07-3c-His6 |
| ShAb10b | ShAb10-3c-His6 |
| ShAb11b | ShAb11-3c-His6 |

RBD variants and other proteins

| | |
|---|---|
| pCoV236 | RBD-His6-K356E (no ShAb02 binding) |
| pCoV237 | RBD-His6-F377S-K378E (no ShAb01 binding) |
| pCoV238 | RBD-His6-D428K (no ShAb01 binding) |
| pCoV239 | RBD-His6-L452R-T478K (Delta variant) |
| pCoV241 | RBD-His6-L452Q-F490S (C.37 variant) |
| pCoV242 | RBD-His6-F490S |
| pCoV243 | RBD-His6-L452Q |
| pCoV244 | RBD-His6-F486L |
| pHu-3c-Fc-Hole | For making one-armed ShAb-Fc |
| pHu-3c-Fc-Knob | For making one-armed ShAb-Fc |
| pCoV240 | His8-3c-RBD-GSGGSG-NTD |

### 2. Results

i. Immunization of Nurse Sharks with SARS-CoV-2 Immunogens

Nurse sharks “Red” and “Pink” were immunized with recombinant SARS-CoV-2 RBD protein (FIG. 13A), nurse sharks “Blue” and “Purple” were immunized with SARS-CoV-2 spike-ferritin nanoparticles (FIG. 15A), and nurse sharks “Green” and “Yellow” were immunized with RBD-ferritin nanoparticles (FIG. 15B). Small bleeds were taken two weeks after each boost and used to monitor antigen-specific IgNAR titers by binding ELISA. All six animals responded to immunization and showed significant increases in IgNAR binding to immobilized RBD or spike protein when compared to their pre-bleed plasma sample. However, the timing and magnitude of the response differed between individual animals, with responses in the RBD immunized animals delayed, compared to the two sharks immunized with SpFN.

ii. Isolation of SARS-CoV-2 S-Reactive Nanobodies

Given the binding ELISA data, 8 separate libraries were built using PBLs harvested at bleed 3 then bleed 5 for “Red” and “Pink”, bleed 4 for “Purple” and “Blue,” and bleed 5 and 6 combined for “Green” and “Yellow.” The resulting libraries all exceeded 10¹⁰ members in size. Keeping them separate at all stages, each library was subject to 2-3 rounds of selection on RBD- or spike-coated immunotubes, reducing antigen coating densities and increasing wash stringencies to favor selection of high affinity clones. A minimum of 200 clones from each library were screened for target binding; no antigen-positive clones were retrieved from the “Pink” bleed 3 library but for the other libraries 25-30% of clones screened bound their respective target (RBD or spike). Plasmid was prepared for all antigen-positive clones and their inserts sequenced, this yielded 29 novel VNAR clones (Table 1). Both type I and type II VNARs were present in the clone set and CDR3s ranged in length from 14-26 amino acids

Table 1 summarizes the information related to the sharks, immunizations, VNAR, panning targets, bleeds, and VNAR properties. Three separate groups of sharks were immunized, each group with a different immunogen (FIG. 13A, FIG. 15A, and FIG. 15B). The initial set of sharks named “Pink” and “Red” were immunized with RBD protein (FIG. 13A). Subsequent studies included immunization of sharks “Purple” and “Blue” with SpFN (FIG. 15A) and sharks “Green” and “Yellow” with RFN (FIG. 15B). IgNAR responses were assayed by ELISA against RBD and S-2P following immunizations, and a single phage library was generated from each of the six individual sharks (Table 1). Each of the libraries were screened at different times and using four different panning targets: RBD, NTD, SpFN, and RFN. A total of 31 VNARs (or ShAbs) were identified, of which ShAb01, ShAb02, ShAb09, ShAb17, ShAb18, ShAb19, ShAb20, ShAb21, ShAb22, ShAb23, ShAb24 ShAb29, and ShAb31 are RBD-targeting VNARs, and ShAb03, ShAb04, ShAb05, ShAb06, ShAb07, ShAb08, ShAb10, ShAb11, ShAb12, ShAb13, ShAb14, ShAb15 and ShAb16 are NTD-targeting VNARs. Based on competition assays, ShAb01, ShAb09, ShAb8, ShAb21, ShAb23 and ShAb24 are characterized as being the ShAb01 family members and ShAb02, ShAb19, ShAb22 and ShAb29 are characterized as being the ShAb02 family members.

TABLE 1
VNAR IDs, library origin, and clone characteristics.

| | ShAb | | Library, | Panning | Bleed | VNAR | CDR3 | |
|---|---|---|---|---|---|---|---|---|
| Immunogen | Name | Shark | pan | Target | Week | type | length | Specificity |
| RBD | ShAb01 | Pink | bleed 5, | RBD | 15 | type II | 20 aa | RBD |
| | | | pan 2 | | | | | |
| RBD | ShAb02 | Red | bleed 3, | RBD | 10 | type II | 17 aa | RBD |
| | | | pan 3 | | | | | |
| SpFN | ShAb03 | Blue | bleed 4, | SpFN | 10 | — | 25 aa | NTD |
| | | | pan 2 | | | | | |
| SpFN | ShAb04 | Blue | bleed 4, | SpFN | 10 | type II | 15 aa | NTD |
| | | | pan 2 | | | | | |
| SpFN | ShAb05 | Blue | bleed 4, | SpFN | 10 | type II | 18 aa | NTD |
| | | | pan 2 | | | | | |
| SpFN | ShAb06 | Purple | bleed 4, | SpFN | 10 | type I | 21 aa | NTD |
| | | | pan 2 | | | | | |
| SpFN | ShAb07 | Purple | bleed 4, | SpFN | 10 | type I | 21 aa | NTD |
| | | | pan 2 | | | | | |
| SpFN | ShAb08 | Purple | bleed 4, | SpFN | 10 | type II | 15 aa | NTD |
| | | | pan 2 | | | | | |
| — | ShAb09 | — | bleed 4, | SpFN | 10 | type II | 20 aa | RBD |
| | | | pan 3 | | | | | |
| SpFN | ShAb10 | Purple | bleed 4, | SpFN | 10 | type II | 16 aa | NTD |
| | | | pan 3 | | | | | |
| SpFN | ShAb11 | Purple | bleed 4, | SpFN | 10 | type II | 16 aa | NTD |
| | | | pan 3 | | | | | |
| SpFN | ShAb12 | Blue | bleed 4, | NTD | 10 | type II | 14 aa | NTD |
| | | | pan 2 | | | | | |
| SpFN | ShAb13 | Blue | bleed 4, | NTD | 10 | type I | 20 aa | NTD |
| | | | pan 2 | | | | | |
| SpFN | ShAb14 | Purple | bleed 4, | NTD | 10 | type I | 26 aa | NTD |
| | | | pan 2 | | | | | |
| SpFN | ShAb15 | Purple | bleed 4, | NTD | 10 | type I | 21 aa | NTD |
| | | | pan 2 | | | | | |
| SpFN | ShAb16 | Purple | bleed 4, | NTD | 10 | type I | 22 aa | NTD |
| | | | pan 2 | | | | | |
| RFN | ShAb17 | Green | bleed 5 + 6, | SpFN | 38 + 40 | type I | 20 aa | RBD |
| | | | pan 3 | | | | | |
| RFN | ShAb18 | Green | bleed 5 + 6, | SpFN | 38 + 40 | type I | 25 aa | RBD |
| | | | pan 3 | | | | | |
| RFN | ShAb19 | Yellow | bleed 5 + 6, | SpFN | 38 + 40 | type I | 18 aa | RBD |
| | | | pan 3 | | | | | |
| RFN | ShAb20 | Yellow | bleed 5 + 6, | SpFN | 38 + 40 | type II | 23 aa | RBD |
| | | | pan 3 | | | | | |
| RFN | ShAb21 | Yellow | bleed 5 + 6, | SpFN | 38 + 40 | type I | 20 aa | RBD |
| | | | pan 3 | | | | | |
| SpFN | ShAb22 | Blue | bleed 4, | RFN | 10 | type II | 17 aa | RBD |
| | | | pan 3 | | | | | |
| SpFN | ShAb23 | Blue | bleed 4, | RFN | 10 | type II | 20 aa | RBD |
| | | | pan 3 | | | | | |
| SpFN | ShAb24 | Blue | bleed 4, | RFN | 10 | type II | 19 aa | RBD |
| | | | pan 3 | | | | | |
| SpFN | ShAb25 | Blue | bleed 4, | RFN | 10 | type II | 16 aa | Not |
| | | | pan 3 | | | | | defined |
| SpFN | ShAb26 | Blue | bleed 4, | RFN | 10 | type I | 22 aa | Not |
| | | | pan 3 | | | | | defined |
| SpFN | ShAb27 | Blue | bleed 4, | RFN | 10 | type II | 19 aa | Not |
| | | | pan 3 | | | | | defined |
| SpFN | ShAb28 | Purple | bleed 4, | RFN | 10 | type II | 23 aa | Not |
| | | | pan 3 | | | | | defined |
| SpFN | ShAb29 | Purple | bleed 4, | RFN | 10 | type II | 26 aa | RBD |
| | | | pan 3 | | | | | |
| SpFN | ShAb30 | Blue | bleed 4, | RFN | 10 | type I | 22 aa | Not |
| | | | pan 3 | | | | | defined |
| RBD | ShAb31 | Red | — | RBD | — | — | 17 aa | RBD |

Each of the 31 VNARs (or ShAbs) was sequenced and their respective CDR1, CDR3, HV2 and HV4 were identified with their sequences summarized in Table 2 below.

TABLE 2
VNAR Sequences

| | Nucleotide | Amino Acid | CDR1 | CDR3 | HV2 | HV4 |
|---|---|---|---|---|---|---|
| | Sequence | Sequence | Sequence | Sequence | Sequence | Sequence |
| VNAR ID | (SEQ ID NO:) | (SEQ ID NO:) | (SEQ ID NO:) | (SEQ ID NO:) | (SEQ ID NO:) | (SEQ ID NO:) |
| ShAb01 | 31 | 1 | 246 | 276 | 306 | 336 |
| ShAb02 | 32 | 2 | 247 | 277 | 307 | 337 |
| ShAb03 | 33 | 3 | 248 | 278 | 308 | 338 |
| ShAb04 | 34 | 4 | 249 | 279 | 309 | 339 |
| ShAb05 | 35 | 5 | 250 | 280 | 310 | 340 |
| ShAb06 | 36 | 6 | 251 | 281 | 311 | 341 |
| ShAb07 | 37 | 7 | 252 | 282 | 312 | 342 |
| ShAb08 | 38 | 8 | 253 | 283 | 313 | 343 |
| ShAb09 | 39 | 9 | 254 | 284 | 314 | 344 |
| ShAb10 | 40 | 10 | 255 | 285 | 315 | 345 |
| ShAb11 | 41 | 11 | 256 | 286 | 316 | 346 |
| ShAb12 | 42 | 12 | 257 | 287 | 317 | 347 |
| ShAb13 | 43 | 13 | 258 | 288 | 318 | 348 |
| ShAb14 | 44 | 14 | 259 | 289 | 319 | 349 |
| ShAb15 | 45 | 15 | 260 | 290 | 320 | 350 |
| ShAb16 | 46 | 16 | 261 | 291 | 321 | 351 |
| ShAb17 | 47 | 17 | 262 | 292 | 322 | 352 |
| ShAb18 | 48 | 18 | 263 | 293 | 323 | 353 |
| ShAb19 | 49 | 19 | 264 | 294 | 324 | 354 |
| ShAb20 | 50 | 20 | 265 | 295 | 325 | 355 |
| ShAb21 | 51 | 21 | 266 | 296 | 326 | 356 |
| ShAb22 | 52 | 22 | 267 | 297 | 327 | 357 |
| ShAb23 | 53 | 23 | 268 | 298 | 328 | 358 |
| ShAb24 | 54 | 24 | 269 | 299 | 329 | 359 |
| ShAb25 | 55 | 25 | 270 | 300 | 330 | 360 |
| ShAb26 | 56 | 26 | 271 | 301 | 331 | 361 |
| ShAb27 | 57 | 27 | 272 | 302 | 332 | 362 |
| ShAb28 | 58 | 28 | 273 | 303 | 333 | 363 |
| ShAb29 | 59 | 29 | 274 | 304 | 334 | 364 |
| ShAb30 | 60 | 30 | 275 | 305 | 335 | 365 |
| ShAb31 | — | 394 | 395 | 396 | 397 | 398 |

iii. Characterization of ShAb01 and ShAb02

The initial set of VNARs analyzed was based on screening of sharks “Pink” and “Red” using RBD. This led to the identification of two ShAb VNARs, AliB5-2D8 and MoB3-3D8, as described in Example 1. AliB5-2D8 and MoB3-3D8 were renamed ShAb01 and ShAb02, respectively, and were extensively characterized (FIG. 13A-13M).

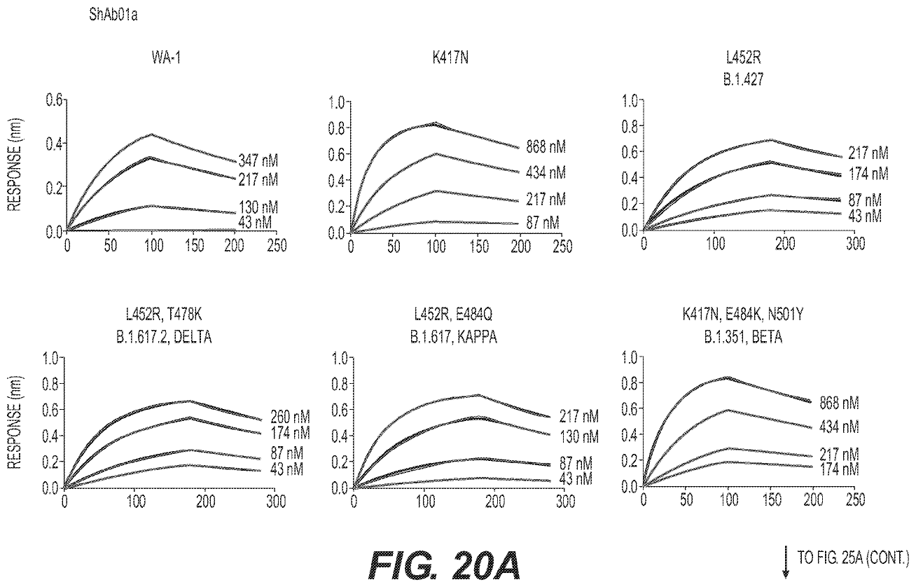

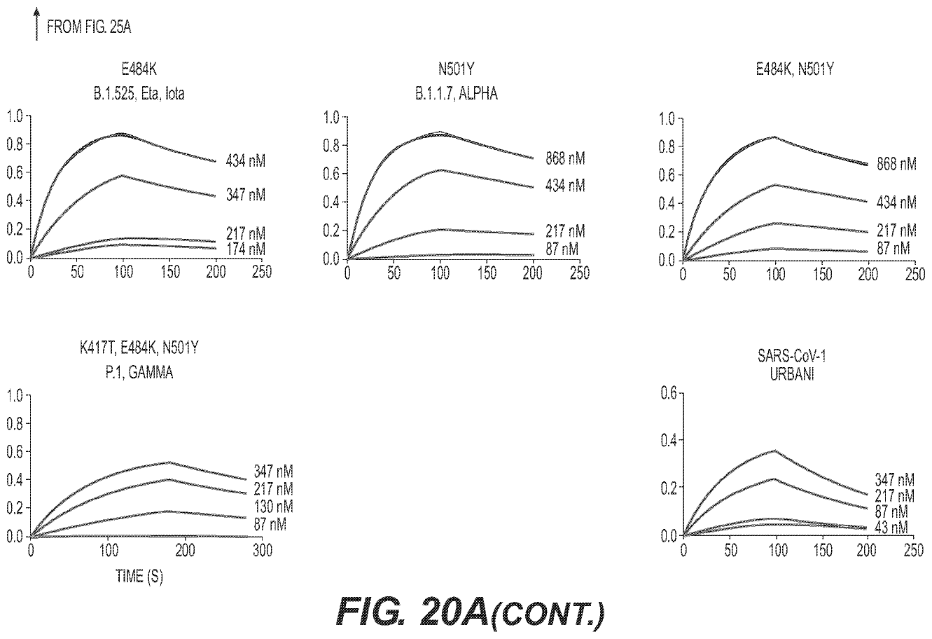

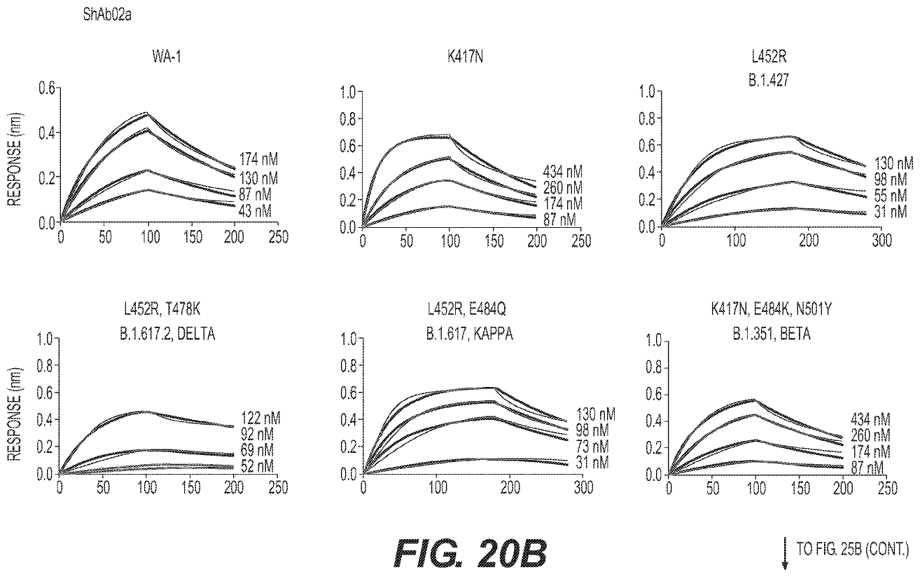

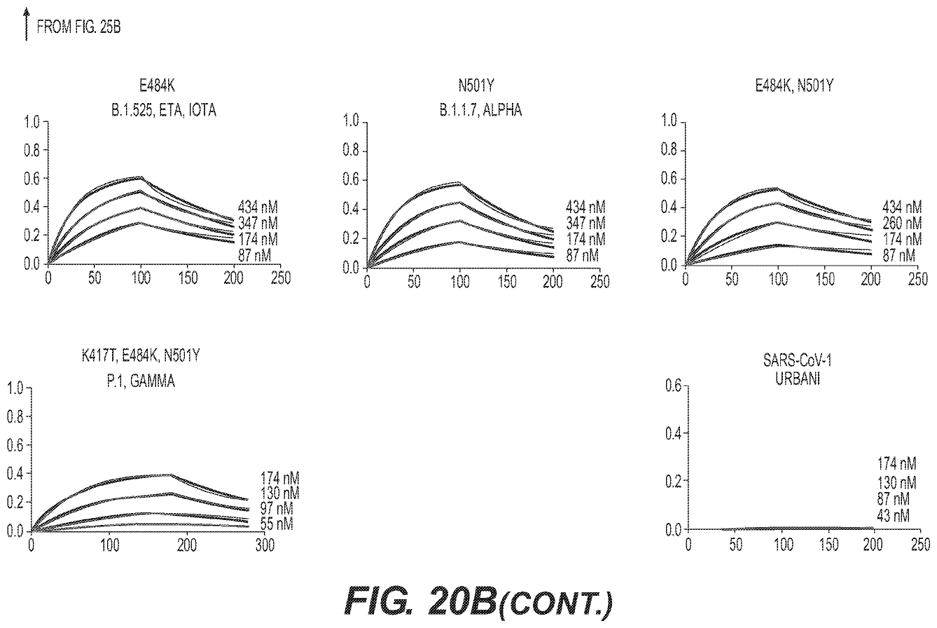

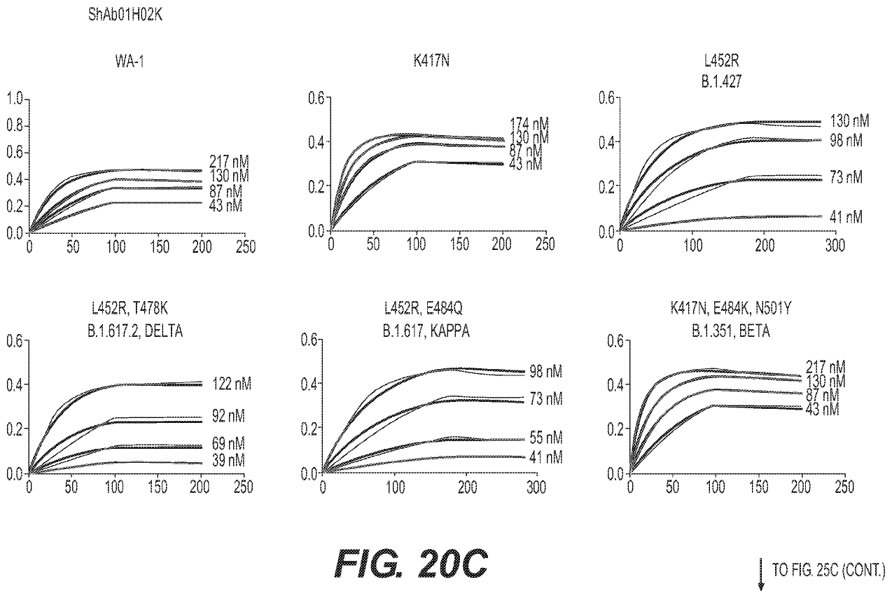

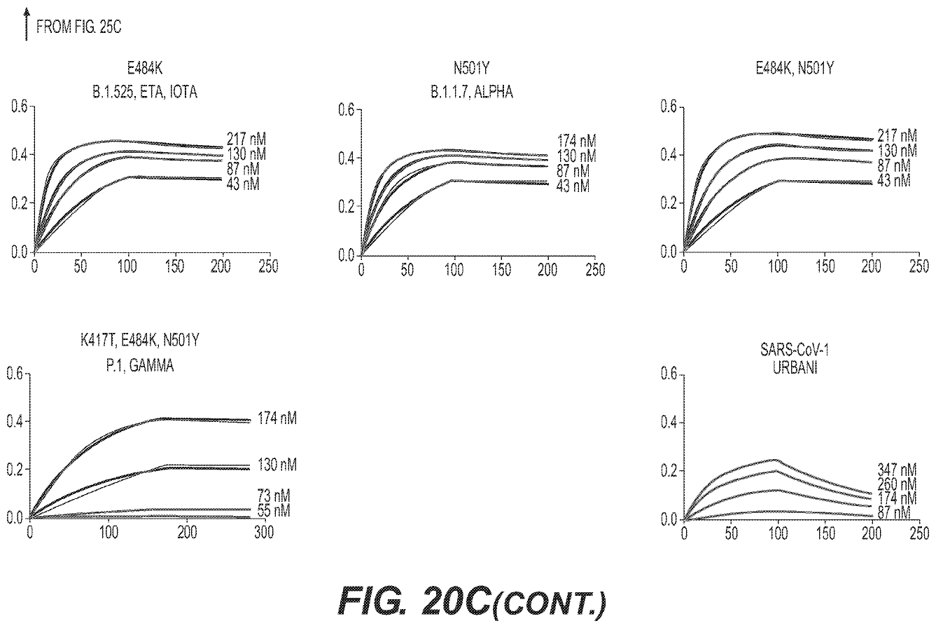

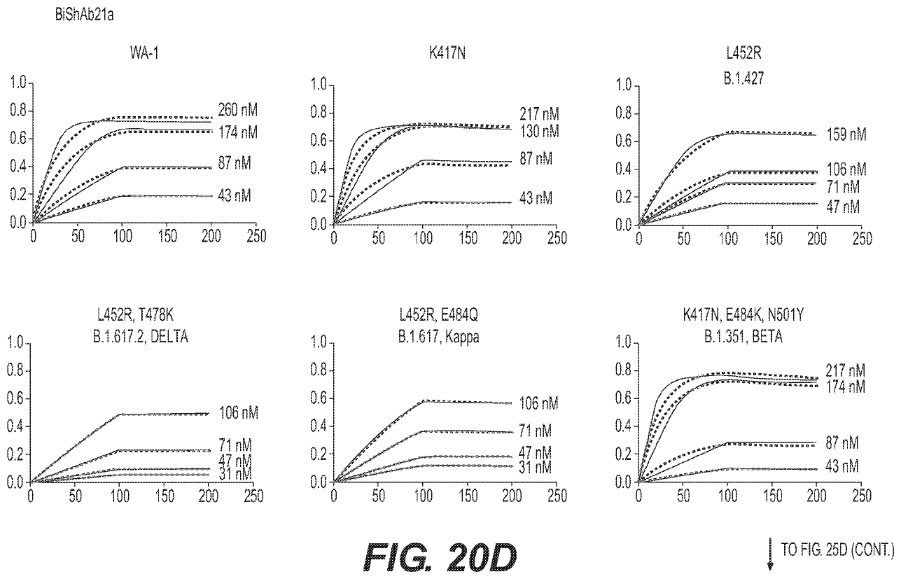

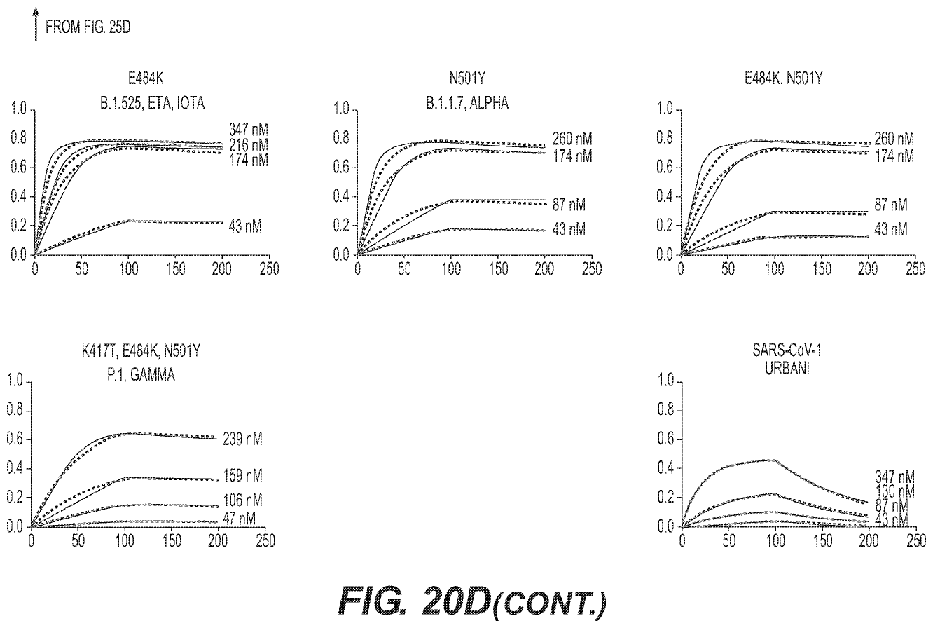

These two ShAb VNARs were expressed as human Fc fusion chimeras and characterized for binding by ELISA (FIG. 13C) and BLI (FIG. 13D, FIG. 20). Both ShAb01a (ShAb01-human Fc fusion chimera) and ShAb02a (ShAb02-human Fc fusion chimera) showed robust binding to both RBD and S-2P molecules, with affinity in the nanomolar range (FIG. 13D). Further assessment of ShAB01a and ShAb02a binding to RBD mutants showed that affinity was largely unaffected by RBD mutations observed in SARS-CoV-2 variants such as Alpha (B.1.1.7), Beta (B.1.351), or Delta (B.1.617.2). FIG. 20A-20B. Assessment of ShAb01a and ShAB02a for ACE2-blocking, showed that ShAb01a was able to potentially block ACE2 binding to both RBD and S-2P (FIG. 13E), while ShAb02a showed some ability to block ACE2. ShAb01a and ShAb02a were assessed for their ability to neutralize pseudoviruses (FIG. 13F). Both ShAb01a and ShAb02a molecules neutralized the viruses with high potency with ShAb02a showing ng ml-potency against SARS-CoV-2 variants, while ShAb02a showed robust neutralization against SARS-CoV-2 variants and the related sarbecovirus SARS-CoV-1.

iv. Characterization of Shark-Derived SARS-CoV-2 S-Reactive Nanobodies (ShAbs)

The selected VNARs were subcloned into a mammalian expression vector upstream of and in frame with DNA sequence encoding a human IgG1 Fc domain (Table 3). This allowed expression in mammalian cells and facilitated the ShAb-Fc chimeras in biochemical assays where reagents against human antibodies are readily available. The purified proteins were evaluated for their binding characteristics using enzyme-linked immunosorbent assay and biolayer interferometry.

TABLE 3
Production levels of ShAb immunotherapeutics via
transient transfection in mammalian cells.

| ID | Yield (mg protein/L culture) |
|---|---|
| Shab01 | 59.3 |
| Shab01a | 38.0 |
| Shab01b | 46.7 |
| Shab02b | 11.8 |
| Shab02 | 26.4 |
| Shab02a | 56.8 |
| Shab03 | 130.0 |
| Shab03a | 86.2 |
| Shab04a | 182.1 |
| Shab05a | 54.0 |
| Shab06a | 208.0 |
| Shab07a | 255.4 |
| Shab08a | 224.6 |
| Shab09a | 343.8 |
| Shab10a | 165.9 |
| Shab11a | 325.6 |
| Shab04 | 341.0 |
| Shab05 | 183.5 |
| Shab06 | 346.2 |
| Shab07 | 379.0 |
| Shab08 | 363.9 |
| Shab09 | 376.4 |
| Shab10 | 357.9 |
| Shab11 | 291.0 |
| Shab12a | 203.3 |
| Shab13a | 276.5 |
| Shab14a | 62.4 |
| Shab15a | 45.9 |
| Shab16a | 90.3 |
| Shab17a | 28.5 |
| Shab18a | 26.1 |
| Shab19a | 269.0 |
| Shab20a | 69.2 |
| Shab21a | 89.5 |
| Shab22a | 243.6 |
| Shab23a | 123.5 |
| Shab24a | 541.8 |
| Shab29a | 324.2 |
| ShAb01H02K | 514.0 |
| ShAb01H06K | 576.3 |
| ShAb06H02K | 461.7 |
| ShAb01H11K | 204.2 |
| ShAb11H02K | 190.2 |
| ShAb06H11K | 186.1 |
| BiShAb21a | 173.3 |
| BiShAb21x | 5.6 |
| BiShAb21y | 18.8 |
| BiShAb21z | 3.5 |
| BiShAb21-Foldon | 3.3 |
| ShAb01-Foldon | 6.3 |
| ShAb02-Foldon | 11.7 |
| TrishAb216a | 41.0 |
| TrishAb217a | 165.9 |

The initial identified VNAR molecules, including ShAb01a, ShAb02a, showed strong binding to RBD (Table 4). Subsequent VNARs were identified from sharks “Green,” “Yellow,” “Purple,” and “Blue,” and showed affinity to either the RBD molecule (Table 5) or the NTD molecule (Table 6) with nanomolar affinity. Epitope competition with ShAb01a, ShAb02a, and ACE2 classified the RBD-targeting ShAbs as falling into two epitope groups (FIG. 13I). Epitope competition studies using ShAb01a and ShAb02a showed that all of these additional antibodies blocked either ShAb01a or ShAB02a binding (FIG. 13G, FIG. 13H, FIG. 13I). ShAbs that competed with ShAb01a showed some ACE2 blocking ability (e.g., ShAb09a and ShAb23a displayed robust ACE2-blocking ability), while those that competed with ShAb02a did not show any ACE2 blocking activity at 30 μg/ml concentration (FIG. 13G-I). In addition to the RBD-targeting ShAbs, 11 ShAbs including ShAb34a, ShAb06a, ShAb07a, ShAb08a, and ShAb10a-16a showed binding to SARS-CoV-2 NTD. All of the ShAbs tested showed high levels of binding to SARS-CoV-2 S-2P indicative that they could bind to the prefusion form of the Spike trimer molecule. In all cases, RBD-targeting and NTD-targeting ShAbs showed nanomolar affinity.

TABLE 4
Biolayer Interferometry measurement of ShAb-based immunotherapeutics
to SARS-COV-2 RBD variants and SARS-COV-1 RBD.

| | ShAb01a (AliB5-Fc) | | | ShAb02a (MoB3-Fc) | | | BiSh21a | | | ShAb1H2K | | |
|---|---|---|---|---|---|---|---|---|---|---|---|---|
| | K_{D} | kₒₙ | k_{off} | K_{D} | kₒₙ | k_{off} | K_{D} | kₒₙ | k_{off} | K_{D} | kₒₙ | k_{off} |
| | (nM) | (1/Ms) | (1/s) | (nM) | (1/Ms) | (1/s) | (nM) | (1/Ms) | (1/s) | (nM) | (1/Ms) | (1/s) |
| SARS- | 47.1 | 4.16 × | 1.96 × | 85.7 | 8.12 × | 6.96 × | 0.46 | 1.36 × | 6.24 × | 0.76 | 1.53 × | 1.17 × |
| CoV-2 | | 10⁴ | 10⁻³ | | 10⁴ | 10⁻³ | | 10⁵ | 10⁻⁵ | | 10⁵ | 10⁻⁴ |
| RBD | | | | | | | | | | | | |
| K417N | 54.9 | 4.51 × | 2.48 × | 53.9 | 1.51 × | 8.15 × | 1.67 | 2.11 × | 3.52 × | 1.29 | 3.53 × | 4.57 × |
| | | 10⁴ | 10⁻³ | | 10⁵ | 10⁻³ | | 10⁵ | 10⁻⁴ | | 10⁵ | 10⁻⁴ |
| L452R | 35.1 | 5.74 × | 2.01 × | 29.8 | 1.31 × | 3.91 × | 1.23 | 1.09 × | 1.33 × | 0.008 | 1.56 × | 1.21 × |
| | | 10⁴ | 10⁻³ | | 10⁵ | 10⁻³ | | 10⁵ | 10⁻⁴ | | 10⁵ | 10⁻⁵ |
| E484K | 70.0 | 3.73 × | 2.61 × | 49.6 | 1.31 × | 6.50 × | 1.98 | 2.10 × | 4.15 × | 1.65 | 3.10 × | 5.11 × |
| | | 10⁴ | 10⁻³ | | 10⁵ | 10⁻³ | | 10⁵ | 10⁻⁴ | | 10⁵ | 10⁻⁴ |
| N501Y | 53.9 | 4.12 × | 2.22 × | 72.4 | 1.16 × | 8.36 × | 1.79 | 6.39 × | 3.70 × | 1.51 | 3.41 × | 5.13 × |
| | | 10⁴ | 10⁻³ | | 10⁵ | 10⁻³ | | 10⁶ | 10⁻⁴ | | 10⁵ | 10⁻⁴ |
| L452R- | 73.0 | 3.47 × | 2.53 × | 49.0 | 1.14 × | 5.57 × | 2.33 | 1.77 × | 4.12 × | 1.85 | 2.77 × | 5.12 × |
| T478K | | 10⁴ | 10⁻³ | | 10⁵ | 10⁻³ | | 10⁵ | 10⁻⁴ | | 10⁵ | 10⁻⁴ |
| L452R- | 38.9 | 6.28 × | 2.45 × | 14.9 | 1.88 × | 2.80 × | <0.0001 | 3.05 × | <1 × | <0.0001 | 2.24 × | <1 × |
| E484Q | | 10⁴ | 10⁻³ | | 10⁵ | 10⁻³ | | 10⁵ | 10⁻⁷ | | 10⁵ | 10⁻⁷ |
| E484K- | 31.4 | 8.63 × | 2.71 × | 29.3 | 1.68 × | 4.93 × | 4.44 | 5.36 × | 2.38 × | 2.16 | 1.51 × | 3.26 × |
| N501Y | | 10⁴ | 10⁻³ | | 10⁵ | 10⁻³ | | 10⁴ | 10⁻⁴ | | 10⁵ | 10⁻⁴ |
| K417N- | 70.8 | 3.73 × | 2.61 × | 71.6 | 9.68 × | 6.93 × | 2.12 | 2.06 × | 4.36 × | 1.83 | 2.94 × | 5.38 × |
| E484K- | | 10⁴ | 10⁻³ | | 10⁵ | 10⁻³ | | 10⁵ | 10⁻⁴ | | 10⁵ | 10⁻⁴ |
| N501Y | | | | | | | | | | | | |
| K417T- | 86.3 | 3.11 × | 2.68 × | 90.4 | 6.50 × | 5.88 × | 6.51 | 7.80 × | 5.08 × | 4.15 | 7.35 × | 3.05 × |
| E484K- | | 10⁴ | 10⁻³ | | 10⁵ | 10⁻³ | | 10⁴ | 10⁻⁴ | | 10⁴ | 10⁻⁴ |
| N501Y | | | | | | | | | | | | |
| SARS- | 198 | 3.77 × | 7.47 × | — | — | — | 102 | 1.03 × | 1.06 × | 123 | 6.95 × | 8.54 × |
| CoV-1 | | 10⁴ | 10⁻³ | | | | | 10⁵ | 10⁻² | | 10⁴ | 10⁻³ |
| RBD | | | | | | | | | | | | |

TABLE 5
Biolayer Interferometry measurement of
ShAbs to SARS-COV-2 RBD WA-1 strain.

| | SARS-CoV-2 RBD WA-1 | | |
|---|---|---|---|
| | KD (nM) | kon (1/Ms) | koff (1/s) |
| ShAb09a | 70.4 | 3.55E+04 | 2.14E−03 |
| ShAb17a | 61.9 | 7.5E+04 | 4.67E−3 |
| ShAb18a | 4.1 | 1.40E+05 | 5.70E−04 |
| ShAb19a | 47.7 | 9.01E+04 | 4.30E−03 |
| ShAb20a | 13.1 | 1.08E+05 | 1.41E−03 |
| ShAb21a | 25.7 | 1.07E+04 | 2.74E−03 |
| ShAb22a | 27.7 | 5.40E+04 | 1.49E−03 |
| ShAb23a | 18.1 | 1.01E+05 | 1.83E−03 |
| ShAb24a | 56.7 | 8.20E+04 | 4.65E−03 |
| ShAb25a | 382.4 | 8.85E+04 | 3.38E−02 |
| ShAb26a | 6.4 | 4.55E+04 | 2.92E−04 |
| ShAb27a | 62.9 | 1.82E+05 | 1.14E−02 |
| ShAb28a | 388.5 | 8.85E+04 | 3.38E−02 |
| ShAb29a | 106.0 | 6.83E+04 | 7.22E−03 |

TABLE 6
Biolayer interferometry measurement of ShAb
affinity to SARS-CoV-2 NTD WA-1 strain.

| | SARS-CoV-2 NTD WA-1 | | |
|---|---|---|---|
| | KD (nM) | kon (1/Ms) | koff (1/s) |
| ShAb04a | 44.2 | 3.13E+04 | 1.39E−03 |
| ShAb06a | 105.0 | 9.70E+04 | 1.02E−02 |
| ShAb07a | 82.8 | 8.94E+04 | 7.40E−03 |
| ShAb08a | 96.9 | 2.36E+04 | 2.74E−03 |
| ShAb10a | 148.1 | 9.75E+04 | 1.98E−03 |
| ShAb11a | 8.4 | 9.04E+04 | 7.56E−04 |
| ShAb12a | 178 | 3.10E+04 | 5.53E−03 |
| ShAb13a | 55.7 | 4.07E+04 | 2.27E−03 |
| ShAb14a | 2.3 | 7.55E+04 | 1.76E−04 |
| ShAb15a | 73.8 | 1.01E+05 | 7.45E−04 |
| ShAb16a | 8.5 | 8.40E+04 | 7.13E−04 |
| ShAb01H06K | 104.2 | 9.04E+04 | 7.56E−04 |
| ShAb06H02K | 74.6 | 1.14E+05 | 8.50E−03 |

v. Characterization of Nanobody Neutralization Responses

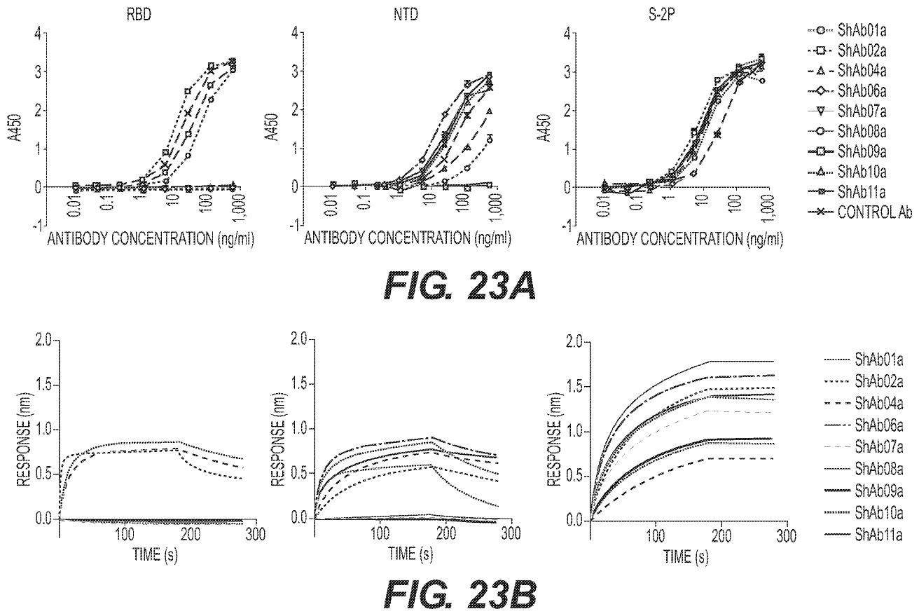

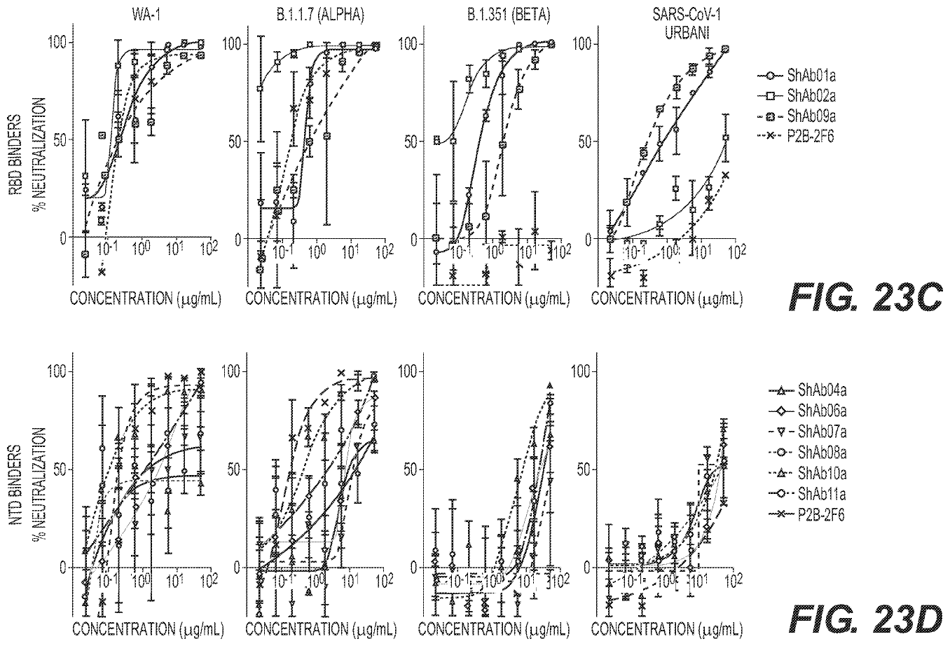

ShAb neutralization activity against SARS-CoV-2 and two variants of concern, B.1.1.7 (Alpha) and B.1.351 (Beta), was assessed using a pseudovirus neutralization assay (Table 7). Neutralization of the three RBD specific ShAbs, ShAb01a, ShAb02a, and ShAb09a, showed potent neutralization of SARS-CoV-2 pseudoviruses (FIG. 13F, FIG. 23C) which followed a similar pattern as the RBD-specific binding responses via Octet BLI (FIG. 13D; FIG. 23A). All three RBD ShAbs showed cross-reactivity against SARS-CoV-2 and variants, B.1.1.7 and B.1.351 (FIG. 13F; FIG. 23C). The NTD-specific ShAbs displayed a range of neutralization against WA-1 strain, with ShAb10a, ShAb11a and ShAb6a the most potent (FIG. 23D). This level of neutralization was reduced against Alpha, Beta, and SARS-CoV-1 which is understandable given the extensive sequence and structural changes in the NTD across these viruses (FIG. 23D).

TABLE 7
Pseudovirus neutralization IC50 values for SARS-COV-2 variants and SARS-COV-1.

| | IC50 (μg/mL) | | | | IC80 (μg/mL) | | | |
|---|---|---|---|---|---|---|---|---|
| Sample Name | WA-1 | Alpha | Beta | SARS-COV-1 | WA-1 | Alpha | Beta | SARS-COV-1 |
| ShAb01a | 0.228 | 0.463 | 0.457 | 0.728 | 1.14 | 0.617 | 1.202 | 8.62 |
| ShAb02a | 0.038 | <0.023 | 0.043 | 11.6 | 0.127 | 0.028 | 0.301 | >50.0 |
| ShAb04a | 3.91 | 18.6 | 37.0 | 28.6 | >50.0 | >50.0 | >50.0 | >50.0 |
| ShAb05a | 1.89 | 31.0 | >50.0 | >50.0 | >50.0 | >50.0 | >50.0 | >50.0 |
| ShAb06a | 2.35 | 6.81 | 27.2 | >50.0 | 15.0 | 36.3 | >50.0 | >50.0 |
| ShAb07a | 2.07 | 13.7 | >50.0 | 38.9 | >50.0 | >50.0 | >50.0 | >50.0 |
| ShAb08a | 3.04 | 10.5 | >50.0 | 48.3 | >50.0 | >50.0 | >50.0 | >50.0 |
| ShAb09a | 0.142 | 0.785 | 1.28 | 0.174 | 1.58 | 2.98 | 5.82 | 1.67 |
| ShAb10a | 0.116 | 0.416 | 8.15 | 23.2 | 0.551 | 2.89 | 28.6 | >50.0 |
| ShAb11a | 0.910 | 1.84 | 22.2 | 42.4 | 12.7 | 25.8 | 44.8 | >50.0 |
| ShAb12a | 3.21 | 3.95 | 17.3 | 2.19 | 21.0 | 14.3 | >50.0 | 20.1 |
| ShAb13a | 7.02 | 13.4 | 20.2 | >50.0 | >50.0 | >50.0 | >50.0 | >50.0 |
| ShAb01H02K | <0.023 | <0.023 | 0.011 | 1.81 | 0.055 | 0.046 | 0.021 | 16.5 |
| ShAb01H06K | 0.065 | 1.37 | 1.28 | 3.81 | 0.763 | 2.65 | 6.17 | 28.0 |
| ShAb01H11K | 0.324 | 0.386 | 0.173 | 0.669 | 1.81 | 1.81 | 0.582 | 2.43 |
| ShAb06H02K | 0.030 | 0.109 | 0.180 | >50.0 | 0.206 | 0.225 | 0.424 | >50.0 |
| ShAb06H11K | 3.56 | 7.52 | 1.48 | 33.2 | 27.8 | >50.0 | >20.0 | >50.0 |
| ShAb11H02K | 0.056 | 0.034 | 0.070 | >50.0 | 0.107 | 0.098 | 0.127 | >50.0 |
| ShAb01F | 0.309 | 0.119 | 0.075 | 0.404 | 0.488 | 0.328 | 0.164 | 1.91 |
| ShAb02F | 0.289 | 0.035 | 0.397 | >5.0 | 1.77 | 0.104 | 1.80 | >5.0 |
| BiShAb21a | 0.024 | 0.022 | 0.026 | 2.41 | 0.042 | 0.041 | 0.045 | >5.0 |
| TriShAb216 | 0.034 | >1.0 | 0.281 | 1.17 | 0.182 | >1.0 | >1.0 | >2.0 |
| TriShAb217 | 0.174 | 0.244 | 0.180 | 0.743 | 0.564 | 0.564 | 0.772 | >1.0 |
| BiSh21F | 0.079 | 0.065 | 0.073 | >2.0 | 0.482 | 0.234 | 0.193 | >2.0 |
| ShAb18a | 0.447 | 0.196 | 0.494 | >5.0 | 4.47 | 2.22 | 1.83 | >5.0 |
| ShAb19a | 0.845 | 4.13 | 2.13 | 0.089 | >5.0 | >5.0 | >5.0 | 0.222 |
| ShAb21a | 1.59 | 1.36 | 0.676 | >5.0 | >2.0 | >2.0 | >2.0 | >5.0 |
| ShAb22a | 0.197 | 0.525 | 1.3 | 0.415 | 1.23 | 0.97 | 3.22 | 2.71 |
| ShAb23a | 0.033 | 0.290 | 0.024 | 0.105 | 0.21 | 1.18 | 0.104 | 0.57 |
| ShAb24a | >2.0 | 2.1 | 2.3 | 0.575 | >2.0 | >5.0 | >5.0 | 4.09 |
| ShAb29a | >5.0 | 3.3 | 3.0 | 0.120 | >5.0 | >5.0 | >5.0 | 1.42 |

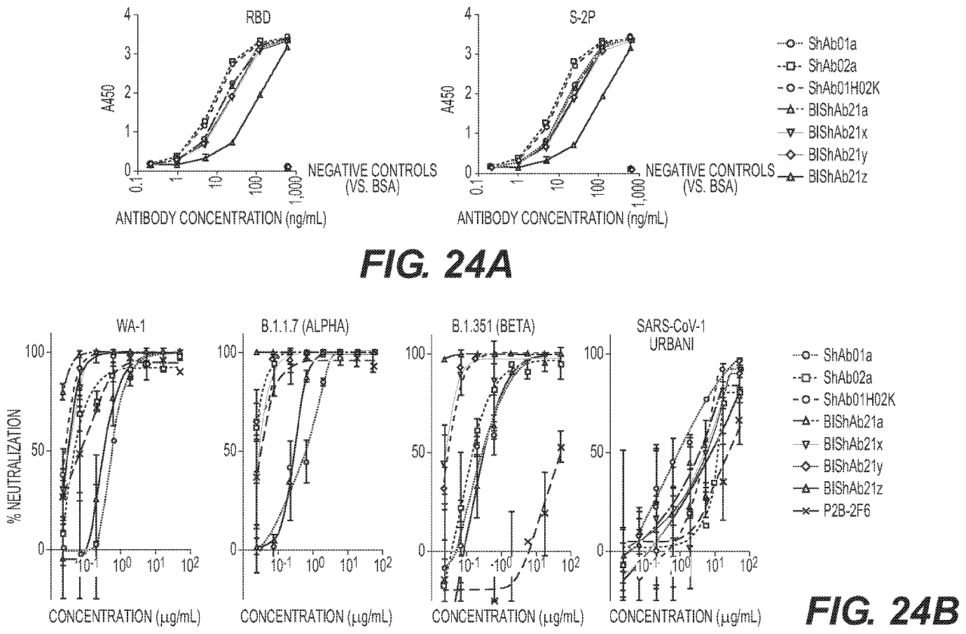

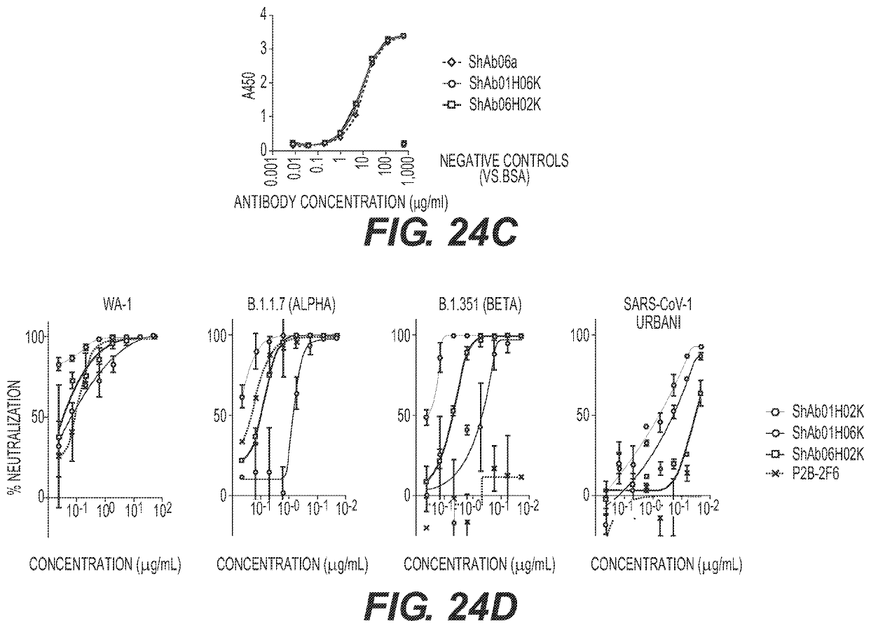

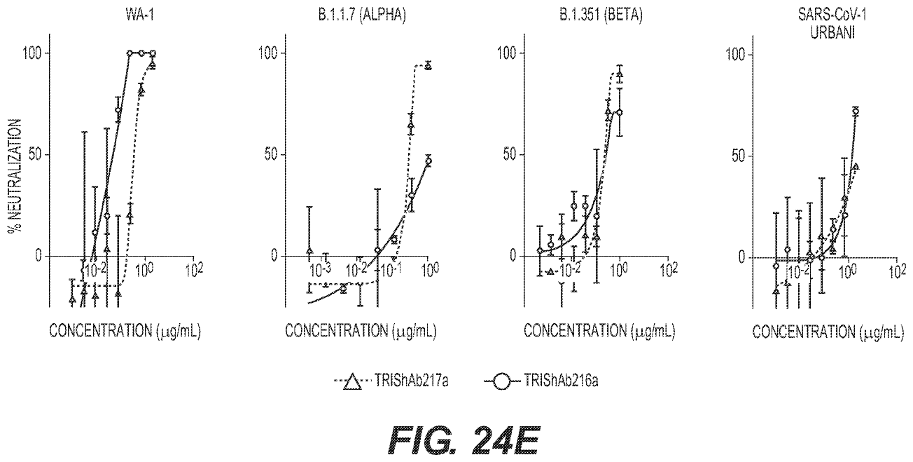

In addition to the ShAb01-ShAb02 multi-specific antibodies which showed additive or synergistic effects when combined, NTD-targeting ShAbs were also combined with either ShAb01 or ShAb02. A number of embodiments were produced (Table 3), such as ShAb01H06K and ShAB06H02K which combines both RBD- and NTD-targeting ShAbs into a single immunotherapeutic molecule (FIG. 24A-24C).

vi. Passive Protection in K18-ACE2 Transgenic Mice Against SARS-CoV-2 Challenge

In order to assess the ability of the ShAb molecules to protect against SARS-CoV-2 infection, an in vivo protection study using K18-ACE2 transgenic mice was carried out with ShAb01a and ShAb02a. Both ShAb01a and ShAb02a showed protection in this animal model (FIG. 13J-13M). ShAb01a protection was almost complete, with minimal weight loss (FIG. 13J), minimal death (FIG. 13K), low clinical scores (FIG. 13L), and undetectable virus in lung bronchoalveolar lavage (BAL) at 2 days post infection (FIG. 13M). In the ShAb02a group, animals were protected, as compared to the antibody isotype control, but the protection was less effective.

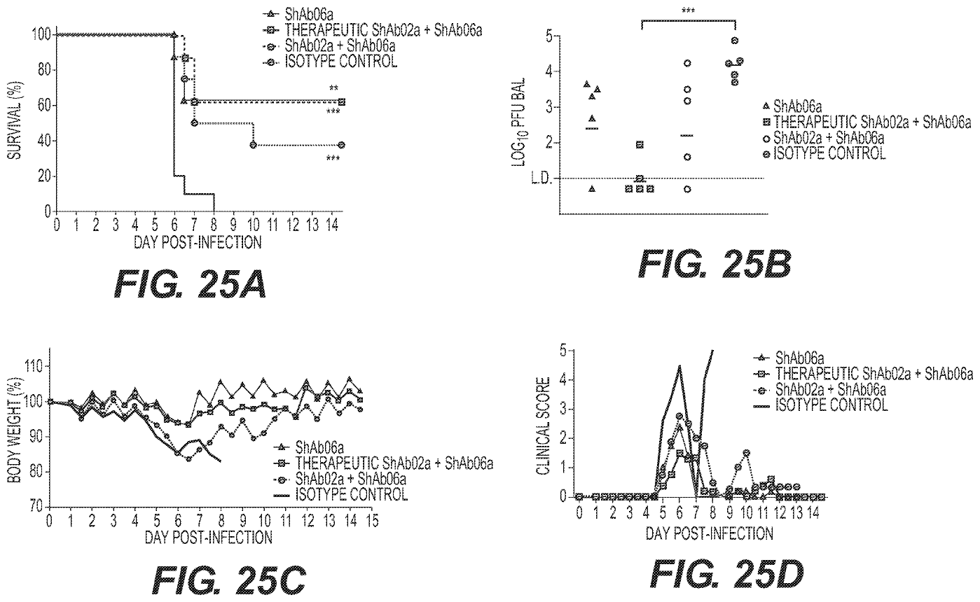

The same lethal SARS-CoV-2 challenge model in K18-ACE2 transgenic mice was also utilized to test the efficacy of passive nanobody immunity of NTD-targeting ShAb06a alone or in combination with ShAb02a. The dose of SARS-CoV-2 (1.25×10⁴ PFU) was titrated to establish significant weight loss observed following infection with SARS-CoV-2 USA-WA1/2020 strain. ShAbs were purified and characterized as described elsewhere. NTD-targeting ShAb06a alone or in combination with ShAb02a were passively transferred 24 hours prior to inoculation with SARS-CoV-2 (FIG. 25A-25D). One additional group of mice were first inoculated with SARS-CoV-2 followed 24 hours later by passive transfer of a mixture of ShAb02a and ShAb06a to assess the ability of the ShAb molecules to provide therapeutic effects. BAL was conducted 2 days after challenge and the SARS-CoV-2 viral titer was determined (FIG. 25B). Animal weight was measured twice daily for 14 days after challenge, and animals that lost greater than 25% weight during the study were euthanized. All study groups that received ShAbs either before or after infection showed significant protection against illness and death. Animals that received ShAb06a and therapeutic combination ShAb02a and ShAb06a, acted similarly, both in percent body weight (FIG. 25C) and survival (FIG. 25A). Their performance in inhibiting virus replication in the lungs differed; animals that received the therapeutic combination of ShAb02a and 06a showed minimal levels of virus in the BAL, which was significantly different compared to the isotype control group (FIG. 25B).

vii. Crystal Structure of ShAb01 and ShAb02 VNARs in Complex with SARS-CoV-2 RBD

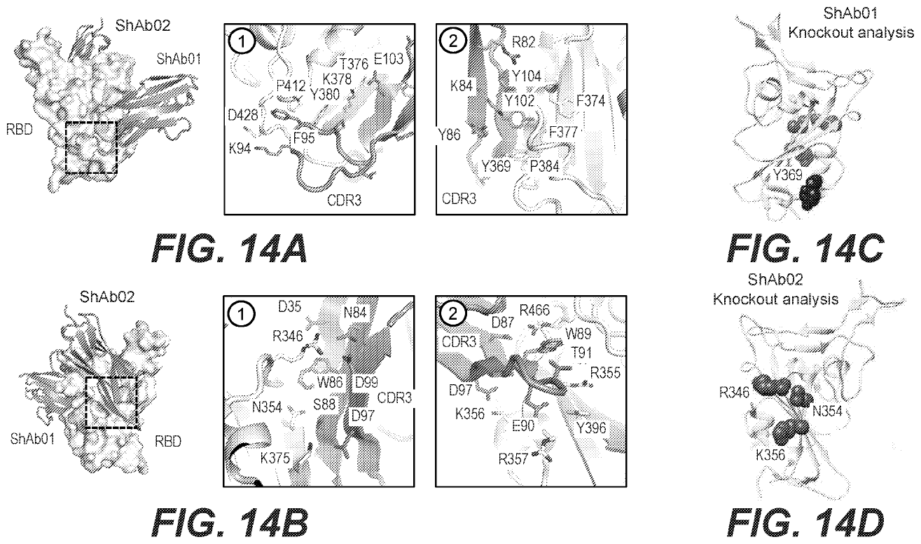

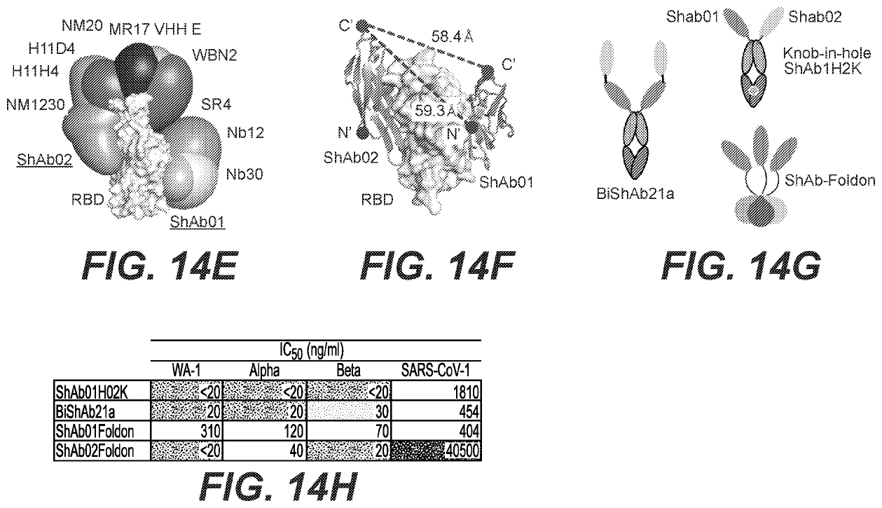

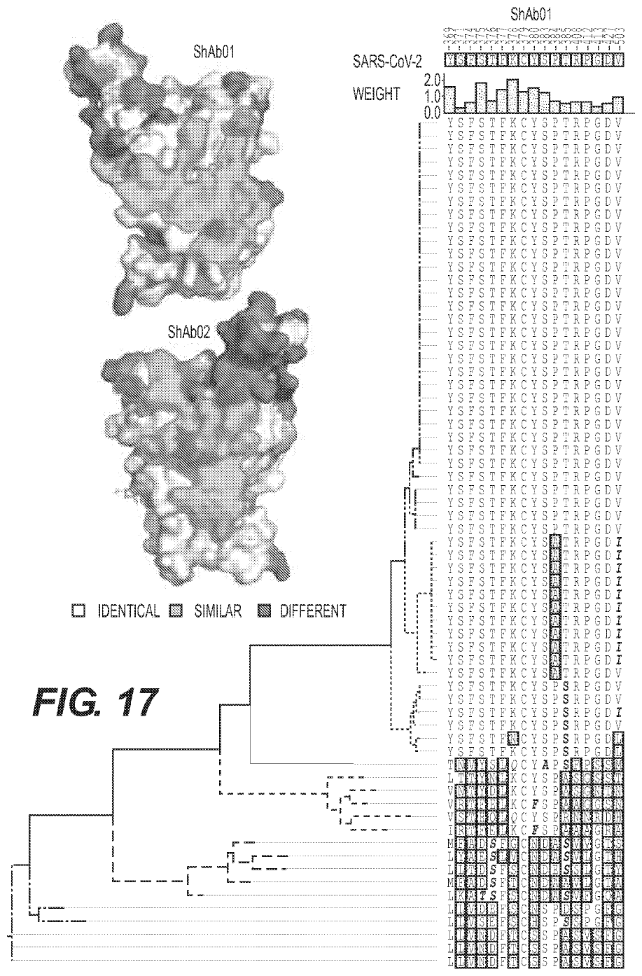

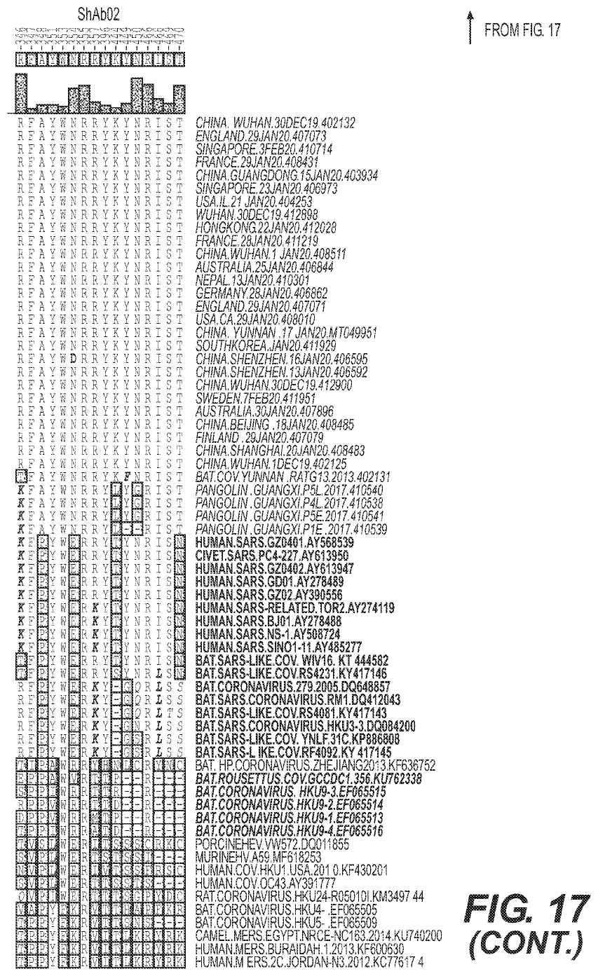

To understand the detailed recognition of shark IgNAR to SARS-CoV-2 spike RBD, structural and mutagenesis studies were carried out. The crystal structure of SARS-CoV-2 RBD in complex with ShAb01 VNAR and ShAb02 VNAR at a resolution of 2.6 Å by X-ray crystallography was determined (FIG. 14A-14B and Tables 8-10). In addition, alanine scanning analysis identified a set of amino acids important for nanobody binding (FIG. 14C-14D). ShAb01 and ShAb02 are both Type-II VNARs with typical β-sheet topology and extensive contact residues (FIG. 14A-14B, FIG. 17).

TABLE 8
Crystallographic data collection and refinement statistics.

| | SARS-CoV2-RBD + ShAb01 + ShAb02 | |
|---|---|---|
| Resolution range | 57.8-2.523 | (2.613-2.523) |
| Space group | P 2₁ 2₁ 2₁ | |
| Unit cell | 51.05, 62.79, 147.86, 90, 90, 90, | |
| Total reflections | 216,995 | (21,170) |
| Unique reflections | 16,633 | (1,609) |
| Multiplicity | 13.0 | (13.2) |
| Completeness (%) | 99.49 | (95.51) |
| Mean I/sigma(I) | 11.57 | (0.92) |
| Wilson B-factor | 66.63 | |
| R-merge | 0.2789 | (3.116) |
| R-meas | 0.2907 | (3.241) |
| R-pim | 0.0808 | (0.8802) |
| CC½ | 0.996 | (0.375) |
| CC* | 0.999 | (0.738) |
| Reflections used in refinement | 16573 | (1554) |
| Reflections used for R-free | 830 | (78) |
| R-work | 0.2149 | (0.4236) |
| R-free | 0.2728 | (0.4204) |
| CC(work) | 0.951 | (0.591) |
| CC(free) | 0.871 | (0.418) |

| | |
|---|---|
| Number of non-hydrogen | 3345 |
| atoms | |
| macromolecules | 3310 |
| ligands | 28 |
| solvent | 7 |
| Protein residues | 421 |
| RMS(bonds) | 0.003 |
| RMS(angles) | 0.56 |
| Ramachandran favored (%) | 92.29 |
| Ramachandran allowed (%) | 7.23 |
| Ramachandran outliers (%) | 0.48 |
| Rotamer outliers (%) | 3.54 |
| Clash score | 5.53 |
| Average B-factor | 82.62 |
| macromolecules | 82.36 |
| ligands | 119.54 |
| solvent | 58.75 |
| Number of TLS groups | 15 |

TABLE 9
ShAb01 interface with SARS-COV-2 RBD.

| | | | Distance |
|---|---|---|---|
| | ShAb01 | RBD | (Å) |
| Hydrogen | N:ALA 1[O] | A:LYS 378[NZ] | 2.90 |
| bonds | N:LEU 99[O] | A:SER 383[OG] | 2.31 |
| | N:GLY 100[O] | A:TYR 369[OH] | 2.68 |
| | N:SER 101[OG] | A:CYS 379[N] | 2.81 |
| | N:TYR 102[O] | A:PHE 377[N] | 3.13 |
| | N:GLY 98[N] | A:CYS 379[O] | 2.51 |
| | N:SER 101[OG] | A:CYS 379[O] | 3.51 |
| | N:TYR 102[N] | A:PHE 377[O] | 3.20 |
| | N:TYR 102[OH] | A:TYR 369[O] | 3.07 |
| | N:TYR 102[OH] | A:SER 371[O] | 3.57 |
| | N:TYR 104[N] | A:SER 375[O] | 2.97 |
| Salt Bridge | N:GLU 103[OE1] | A:LYS 378[NZ] | 2.99 |

TABLE 10
ShAb02 interface with SARS-COV-2 RBD.

| | | | Distance |
|---|---|---|---|
| | ShAb02 | RBD | (Å) |
| Hydrogen | Z:GLU 90[N] | A:ARG 355[O] | 3.67 |
| bonds | Z:LYS 51[N] | A:ASN 450[OD1] | 2.80 |
| | Z:SER 61[N] | A:THR 470[OG1] | 3.10 |
| | Z:SER 61[OG] | A:THR 470[OG1] | 3.57 |
| | Z:ASP 99[OD1] | A:ARG 346[NH1] | 2.94 |
| | Z:TYR 37[OH] | A:ARG 346[NH2] | 2.34 |
| | Z:TYR 101[OH] | A:ARG 346[NH2] | 3.71 |
| | Z:ASP 87[OD1] | A:ASN 354[ND2] | 3.65 |
| | Z:SER 88[O] | A:ASN 354[ND2] | 3.81 |
| | Z:SER 88[O] | A:ARG 355[N] | 2.66 |
| | Z:GLU 90[OE1] | A:ARG 357[NE] | 22.90 |
| | Z:GLU 90[OE2] | A:TYR 396[OH] | 3.70 |
| | Z:ILE 49[O] | A:ASN 450[ND2] | 3.21 |
| | Z:ASP 87[OD1] | A:ARG 466[NH1] | 3.03 |
| | Z:ASP 87[OD2] | A:ARG 466[NH2] | 3.13 |
| | Z:SER 61[OG] | A:THR 470[N] | 3.09 |
| | Z:VAL 59[O] | A:THR 470[OG1] | 2.82 |
| Salt Bridge | Z:ASP 99[OD1] | A:ARG 346[NH1] | 2.94 |
| | Z:GLU 90[OE1] | A:ARG 357[NE] | 2.90 |
| | Z:ASP 87[OD2] | A:ARG 466[NH1] | 3.90 |
| | Z:ASP 87[OD1] | A:ARG 466[NH1] | 3.03 |
| | Z:ASP 87[OD2] | A:ARG 466[NH2] | 3.13 |
| | Z:ASP 87[OD1] | A:ARG 466[NH2] | 3.63 |

ShAb01 VNAR binds to the RBD with a total buried surface area (BSA) of 1777 Å² primarily engaging the CDR3 loop (1457 Å²) and the first β-sheet to recognize one side of the SARS-CoV2 RBD (FIG. 14A). Within the CDR3 loop, a β-sheet (Gly100-Glu104) forms anti-parallel interactions with an RBD β-sheet (Ser375-Ser379), and the CDR 3 tip (Trp87-Gly100) interacts with multiple loop regions of the RBD including Phe374-Thr385, Ala411-Gln414, and Asp427-Phe429. In addition, the N-terminal residues of ShAb01 bind to a pocket located between RBD Asp405-Ala411 and β-sheet Ser375-Tyr380 increasing the overall ShaAb01-RBD interaction.

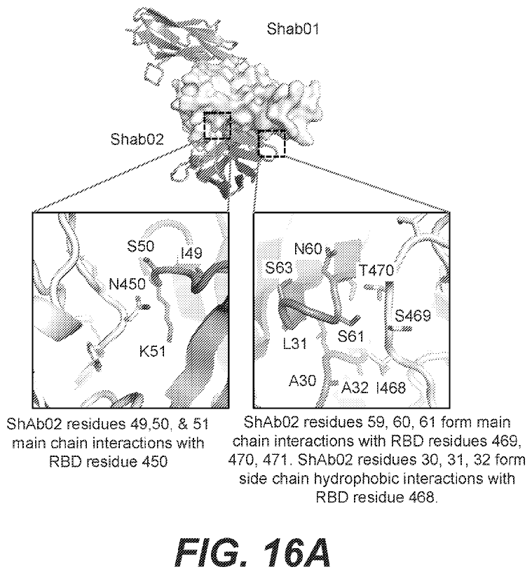

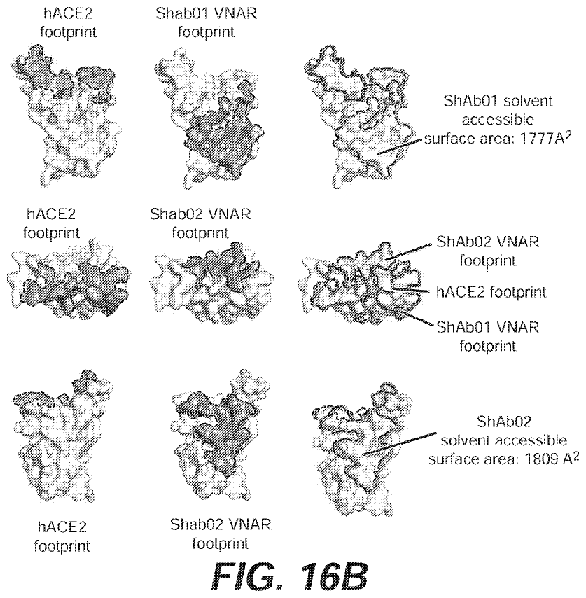

ShAb02 binds to the opposite face of SARS-CoV-2 RBD in relation to the ShAb01 epitope (FIG. 14B). ShAb02 forms multiple interactions with the RBD using CDR1 (303 Å²), HV2 (293 Å²), and CDR3 (1066 Å²) with a total BSA interface of 1810 Å². The major contact region between ShAb02 and RBD is through charged residues of the CDR3 loop. Notably, the SARS-CoV-2 RBD Arg346 forms a hydrogen bond with Tyr101, and a salt bridge with Asp99, and forms pi-pi interactions with ShAb02 Trp86 (FIG. 14B). ShAb02 CDR1 also engages RBD residues Ile468-Glu417 through main chain interactions, with the HV2 loop also using main chain interactions with RBD residues Gly447-Asn450 (FIG. 16A).

Alanine scanning mutagenesis highlighted Tyr369 as an important residue for ShAb01-RBD recognition, and Asn354, Arg346, and Lys356 as important residues for the ShAB02-RBD recognition (FIG. 14C-14D). Each of these amino acids form important contacts with the ShAb molecules. Comparison of the ShAb01 and 02 epitopes (FIG. 16B) to the ACE2 binding site shows that there is overlap between both ShAb01 and ShAb02. In the context of the ShAb-Fc fusion chimera, the C-terminus of ShAb01 is located proximal to the RBD which likely explains the robust ACE2-blocking activity. The ShAb02 C-terminus is distal to the RBD-ACE2 binding site which may explain the lower level of ACE2 blocking (FIG. 13E).

Numerous monoclonal antibodies and nanobodies have been identified that can neutralize SARS-CoV-2. The ShAb01 and ShAb02 binding epitopes were assessed to compare to those previously described. The ShAb01 binds to the same face of the RBD as described for mAb CR3022 and falls into the Class 4 grouping of RBD-targeting antibodies. Comparison of the ShAb01 epitope indicates some similarities with other nanobodies, but the epitope is more extended than other nanobodies and the CDR3 interacts with residues proximal to Asp427 which is not seen with other nanobodies. Even with nanobodies WNB10, NB30, or VHH V which have significant epitope overlap, the extended CDR3 loop of ShAb01 allows the binding epitope to extend to a highly sequence-conserved region of the RBD (FIG. 17).

ShAb02 binds to the face of the RBD molecule that is targeted by antibodies designated as Class 3, in common with therapeutic antibodies with EUA for COVID-19 treatment including mAbs S309 and REGN10987. Comparison of ShAb02 with other nanobodies indicates that the ShAb02 epitope is unique in regard to nanobody RBD-targeting. Most nanobodies bind to a region above the ShAb02 epitope, closer to the ACE2 binding site. ShAb02 binds to a section of the RBD (residues 346-357) that is not recognized by other nanobodies. Residues identified by the alanine scanning mutagenesis experiments identified residues 346, 354 and 356 as important for ShAb02 recognition (FIG. 14D). Sequence analysis of related sarbecovirus RBDs indicates that sequence variation within the ShAb02 epitope is limited within SARS-CoV-2 variants but is typical for other groups of sarbecoviruses including SARS-CoV-1 (FIG. 17).

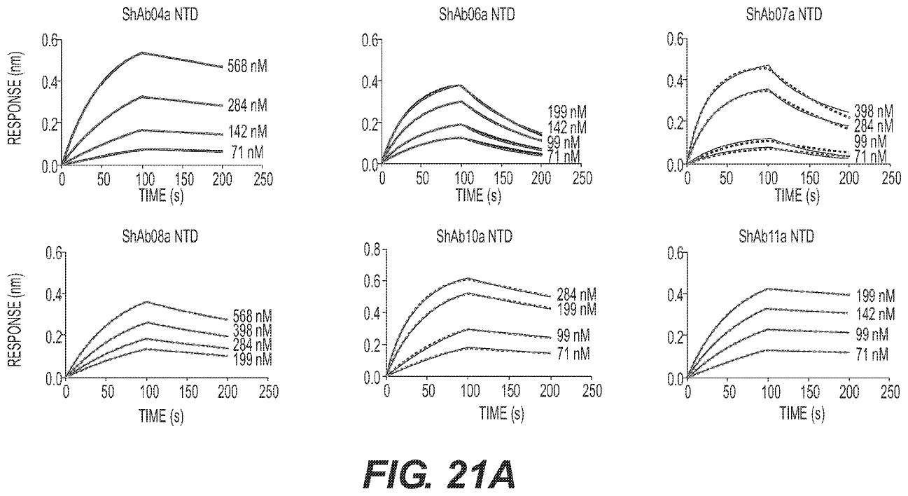

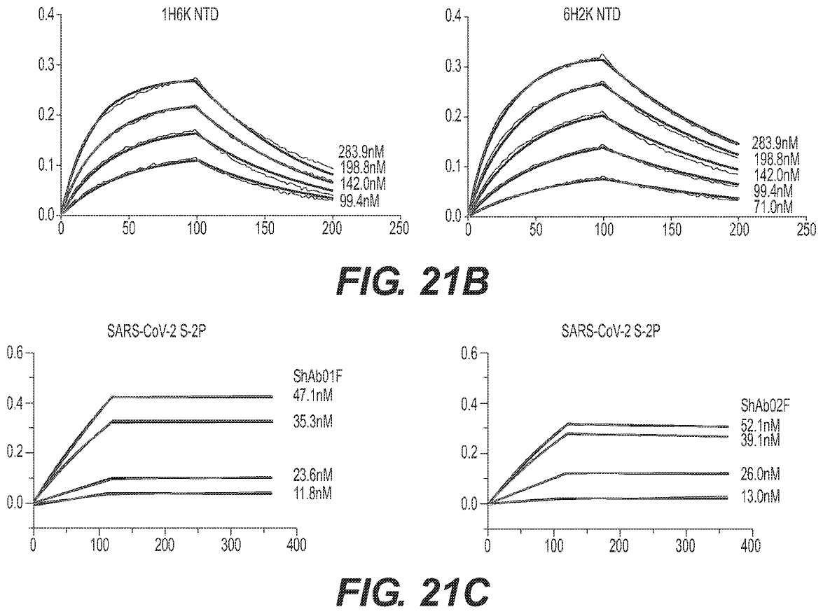

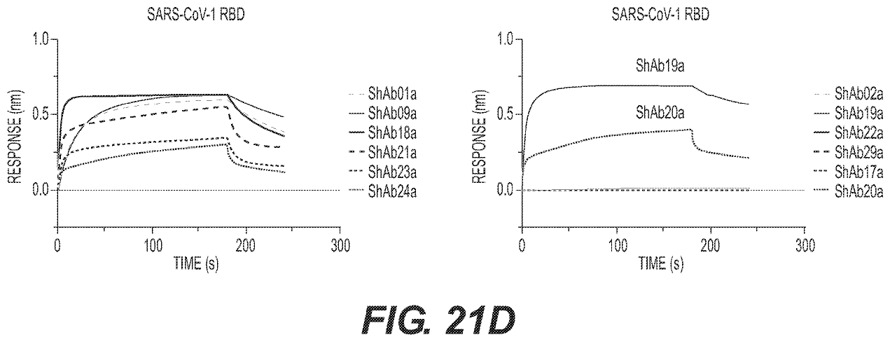

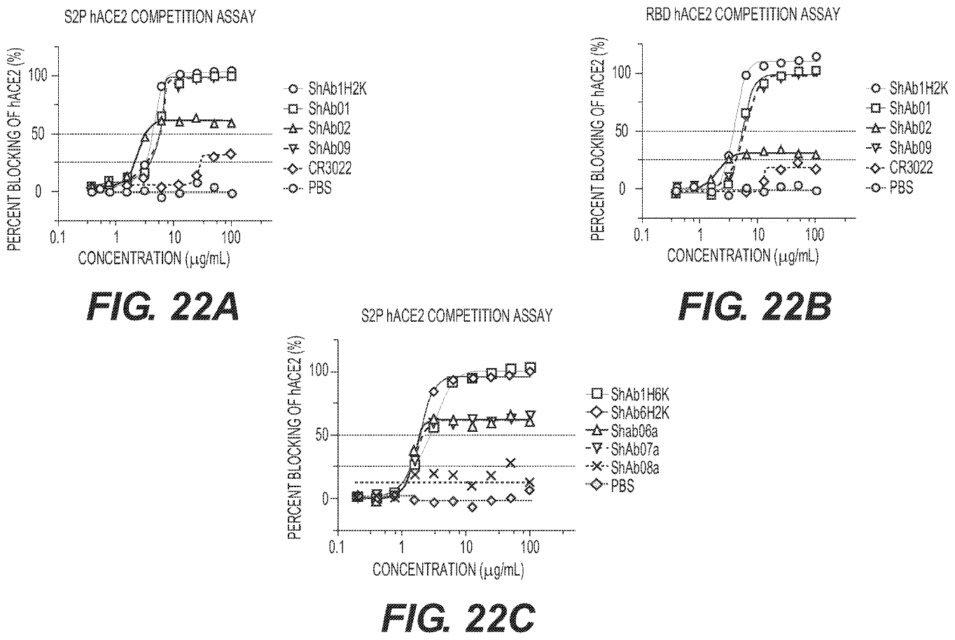

Given the non-overlapping nature of ShAb01 and ShAb02, the distances between the N- and C-termini of the VNAR molecules were analyzed (FIG. 14F) to generate multi-domain or multi-specific molecules (FIG. 14G, FIG. 21 and FIG. 22) that would contain both ShAb01 and ShAb02 VNARs. These combinations would inherently boost the RBD binding surface area, combine the sarbecovirus breadth of binding and ACE2-blocking of ShAb01, with the potency of SARS-CoV-2 neutralization of ShAb02. In addition, the linker between ShAb01 and ShAb02 would physically pass across the ACE2 binding site, to further enable blocking of the ACE2-RBD interaction (FIG. 19A-19C). A number of these multivalent immunotherapeutics such as ShAb01H02K or BiShAb21a showed synergistic improvements in antigen-affinity (FIG. 20A-20D, FIG. 24A, FIG. 24C), ACE2-blocking (FIG. 22A-22C), and pseudovirus neutralization (FIG. 24B, FIG. 24D-24E). These engineered molecules display IC50 pseudovirus neutralization levels of <20 ng ml-1 for SARS-CoV-2 variants, and 450 ng ml-1 of SARS-CoV-1.

In addition to a simple Gly-Ser linker between ShAb01 and ShAb02, the linker sequence was adjusted to incorporate regions of the ACE2 protein that are important to RBD binding in embodiments BiShAb21x, BiShAb21y, and BiShAb21z (FIG. 24A-24B).

### Example 3. Construction of ShAb VNAR-Derived Immunotherapeutic Molecules

The ShAb VNARs selected in Examples 1 and 2 were used to generate ShAb VNAR chimeras as VNAR-conjugate immunotherapeutic molecules based on various designs depicted in FIG. 9A-9F and FIG. 18A-18H as summarized below in Table 11. They were used in various assays, including biolayer interferometry binding, ACE2 inhibition assays, and pseudovirus neutralization assay, some of which are disclosed in Examples 1 and 2. Table 11 below summarizes some of the exemplary VNAR-conjugate immunotherapeutic molecules. Overall, there are 11 VNAR-Fc chimeras (ShAb01-ShAB-11), 29 VNAR-3c-Fc chimeras (ShAb01-ShAb29), 4 VNAR-3c-His8 chimeras (ShAb01, ShAb02, ShAb06, ShAb07), 1 VNAR-Ferritin chimera (ShAb02), 1 VNAR-LS chimera (ShAb02), and 1 LS-VNAR chimera (ShAb02). With the “beads on a string” design (see e.g., FIG. 9B-9C), there are 7 structures:
(1) ShAb02 VNAR to ShAb01 VNAR to trimerization domain (T4-fibritin) foldon (BiShAb21F; construct 02VNAR-25-01VNAR-KESlinker-Foldon-3C-His8-Strep);
(2) ShAb02 VNAR to ShAb10 VNAR linked to human Fc (BiShAb210a; construct 02VNAR-25-10VNAR-3c-Fc);
(3) ShAb10 VNAR to ShAb02 VNAR linked to human Fc (BiSHAb102a; construct 10VNAR-25-02VNAR-3c-Fc);
(4) ShAb02 VNAR to ShAb11 VNAR linked to human Fc (BiShAb211a; construct 02VNAR-25-11VNAR-3c-Fc);
(5) ShAb11 VNAR to ShAb02 VNAR linked to human Fc (BiSHAb112a; construct 11VNAR-25-02VNAR-3c-Fc);
(6) ShAb02 VNAR to ShAb01 VNAR to ShAb06 VNAR linked to human Fc (TrishAb216a; construct 02VNAR-25-01IVNAR-24-06VNAR-3c-Fe) and
(7) ShAb02 VNAR to ShAb VNAR to ShAb07 VNAR linked to human Fe (TrishAb217a; construct 02VNAR-25-0IVNAR-24-07VNAR-3c-Fe).

TABLE 11
List of exemplary ShAb VNAR-conjugate immunotherapeutic molecules.

| | | Nucleotide | Amino Acid |
|---|---|---|---|
| | | Sequence | Sequence (SEQ |
| Clone Name | Construct | (SEQ ID NO:) | ID NO:) |
| ShAb01 | ShAb01 VNAR-Fc | 134 | 61 |
| ShAb01a | ShAb01 VNAR-3c-Fc | 135 | 62 |
| ShAb01b | ShAb01 VNAR-3c-His8 | 136 | 63 |
| ShAb02 | ShAb02 VNAR-Fc | 137 | 64 |
| ShAb02a | ShAb02 VNAR-3c-Fc | 138 | 65 |
| ShAb02b | ShAb02 VNAR-3c-His8 | 139 | 66 |
| ShAb02c | His8-3c-ShAb02 VNAR-Ferritin | 140 | 67 |
| ShAb02d | His8-3c-ShAb02 VNAR-6-LS | 141 | 68 |
| ShAb02e | LS-15-ShAb02 VNAR-3c-His | 142 | 69 |
| ShAb03 | ShAb03 VNAR-Fc | 143 | 70 |
| ShAb03a | ShAb03 VNAR-3c-Fc | 144 | 71 |
| ShAb04 | ShAb04 VNAR-Fc | 145 | 72 |
| ShAb04a | ShAb04 VNAR-3c-Fc | 146 | 73 |
| ShAb05 | ShAb05 VNAR-Fc | 147 | 74 |
| ShAb05a | ShAb05 VNAR-3c-Fc | 148 | 75 |
| ShAb06 | ShAb06 VNAR-Fc | 149 | 76 |
| ShAb06a | ShAb06 VNAR-3c-Fc | 150 | 77 |
| ShAb06b | ShAb06 VNAR-3c-His6 | 151 | 78 |
| ShAb07 | ShAb07 VNAR-Fc | 152 | 79 |
| ShAb07a | ShAb07 VNAR-3c-Fc | 153 | 80 |
| ShAb07b | ShAb07 VNAR-3c-His6 | 154 | 81 |
| ShAb08 | ShAb08 VNAR-Fc | 155 | 82 |
| ShAb08a | ShAb08 VNAR-3c-Fc | 156 | 83 |
| ShAb09 | ShAb09 VNAR-Fc | 157 | 84 |
| ShAb09a | ShAb09 VNAR-3c-Fc | 158 | 85 |
| ShAb10 | ShAb10 VNAR-Fc | 159 | 86 |
| ShAb10a | ShAb10 VNAR-3c-Fc | 160 | 87 |
| ShAb10b | ShAb10 VNAR-3c-His6 | 161 | 88 |
| ShAb11 | ShAb11 VNAR-Fc | 162 | 89 |
| ShAb11a | ShAb11 VNAR-3c-Fc | 163 | 90 |
| ShAb11b | ShAb11 VNAR-3c-His6 | 164 | 91 |
| ShAb12a | ShAb12 VNAR-3c-Fc | 165 | 92 |
| ShAb13a | ShAb13 VNAR-3c-Fc | 166 | 93 |
| ShAb14a | ShAb14 VNAR-3c-Fc | 167 | 94 |
| ShAb15a | ShAb15 VNAR-3c-Fc | 168 | 95 |
| ShAb16a | ShAb16 VNAR-3c-Fc | 169 | 96 |
| ShAb17a | ShAb17 VNAR-3c-Fc | 170 | 97 |
| ShAb17b | ShAb17 VNAR-3c-His6 | 171 | 98 |
| ShAb18a | ShAb18 VNAR-3c-Fc | 172 | 99 |
| ShAb19a | ShAb19 VNAR-3c-Fc | 173 | 100 |
| ShAb19b | ShAb19 VNAR-3c-His6 | 174 | 101 |
| ShAb20a | ShAb20 VNAR-3c-Fc | 175 | 102 |
| ShAb20b | ShAb20 VNAR-3c-His6 | 176 | 103 |
| ShAb21a | ShAb21 VNAR-3c-Fc | 177 | 104 |
| ShAb22a | ShAb22 VNAR-3c-Fc | 178 | 105 |
| ShAb23a | ShAb23 VNAR-3c-Fc | 179 | 106 |
| ShAb23b | ShAb23 VNAR-3c-His6 | 180 | 107 |
| ShAb24a | ShAb24 VNAR-3c-Fc | 181 | 108 |
| ShAb24b | ShAb24 VNAR-3c-His6 | 182 | 109 |
| ShAb25a | ShAb25 VNAR-3c-Fc | 183 | 110 |
| ShAb26a | ShAb26 VNAR-3c-Fc | 184 | 111 |
| ShAb27a | ShAb27 VNAR-3c-Fc | 185 | 112 |
| ShAb28a | ShAb28 VNAR-3c-Fc | 186 | 113 |
| ShAb29a | ShAb29 VNAR-3c-Fc | 187 | 114 |
| ShAb30a | B-3E9x_3c-Fc | 188 | 115 |
| BiShAb21a | ShAb02 VNAR-25aa-ShAb01 | 189 | 116 |
| | VNAR-3c-Fc | | |
| BiShAb21x | ShAb02 VNAR-(15aa- | 190 | 117 |
| | Helix2(AA53-90)-4-Helix1(AA19- | | |
| | 45)-12)-ShAb01 VNAR-3c-Fc | | |
| BiShAb21y | ShAb02 VNAR-(21aa- | 191 | 118 |
| | Helix1 + 2(AA19-90)-12)-ShAb01 | | |
| | VNAR-3c-Fc | | |
| BiShAb21z | ShAb02 VNAR-(21aa- | 192 | 119 |
| | Helix1(AA19-45)-12)-ShAb01 | | |
| | VNAR-3c-Fc | | |
| BiShAb21F | ShAb02 VNAR-25aa-ShAb01 | 193 | 120 |
| | VNAR-KESlinker-Foldon-3C- | | |
| | His8-Strep | | |
| BiShAb210a | ShAb02 VNAR-25aa-ShAb10 | 194 | 121 |
| | VNAR-3c-Fc | | |
| BiShAb102a | ShAb10 VNAR-25aa-ShAb02 | 195 | 122 |
| | VNAR-3c-Fc | | |
| BiShAb211a | ShAb02 VNAR-25aa-ShAb11 | 196 | 123 |
| | VNAR-3c-Fc | | |
| BiShAb112a | ShAb11 VNAR-25aa-ShAb02 | 197 | 124 |
| | VNAR-3c-Fc | | |
| ShAb01-Foldon | ShAb01 VNAR-KESlinker- | 198 | 125 |
| | Foldon-3c-His8-Strep | | |
| ShAb02-Foldon | ShAb02 VNAR-KESlinker- | 199 | 126 |
| | Foldon-3c-His8-Strep | | |
| TrishAb216a | ShAb02 VNAR-25aa-ShAb01 | 200 | 127 |
| | VNAR-24aa-ShAb06 VNAR-3c- | | |
| | Fc | | |
| TrishAb217a | ShAb02 VNAR-25aa-ShAb01 | 201 | 128 |
| | VNAR-24aa-ShAb07 VNAR-3c- | | |
| | Fc | | |
| TriShAb2110a | ShAb02 VNAR-25aa-ShAb01 | 202 | 129 |
| | VNAR-24aa-ShAb10 VNAR-3c- | | |
| | Fc | | |
| TriShAb2111a | ShAb02 VNAR-25aa-ShAb01 | 203 | 130 |
| | VNAR-24aa-ShAb11 VNAR-3c- | | |
| | Fc | | |
| BiShAb0101a | ShAb01 VNAR-25aa-ShAb01 | 204 | 131 |
| | VNAR-3c-Fc | | |
| BiShAb0202a | ShAb02 VNAR-25aa-ShAb02 | 205 | 132 |
| | VNAR-3c-Fc | | |
| CCShAb21 | ShAb02 VNAR-KES-helical | 206 | 133 |
| | trimer-KES ShAb01 VNAR-3c- | | |
| | His8 | | |
| ShAb01aHole | ShAb01 VNAR-3c-Fc-Hole | 378 | 366 |
| ShAb02aKnob | ShAb02 VNAR-3c-Fc-Knob | 379 | 367 |
| ShAb06aHole | ShAb06 VNAR-3c-Fc-Hole | 380 | 368 |
| ShAb06aKnob | ShAb06 VNAR-3c-Fc-Knob | 381 | 369 |
| ShAb11a-Hole | ShAb11 VNAR-3c-Fc-Hole | 382 | 370 |
| ShAb11a-Knob | ShAb11 VNAR-3c-Fc-Knob | 383 | 371 |
| ShAb07a-Knob | ShAb07 VNAR-3c-Fc-Knob | 384 | 372 |
| ShAb08a-Hole | ShAb08 VNAR-3c-Fc-Hole | 385 | 373 |
| ShAb09a-Hole | ShAb09 VNAR-3c-Fc-Hole | 386 | 374 |
| ShAb10a-Knob | ShAb10 VNAR-3c-Fc-Knob | 387 | 375 |
| ShAb19a-Knob | ShAb19 VNAR-3c-Fc-Knob | 388 | 376 |
| ShAb23a-Hole | ShAb23 VNAR-3c-Fc-Hole | 389 | 377 |
| TriShAb010101a | ShAb01 VNAR-25aa-ShAb01 | | 390 |
| | VNAR-24aa-ShAb01 VNAR-3c- | | |
| | Fc | | |
| TriShAb020202a | ShAb02 VNAR-25aa-ShAb02 | | 391 |
| | VNAR-24aa-ShAb02 VNAR-3c- | | |
| | Fc | | |
| TriShAb191919a | ShAb19 VNAR-25aa-ShAb19 | | 392 |
| | VNAR-24aa-ShAb19 VNAR-3c- | | |
| | Fc | | |
| TriShAb232323a | ShAb23 VNAR-25aa-ShAb23 | | 393 |
| | VNAR-24aa-ShAb23 VNAR-3c- | | |
| | Fc | | |

What is claimed is:

1. A SARS-CoV-2 coronavirus binding agent comprising at least one targeting moiety comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 394, or an amino acid sequence having at least 90% sequence identity with one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 394, wherein the binding agent binds to a Spike glycoprotein of SARS-CoV-2 coronavirus.

2. A SARS-CoV-2 coronavirus binding agent comprising at least one targeting moiety comprising a complementarity determining region 1 (CDR1) and a CDR3, wherein the CDR1 comprises an amino acid sequence selected from any one of SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, and SEQ ID NO: 395, and the CDR3 comprises an amino acid sequence selected from any one of SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, and SEQ ID NO: 396.

3. The SARS-CoV-2 coronavirus binding agent of claim 1 or 2, wherein the at least one targeting moiety is a full-length antibody, a single-domain antibody, a recombinant heavy-chain-only antibody (V_{HH}), a single-chain antibody (scFv), a shark heavy-chain-only antibody (VNAR), a microprotein, a darpin, an anticalin, an adnectin, an aptamer, a Fv, a Fab, a Fab′, a F(ab′)₂, a peptide mimetic molecule, a natural ligand for a receptor, or a synthetic molecule.

4. The SARS-CoV-2 coronavirus binding agent of any one of claims 1-3, wherein the at least one targeting moiety is a single-domain antibody.

5. The SARS-CoV-2 coronavirus binding agent of claim 3, wherein the at least one targeting moiety comprises a V_{HH}, such as a humanized V_{HH}, a shark V_{HH}, or a camelid V_{HH}.

6. The SARS-CoV-2 coronavirus binding agent of any one of claims 1-5, wherein the at least one targeting moiety comprises:
a CDR1 comprising the amino acid sequence of SEQ ID NO: 246 and a CDR3 comprising the amino acid sequence of SEQ ID NO: 276;
a CDR1 comprising the amino acid sequence of SEQ ID NO: 247 and a CDR3 comprising the amino acid sequence of SEQ ID NO: 277;
a CDR1 comprising the amino acid sequence of SEQ ID NO: 248 and a CDR3 comprising the amino acid sequence of SEQ ID NO: 278;
a CDR1 comprising the amino acid sequence of SEQ ID NO: 249 and a CDR3 comprising the amino acid sequence of SEQ ID NO: 279;
a CDR1 comprising the amino acid sequence of SEQ ID NO: 250 and a CDR3 comprising the amino acid sequence of SEQ ID NO: 280;
a CDR1 comprising the amino acid sequence of SEQ ID NO: 251 and a CDR3 comprising the amino acid sequence of SEQ ID NO: 281;
a CDR1 comprising the amino acid sequence of SEQ ID NO: 252 and a CDR3 comprising the amino acid sequence of SEQ ID NO: 282;
a CDR1 comprising the amino acid sequence of SEQ ID NO: 253 and a CDR3 comprising the amino acid sequence of SEQ ID NO: 283;
a CDR1 comprising the amino acid sequence of SEQ ID NO: 254 and a CDR3 comprising the amino acid sequence of SEQ ID NO: 284;
a CDR1 comprising the amino acid sequence of SEQ ID NO: 255 and a CDR3 comprising the amino acid sequence of SEQ ID NO: 285;
a CDR1 comprising the amino acid sequence of SEQ ID NO: 256 and a CDR3 comprising the amino acid sequence of SEQ ID NO: 286;
a CDR1 comprising the amino acid sequence of SEQ ID NO: 257 and a CDR3 comprising the amino acid sequence of SEQ ID NO: 287;
a CDR1 comprising the amino acid sequence of SEQ ID NO: 258 and a CDR3 comprising the amino acid sequence of SEQ ID NO: 288;
a CDR1 comprising the amino acid sequence of SEQ ID NO: 259 and a CDR3 comprising the amino acid sequence of SEQ ID NO: 289;
a CDR1 comprising the amino acid sequence of SEQ ID NO: 260 and a CDR3 comprising the amino acid sequence of SEQ ID NO: 290;
a CDR1 comprising the amino acid sequence of SEQ ID NO: 261 and a CDR3 comprising the amino acid sequence of SEQ ID NO: 291;
a CDR1 comprising the amino acid sequence of SEQ ID NO: 262 and a CDR3 comprising the amino acid sequence of SEQ ID NO: 292;
a CDR1 comprising the amino acid sequence of SEQ ID NO: 263 and a CDR3 comprising the amino acid sequence of SEQ ID NO: 293;
a CDR1 comprising the amino acid sequence of SEQ ID NO: 264 and a CDR3 comprising the amino acid sequence of SEQ ID NO: 294;
a CDR1 comprising the amino acid sequence of SEQ ID NO: 265 and a CDR3 comprising the amino acid sequence of SEQ ID NO: 295;
a CDR1 comprising the amino acid sequence of SEQ ID NO: 266 and a CDR3 comprising the amino acid sequence of SEQ ID NO: 296;
a CDR1 comprising the amino acid sequence of SEQ ID NO: 267 and a CDR3 comprising the amino acid sequence of SEQ ID NO: 297;
a CDR1 comprising the amino acid sequence of SEQ ID NO: 268 and a CDR3 comprising the amino acid sequence of SEQ ID NO: 298;
a CDR1 comprising the amino acid sequence of SEQ ID NO: 269 and a CDR3 comprising the amino acid sequence of SEQ ID NO: 299;
a CDR1 comprising the amino acid sequence of SEQ ID NO: 270 and a CDR3 comprising the amino acid sequence of SEQ ID NO: 300;
a CDR1 comprising the amino acid sequence of SEQ ID NO: 271 and a CDR3 comprising the amino acid sequence of SEQ ID NO: 301;
a CDR1 comprising the amino acid sequence of SEQ ID NO: 272 and a CDR3 comprising the amino acid sequence of SEQ ID NO: 302;
a CDR1 comprising the amino acid sequence of SEQ ID NO: 273 and a CDR3 comprising the amino acid sequence of SEQ ID NO: 303;
a CDR1 comprising the amino acid sequence of SEQ ID NO: 274 and a CDR3 comprising the amino acid sequence of SEQ ID NO: 304;
a CDR1 comprising the amino acid sequence of SEQ ID NO: 275 and a CDR3 comprising the amino acid sequence of SEQ ID NO: 305; or
a CDR1 comprising the amino acid sequence of SEQ ID NO: 395 and a CDR3 comprising the amino acid sequence of SEQ ID NO: 396.

7. The SARS-CoV-2 coronavirus binding agent of claim 6, wherein:
the at least one targeting moiety having the CDR1 of SEQ ID NO: 246 and the CDR3 of SEQ ID NO: 276 further comprises a hypervariable region 2 (HV2) comprising the amino acid sequence of SEQ ID NO: 306 and a hypervariable region 4 (HV4) comprising the amino acid sequence of SEQ ID NO: 336;
the at least one targeting moiety having the CDR1 of SEQ ID NO: 247 and the CDR3 of SEQ ID NO: 277 further comprises an HV2 comprising the amino acid sequence of SEQ ID NO: 307 and an HV4 comprising the amino acid sequence of SEQ ID NO: 337;
the at least one targeting moiety having the CDR1 of SEQ ID NO: 248 and the CDR3 of SEQ ID NO: 278 further comprises an HV2 comprising the amino acid sequence of SEQ ID NO: 308 and an HV4 comprising the amino acid sequence of SEQ ID NO: 338;
the at least one targeting moiety having the CDR1 of SEQ ID NO: 249 and the CDR3 of SEQ ID NO: 279 further comprises a HV2 comprising the amino acid sequence of SEQ ID NO: 309 and an HV4 comprising the amino acid sequence of SEQ ID NO: 339;
the at least one targeting moiety having the CDR1 of SEQ ID NO: 250 and the CDR3 of SEQ ID NO: 280 further comprises a HV2 comprising the amino acid sequence of SEQ ID NO: 310 and an HV4 comprising the amino acid sequence of SEQ ID NO: 340;
the at least one targeting moiety having the CDR1 of SEQ ID NO: 251 and the CDR3 of SEQ ID NO: 281 further comprises a HV2 comprising the amino acid sequence of SEQ ID NO: 311 and an HV4 comprising the amino acid sequence of SEQ ID NO: 341;
the at least one targeting moiety having the CDR1 of SEQ ID NO: 252 and the CDR3 of SEQ ID NO: 282 further comprises a HV2 comprising the amino acid sequence of SEQ ID NO: 312 and an HV4 comprising the amino acid sequence of SEQ ID NO: 342;
the at least one targeting moiety having the CDR1 of SEQ ID NO: 253 and the CDR3 of SEQ ID NO: 283 further comprises a HV2 comprising the amino acid sequence of SEQ ID NO: 313 and an HV4 comprising the amino acid sequence of SEQ ID NO: 343;
the at least one targeting moiety having the CDR1 of SEQ ID NO: 254 and the CDR3 of SEQ ID NO: 284 further comprises a HV2 comprising the amino acid sequence of SEQ ID NO: 314 and an HV4 comprising the amino acid sequence of SEQ ID NO: 344;
the at least one targeting moiety having the CDR1 of SEQ ID NO: 255 and the CDR3 of SEQ ID NO: 285 further comprises a HV2 comprising the amino acid sequence of SEQ ID NO: 315 and an HV4 comprising the amino acid sequence of SEQ ID NO: 345;
the at least one targeting moiety having the CDR1 of SEQ ID NO: 256 and the CDR3 of SEQ ID NO: 286 further comprises a HV2 comprising the amino acid sequence of SEQ ID NO: 316 and an HV4 comprising the amino acid sequence of SEQ ID NO: 346;
the at least one targeting moiety having the CDR1 of SEQ ID NO: 257 and the CDR3 of SEQ ID NO: 287 further comprises a HV2 comprising the amino acid sequence of SEQ ID NO: 317 and an HV4 comprising the amino acid sequence of SEQ ID NO: 347;
the at least one targeting moiety having the CDR1 of SEQ ID NO: 258 and the CDR3 of SEQ ID NO: 288 further comprises a HV2 comprising the amino acid sequence of SEQ ID NO: 318 and an HV4 comprising the amino acid sequence of SEQ ID NO: 348;
the at least one targeting moiety having the CDR1 of SEQ ID NO: 259 and the CDR3 of SEQ ID NO: 289 further comprises a HV2 comprising the amino acid sequence of SEQ ID NO: 319 and an HV4 comprising the amino acid sequence of SEQ ID NO: 349;
the at least one targeting moiety having the CDR1 of SEQ ID NO: 260 and the CDR3 of SEQ ID NO: 290 further comprises a HV2 comprising the amino acid sequence of SEQ ID NO: 320 and an HV4 comprising the amino acid sequence of SEQ ID NO: 350;
the at least one targeting moiety having the CDR1 of SEQ ID NO: 261 and the CDR3 of SEQ ID NO: 291 further comprises a HV2 comprising the amino acid sequence of SEQ ID NO: 321 and an HV4 comprising the amino acid sequence of SEQ ID NO: 351;
the at least one targeting moiety having the CDR1 of SEQ ID NO: 262 and the CDR3 of SEQ ID NO: 292 further comprises a HV2 comprising the amino acid sequence of SEQ ID NO: 322 and an HV4 comprising the amino acid sequence of SEQ ID NO: 352;
the at least one targeting moiety having the CDR1 of SEQ ID NO: 263 and the CDR3 of SEQ ID NO: 293 further comprises a HV2 comprising the amino acid sequence of SEQ ID NO: 323 and an HV4 comprising the amino acid sequence of SEQ ID NO: 353;
the at least one targeting moiety having the CDR1 of SEQ ID NO: 264 and the CDR3 of SEQ ID NO: 294 further comprises a HV2 comprising the amino acid sequence of SEQ ID NO: 324 and an HV4 comprising the amino acid sequence of SEQ ID NO: 354;
the at least one targeting moiety having the CDR1 of SEQ ID NO: 265 and the CDR3 of SEQ ID NO: 295 further comprises a HV2 comprising the amino acid sequence of SEQ ID NO: 325 and an HV4 comprising the amino acid sequence of SEQ ID NO: 355;
the at least one targeting moiety having the CDR1 of SEQ ID NO: 266 and the CDR3 of SEQ ID NO: 296 further comprises a HV2 comprising the amino acid sequence of SEQ ID NO: 326 and an HV4 comprising the amino acid sequence of SEQ ID NO: 356;
the at least one targeting moiety having the CDR1 of SEQ ID NO: 267 and the CDR3 of SEQ ID NO: 297 further comprises a HV2 comprising the amino acid sequence of SEQ ID NO: 327 and an HV4 comprising the amino acid sequence of SEQ ID NO: 357;
the at least one targeting moiety having the CDR1 of SEQ ID NO: 268 and the CDR3 of SEQ ID NO: 298 further comprises a HV2 comprising the amino acid sequence of SEQ ID NO: 328 and an HV4 comprising the amino acid sequence of SEQ ID NO: 358;
the at least one targeting moiety having the CDR1 of SEQ ID NO: 269 and the CDR3 of SEQ ID NO: 299 further comprises a HV2 comprising the amino acid sequence of SEQ ID NO: 329 and an HV4 comprising the amino acid sequence of SEQ ID NO: 359;
the at least one targeting moiety having the CDR1 of SEQ ID NO: 270 and the CDR3 of SEQ ID NO: 300 further comprises a HV2 comprising the amino acid sequence of SEQ ID NO: 330 and an HV4 comprising the amino acid sequence of SEQ ID NO: 360;
the at least one targeting moiety having the CDR1 of SEQ ID NO: 271 and the CDR3 of SEQ ID NO: 301 further comprises a HV2 comprising the amino acid sequence of SEQ ID NO: 331 and an HV4 comprising the amino acid sequence of SEQ ID NO: 361;
the at least one targeting moiety having the CDR1 of SEQ ID NO: 272 and the CDR3 of SEQ ID NO: 302 further comprises a HV2 comprising the amino acid sequence of SEQ ID NO: 332 and an HV4 comprising the amino acid sequence of SEQ ID NO: 362;
the at least one targeting moiety having the CDR1 of SEQ ID NO: 273 and the CDR3 of SEQ ID NO: 303 further comprises a HV2 comprising the amino acid sequence of SEQ ID NO: 333 and an HV4 comprising the amino acid sequence of SEQ ID NO: 363;
the at least one targeting moiety having the CDR1 of SEQ ID NO: 274 and the CDR3 of SEQ ID NO: 304 further comprises a HV2 comprising the amino acid sequence of SEQ ID NO: 334 and an HV4 comprising the amino acid sequence of SEQ ID NO: 364;
the at least one targeting moiety having the CDR1 of SEQ ID NO: 275 and the CDR3 of SEQ ID NO: 305 further comprises a HV2 comprising the amino acid sequence of SEQ ID NO: 335 and an HV4 comprising the amino acid sequence of SEQ ID NO: 365; and
the at least one targeting moiety having the CDR1 of SEQ ID NO: 395 and the CDR3 of SEQ ID NO: 396 further comprises a HV2 comprising the amino acid sequence of SEQ ID NO: 397 and an HV4 comprising the amino acid sequence of SEQ ID NO: 398.

8. The SARS-CoV-2 coronavirus binding agent of any one of claims 1-7, wherein the binding agent binds to:
(a) the Spike glycoprotein of SARS-CoV-2 coronavirus (WA-1 strain) with a dissociation constant (K_{D}) of about 150 nM or less, about 100 nM or less, about 50 nM or less, or about 10 nM or less;
(b) the Spike glycoprotein of SARS-CoV-2 coronavirus (B.1.1.7 variant) with a dissociation constant (K_{D}) of about 150 nM or less, about 100 nM or less, about 50 nM or less, or about 10 nM or less;
(c) the Spike glycoprotein of SARS-CoV-2 coronavirus (B.1.351 variant) with a dissociation constant (K_{D}) of about 150 nM or less, about 100 nM or less, about 50 nM or less, or about 10 nM or less; and/or
(d) the Spike glycoprotein of SARS-CoV-2 coronavirus (B.1.617.2 variant) with a dissociation constant (K_{D}) of about 150 nM or less, about 100 nM or less, about 50 nM or less, or about 10 nM or less.

9. The SARS-CoV-2 coronavirus binding agent of any one of claims 1-8, wherein the binding agent has:
(a) a pseudovirus neutralization IC50 (μg/mL) value for SARS-CoV-2 (WA-1 strain) of 1 or less, 0.5 or less, 0.2 or less, 0.1 or less, 0.05 or less, or 0.03 or less;
(b) a pseudovirus neutralization IC50 (μg/mL) value for SARS-CoV-2 (B.1.1.7 variant) of 1 or less, 0.5 or less, 0.2 or less, 0.1 or less, 0.05 or less, or 0.03 or less;
(c) a pseudovirus neutralization IC50 (μg/mL) value for SARS-CoV-2 (B.1.351 variant) of 1 or less, 0.5 or less, 0.2 or less, 0.1 or less, 0.05 or less, or 0.03 or less; and/or
(d) a pseudovirus neutralization IC50 (μg/mL) value for SARS-CoV-2 (B.1.617.2 variant) of 1 or less, 0.5 or less, 0.2 or less, 0.1 or less, 0.05 or less, or 0.03 or less.

10. A chimeric protein comprising:
(a) the SARS-CoV-2 coronavirus binding agent of any one of claims 1-9; and
(b) a heterologous protein, such as a Fc domain, a ferritin, a lumazine synthase, an antibody that binds to human serum albumin, or a combination thereof.

11. The chimeric protein of claim 10, further comprising a linker and/or a leader sequence, wherein the linker sequence connects the SARS-CoV-2 coronavirus binding agent and the heterologous protein.

12. The chimeric protein of claim 11, wherein the linker comprises the amino acid sequence of SEQ ID NO: 207, SEQ ID NO: 208, SEQ ID NO: 209, SEQ ID NO: 210, SEQ ID NO: 211, SEQ ID NO: 212, or SEQ ID NO: 213, or an amino acid sequence having at least 90% sequence identity with one of SEQ ID NO: 207, SEQ ID NO: 208, SEQ ID NO: 209, SEQ ID NO: 210, SEQ ID NO: 211, SEQ ID NO: 212, and SEQ ID NO: 213.

13. The chimeric protein of claim 11, wherein the leader sequence comprises the amino acid sequence of SEQ ID NO: 214 or SEQ ID NO: 215, or an amino acid sequence having at least 90% sequence identity with the amino acid sequence of SEQ ID NO: 214 or SEQ ID NO: 215.

14. The chimeric protein of any one of claims 10-13, wherein the Fc domain comprises a human Fc domain or a human IgM domain.

15. The chimeric protein of any one of claims 10-14, wherein the human Fc domain comprises a human IgG1 Fc domain.

16. The chimeric protein of any one of claims 10-15, wherein the heterologous protein comprises at least two Fc domains.

17. The chimeric protein of any one of claims 10-16, wherein the chimeric protein comprises the amino acid sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 390, SEQ ID NO: 391, SEQ ID NO: 392, or SEQ ID NO: 393, or an amino acid sequence having at least 90% sequence identity with one of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 390, SEQ ID NO: 391, SEQ ID NO: 392, or SEQ ID NO: 393, wherein the chimeric protein binds to a spike glycoprotein of SARS-CoV-2 coronavirus.

18. The chimeric protein of any one of claims 10-17, wherein the chimeric protein is a multivalent construct comprising at least two SARS-CoV-2 coronavirus binding agents of any one of claims 1-9.

19. The chimeric protein of any one of claims 10-18, wherein the chimeric protein is a multispecific construct that binds to at least two different regions of the spike glycoprotein of SARS-CoV-2 coronavirus, wherein the at least two different regions of the spike glycoprotein of SARS-CoV-2 coronavirus include the receptor binding domain and/or the N-terminus domain.

20. The chimeric protein of any one of claims 10-19, wherein the chimeric protein comprises the amino acid sequence of SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, or SEQ ID NO: 377, or a combination thereof.

21. The chimeric protein of claim 20, wherein the chimeric protein comprises:
a) the amino acid sequence of SEQ ID NO: 366 and the amino acid sequence of SEQ ID NO: 367;
b) the amino acid sequence of SEQ ID NO: 366 and the amino acid sequence of SEQ ID NO: 369;
c) the amino acid sequence of SEQ ID NO: 367 and the amino acid sequence of SEQ ID NO: 368;
d) the amino acid sequence of SEQ ID NO: 366 and the amino acid sequence of SEQ ID NO: 371;
e) the amino acid sequence of SEQ ID NO: 367 and the amino acid sequence of SEQ ID NO: 370; or
f) the amino acid sequence of SEQ ID NO: 368 and the amino acid sequence of SEQ ID NO: 371.

22. A nucleic acid molecule encoding the SARS-CoV-2 coronavirus binding agent of any one of claims 1-9.

23. The nucleic acid molecule of claim 22, wherein the nucleic acid molecule comprises the nucleotide sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or SEQ ID NO: 60.

24. A nucleic acid molecule encoding the chimeric protein of any one of claims 10-21.

25. The nucleic acid molecule of claim 24, wherein nucleic acid molecule comprises the nucleotide sequence of SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, or SEQ ID NO: 389.

26. A host cell comprising the nucleic acid molecule of any one of claims 22-25.

27. A composition comprising one or more of the SARS-CoV-2 coronavirus binding agents of any one of claims 1-9 or the chimeric protein of any one of claims 10-21.

28. The composition of claim 27, further comprising a pharmaceutically acceptable excipient.

29. The composition of claim 27 or 28, wherein the composition comprises a therapeutically effective amount of the SARS-CoV-2 coronavirus binding agent or the chimeric protein.

30. The composition of any one of claims 27-29, wherein the composition is formulated for subcutaneous, intravenous, intraarterial, or intramuscular injection.

31. A method for treating or preventing SARS-CoV-2 coronavirus infection, comprising administering to a subject in need thereof an effective amount of a composition comprising the SARS-CoV-2 coronavirus binding agent of any one of claims 1-9.

32. A method for treating or preventing SARS-CoV-2 coronavirus infection, comprising administering to a subject in need thereof an effective amount of a composition comprising the chimeric protein of any one of claims 10-21.

33. The method of claim 31 or 32, wherein the composition further comprises a pharmaceutically acceptable excipient.

34. The method of any one of claims 31-33, wherein the composition is formulated for subcutaneous, intravenous, intraarterial, or intramuscular injection.

35. The method of any one of claims 31-34, wherein the subject is a human.

36. The method of claim 35, wherein the subject is diagnosed with or suspected of having a SARS-CoV-2 infection.

37. A method for diagnosing SARS-CoV-2 coronavirus infection or detecting SARS-CoV-2 coronavirus in a sample, comprising using the SARS-CoV-2 coronavirus binding agent of any one of claims 1-9 to detect SARS-CoV-2 coronavirus.

38. The method of claim 37, comprising:
a) contacting the sample with the SARS-CoV-2 coronavirus binding agent; and
b) determining the presence of the SARS-CoV-2 coronavirus infection or SARS-CoV-2 coronavirus in the sample upon detection of binding between a spike protein of the SARS-CoV-2 coronavirus in the sample and said SARS-CoV-2 coronavirus binding agent.

39. A method for diagnosing SARS-CoV-2 coronavirus infection or detecting SARS-CoV-2 coronavirus in a sample, comprising using the chimeric protein of any one of claims 10-21 to detect SARS-CoV-2 coronavirus.

40. The method of claim 39, comprising:
a) contacting the sample with the chimeric protein; and
b) determining the presence of the SARS-CoV-2 coronavirus infection or SARS-CoV-2 coronavirus in the sample upon detection of binding between a spike protein of the SARS-CoV-2 coronavirus in the sample and the chimeric protein.

41. The method of any one of claims 37-40, wherein the sample is from a subject suspected of having a SARS-CoV-2 coronavirus infection.

42. The SARS-CoV-2 coronavirus binding agent of any one of claims 1-9, wherein the binding agent neutralizes SARS-CoV-2 coronavirus in vitro or in vivo.

43. The SARS-CoV-2 coronavirus binding agent of claim 42, wherein the binding agent comprises the amino acid sequence of SEQ ID NO: 62 or SEQ ID NO: 65.

44. The chimeric protein of any one of claims 10-21, wherein the SARS-CoV-2 coronavirus binding agent neutralizes SARS-CoV-2 coronavirus in vitro or in vivo.

45. The chimeric protein of claim 44, wherein the chimeric protein comprises the amino acid sequence of SEQ ID NO: 116, SEQ ID NO: 117, SEQ ID NO: 118, SEQ ID NO: 119, SEQ ID NO: 127 or SEQ ID NO: 128.

46. The chimeric protein of claim 44, wherein the chimeric protein comprises:
a) the amino acid sequence of SEQ ID NO: 366 and the amino acid sequence of SEQ ID NO: 367;
b) the amino acid sequence of SEQ ID NO: 366 and the amino acid sequence of SEQ ID NO: 369; or
c) the amino acid sequence of SEQ ID NO: 367 and the amino acid sequence of SEQ ID NO: 368.
